(19) United States
(12) Patent Application Publication
(10) Pub. No.: US 2022/0380760 A1
(43) Pub. Date: Dec. 1, 2022

(54) DISRUPTING GENOMIC COMPLEX ASSEMBLY IN FUSION GENES

(71) Applicant: FLAGSHIP PIONEERING INNOVATIONS V, INC., Cambridge, MA (US)

(72) Inventors: Marija Tadin Strapps, Boston, MA (US); Thomas Kang-Hsi Ni, Waltham, MA (US); David Arthur Berry, Newton, MA (US); Laura Gabriela Lande, Chestnut Hill, MA (US); Abigail Elizabeth Witt, Charlestown, MA (US); Adam Walter Scheidegger, Boston, MA (US)

(21) Appl. No.: 17/285,399

(22) PCT Filed: Oct. 15, 2019
(86) PCT No.: PCT/US2019/056381

§ 371 Date: Apr. 14, 2021

Related U.S. Application Data
(60) Provisional application No. 62/745,812, filed on Oct. 15, 2018.

(51) Int. Cl. C12N 15/113; C07K 14/82

(52) U.S. Cl. CPC C12N 15/113; C07K 2319/81; C12N 2310/20; C07K 14/82

(57) ABSTRACT

The present disclosure relates generally to disruption of genomic complexes associated with fusion genes via a disrupting agent comprising a targeting moiety and/or an effector, e.g., disrupting, moiety. Described herein are experiments directed at identifying target anchor sequences proximal to fusion genes, e.g., fusion oncogenes; targeting the genomic complexes, e.g., CFLs, comprising said target anchor sequences for disruption (e.g., inhibiting their formation and/or destabilizing them) using disrupting agents; and evaluating the effects of disruption on fusion gene expression and other cell (e.g., cancer cell) characteristics (e.g., growth, viability, etc.).

### CROSS-REFERENCE TO RELATED APPLICATIONS

The present application claims priority to and benefit from U.S. provisional application U.S.S.N. 62/745,812 (filed Oct. 15, 2018) the contents of which is hereby incorporated by reference in its entirety.

### BACKGROUND

One cause of cancer is the inappropriate expression or activity of certain genes, e.g., fusion genes, which can be created by a gross chromosomal rearrangement.

### SUMMARY

The three-dimensional structure of the genome plays a deterministic role in the regulation of transcription, through the formation of genomic complexes that control the spatial proximity between target genes and their cis- and trans acting regulators. Deviation from a wild-type chromatin architecture can lead to disease, such as cancer. For example, gross chromosomal rearrangements can create an oncogenic fusion protein situated in a cancer fusion loop (CFL), a chromatin region that promotes high expression of the oncogenic fusion protein through the pathological proximity of strong transcriptional drivers to otherwise non-active or less active gene bodies. As another example, cancer cells sometimes comprise a cancer-specific anchor sequence that wild-type cells lack (e.g., in the absence of a translocation). The cancer specific anchor sequence can force the interaction between a strong transcriptional driver, such as an enhancer or a super enhancer, with an otherwise less active gene body. This can lead to high expression of an oncogene. As shown herein, by specifically disrupting an unwanted loop in a cancer cell (e.g., using a site-specific disrupting agent), one can treat the cancer, e.g., by reducing the aberrant expression of an oncogenic gene (e.g., fusion oncogene) in the genomic complex.

Additional features of any of the aforesaid methods or compositions include one or more of the following enumerated embodiments.

Those skilled in the art will recognize, or be able to ascertain using no more than routine experimentation, many equivalents to the specific embodiments of the invention described herein. Such equivalents are intended to be encompassed by the following enumerated embodiments.

All publications, patent applications, patents, and other references (e.g., sequence database reference numbers) mentioned herein are incorporated by reference in their entirety. For example, all GenBank, Unigene, and Entrez sequences referred to herein, e.g., in any Table herein, are incorporated by reference. Unless otherwise specified, the sequence accession numbers specified herein, including in any Table herein, refer to the database entries current as of Oct. 15, 2019. When one gene or protein references a plurality of sequence accession numbers, all of the sequence variants are encompassed.

### Enumerated Embodiments

1. A method of decreasing expression, (e.g., transcription) of a gene (e.g., an oncogene, e.g., a fusion oncogene) in a cell (e.g., a cancer cell), comprising:
contacting the cell with a site-specific disrupting agent that binds, e.g., binds specifically, to a first and/or second anchor sequence, or a component of a genomic complex associated with the first and/or second anchor sequence, in the cell, in an amount sufficient to decrease expression of the gene,
wherein the cell comprises a nucleic acid, said nucleic acid comprising:
i) the gene;
ii) a breakpoint (e.g., a breakpoint resulting from a gross chromosomal rearrangement), located proximal to the gene;
iii) the first anchor sequence, which is located proximal to the breakpoint and/or the gene, and
iv) the second anchor sequence, which is located proximal to the breakpoint and/or the gene, thereby decreasing expression of the gene.
2. A method of modifying a chromatin structure of a nucleic acid in a cell (e.g., a cancer cell), comprising:
contacting the cell with a site-specific disrupting agent that binds, e.g., binds specifically, to a first and/or second anchor sequence, or a component of a genomic complex associated with the first and/or second anchor sequence, in the cell,
wherein the nucleic acid comprises:
i) a breakpoint (e.g., a breakpoint resulting from a gross chromosomal rearrangement),
ii) the first anchor sequence, which is located proximal to the breakpoint, and
iii) the second anchor sequence, which is located proximal to the breakpoint,
thereby modifying the chromatin structure of the nucleic acid.
3. A method of modifying a chromatin structure of a nucleic acid in a cell (e.g., a cancer cell), comprising:
altering a topology of an anchor sequence-mediated conjunction, e.g., a loop, said conjunction comprising a first anchor sequence and a second anchor sequence that form the conjunction,
wherein the nucleic acid comprises:
i) a breakpoint (e.g., a breakpoint resulting from a gross chromosomal rearrangement),
ii) the first anchor sequence, which is located proximal to the breakpoint, and
iii) the second anchor sequence, which is located proximal to the breakpoint,
thereby modifying the chromatin structure of the nucleic acid.
4. A method of modifying a chromatin structure of a nucleic acid in a cell (e.g., a cancer cell), comprising:
altering a first and/or second anchor sequence, or a component of a genomic complex associated with the first and/or second anchor sequence,
wherein the nucleic acid comprises:
i) a breakpoint (e.g., a breakpoint resulting from a gross chromosomal rearrangement),
ii) a gene (e.g., a fusion gene, e.g., a fusion oncogene), e.g., located proximal to the breakpoint,
iii) the first anchor sequence, which is located proximal to the breakpoint and/or the gene, and
iv) the second anchor sequence, which is located proximal to the breakpoint and/or the gene,
thereby modifying the chromatin structure of the nucleic acid.
5. A method of modifying a chromatin structure of a nucleic acid in a cell (e.g., a cancer cell), comprising:
contacting the cell with a site-specific disrupting agent that binds, e.g., binds specifically, to a genomic sequence element (e.g., anchor sequence, promoter, or enhancer) proximal to a breakpoint,
wherein the nucleic acid comprises:
i) the breakpoint (e.g., a breakpoint resulting from a gross chromosomal rearrangement),
ii) a gene (e.g., a fusion gene, e.g., a fusion oncogene), e.g., located proximal to the breakpoint,
iii) the genomic sequence element (e.g., anchor sequence, promoter, or enhancer), which is located proximal to the gene,
thereby modifying the chromatin structure of the nucleic acid.
6. The method of embodiment 5, wherein the site-specific disrupting agent comprises an epigenetic modifying moiety chosen from a DNA methyltransferase (e.g., MQ1 or a functional variant or fragment thereof) or a transcription repressor (e.g., KRAB or a functional variant or fragment thereof).
10. The method of any of embodiments 2-4, wherein the first and/or second anchor sequence is proximal to a gene (e.g., an oncogene, e.g., a fusion oncogene).
11. A cell modified by the method of or comprising the modified chromatin structure of any of embodiments 1-10.
12. A cell comprising a nucleic acid, said nucleic acid comprising:
i) a gene;
ii) a breakpoint (e.g., a breakpoint resulting from a gross chromosomal rearrangement), located proximal to the gene;
iii) a first anchor sequence, which is located proximal to the breakpoint and/or the gene; and
iv) a second anchor sequence, which is located proximal to the breakpoint and/or the gene;
   wherein the cell comprises a non-naturally occurring, site-specific modification to the first and/or second anchor sequence, or to a component of a genomic complex associated with the first and/or second anchor sequence (e.g., compared to the cell prior to the modification), wherein the site-specific modification occurs preferentially at the first and/or second anchor sequence or the component of the genomic complex,
   wherein the site-specific modification leads to downregulation of the gene.
   13. The cell of embodiment 12, which is present in a mixture of cells comprising one or more cells that lack the non-naturally occurring modification to the anchor sequence or the component of the genomic complex.
   14. The cell of embodiment 12 or 13, wherein the non-naturally occurring modification comprises a modification to first anchor sequence or the second anchor sequence (or both), e.g., to the DNA sequence or chromatin structure of the first anchor sequence or the second anchor sequence (or both).
   15. The cell of any of embodiments 12-14, wherein the modification is chosen from a DNA sequence modification (e.g., deletion), or an epigenetic modification, e.g., DNA methylation or a histone modification.
   16. A method of treating a cancer in a subject, comprising:
   administering to the subject a site-specific disrupting agent that binds, e.g., binds specifically, to a first anchor sequence, or a component of a genomic complex associated with the first anchor sequence, in a cell, in an amount sufficient to treat the cancer,
   wherein the cell comprises a nucleic acid, said nucleic acid comprising:
   i) an oncogene (e.g., a fusion oncogene);
   ii) a breakpoint (e.g., a breakpoint resulting from a gross chromosomal rearrangement), located proximal to the oncogene;
   iii) a first anchor sequence, which is located proximal to the breakpoint and/or the oncogene; and
   iv) a second anchor sequence, which is located proximal to the breakpoint and/or the oncogene;
   wherein the site-specific disrupting agent is administered in an amount sufficient to decrease expression of the oncogene,
   thereby treating the cancer.
   17. The method or cell of any of embodiments 1-4 or 10-16, wherein the anchor sequence (e.g., the first and/or second anchor sequence) is a cancer-specific anchor sequence.
   18. A composition comprising a targeting moiety that binds, e.g., binds specifically, to a first anchor sequence that is proximal to a breakpoint (e.g., a breakpoint resulting from a gross chromosomal rearrangement), or to a component of a genomic complex that is associated with the anchor sequence.
   19. The composition of embodiment 18, which can introduce a site-specific modification to the first anchor sequence or to the component of the genomic complex associated with the anchor sequence (e.g., compared to the cell prior to the modification).
   20. A site-specific disrupting agent, comprising:
   a DNA- or RNA-binding moiety that binds, e.g., binds specifically, to a target anchor sequence or to a component of a genomic complex associated with the target anchor sequence, wherein the target anchor sequence is proximal to a breakpoint (e.g., a breakpoint resulting from a gross chromosomal rearrangement), e.g., with sufficient affinity that it competes with binding of an endogenous nucleating polypeptide to the target anchor sequence.
   21. A site-specific disrupting agent, comprising a DNA-binding moiety that binds, e.g., binds specifically, to a sequence bound by a gRNA of any of Tables 5-8, or to a sequence referred to in Table 9.
   22. A site-specific disrupting agent, comprising:
a targeting moiety that binds, e.g., binds specifically, to a genomic sequence element (e.g., anchor sequence, promoter, or enhancer) proximal to an IGH fusion oncogene (e.g., comprising or proximal to a breakpoint (e.g., a breakpoint resulting from a gross chromosomal rearrangement)),
wherein binding of the site-specific disrupting agent decreases expression of the IGH fusion oncogene.
   23. The site-specific disrupting agent or method of any of embodiments 5, 6, or 22, wherein the genomic sequence element is upstream from the IGH fusion oncogene.
   24. The site-specific disrupting agent or method of any of embodiments 5, 6, 22 or 23, wherein the genomic sequence element is an enhancer, e.g., that is or is part of a super enhancer.
   25. The site-specific disrupting agent of any of embodiments 22-24, wherein the targeting moiety is or comprises a CRISPR/Cas molecule, a TAL effector molecule, or a Zn finger molecule.
   26. The site-specific disrupting agent or method of any of embodiments 5, 6, 22, 23, or 25, wherein the genomic sequence element is an anchor sequence.
   27. A reaction mixture comprising:
   a) a nucleic acid comprising:
i) a gene (e.g., an oncogene, e.g., a fusion oncogene);
ii) a breakpoint (e.g., a breakpoint resulting from a gross chromosomal rearrangement), located proximal to the gene; and
iii) a target anchor sequence (e.g., target cancer-specific anchor sequence), which is located proximal to the breakpoint and/or the gene, and
   b) a first agent (e.g., a probe or a site-specific disrupting agent) that binds, e.g., binds specifically, to the target anchor sequence or to a component of a genomic complex associated with the anchor sequence.
   28. A method of decreasing expression (e.g., transcription) of a gene (e.g., an oncogene, e.g., a fusion oncogene) in a cell (e.g., a cancer cell), comprising:
contacting the cell with a site-specific disrupting agent that binds, e.g., binds specifically, to a cancer-specific anchor sequence, a second anchor sequence which associates with the cancer-specific anchor sequence in the cell (e.g., in an anchor sequence-mediated conjunction), or a component of a genomic complex associated with the cancer-specific anchor sequence or the second anchor sequence, in the cell, in an amount sufficient to decrease expression of the gene,
wherein the cell comprises a nucleic acid, said nucleic acid comprising:
i) the gene;
ii) the cancer-specific anchor sequence, which is located proximal to the gene; and
iii) the second anchor sequence, which is located proximal to the gene;
   thereby decreasing expression of the gene.
   29. A method of decreasing expression (e.g., transcription) of a gene (e.g., an oncogene, e.g., a fusion oncogene) in a cell (e.g., a cancer cell), comprising:
contacting the cell with a site-specific disrupting agent that binds, e.g., binds specifically, to a cancer-specific anchor sequence or a component of a genomic complex associated with the cancer-specific anchor sequence, in the cell, in an amount sufficient to decrease expression of the gene,
wherein the cell comprises a nucleic acid, said nucleic acid comprising:
i) the gene;
ii) the cancer-specific anchor sequence, which is located proximal to the gene; and
iii) a second anchor sequence, which is located proximal to the gene;
   thereby decreasing expression of the gene.
   30. A method of modifying a chromatin structure of a nucleic acid in a cell (e.g., a cancer cell), comprising:
contacting the cell with a site-specific disrupting agent that binds, e.g., binds specifically, to a cancer-specific anchor sequence, or a component of a genomic complex associated with the cancer-specific anchor sequence, in the cell, in an amount sufficient to modify the chromatin structure of the nucleic acid;
   thereby modifying the chromatin structure of the nucleic acid.
   31. A method of modifying a chromatin structure of a nucleic acid in a cell (e.g., a cancer cell), comprising:
altering a topology of an anchor sequence-mediated conjunction, e.g., a loop, said conjunction comprising a cancer-specific anchor sequence and a second anchor sequence that form the conjunction;
   thereby modifying the chromatin structure of the nucleic acid.
   32. A method of modifying a chromatin structure of a nucleic acid in a cell (e.g., a cancer cell), comprising:
   altering a cancer-specific anchor sequence, or a component of a genomic complex associated with the cancer-specific anchor sequence,
   thereby modifying the chromatin structure of the nucleic acid.
   33. The method of any of embodiments 30-32, wherein the cancer-specific anchor sequence is proximal to a gene (e.g., an oncogene, e.g., a fusion oncogene).
   34. A cell made by or comprising the modified chromatin structure of the method of any of embodiments 28-33.
   35. A cell comprising a nucleic acid, said the nucleic acid comprising:
i) a gene;
ii) a cancer-specific anchor sequence, which is located proximal to the gene; and
iii) a second anchor sequence (e.g., a second cancer-specific anchor sequence), which is located proximal to the gene;
wherein the cell comprises a non-naturally occurring, site-specific modification to the cancer-specific anchor sequence, or to a component of a genomic complex associated with the cancer-specific anchor sequence (e.g., compared to the cell prior to the modification), wherein the site-specific modification occurs preferentially at the cancer-specific anchor sequence or the component of the genomic complex, and wherein prior to the site specific modification the cancer-specific anchor sequence and the second anchor sequence associate in the cell (e.g., in an anchor sequence-mediated conjunction).
   36. The cell of embodiment 35, which is present in a mixture of cells comprising one or more cells that lack the non-naturally occurring modification to the anchor sequence or the component of the genomic complex.
   37. The cell of embodiment 35 or 36, wherein the non-naturally occurring modification comprises a modification to the first cancer-specific anchor sequence or the second anchor sequence (or both), e.g., to the DNA sequence or chromatin structure of the first cancer-specific anchor sequence or the second anchor sequence (or both).
   38. A method of treating a cancer in a subject, comprising:
administering to the subject a site-specific disrupting agent that binds, e.g., binds specifically, to a cancer-specific anchor sequence, or a component of a genomic complex associated with the cancer-specific anchor sequence, in the cell, in an amount sufficient to treat the cancer,
wherein the cell comprises a nucleic acid, said nucleic acid comprising:
i) an oncogene (e.g., a fusion oncogene);
ii) a cancer-specific anchor sequence, which is located proximal to the gene; and
iii) a second anchor sequence (e.g., a second cancer-specific anchor sequence), which is located proximal to the gene and which associates with the cancer-specific anchor sequence in the cell (e.g., in an anchor sequence-mediated conjunction);
   thereby treating the cancer.
   39. The method of any of embodiments 28-38, wherein the nucleic acid further comprises a breakpoint, e.g., a breakpoint resulting from a gross chromosomal rearrangement, located proximal to the cancer-specific anchor sequence.
   40. A composition comprising a targeting moiety that binds a target cancer-specific anchor sequence, or to a component of a genomic complex that is associated with the cancer-specific anchor sequence.
   41. The composition of embodiment 40, which can introduce a site-specific modification to the cancer-specific anchor sequence or to the component of the genomic complex associated with the anchor sequence (e.g., compared to the cell prior to the modification).
   42. A site-specific disrupting agent, comprising:
   a DNA- or RNA-binding moiety that binds, e.g., binds specifically, to a target cancer-specific anchor sequence or to a component of a genomic complex associated with the target cancer-specific anchor sequence, e.g., with sufficient affinity that it competes with binding of an endogenous nucleating polypeptide to the target cancer-specific anchor sequence.
   43. A reaction mixture comprising:
a) a nucleic acid comprising:
   i) a gene (e.g., an oncogene, e.g., a fusion oncogene);
   ii) a target cancer-specific anchor sequence, which is located proximal to the gene, and
b) a first agent (e.g., a probe or a site-specific disrupting agent) that binds, e.g., binds specifically, to the target cancer-specific anchor sequence or to a component of a genomic complex associated with the target cancer-specific anchor sequence.
   44. The method, cell, site-specific disrupting agent, reaction mixture, or composition of any of embodiments 1-43, wherein one or more of:
a) the cell is from a tumor (e.g., a solid tumor or liquid tumor);
b) the cell is not from a cell line;
c) the cell does not comprise adenovirus DNA, e.g., is not an adenovirus-transformed cell line;
d) the gene is other than MYC, SHMT2, CDK6, FOXJ3, RAS, HER1, HER2, JUN, FOS, SRC, or RAF, or does not comprise a portion of MYC, SHMT2, CDK6, FOXJ3, RAS, HER1, HER2, JUN, FOS, SRC, or RAF; or
e) the method further comprises a step of acquiring information that the cell comprises a cancer-specific anchor sequence.
   45. The method, cell, site-specific disrupting agent, reaction mixture, or composition of any of embodiments 1-44, wherein the genomic sequence element or anchor sequence, e.g., cancer-specific anchor sequence, is demethylated compared to the corresponding DNA sequence in a non-cancer cell.
   46. The method, cell, site-specific disrupting agent, reaction mixture, or composition of any of embodiments 1-45, wherein the genomic sequence element or anchor sequence, e.g., cancer-specific anchor sequence, comprises a genetic modification (e.g., a substitution or deletion) compared to the corresponding DNA sequence in a non-cancer cell.
   47. The method, cell, site-specific disrupting agent, reaction mixture, or composition of any of embodiments 1-46, wherein the genomic sequence element or anchor sequence, e.g., cancer-specific anchor sequence, is proximal to a gross chromosomal rearrangement, e.g., a translocation.
   48. The method, cell, site-specific disrupting agent, reaction mixture, or composition of any of embodiments 1-27, 39, or 47, wherein the gross chromosomal rearrangement comprises a translocation, deletion (e.g., interstitial deletion or terminal deletion), inversion, insertion, amplification (e.g., duplication), e.g., a tandem amplification or tandem duplication, chromosome end-to-end fusion, chromothripsis, or any combination thereof.
   49. The method, cell, site-specific disrupting agent, reaction mixture, or composition of any of embodiments 1-27, 39, 47, or 48, wherein the gross chromosomal rearrangement comprises an inter-chromosomal rearrangement or an intra-chromosomal rearrangement.
   50. The method, cell, site-specific disrupting agent, reaction mixture, or composition of any of embodiments 1-27, 39, or 47-49, wherein the breakpoint is located in a transcribed region (e.g., in an intron, an exon, a 5′ UTR, or a 3′ UTR) or in a non-transcribed region.
   51. The method, cell, site-specific disrupting agent, reaction mixture, or composition of any of embodiments 1-27, 39, or 47-50, wherein the breakpoint is in chromosome 1, 2, 3, 4, 5, 6, 7, 8, 9, 10, 11, 12, 13, 14, 15, 16, 17, 18, 19, 20, 21, 22, X, or Y, e.g., 14.
   52. The method, cell, site-specific disrupting agent, reaction mixture, or composition of any of embodiments 1-27, 39, or 47-51, wherein the gross chromosomal rearrangement is a translocation of at least 1, 2, 5, 10, 20, 50, or 100 MB.
   53. The method, cell, site-specific disrupting agent, reaction mixture, or composition of any of embodiments 1-27, 39, or 47-52, wherein the gross chromosomal rearrangement results in formation of a fusion oncogene.
   54. The method, cell, site-specific disrupting agent, reaction mixture, or composition of any of embodiments 1-27, 39, or 47-52, wherein the gross chromosomal rearrangement does not result in formation of a fusion oncogene.
   55. The method, cell, site-specific disrupting agent, reaction mixture, or composition of any of embodiments 1-27, 39, or 47-54, wherein the breakpoint, gene (e.g., the entire gene or a portion thereof, e.g., the transcriptional start site of the gene), and anchor sequence are within a 10, 20, 50, 100, 200, 500, 1,000, 2,000, or 3,000 kb region.
   56. The method or cell of any of embodiments 1-17, 28, 29, 34-39, or 48-55 wherein the nucleic acid further comprises an internal enhancing sequence which is located at least partially between the first anchor sequence (e.g., the cancer-specific anchor sequence) and the second anchor sequence.
   57. The method or cell of any of embodiments 1-17, 28, 29, 34-39, or 48-56, wherein the nucleic acid further comprises one or more repressor signals, e.g., one or more silencing sequences, wherein the one or more repressor signals are located outside an anchor-sequence mediated conjunction formed by the first anchor sequence (e.g., the cancer-specific anchor sequence) and the second anchor sequence.
   58. The method or cell of any of embodiments 1-17, 28, 29, 34-39, or 48-57, wherein the breakpoint is located within the first anchor sequence or the second anchor sequence.
   59. The method, cell, or reaction mixture, of any of embodiments 1-17, 27-39, or 48-58, wherein the nucleic acid comprises an anchor sequence mediated conjunction, e.g., a loop.
   60. The method, cell, or reaction mixture of any of embodiments 1-17, 27-39, or 48-59, wherein the nucleic acid is an anchor sequence mediated conjunction, e.g., is a loop.
   61. The method, cell, or reaction mixture, of any of embodiments 1-17, 27-39, or 48-59, wherein the nucleic acid comprises an anchor sequence mediated conjunction (e.g., a loop) and further comprises sequence adjacent to the anchor sequence mediated conjunction, e.g., on one or both sides of the anchor sequence mediated conjunction.
   62. The method, cell, or reaction mixture of any of embodiments 59-61, wherein the anchor sequence mediated conjunction comprises at least a portion of the gene, e.g., wherein the anchor sequence mediated conjunction comprises the transcriptional start site of the gene or wherein the anchor sequence mediated conjunction comprises the entire gene.
   63. The method, cell, or reaction mixture of any of embodiments 59-62, wherein the anchor sequence mediated conjunction comprises at least a portion of a promoter of the gene, e.g., wherein the anchor sequence mediated conjunction comprises the entire promoter.
   64. The method, cell or reaction mixture of any of embodiments 59-63, wherein the anchor sequence mediated conjunction comprises the breakpoint. 65. The method, cell or reaction mixture of any of embodiments 59-63, wherein the breakpoint is outside of the anchor sequence mediated conjunction.
   66. The method or cell of any of embodiments 1-17 or 48-65, wherein the breakpoint is between the first anchor sequence (e.g., the cancer-specific anchor sequence) and the second anchor sequence.
   67. The method, reaction mixture, or cell composition of any of embodiments 1-17, 27-39, or 43-66, wherein the nucleic acid is a part of a chromosome.
   68. The method or cell of any of embodiments 1-17, 28, 29, 34-39, or 48-67, wherein the first anchor sequence (e.g., the cancer-specific anchor sequence) and the second anchor sequence are comprised by an anchor sequence mediated conjunction.
   69. The method, cell, site-specific disrupting agent, reaction mixture, or composition of any of embodiments 1-68, wherein the genomic sequence element or first anchor sequence (e.g., cancer-specific anchor sequence) is not in a promoter.
   70. The method, cell, site-specific disrupting agent, reaction mixture, or composition of any of embodiments 1-69, wherein the genomic sequence element or anchor sequence (e.g., first and/or second anchor sequence, e.g., cancer-specific anchor sequence) is at least 100 bp, 200 bp, 500 bp, 1 kb, 1.5 kb, 2 kb, or 2.5 kb away from a transcriptional start site.
   71. The method, cell, site-specific disrupting agent, reaction mixture, or composition of any of embodiments 1-70, wherein the genomic sequence element or anchor sequence (e.g., first and/or second anchor sequence, e.g., cancer-specific anchor sequence) is at least 3, 4, 5, 6, 7, 8, 9, or 10 kb away from a transcriptional start site.
   72. The method, cell, or reaction mixture of any of embodiments 1, 4-17, 28, 29, 33-39, or 43-71, wherein the gene is situated within an anchor sequence-mediated conjunction that comprises the first anchor sequence, second anchor sequence, and one or more transcriptional control sequences (e.g., a promoter and/or an enhancing sequence).
   73. The method, cell, or reaction mixture of embodiment 72, wherein the gene is separated from the transcriptional control sequence, e.g., enhancing sequence, by about 100 bp, 300 bp, 500 bp, 600 bp, 700 bp, 800 bp, 900 bp, 1 kb, 5 kb, 10 kb, 15 kb, 20 kb, 25 kb, 30 kb, 35 kb, 40 kb, 45 kb, 50 kb, 55 kb, 60 kb, 65 kb, 70 kb, 75 kb, 80 kb, 85 kb, 90 kb, 95 kb, 100 kb, 125 kb, 150 kb, 175 kb, 200 kb, 225 kb, 250 kb, 275 kb, 300 kb, 350 kb, 400 kb, 500 kb, 600 kb, 700 kb, 800 kb, 900 kb, 1 Mb, 2 Mb, 3 Mb, 4 Mb, 5 Mb, 6 Mb, 7 Mb, 8 Mb, 9 Mb, 10 Mb, 15 Mb, 20 Mb, 25 Mb, 50 Mb, 75 Mb, 100 Mb, 200 Mb, 300 Mb, 400 Mb, 500 Mb, or any size therebetween, e.g., at least 300 base pairs.
   74. The method or cell of any of embodiments 1-17, 28, 29, 34-39, or 48-73, wherein the first and/or second anchor sequence is located within 1 Mb, within 900 kb, within 800 kb, within 700 kb, within 600 kb, within 500 kb, within 450 kb, within 400 kb, within 350 kb, within 300 kb, within 250 kb, within 200 kb, within 180 kb, within 160 kb, within 140 kb, within 120 kb, within 100 kb, within 90 kb, within 80 kb, within 70 kb, within 60 kb, within 50 kb, within 40 kb, within 30 kb, within 20 kb, within 10 kb, within 9 kb, within 8 kb, within 7 kb, within 6 kb, within 5 kb, within 4 kb, within 3 kb, within 2 kb, or within 1 kb, e.g., within 500 kb, of an external transcriptional control sequence, e.g., a silencing or repressive sequence.
   75. The method, cell, or reaction mixture of any of embodiments 1, 4-17, 22-29, 33-39, or 43-74, wherein the genomic sequence element or anchor sequence (e.g., the first anchor sequence and/or cancer-specific anchor sequence) is located upstream of the gene.
   76. The method, cell, or reaction mixture of any of embodiments 1, 4-17, 22-29, 33-39, or 43-74, wherein the anchor sequence (e.g., the first anchor sequence and/or cancer-specific anchor sequence) is located downstream of the gene.
   77. The method, cell, or reaction mixture of any of embodiments 1, 4-17, 22-29, 33-39, or 43-74, wherein the anchor sequence (e.g., the first anchor sequence and/or cancer-specific anchor sequence) is located within the gene.
   78. The method, cell, or reaction mixture of any of embodiments 1, 4-17, 22-29, 33-39, or 43-77, wherein the second anchor sequence is located upstream of the gene.
   79. The method, cell, or reaction mixture of any of embodiments 1, 4-17, 28, 29, 33-39, or 43-77, wherein the second anchor sequence is located downstream of the gene.
   80. The method, cell, or reaction mixture of any of embodiments 1, 4-17, 28, 29, 33-39, or 43-77, wherein the second anchor sequence is located within the gene.
   81. The method, cell, site-specific disrupting agent, reaction mixture, or composition of any of embodiments 1-22 or 48-80, wherein the target anchor sequence or first anchor sequence (e.g., the cancer-specific anchor sequence) is located between the breakpoint and the centromere.
   82. The method, cell, site-specific disrupting agent, reaction mixture, or composition of any of embodiments 1-22 or 48-80, wherein the target anchor sequence or first anchor sequence (e.g., the cancer-specific anchor sequence) is located between the breakpoint and the telomere.
   83. The method, cell, site-specific disrupting agent, reaction mixture, or composition of any of embodiments 1-17, 20, 27-, or 48-82, wherein the target anchor sequence or second anchor sequence is located between the breakpoint and the centromere.
   84. The method, cell, site-specific disrupting agent, reaction mixture, or composition of any of embodiments 1-17, 20, 27, or 48-82, wherein the target anchor sequence or second anchor sequence is located between the breakpoint and the telomere.
   85. The method, cell, site-specific disrupting agent, reaction mixture, or composition of any of embodiments 1-17, 19, 20, 22, 23, 25-84, wherein the anchor sequence (e.g., the first anchor sequence and/or the cancer-specific anchor sequence) is located in a transcribed region (e.g., in an intron, an exon, a 5′ untranslated region, or a 3′ untranslated region) or in a non-transcribed region.
   86. The method, cell, or reaction mixture of any of embodiments 1, 4-17, 28, 29, 33-39, or 43-85, wherein the second anchor sequence is located in a transcribed region (e.g., in an intron, an exon, a 5′ untranslated region, or a 3′ untranslated region) or in a non-transcribed region.
   87. The method, cell, site-specific disrupting agent, reaction mixture, or composition of any of embodiments 1-17, 19, 20, 22, 23, 25-86, wherein the anchor sequence (e.g., the first anchor sequence and/or cancer-specific anchor sequence) comprises a CTCF binding motif, BORIS binding motif, cohesin binding motif, USF 1 binding motif, YY1 binding motif, TATA-box, or ZNF143 binding motif.
   88. The method, cell, or reaction mixture of any of embodiments 1, 4-17, 28, 29, 33-39, or 43-87, wherein the second anchor sequence comprises a CTCF binding motif, BORIS binding motif, cohesin binding motif, USF 1 binding motif, YY1 binding motif, TATA-box, or ZNF143 binding motif.
   89. The method, cell, site-specific disrupting agent, reaction mixture, or composition of any of embodiments 1-17, 19, 20, 22, 23, 25-88, wherein the anchor sequence (e.g., the first anchor sequence and/or cancer-specific anchor sequence) is adjacent to a CTCF binding motif, BORIS binding motif, cohesin binding motif, USF 1 binding motif, YY1 binding motif, TATA-box, or ZNF143 binding motif.
   90. The method, cell, or reaction mixture of any of embodiments 1, 4-17, 28, 29, 33-39, or 43-89, wherein the second anchor sequence is adjacent to a CTCF binding motif, BORIS binding motif, cohesin binding motif, USF 1 binding motif, YY1 binding motif, TATA-box, or ZNF143 binding motif.
   91. The method, cell, site-specific disrupting agent, reaction mixture, or composition of any of embodiments 1-17, 19, 20, 22, 23, 25-90, wherein the anchor sequence (e.g., first anchor sequence and/or cancer-specific anchor sequence) comprises methylated DNA.
   92. The method or composition of any of embodiments 3, 28, 31, 35, 38, and 48-91, wherein the anchor sequence mediated conjunction is a cancer fusion loop (CFL).
   93. The method or composition of any of embodiments 3, 28, 31, 35, 38, and 48-92, wherein the anchor sequence mediated conjunction is a cancer-specific anchor sequence mediated conjunction.
   94. The site-specific disrupting agent of any of embodiments 20, 21, 22, 23, 25, 26, 42, 48-55, 69-71, 81-83, 88, 89, or 91 wherein the anchor sequence comprises or is comprised partly or completely by a sequence bound by a gRNA of any of Tables 5-8, or is comprised partly or completely by a sequence referred to in Table 9.
   95. The site-specific disrupting agent of any of 20, 21, 22, 23, 25, 26, 42, 48-55, 69-71, 81-83, 88, 89, 91, or 94, wherein the anchor sequence comprises a sequence selected from SEQ ID NOs: 1 or 2.
   96. The method, cell, or reaction mixture of any of embodiments 1, 4-17, 28, 29, 33-39, or 43-91, wherein the gene comprises a transcription factor, e.g., a full length transcription factor or a transcriptionally active fragment thereof.
   97. The method, cell, or reaction mixture of any of embodiments 1, 4-17, 28, 29, 33-39, 43-89, or 96, wherein the gene comprises a kinase, e.g., a full length kinase or a fragment thereof having kinase activity.
   98. The method, cell, or reaction mixture of embodiment 97, wherein the kinase is a constitutively active kinase.
   99. The method, cell, or reaction mixture of any of embodiments 1, 4-17, 28, 29, 33-39, 43-89, or 96-98, wherein the gene comprises a transmembrane receptor, e.g., a full length transmembrane receptor or a transmembrane fragment thereof.
   100. The method, cell, or reaction mixture of any of embodiments 1, 4-17, 28, 29, 33-39, 43-89, or 96-99, wherein the gene comprises a cell cycle regulator (e.g., full length cell cycle regulator or an active fragment thereof), a pro-survival factor (e.g., full length pro-survival factor or an active fragment thereof), or a migration protein (e.g., full length migration protein or an active fragment thereof).
   101. The method, cell, or reaction mixture of any of embodiments 1, 4-17, 28, 29, 33-39, 43-89, or 96-100, wherein the gene comprises a coiled-coil domain, paired box domain, DNA-binding domain, or transactivating domain.
   102. The method, cell, or reaction mixture of any of embodiments 1, 4-17, 28, 29, 33-39, 43-89, or 96-102, wherein the gene comprises a fusion oncogene that is a fusion between a first fusion partner gene and a second fusion partner gene, e.g., wherein the fusion oncogene comprises one or more exons from the first fusion partner gene and one or more exons from the second fusion partner gene.
   103. The method, cell, or reaction mixture of embodiment 102, wherein the first or second fusion partner gene comprises IGH or a functional fragment or variant thereof.
   104. The method, cell, or reaction mixture of either of embodiments 102 or 103, wherein the first or second fusion partner gene comprises MYC or a functional fragment or variant thereof.
   105. The method, cell, or reaction mixture of either of embodiments 102 or 103, wherein the first or second fusion partner gene comprises BCL2 or a functional fragment or variant thereof.
   106. The method, cell, or reaction mixture of either of embodiments 102 or 103, wherein the first or second fusion partner gene comprises CCND1 or a functional fragment or variant thereof.
   107. The method, cell, or reaction mixture of either of embodiments 102 or 103, wherein the first or second fusion partner gene comprises BCL6 or a functional fragment or variant thereof.
   108. The method, cell, or reaction mixture of embodiment 102, wherein the first fusion partner gene comprises a first transcription factor and the second fusion partner gene comprises a second transcription factor.
   109. The method, cell, or reaction mixture of embodiment 102, wherein the first fusion partner gene comprises a kinase and the second fusion partner gene comprises a transmembrane receptor.
   110. The method, cell, or reaction mixture of any of embodiments 1, 4-17, 28, 29, 33-39, 43-89, or 96-109, wherein the gene is a gene that is not present in a non-cancerous cell, e.g., a wild-type cell.
   111. The method, cell, or reaction mixture of any of embodiments 1, 4-17, 28, 29, 33-39, 43-89, or 96-110, wherein the gene is not present in the Genome Reference Consortium human genome (build 38).
   112. The method, cell, or reaction mixture of any of embodiments 1, 4-17, 28, 29, 33-39, 43-89, or 96-111, wherein the gene produces a product that is not present in a wild-type cell.
   113. The method, cell, or reaction mixture of any of embodiments 1, 4-17, 28, 29, 33-39, 43-89, or 96-112, wherein the gene produces a product that is not present in a non-cancer cell of the same tissue type as the cancer cell.
   114. The method, cell, or reaction mixture of any of embodiments 1, 4-17, 28, 29, 33-39, 43-89, or 96-113, wherein expression of the gene in the cell (e.g., cancer cell) is deregulated, e.g., increased, e.g., compared to its expression in a non-cancer cell of the same tissue type as the cancer cell.
   115. The method, cell, or reaction mixture of any of embodiments 1, 4-17, 28, 29, 33-39, 43-89, or 96-114, wherein the gene comprises a fusion oncogene of Table 1.
   116. The method, cell, or reaction mixture of any of embodiments 1, 4-17, 28, 29, 33-39, 43-89, or 96-115, wherein the gene comprises a fusion oncogene chosen from: CCDC6-RET, PAX3-FOXO, BRC-ABL1, IGH-CCND1, IGH-MYC, IGH-BCL2, or EML4-ALK.
   117. The method, cell, or reaction mixture of any of embodiments 1, 4-17, 28, 29, 33-39, 43-89, or 96-116, wherein expression of the gene in the cell, e.g., cancer cell, is reduced to less than 80%, 70%, 60%, 50%, 40%, 30%, or 20% of a reference level, e.g., wherein the reference is expression level of the same gene in an otherwise similar, untreated cell (e.g., untreated cancer cell).
   118. The method, cell, or reaction mixture of any of embodiments 1, 4-17, 28, 29, 33-39, 43-89, or 96-117, wherein expression of the gene in a non-cancer cell contacted with the site-specific binding agent changes (e.g., increases or decreases) less than 10%, 20%, or 30% relative to a reference level, e.g., wherein the reference is expression level of the same gene in an otherwise similar, untreated non-cancer cell.
   119. The method, cell, or reaction mixture of any of embodiments 110-118, wherein the gene is a fusion oncogene, and wherein the non-cancer cell comprises first and second endogenous genes corresponding to the fusion oncogene, and wherein expression of the first and/or second endogenous genes in the non-cancer cell changes (e.g., increases or decreases) less than 10%, 20%, or 30% relative to a reference level, e.g., wherein the reference is expression level of the endogenous gene an otherwise similar, untreated non-cancer cell.
   120. The method or cell of any of embodiments 1, 5-9, 11, 16, 17, 28, 29, 34, 38, 39, 48-91, and 96-119, wherein the site-specific disrupting agent binds, e.g., binds specifically, to a first anchor sequence, e.g., a target cancer-specific anchor sequence, or a component of a genomic complex associated with the first anchor sequence, e.g., target cancer-specific anchor sequence, and wherein the site-specific disrupting agent alters (e.g., decreases) expression of the gene in a cancer cell more than the site-specific disrupting agent alters (e.g., decreases) expression of the gene (or one or two endogenous genes corresponding to the gene, e.g., fusion oncogene) in a non-cancer cell.
   121. The method or cell of embodiment 120, wherein the percentage decrease in the cancer cell is at least 10%, 20%, 30%, 40%, 50%, 60%, 70%, 80%, 90%, 2-fold, 3-fold, 4-fold, 5-fold, or 10-fold larger than the percentage decrease in the non-cancer cell.
   122. The method or cell of embodiment 120 or 121, wherein the site-specific disrupting agent does not alter (e.g., does not decrease) the expression of a gene (e.g., proto-oncogene and/or an endogenous gene corresponding to the fusion oncogene) in a non-cancerous cell.
   123. The method, cell, or reaction mixture of any of embodiments 120-122, wherein expression is measured by detecting mRNA levels, e.g., using a quantitative RT-PCR assay, e.g., using an assay of Example 1.
   124. The method, cell, or reaction mixture of any of embodiments 120-123, wherein expression is measured by detecting protein levels, e.g., using FACS, Western blot, or ELISA.
   125. The method, cell, or reaction mixture of any of embodiments 3, 10, 11, 17, 48-93, or 96-124 wherein altered topology of the anchor sequence-mediated conjunction decreases expression, e.g., transcription, of the gene.
   126. The method or composition of any of embodiments 4, 10-15, 32, 33, 39, 48-91, or 96-125, wherein altering the anchor sequence comprises altering the DNA sequence or methylation of the target anchor sequence.
   127. The method or composition of any of any of embodiments 4, 10-15, 32, 33, 39, 48-91, or 96-125, wherein altering the component of a genomic complex associated with the anchor sequence comprises altering chromatin structure at the anchor sequence.
   128. The method, cell, site-specific disrupting agent, reaction mixture, or composition of any of embodiments 1-127, wherein the DNA sequence of the genomic sequence element or first and/or second anchor sequence, e.g., target anchor sequence, is altered.
   129. The method, cell, site-specific disrupting agent, reaction mixture, or composition of any of embodiments 1-128, wherein the chromatin structure of the first and/or second anchor sequence, e.g., target anchor sequence, is altered.
   130. The method, cell, site-specific disrupting agent, reaction mixture, or composition of any of embodiments 1-129, wherein DNA methylation of the first and/or second anchor sequence (e.g., target anchor sequence) is altered (e.g., increased or decreased).
   131. The method, reaction mixture, or cell of any of embodiments 1, 4-17, 28, 29, 33-39, 43-91, or 96-117, wherein interaction of an enhancer with the gene is reduced.
   132. The method or composition of embodiment 131, wherein the enhancer is at least 5, 6, 7, 8, 9, 10, 15, 20, 25, 30, 35, 40, 45, 50, 60, 70, 80, 90, 100, 200, 300, 400, 500, 1000, 1500, 2000, 3000, 4000, or 5000 kb distant from the gene.
   133. The method or composition of embodiment 131, wherein, prior to contacting with the site-specific modifying agent, the enhancer was within the same anchor mediated sequence conjunction as the gene.
   134. The method, cell, or reaction mixture of any of embodiments 1, 4-17, 28, 29, 33-39, 43-91, or 96-133, wherein interaction of a silencing element with the gene is increased.
   135. The method, cell, or composition of embodiment 4, 10-15, 32, 33, 39, 48-91, or 96-134, wherein introducing a site-specific modification or altering an anchor sequence or component of the genomic complex associated with the anchor sequence comprises altering an epigenetic modification present at the anchor sequence or a component of a genomic complex associated with the anchor sequence.
   136. The method of embodiment 135, wherein the epigenetic modification is selected from DNA methylation or a histone modification (e.g., histone methylation or histone acetylation).
   137. The method, cell, reaction mixture, or site-specific disrupting agent of any of embodiments 1, 2, 11, 12, 15-20, 23-29, or 32-121 wherein the site-specific disrupting agent comprises a DNA-binding moiety that binds the anchor sequence.
   138. The method, cell, reaction mixture, or site-specific disrupting agent of any of embodiments 1, 2, 16, 17, 20-30, 33-39, or 42-137 wherein the site specific disrupting agent comprises an RNA-binding moiety that binds a non-coding RNA comprised by the genomic complex.
   139. The method, cell, reaction mixture, or site-specific disrupting agent of any of embodiments 1, 2, 16, 17, 20-30, 33-39, or 42-138 wherein the site-specific disrupting agent comprises a protein-binding moiety that binds a nucleating protein comprised by the genomic complex, wherein optionally the site specific disrupting agent also binds DNA of the genomic complex.
   140. The method of any of embodiments 1-10, 16, 17, 28-33, 38, 39, 44-93, or 96-139, which comprises:
   a) substituting, adding, or deleting one or more nucleotides to the anchor sequence (e.g., first and/or second anchor sequence, and/or target anchor sequence) (e.g., using a Cas9, ZFN, or TALEN);
   b) epigenetically modifying the anchor sequence (e.g., first and/or second anchor sequence, and/or target anchor sequence) (e.g., altering DNA methylation or histone modification); or
   c) sterically hindering formation of an anchor sequence-mediated conjunction (e.g., using dCas9 or an oligonucleotide).
   141. The method of any of embodiments 1-10, 16, 17, 28-33, 38, 39, 44-93, or 96-140, which comprises: deleting one or more nucleotides (e.g., all of the nucleotides) of the anchor sequence (e.g., first and/or second anchor sequence, and/or target anchor sequence) (e.g., using a Cas9, ZFN, or TALEN).
   142. The method, cell, reaction mixture, or site-specific disrupting agent of any of embodiments 1, 2, 16, 17, 20-30, 33-39, or 42-141, wherein the site-specific disrupting agent comprises an effector moiety that:
   (i) is a chemical, e.g., a chemical that modulates a cytosine (C) or an adenine(A) (e.g., sodium bisulfite or ammonium bisulfite);
   (ii) has enzymatic activity (e.g., methyltransferase, nuclease (e.g., Cas9, ZFN, or TALEN), or deaminase); or
   (iii) sterically hinders formation of the anchor sequence-mediated conjunction, e.g., ssDNA oligonucleotides, locked nucleic acids (LNAs), peptide oligonucleotide conjugates (e.g., membrane translocating polypeptides with nucleic acid side chains), bridged nucleic acids (BNAs), polyamides, or antisense oligonucleotide-conjugates comprising a DNA binding molecule.
   143. The method of any of embodiments 1, 2, 16, 17, 28-30, 38, 39, 48-91, or 96-142 which further comprises contacting the cell or nucleic acid with a second site-specific disrupting agent.
   144. The method of embodiment 143, wherein the first site-specific disrupting agent and the second site-specific disrupting agent bind to the same anchor sequence.
   145. The method of embodiment 144, wherein the first site-specific disrupting agent and the second site-specific disrupting agent bind to different binding sites on the same anchor sequence, e.g., bind to adjacent binding sites on the same anchor sequence.
   146. The method of embodiment 143, wherein the first site-specific disrupting agent and the second site-specific disrupting agent bind to different target anchor sequences, e.g., wherein the first site-specific disrupting agent binds to the first anchor sequence (e.g., the cancer-specific anchor sequence) and the second site-specific disrupting agent binds to the second anchor sequence.
   147. The method of embodiment 146, wherein the two different target anchor sequences are in the same anchor sequence mediated conjunction.
   148. The method of embodiment 146, wherein the two different target anchor sequences are in different anchor sequence mediated conjunctions.
   149. The method of embodiment 143, wherein the first site-specific disrupting agent binds a site on a first side of the breakpoint (e.g., between the breakpoint and the centromere) and the second site-specific disrupting agent binds a site on the second side of the breakpoint (e.g., between the breakpoint and the telomere).
   150. The method of any of embodiments 143-149, wherein the distance between the site bound by the first site-specific disrupting agent and the second site-specific disrupting agent is about 1-5, 5-10, 10-20, 20-50, 50-100, 100-200, 200-500, or 500-1000 bp.
   151. The method of embodiment 143, which further comprises contacting the nucleic acid with a third site-specific disrupting agent and optionally a fourth site-specific disrupting agent.
   152. The method, cell, reaction mixture, or site-specific disrupting agent of any of embodiments 1, 2, 5-9, 16, 17, 20-30, 33-39, or 42-151, wherein the site-specific disrupting agent comprises a disrupting moiety associated with a DNA-binding moiety, e.g., as part of the same fusion protein.
   153. The method, cell, reaction mixture, or site-specific disrupting agent of embodiment 152, wherein when the DNA-binding moiety is bound at the one or more anchor sequences (e.g., first and/or second anchor sequences, and/or target anchor sequences), dimerization of an endogenous nucleating polypeptide is reduced when the negative effector moiety is present as compared with when it is absent.
   154. The method, cell, reaction mixture, or site-specific disrupting agent of embodiment 152 or 153, wherein the disrupting moiety comprises a dimerization domain, e.g., a dimerization portion of an endogenous nucleating polypeptide or a variant thereof.
   155. The method, cell, reaction mixture, or site-specific disrupting agent of any of embodiments 21, 137, or 152-154, wherein the DNA binding moiety comprises a polymer, e.g., a polyamide, an oligonucleotide (e.g., an oligonucleotide comprising a chemical modification), or a peptide nucleic acid.
   156. The method, cell, reaction mixture, or site-specific disrupting agent of any of embodiments 21, 137, or 152-155, wherein the DNA binding moiety comprises a peptide or polypeptide, e.g., a zinc finger polypeptide, a transcription activator-like effector nuclease (TALEN) polypeptide, or a Cas9 polypeptide.
   157. The method, cell, reaction mixture, or site-specific disrupting agent of any of embodiments 21, 137, or 152-156, wherein the DNA binding moiety comprises a peptide-nucleic acid mixmer or a small molecule.
   158. The method or cell of any of embodiments 1-17, 28-39, 48-93, or 96-157, wherein the cell is a mammalian cell, a primary cell, a somatic cell, an adult cell, a non-embryonic cell, or any combination thereof.
   159. The method or cell of any of embodiments 1-17, 28-39, 48-93, or 96-158, wherein the cell is a cancer cell of Table 1.
   160. A reaction mixture comprising a cancer cell and a site-specific disrupting agent described herein, e.g., a site-specific disrupting agent of any of embodiments 20-26, 42, 44-55, 69-71, 81-85, 87, 89, 91, 94, 95, 118, 128-130, 137-139, 142, or 152-157, e.g., wherein the cancer cell is from a cancer of Table 1.
   161. The reaction mixture of embodiment 27, 33, 48-55, 59-65, 67, 69-73, 75-91, 96-119, 123-125, 128-134, 137-157, or 160, wherein the nucleic acid is in a cell.
   162. The reaction mixture of embodiment 27, 33, 48-55, 59-65, 67, 69-73, 75-91, 96-119, 123-125, 128-134, 137-157, or 160, wherein the nucleic acid is not in a cell, e.g., is a purified nucleic acid.
   163. The method or composition of any of embodiments 1-11, 16, 17, or 27-162, wherein the cancer is a cancer of Table 1.
   164. The method, cell, or reaction mixture of any of embodiments 1, 4-17, 27-29, 33-39, 43-91, or 96-133, wherein the gene is a gene of Table 1 and the cancer is a cancer of the same row of Table 1.
   165. The method of any of embodiments 16, 17, 38, 44-93, 96-159, 163, or 164, wherein the site-specific disrupting agent comprises a polypeptide, and wherein administering the site-specific disrupting agent to the subject comprises administering the site-specific disrupting agent to the subject, e.g., under conditions that allow the site-specific disrupting agent to enter the cell, e.g., by crossing the cell membrane.
   166. The method of any of embodiments 16, 17, 38, 44-93, 96-159, 163, or 164, wherein administering the site-specific disrupting agent to the subject comprises administering a nucleic acid (e.g., DNA or RNA) encoding the site-specific disrupting agent to the subject under conditions that allow expression of the site-specific disrupting agent in a cell of the subject.
   167. The method of any of embodiments 1, 2, 5-10, 16, 17, 28-30, 38, 39, 48-91, or 96-166, which comprises delivering the site-specific disrupting agent to a cell ex vivo, wherein optionally the method further comprises: (i) prior to the step of delivering, a step of removing the cell from a subject, and/or the method further comprises: (ii) after the step of delivering, a step of administering the cell to a subject.
   168. The cell, reaction mixture, or method of any of embodiments 1, 4-17, 28, 29, 33-39, 43-91, or 96-167, wherein the gene comprises CCDC6-RET, PAX3-FOXO, BRC-ABL1, EML4-ALK, ETV6-RUNX1, TMPRSS2-ERG, TCF3-PBX1, KMT2A-AFF1, IGH-CCND1, IGH-MYC, IGH-BCL2, or EWSR1-FLI1.
   169. The cell, reaction mixture, or method of any of embodiments 1, 4-11, 16, 17, 27-29, 33-39, 43-91, or 96-168, wherein the gene comprises CCDC6-RET and the cancer comprises a thyroid cancer or a lung cancer.
   170. The cell, reaction mixture, or method of any of embodiments 1, 4-11, 16, 17, 27-29, 33-39, 43-91, or 96-168, wherein the gene comprises PAX3-FOXO and the cancer comprises a rhabdomyosarcoma, e.g., an alveolar rhabdomyosarcoma and/or a pediatric rhabdomyosarcoma.
   171. The cell, reaction mixture, or method of any of embodiments 1, 4-11, 16, 17, 27-29, 33-39, 43-91, or 96-168, wherein the gene comprises BRC-ABL1 and the cancer comprises a leukemia, e.g., a CML.
   172. The cell, reaction mixture, or method of any of embodiments 1, 4-11, 16, 17, 27-29, 33-39, 43-91, or 96-168, wherein the gene comprises EML4-ALK and the cancer comprises a lung cancer.
   173. The cell, reaction mixture, or method of any of embodiments 1, 4-11, 16, 17, 27-29, 33-39, 43-91, or 96-168, wherein the gene comprises ETV6-RUNX1 and the cancer comprises an ALL, e.g., a pediatric ALL.
   174. The cell, reaction mixture, or method of any of embodiments 1, 4-11, 16, 17, 27-29, 33-39, 43-91, or 96-168, wherein the gene comprises TMPRSS2-ERG and the cancer comprises prostate cancer.
   175. The cell, reaction mixture, or method of any of embodiments 1, 4-11, 16, 17, 27-29, 33-39, 43-91, or 96-168, wherein the gene comprises TCF3-PBX1 and the cancer comprises a lung cancer or an ALL (e.g., pediatric ALL).
   176. The cell, reaction mixture, or method of any of embodiments 1, 4-11, 16, 17, 27-29, 33-39, 43-91, or 96-168, wherein the gene comprises KMT2A-AFF1 and the cancer comprises ALL, e.g., pediatric ALL.
   177. The cell, reaction mixture, or method of any of embodiments 1, 4-11, 16, 17, 27-29, 33-39, 43-91, or 96-168, wherein the gene comprises EWSR1-FLI1 and the cancer comprises Ewing sarcoma.
   178. The cell, reaction mixture, or method of any of embodiments 1, 4-11, 16, 17, 27-29, 33-39, 43-91, or 96-168, wherein the gene comprises IGH-CCND1 and the cancer comprises lymphoma (e.g., diffuse large B cell lymphoma (DLBCL) or Burkitt's lymphoma).
   179. The cell, reaction mixture, or method of any of embodiments 1, 4-11, 16, 17, 27-29, 33-39, 43-91, or 96-168, wherein the gene comprises IGH-MYC and the cancer comprises lymphoma (e.g., diffuse large B cell lymphoma (DLBCL) or Burkitt's lymphoma).
   180. The cell, reaction mixture, or method of any of embodiments 1, 4-11, 16, 17, 27-29, 33-39, 43-91, or 96-168, wherein the gene comprises IGH-BCL2 and the cancer comprises lymphoma (e.g., diffuse large B cell lymphoma (DLBCL) or Burkitt's lymphoma).
   181. A method of evaluating a subject as being more suitable or less suitable for treatment with a site-specific disrupting agent, said method comprising:
a) determining whether the subject comprises a target anchor sequence (e.g., target cancer-specific anchor sequence), which is located proximal to a breakpoint,
b) responsive to a determination that the subject comprises the target anchor sequence, at a level above a reference value, identifying the subject as being more suitable for treatment with the site-specific disrupting agent; or
c) responsive to a determination that the subject comprises the target anchor sequence at a level below a reference value (e.g., does not comprise the target anchor sequence), identifying the subject as being less suitable for treatment with the site-specific disrupting agent.
   182. The method of embodiment 181, which comprises:
a) responsive to a determination that the subject comprises the target anchor sequence at a level above a reference value, administering a site-specific disrupting agent to the subject, or
b) responsive to a determination that the subject comprises the target anchor sequence at a level below a reference value (e.g., does not comprise the target anchor sequence), not administering the site-specific disrupting agent to the subject, e.g., administering a therapy other than the site-specific disrupting agent to the subject, e.g., administering a standard of care therapy to the subject.
   183. A method of treating a subject having a cancer, comprising:
a) determining whether the subject comprises a target anchor sequence (e.g., target cancer-specific anchor sequence), which is located proximal to a breakpoint,
b) responsive to a determination that the subject comprises the target anchor sequence, administering a site-specific disrupting agent to the subject, or
c) responsive to a determination that the subject comprises the target anchor sequence at a level below a reference value (e.g., does not comprise the target anchor sequence), not administering the site-specific disrupting agent to the subject, e.g., administering a therapy other than the site-specific disrupting agent to the subject, e.g., administering a standard of care therapy to the subject.
   184. The method of any of embodiments 181-183, wherein the first agent and the site-specific binding agent bind to the same target anchor sequence.
   185. A method of evaluating a subject as more suitable or less suitable for treatment with a site-specific disrupting agent, comprising:
a) determining whether the subject comprises a target cancer-specific anchor sequence,
b) responsive to a determination that the subject comprises the target cancer-specific anchor sequence at a level above a reference value, identifying the subject as more suitable for treatment with the site-specific disrupting agent; or
c) responsive to a determination that the subject comprises the target cancer-specific anchor sequence at a level below a reference value (e.g., does not comprise the target cancer-specific anchor sequence), identifying the subject as less suitable for treatment with the site-specific disrupting agent.
   186. The method of embodiment 185, which comprises:
a) responsive to a determination that the subject comprises the target cancer-specific anchor sequence at a level above a reference value, administering a site-specific disrupting agent to the subject, or
b) responsive to a determination that the subject comprises the target cancer-specific anchor sequence at a level below a reference value (e.g., does not comprise the target cancer-specific anchor sequence), not administering the site-specific disrupting agent to the subject, e.g., administering a therapy other than the site-specific disrupting agent to the subject, e.g., administering a standard of care therapy to the subject.
   187. A method of treating a subject having a cancer, comprising:
a) determining whether the subject comprises a target cancer-specific anchor sequence,
b) responsive to a determination that the subject comprises the target cancer-specific anchor sequence at a level above a reference value, administering a site-specific disrupting agent to the subject; or
c) responsive to a determination that the subject comprises the target cancer-specific anchor sequence at a level below a reference value (e.g., does not comprise the target cancer-specific anchor sequence), not administering the site-specific disrupting agent to the subject, e.g., administering a therapy other than the site-specific disrupting agent to the subject, e.g., administering a standard of care therapy to the subject.
   188. The method of any of embodiments 185-187, wherein the first agent and the site-specific binding agent binds to the same target cancer-specific anchor sequence.
   189. The method of any of embodiments 181-188, wherein determining whether the subject comprises the target anchor sequence comprises:
i) obtaining or having obtained a biological sample from the subject, wherein the sample comprises a nucleic acid, and
ii) performing or having performed an assay to determine whether a first agent (e.g., a probe or a site-specific disrupting agent) binds to a target anchor sequence (e.g., target cancer-specific anchor sequence) in the nucleic acid, e.g., contacting the first agent with the biological sample and determining a level of binding of the first agent to the nucleic acid.
   190. The method of any of embodiments 181-189, wherein determining whether the subject comprises the target anchor sequence comprises:
i) obtaining or having obtained a biological sample from the subject, wherein the sample comprises a nucleic acid, and
ii) performing or having performed an assay to determine whether the target anchor sequence is present, e.g., by an assay chosen from chromosome conformation capture (3C), Hi-C, or ChIA-PET.

### BRIEF DESCRIPTION OF THE DRAWINGS

FIGS. 1A and 1B show diagrams depicting expression regulation of two exemplary genes in unaltered chromosomes (FIG. 1A) and in chromosomes that have undergone a translocation that has created a fusion gene and a Cancer Fusion Loop (CFL) (FIG. 1B). Centromeres are shown as circles. Dotted line boxes indicate independent genomic regions containing wildtype Gene_A on the first chromosome and Gene_B on the second chromosome. Enhancers are depicted as triangles, and are present within the loop of Gene_A (downstream of the gene) but are not present within the loop of Gene_B. The position of loops are indicated with arcs. FIG. 1A illustrates how in a normal cell, Gene_A is expressed because it is within a loop that comprises an enhancer, while Gene_B is silenced because it is part of a loop with no enhancer. FIG. 1B illustrates how a new CFL contains a fusion oncogene made from the downstream portion of Gene_A and the upstream portion of Gene_B; the loop also contains the enhancer from Gene_A, leading to high expression of the fusion oncogene. Thus, the chromosomal translocation leads to a malignancy.

FIGS. 2A and 2B show diagrams depicting expression regulation of an exemplary gene (e.g., HOXA9 in AML) in an unaltered chromosome (FIG. 2A) and in a chromosome that has developed a cancer-specific anchor sequence, e.g., by mutation or epigenetic alteration (FIG. 2B). Centromeres are shown as circles. Dotted line boxes indicate independent genomic regions containing the gene on the chromosome. Enhancers are depicted as triangles. The positions of loops are indicated with arcs. FIG. 2A illustrates how in a normal cell, the gene is not expressed because it is within a loop that lacks an enhancer. The loop is formed between wild-type anchor sequence 1 (upstream of the gene) and wild-type anchor sequence 2 (downstream of the gene), and the enhancer is outside of the loop and upstream of wild-type anchor sequence 1, thus preventing enhancer-promoter interaction. FIG. 2B illustrates how formation of a new cancer-specific anchor sequence forms a new loop that comprises an enhancer, leading to high expression of the gene, and malignancy. More specifically, a cancer-specific anchor sequence has formed upstream of the enhancers; the cancer-specific anchor sequence forms a loop with wild-type anchor sequence 2, so that the new loop contains the anchor sequences. The DNA that formed wild-type anchor sequence 1 in the wild-type cell is no longer in use as an anchor sequence in the cancer cell.

FIG. 3A shows a graph of CTCF ChIP-SEQ data identifying CTCF binding sites (boxes) near CCDC6 conserved across analyzed data sets based on a variety of cell types. More specifically, the genomic region shown comprises (from left to right), an upstream portion of CCDC6 (where transcription is in the leftward direction), an intergenic region, C10orf40, a second intergenic region, and a downstream region of ANK3 (where transcription is in the leftward direction). The box marked “CCDC6-B” marks a peak of CTCF-binding close to the transcriptional start site of CCDC6. The box marked “CCDC6-A” marks a peak of CTCF-binding in the downstream portion of ANK3. FIG. 3B shows an image of an ethidium bromide stained agarose gel showing DNA products of the T7E1 assay to determine whether Cas9 edited the CCDC6 proximal CTCF sites. From left to right, “NTC” (non-targeting controls) lanes 2001, tracr, and 2998 show an upper band indicating the non-edited DNA at locus CCDC6-A. “CCDC6-A” lanes 20245, 20246, 20247, 20248, and 20245+20248 show an upper and a lower band, indicating edited DNA at this locus. NTC lanes 2001, tracr, and 2998 show an upper band indicating the non-edited DNA at locus CCDC-B. “CCDC6-B” lanes 20249, 20250, 20251, 20252, 20253, 20254, 20249+20254, and 20251+20253 show an upper band and at least one lower band, indicating edited DNA at this locus. FIG. 3C (72 h CCDC6-RET LC2/ad) shows a graph of CCDC6-RET expression determined by RT-PCR analysis of CCDC6-RET cDNA. BR A and BR B indicate two different biological replicates. The X axis indicates the gRNA used to treat the cells: NTC (2001, tracr, and 2998), CCDC6-A (20245, 20246, 20247, 20248, and 20245+20248), and CCDC6-B (20249, 20250, 20251, 20252, 20253, 20254, 20249+20254, and 20251+20253). The left Y axis indicates the ddCt (Log2 Fold Change) in expression of CCDC6-RET mRNA. The right Y axis indicates the % mRNA (level of CCDC6-RET mRNA relative to the control). NTC controls define the baseline used for normalization. Most of the CCDC6-A and CCDC-B samples show a decrease in mRNA levels, with at least 20253, 20254, 20249+20254, and 20251+20253 showing mRNA levels between about −1.0 and −0.5 ddCt.

FIG. 4A shows a graph of CTCF ChIP-SEQ data identifying a CTCF binding site (boxed and marked “PAX3-D”) in PAX3 that is not detected in analyzed data sets based on a variety of cell types (“conserved”) but is present in RH30 cells (“RH30-specific”). Below, vertical lines indicate putative CTCF binding sites based on DNA sequence. More specifically, the genomic region shown comprises (from left to right) an upstream portion of PAX3 (where transcription is in the leftward direction) and an intergenic region. The box marked “PAX3-D” marks a peak of CTCF-binding observed in the transcribed region of PAX3 in RH30 cells but not in the “conserved” data set. Another peak of CTCF-binding observed in the transcribed region of PAX3 in RH30 cells but not in the “conserved” data set is positioned close to the transcriptional start site of PAX3. Other CTCF-binding peaks present in both RH30 cells and the “conserved” data set are on the far right of the figure. CTCF consensus sequences are observed below the PAX3-D peak and several other locations. FIG. 4B shows an image of an ethidium bromide stained agarose gel showing DNA products of the T7E1 assay to determine whether Cas9 edited the PAX3-FOXO1 unique CTCF site. From left to right, “NTC” (non-targeting controls) lanes 2001, tracr, and 2998 show an upper band indicating the non-edited DNA at locus PAX3-D. “PAX3-D” lanes 25924, 25925, 25926, 25927, 25928, 25924+25928, and 25925+25926+25927 show an upper band and at least one lower band, indicating edited DNA at this locus. FIG. 4C (72h PAX3-FOXO1 RH30 PAX3-D CTCF) shows a graph of PAX3-FOXO1 expression determined by RT-PCR analysis of PAX3-FOXO1 cDNA. BR A and BR B indicate two different biological replicates. The X axis indicates the gRNA used to treat the cells: NTC (2001, tracr, and 2998) and PAX3-D (25924, 25925, 25926, 25927, 25928, 25924+25928, and 25925+25926+25927). The left Y axis indicates the ddCt (Log2 Fold Change) in expression of PAX3-FOXO1 mRNA. The right Y axis indicates the % mRNA (level of PAX3-FOXO1 mRNA relative to the control). NTC controls define the baseline used for normalization. The PAX3-D samples show mRNA levels between about −1.0 and −0.5 ddCt.

FIG. 5A (96h PAX3-FOXO1 RH30 PAX3-D) shows a graph of PAX3-FOXO1 expression (evaluated using real-time PCR from cDNA produced from extracted RNA) in rhabdomyosarcoma cells expressing Cas9 96 hours post-transfection with either control gRNA or gRNA targeting the PAX3-FOXO1 proximal CTCF anchor site. The X axis indicates the gRNA used to treat the cells: NTC (2998) and PAX3-D (25924, 25925, 25926, 25927, and 25928). The left Y axis indicates the ddCt (Log2 Fold Change) in expression of PAX3-FOXO1 mRNA. The right Y axis indicates the % mRNA (level of PAX3-FOXO1 mRNA relative to the control). NTC controls define the baseline used for normalization. The PAX3-D samples show mRNA levels between about −1.5 and −0.5 ddCt. FIG. 5B shows a graph of cell proliferation over time (CellTiter-Glo Assay (Promega)) of rhabdomyosarcoma cells expressing Cas9 and transfected with either control gRNA or gRNA targeting the PAX3-FOXO1 proximal CTCF anchor site for the gRNAs shown in FIG. 5A. The X axis indicates time from 0 to 10 days. The Y axis indicates relative luciferase signal as a measure of cell proliferation, where the cells have a signal of 1 at day 0. While the control cells have a signal of between about 12 and 14 after 10 days, the PAX3-D samples have a signal between about 4 and 10 (e.g., between about 4 and 6 for sample 25928) showing an impairment of cell proliferation. FIG. 5C (d10 CellTiler-Glo RH30-Cas9 PAX3-D) shows a graph of viable cell count (CellTiter-Glo Assay (Promega)) ten days after transfection with either control gRNA or gRNA targeting the PAX3-FOXO1 proximal CTCF anchor site of rhabdomyosarcoma cells expressing Cas9 for the gRNAs shown in FIG. 5A. The X axis indicates the PAX3-D CTCF-targeting gRNA or NTC gRNA used. The Y axis indicates relative luciferase signal as a measure of viable cell count. While control cells have a baseline luciferase signal of 1.0, indicating normal viability, the PAX3-D samples have a signal between about 0.4 and 0.7, indicating impaired viability.

FIG. 6 is an illustration of exemplary types of anchor sequence-mediated conjunctions as described herein.

### DEFINITIONS

Agent: As used herein, the term “agent”, may be used to refer to a compound or entity of any chemical class including, for example, a polypeptide, nucleic acid, saccharide, lipid, small molecule, metal, or combination or complex thereof. As will be clear from context to those skilled in the art, in some embodiments, the term may be utilized to refer to an entity that is or comprises a cell or organism, or a fraction, extract, or component thereof. Alternatively or additionally, as those skilled in the art will understand in light of context, in some embodiments, the term may be used to refer to a natural product in that it is found in and/or is obtained from nature. In some embodiments, again as will be understood by those skilled in the art in light of context, the term may be used to refer to one or more entities that is man-made in that it is designed, engineered, and/or produced through action of the hand of man and/or is not found in nature. In some embodiments, an agent may be utilized in isolated or pure form; in some embodiments, an agent may be utilized in crude form. In some embodiments, potential agents may be provided as collections or libraries, for example that may be screened to identify or characterize active agents within them. In some embodiments, the term “agent” may refer to a compound or entity that is or comprises a polymer; in some embodiments, the term may refer to a compound or entity that comprises one or more polymeric moieties. In some embodiments, the term “agent” may refer to a compound or entity that is not a polymer and/or is substantially free of any polymer and/or of one or more particular polymeric moieties. In some embodiments, the term may refer to a compound or entity that lacks or is substantially free of any polymeric moiety.

Altered: As used herein, the term “altered” refers to a detectable difference (e.g., in level, frequency, structure, activity, etc.) of an entity when assessed, for example, across a population in which the entity can be observed, at different time points and/or under different conditions.

Anchor Sequence: The term “anchor sequence” as used herein, refers to a nucleic acid sequence recognized by a conjunction agent (e.g., a nucleating polypeptide) that binds sufficiently to form an anchor sequence-mediated conjunction, e.g., a loop. In some embodiments, an anchor sequence comprises one or more CTCF binding motifs. In some embodiments, an anchor sequence is not located within a gene coding region. In some embodiments, an anchor sequence is located within an intergenic region. In some embodiments, an anchor sequence is not located within either of an enhancer or a promoter. In some embodiments, an anchor sequence is located at least 400 bp, at least 450 bp, at least 500 bp, at least 550 bp, at least 600 bp, at least 650 bp, at least 700 bp, at least 750 bp, at least 800 bp, at least 850 bp, at least 900 bp, at least 950 bp, or at least 1 kb away from any transcription start site. In some embodiments, an anchor sequence is located within a region that is not associated with genomic imprinting, monoallelic expression, and/or monoallelic epigenetic marks. In some embodiments, the anchor sequence has one or more functions selected from binding an endogenous nucleating polypeptide (e.g., CTCF), interacting with a second anchor sequence to form an anchor sequence mediated conjunction (e.g., loop), or insulating against an enhancer that is outside the anchor sequence mediated conjunction. In some embodiments of the present disclosure, technologies are provided that may specifically target a particular anchor sequence or anchor sequences, without targeting other anchor sequences (e.g., sequences that may contain a nucleating polypeptide (e.g., CTCF) binding motif in a different context); such targeted anchor sequences may be referred to as the “target anchor sequence”. In some embodiments, sequence and/or activity of a target anchor sequence is modulated while sequence and/or activity of one or more other anchor sequences that may be present in the same system (e.g., in the same cell and/or in some embodiments on the same nucleic acid molecule—e.g., the same chromosome) as the targeted anchor sequence is not modulated. In some embodiments, the anchor sequence comprises or is a nucleating polypeptide binding motif. In some embodiments, the anchor sequence is adjacent to a nucleating polypeptide binding motif.

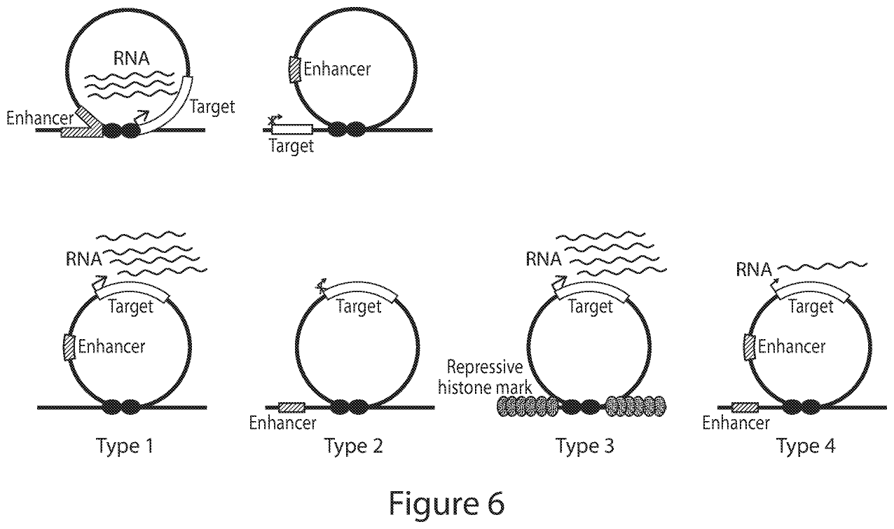

Anchor sequence-mediated conjunction: The term “anchor sequence-mediated conjunction” as used herein (also abbreviated ASMC), refers to a DNA structure, in some cases, a loop, that occurs and/or is maintained via physical interaction or binding of at least two anchor sequences in the DNA by one or more polypeptides, such as nucleating polypeptides, or one or more proteins and/or a nucleic acid entity (such as RNA or DNA), that bind the anchor sequences to enable spatial proximity and functional linkage between the anchor sequences (see, e.g. FIG. 6). In some embodiments, the loop (also referred to herein as a “cancer fusion loop” or “CFL”) is found in a cancer cell, but not in a wild-type or non-cancerous cell from the same cell type as the cancer cell. The CFL can comprises a breakpoint, e.g., as described herein.

Associated with: Two events or entities are “associated” with one another, as that term is used herein, if presence, level, form and/or function of one is correlated with that of the other. For example, in some embodiments, a particular entity (e.g., polypeptide, genetic signature, metabolite, microbe, etc.) is considered to be associated with a particular disease, disorder, or condition, if its presence, level, form and/or function correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof. In some embodiments, a DNA sequence is “associated with” a target genomic complex when the nucleic acid is at least partially within the target genomic complex, and expression of a gene in the DNA sequence is affected by formation or disruption of the target genomic complex.

Breakpoint: As used herein, the term “breakpoint” refers to a site in a chromosome that is different from the corresponding site in a wild-type chromosome as a result of a break in a chromosome. In embodiments, the breakpoint is a site that underwent a gross chromosomal rearrangement (e.g., in the chromosome itself, or in a parent chromosome that subsequently underwent replication). In some embodiments, the breakpoint is a covalent bond connecting a first nucleotide that is part of a first chromosomal region to a second nucleotide that is part of a second chromosomal region, wherein the first and second chromosomal regions are not typically contiguous with each other in a wild-type cell and/or in the Genome Reference Consortium human genome (build 38). In some embodiments, the breakpoint is a break in a chromosome that has not rejoined with another chromosomal region.

Cancer-specific anchor sequence: As used herein, the term “cancer-specific anchor sequence” refers to a nucleic acid sequence recognized by a conjunction agent (e.g., a nucleating polypeptide) that binds sufficiently to form an anchor sequence-mediated conjunction, e.g., a loop, in a cancer cell, but not in a non-cancerous cell of the tissue from which the cancer originated. In some embodiments, a corresponding non-cancerous cell comprises the DNA sequence of the cancer-specific anchor sequence, but that DNA does not form an anchor sequence-mediated conjunction. In some embodiments, technologies are provided that may specifically target a particular cancer-specific anchor sequence or sequences, without targeting other anchor sequences (e.g., other cancer-specific anchor sequences), such a targeted cancer-specific anchor sequence may be referred to as a “target cancer-specific anchor sequence”.

Cluster: As used herein, the term “cluster” refers to a population (e.g., sequence motifs, e.g., cells) that are positioned or are occurring in physical proximity to one another. In some embodiments, sequence motifs in a cluster are within a set distance of one another. In some embodiments, cells in a cluster are adhered to one another, so that the cluster is stable to one or more conditions that would separate non-adherent cells from one another (e.g., mild turbulence, such as by gentle shaking), etc. In some embodiments, a cluster is stable (e.g., remains detectable) over a period of time. In some embodiments, a cluster is observed in a population of cells that is not in liquid culture; in some such embodiments, stability of a particular cluster may be reflected in detection of a cluster at or near a particular physical location over a period of time (e.g., at multiple points in time).

Domain: As used herein, the term “domain” refers to a section or portion of an entity. In some embodiments, a “domain” is associated with a particular structural and/or functional feature of the entity so that, when the domain is physically separated from the rest of its parent entity, it substantially or entirely retains the particular structural and/or functional feature. Alternatively or additionally, in some embodiments, a domain may be or include a portion of an entity that, when separated from that (parent) entity and linked with a different (recipient) entity, substantially retains and/or imparts on the recipient entity one or more structural and/or functional features that characterized it in the parent entity. In some embodiments, a domain is or comprises a section or portion of a molecule (e.g., a small molecule, carbohydrate, lipid, nucleic acid, polypeptide, etc.). In some embodiments, a domain is or comprises a section of a polypeptide. In some such embodiments, a domain is characterized by a particular structural element (e.g., a particular amino acid sequence or sequence motif, alpha-helix character, beta-sheet character, coiled-coil character, random coil character, etc.), and/or by a particular functional feature (e.g., binding activity, enzymatic activity, folding activity, signaling activity, etc.).

Engineered: As used herein, the term “engineered” generally refers to the aspect of having been manipulated by the hand of man. For example, in some embodiments, a polynucleotide is considered to be “engineered” when two or more sequences, that are not linked together in that order in nature, are manipulated by human activity to be directly linked to one another in the engineered polynucleotide. For example, in some embodiments, an engineered polynucleotide comprises a regulatory sequence that is found in nature in operative association with a first coding sequence but not in operative association with a second coding sequence, is linked by human activity so that it is operatively associated with the second coding sequence. Comparably, a cell or organism is considered to be “engineered” if it has been manipulated so that its genetic information is altered (e.g., new genetic material not previously present has been introduced, for example by transformation, mating, somatic hybridization, transfection, transduction, or other mechanism, or previously present genetic material is altered or removed, for example by substitution or deletion mutation, and/or by mating protocols). As is common practice and is understood by those in the art, progeny of an engineered polynucleotide or cell are typically still referred to as “engineered” even though the actual manipulation was performed on a prior entity.

eRNA: As used herein, the term “eRNA” refers to an enhancer RNA, which those skilled in the art will be aware is a type of non-coding RNA that may be transcribed from an enhancer. eRNAs, in some embodiments, may participate in transcription and/or other expression of one or more genes regulated by that enhancer. In some embodiments, eRNAs are involved in forming and/or stabilizing anchor sequence-mediated conjunctions (e.g., genomic loops). In some embodiments, eRNAs are involved in forming anchor sequence-mediated conjunctions between a given enhancer and a given target gene promoter. In some embodiments, eRNAs are inside an anchor sequence-mediated conjunction. In some embodiments, eRNAs are outside of an anchor sequence-mediated conjunction. In some embodiments, eRNAs are part of a genomic complex as described herein. In some embodiments, an eRNA may interact specifically with one or more proteins, for example selected from the group consisting of: anchor sequence nucleating polypeptides such as CTCF and YY1, general transcription machinery components, any protein known to be enriched in or near enhancers (e.g. Mediator, p300, etc.), one or more transcriptional regulators (e.g., enhancer-binding proteins) such as p53, Oct4, etc. In some embodiments, changes in levels of one or more eRNAs may correlate with and/or result in changes of levels of expression of a particular target gene. In some embodiments, for example, knockdown of an eRNA may correlate with and/or cause knockdown of a target gene.

Fusion gene: As used herein, “fusion gene” refers to a gene that comprises a breakpoint between two or more nucleic acid sequences that are operably linked and are normally non-contiguous (e.g., in wild-type and/or non-disease cells, e.g., in the absence of or prior to a gross chromosomal rearrangement). In some embodiments, a fusion gene is produced by a gross chromosomal rearrangement. In some embodiments, a fusion gene comprises a first protein encoding nucleic acid sequence and a second protein encoding nucleic acid sequence or fragments thereof, e.g., a first gene and a second gene or fragments thereof, e.g., that are not normally found in wild-type and/or non-disease cells. In some embodiments, a fusion gene comprises a first protein encoding nucleic acid sequence or fragment thereof (e.g., a gene or a fragment thereof) and a second nucleic acid sequence that does not normally (e.g., in wild-type and/or non-disease cells) encode for a protein. In some embodiments, a fusion gene comprises an enhancer that was proximal or associated with a first gene and a protein encoding sequence of another gene.

Genomic complex: As used herein, the term “genomic complex” is a complex that brings together two genomic sequence elements that are spaced apart from one another on one or more chromosomes, via interactions between and among a plurality of protein and/or other components (potentially including, the genomic sequence elements). In some embodiments, the genomic sequence elements are anchor sequences to which one or more protein components of the complex binds. In some embodiments, a genomic complex may comprise an anchor sequence-mediated conjunction. In some embodiments, a genomic sequence element may be or comprise a CTCF binding motif, a promoter and/or an enhancer. In some embodiments, a genomic sequence element includes at least one or both of a promoter and/or regulatory site (e.g., an enhancer). In some embodiments, complex formation is nucleated at the genomic sequence element(s) and/or by binding of one or more of the protein component(s) to the genomic sequence element(s). As will be understood by those skilled in the art, in some embodiments, co-localization (e.g., conjunction) of the genomic sites via formation of the complex alters DNA topology at or near the genomic sequence element(s), including, in some embodiments, between them. In some embodiments, a genomic complex comprises an anchor sequence-mediated conjunction, which comprises one or more loops. In some embodiments, a genomic complex as described herein is nucleated by a nucleating polypeptide such as, for example, CTCF and/or Cohesin. In some embodiments, a genomic complex as described herein may include, for example, one or more of CTCF, Cohesin, non-coding RNA, enhancer RNA, transcriptional machinery proteins (e.g., RNA polymerase, one or more transcription factors, for example selected from the group consisting of TFIIA, TFIIB, TFIID, TFIIE, TFIIF, TFIIH, etc.), transcriptional regulators (e.g., Mediator, P300, enhancer-binding proteins, repressor-binding proteins, histone modifiers, etc.), etc. In some embodiments, a genomic complex as described herein includes one or more polypeptide components and/or one or more nucleic acid components (e.g., one or more RNA components), which may, in some embodiments, be interacting with one another and/or with one or more genomic sequence elements (e.g., anchor sequences, promoter sequences, regulatory sequences) so as to constrain a stretch of genomic DNA into a topological configuration (e.g., a loop) that it does not adopt when the complex is not formed. In some embodiments, the genomic complex (also referred to herein as a “cancer—specific genomic complex”) is found in a cancer cell, but not in a wild-type or non-cancerous cell from the same cell type as the cancer cell.

“Gross chromosomal rearrangement”: As used herein, this term refers to an event comprising a break at a site in a chromosome, which is optionally rejoined to a different chromosomal region that is not typically contiguous with the site in a wild-type cell. In some embodiments, the site is not contiguous with the different chromosomal region in the Genome Reference Consortium human genome (build 38). Exemplary gross chromosomal rearrangements include, but are not limited to, translocations, inversions, deletions (e.g., interstitial deletion or terminal deletion), insertions, amplifications (e.g., duplications), e.g., a tandem amplification or tandem duplication, chromosome end-to-end fusions, chromothripsis, or any combination thereof. In some embodiments, the deletion is a microdeletion or a larger deletion.

“Improved,” “increased” or “reduced”: As used herein, these terms, or grammatically comparable comparative terms, indicate values that are relative to a comparable reference measurement. For example, in some embodiments, an assessed value achieved with an agent of interest may be “improved” relative to that obtained with a comparable reference agent. Alternatively or additionally, in some embodiments, an assessed value achieved in a subject or system of interest may be “improved” relative to that obtained in the same subject or system under different conditions (e.g., prior to or after an event such as administration of an agent of interest), or in a different, comparable subject (e.g., in a comparable subject or system that differs from the subject or system of interest in presence of one or more indicators of a particular disease, disorder or condition of interest, or in prior exposure to a condition or agent, etc.). In some embodiments, comparative terms refer to statistically relevant differences (e.g., that are of a prevalence and/or magnitude sufficient to achieve statistical relevance). Those skilled in the art will be aware, or will readily be able to determine, in a given context, a degree and/or prevalence of difference that is required or sufficient to achieve such statistical significance.

Loop: The term “loop” (e.g., genomic loop), as used herein, refers to a type of chromatin structure that may be created by co-localization of two or more anchor sequences as an anchor sequence-mediated conjunction. Thus, a genomic loop is formed as a consequence of the interaction of at least two anchor sequences in DNA with one or more proteins, such as nucleating polypeptides, or one or more proteins and/or a nucleic acid entity (such as RNA or DNA), that bind the anchor sequences to enable spatial proximity and functional linkage between the anchor sequences. Those skilled in the art, reading the present specification, will appreciate that a 2D representation of such a structure may be presented as a loop. An “activating loop” is a structure that is open to active gene transcription, for example, a structure comprising a transcription control sequence (enhancing sequence) that enhances transcription. In some embodiments, a loop may be a “repressor loop”, wherein such a loop has a structure that is closed off from active gene transcription, for example, a structure comprising a transcription control sequence (silencing sequence) that represses transcription. In some embodiments, a loop comprises an active gene, wherein an enhancer is inside a given loop and/or repressor is outside the loop. In some embodiments, a loop comprises an inactive gene, wherein a repressor is inside a given loop and/or an enhancer is outside the loop.

Moiety: As used herein, the term a “moiety” refers to a defined chemical group or entity with a particular structure and/or or activity, as described herein.

Nucleating polypeptide: As used herein, the term “nucleating polypeptide” or “conjunction nucleating polypeptide” as used herein, refers to a protein that associates with an anchor sequence directly or indirectly and may interact with one or more conjunction nucleating polypeptides (that may interact with an anchor sequence or other nucleic acids) to form a dimer (or higher order structure) comprised of two or more such conjunction nucleating polypeptides, which may or may not be identical to one another. When conjunction nucleating polypeptides associated with different anchor sequences associate with each other so that the different anchor sequences are maintained in physical proximity with one another, the structure generated thereby is an anchor-sequence-mediated conjunction. That is, the close physical proximity of a nucleating polypeptide-anchor sequence interacting with another nucleating polypeptide-anchor sequence generates an anchor sequence-mediated conjunction (e.g., in some cases, a DNA loop), that begins and ends at the anchor sequence. As those skilled in the art, reading the present specification will immediately appreciate, terms such as “nucleating polypeptide”, “nucleating molecule”, “nucleating protein”, “conjunction nucleating protein”, may sometimes be used to refer to a conjunction nucleating polypeptide. As will similarly be immediately appreciated by those skilled in the art reading the present specification, an assembled collection of two or more conjunction nucleating polypeptides (which may, in some embodiments, include multiple copies of the same agent and/or in some embodiments one or more of each of a plurality of different agents) may be referred to as a “complex”, a “dimer” a “multimer”, etc.

Nucleating polypeptide binding motif: As used herein, the term “nucleating polypeptide binding motif” as used herein, refers to a nucleating polypeptide binding motif in an anchor sequence. Examples of anchor sequences include, but are not limited to, CTCF binding motifs, USF1 binding motifs, YY1 binding motifs, TAF3 binding motifs, and ZNF143 binding motifs.

Operably Linked: As used herein, the term “operably linked” describes a relationship between a first nucleic acid sequence and a second nucleic acid sequence wherein the first nucleic acid sequence can affect the second nucleic acid sequence, e.g., by being co-expressed together, e.g., as a fusion gene, and/or by affecting transcription, epigenetic modification, and/or chromosomal topology. In some embodiments, operably linked means two nucleic acid sequences are comprised on the same nucleic acid molecule. In a further embodiment, operably linked may further mean that the two nucleic acid sequences are proximal to one another on the same nucleic acid molecule, e.g., within 1000, 500, 100, 50, or 10 base pairs of each other or directly adjacent to each other. In an embodiment, a promoter or enhancer sequence that is operably linked to a sequence encoding a protein can promote the transcription of the sequence encoding a protein, e.g., in a cell or cell free system capable of performing transcription. In an embodiment, a first nucleic acid sequence encoding a protein or fragment of a protein that is operably linked to a second nucleic acid sequence encoding a second protein or second fragment of a protein are expressed together, e.g., the first and second nucleic acid sequences comprise a fusion gene and are transcribed and translated together to produce a fusion protein. In an embodiment, a first nucleic acid sequence and a second nucleic acid sequence that are operably linked have common characteristics, e.g., transcription, epigenetic, and/or chromosomal topology characteristics, e.g., of the first or the second nucleic acid sequence and/or of the genomic locus of the first or the second nucleic acid sequence. For example, in some embodiments, a gross chromosomal rearrangement operably links a first nucleic acid sequence and a second nucleic acid sequence, and the operably linked first and second nucleic acid sequence has one or more characteristic of the first nucleic acid sequence and/or the genomic locus of the first nucleic acid sequence (e.g., transcription, epigenetic, and/or chromosomal topology characteristics). In another example, in some embodiments, a gross chromosomal rearrangement operably links a first nucleic acid sequence and a second nucleic acid sequence, and the operably linked first and second nucleic acid sequence has one or more characteristic of the second nucleic acid sequence and/or the genomic locus of the second nucleic acid sequence (e.g., transcription, epigenetic, and/or chromosomal topology characteristics).

Oncogene: As used herein, an oncogene is an allele of a gene, wherein the allele is capable of causing or promoting cancer (e.g., causing or promoting a cancerous cell state, e.g., characterized by dysregulated growth, division, and/or invasion) under appropriate physiological and/or cellular conditions. Many oncogenes are known to those skilled in the art and some oncogenes are known to be associated with particular types of cancers or cell types. A fusion oncogene is a fusion gene that is capable of causing or promoting cancer (e.g., causing or promoting a cancerous cell state, e.g., characterized by dysregulated growth, division, and/or invasion) under appropriate physiological and/or cellular conditions. A number of fusion oncogenes are known to those skilled in the art and some fusion oncogenes are known to be associated with particular types of cancers or cell types.

Pharmaceutical composition: As used herein, the term “pharmaceutical composition” refers to an active agent, e.g., disrupting agent, formulated together with one or more pharmaceutically acceptable carriers. In some embodiments, active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and/or to other mucosal surfaces.

Proximal: As used herein, the term “proximal”, when used with respect to two or more nucleic acid sites, refers to the sites being sufficiently close on a nucleic acid (e.g., a chromosome), e.g., in nucleotide distance and/or three-dimensional structure, such that a modification to one can affect the other. For instance, in some embodiments, an anchor site is proximal to a gene if a modification to the anchor sequence results in a change in expression of the gene. In some embodiments, a breakpoint is proximal to a gene (e.g., fusion oncogene) if formation of the breakpoint led to a change in expression (e.g., increased expression) of the gene, e.g., relative to one of the wild-type genes prior to fusion. In embodiments, the proximity between the sites (e.g., breakpoint and the anchor sequence, and/or the breakpoint and the gene) is less than 10 kb, 20 kb, 30 kb, 40 kb, 50 kb, 60 kb, 70 kb, 80 kb, 90 kb, 100 kb, 500 kb, 1 Mb, 1.5 Mb, 2 Mb, 2.5 Mb, or 3 Mb. In some embodiments, a breakpoint is proximal to a gene if the gene comprises the breakpoint (e.g., when the gene is a fusion gene).

Disrupting agent: As used herein, the term “disrupting agent” (also referred to as “site-specific disrupting agent”) refers to an agent or entity that specifically inhibits, dissociates, degrades, and/or modifies one or more components of a genomic complex as described herein. In some embodiments, a disrupting agent interacts with one or more components of a genomic complex. In some embodiments, a disrupting agent binds (e.g., directly or, in some embodiments, indirectly) to one or more genomic complex components. In some embodiments, a disrupting agent modifies one or more genomic complex components. In some embodiments, a disrupting agent is or comprises an oligonucleotide. In some embodiments, a disrupting agent is or comprises a polypeptide. In some embodiments, a disrupting agent is or comprises an antibody (e.g., a monospecific or multispecific antibody construct) or antibody fragment. In some embodiments, a disrupting agent is directed to a particular genomic location and/or to a genomic complex by a targeting agent, as described herein. In some embodiments, a disrupting agent comprises a genomic complex component or variant thereof. In some embodiments, a disrupting agent is or comprises a disrupting moiety. In some embodiments, a disrupting agent is or comprises a modifying moiety. In some embodiments, a disrupting agent is or comprises one or more effector moieties (e.g., disrupting moieties, modifying moieties, and/or other effector moieties). In some embodiments, the site-specific disrupting agent specifically binds a first site in the genome with higher affinity than a second site in the genome (e.g., relative to any other site in the genome). In some embodiments, the site-specific disrupting agent preferentially inhibits, dissociates, degrades, and/or modifies one or more components of a first genomic complex relative to a second genomic complex (e.g., relative to any other genomic complex).

Sequence targeting polypeptide: As used herein, the term “sequence targeting polypeptide” as used herein, refers to a protein, such as an enzyme, e.g., Cas9, that recognizes or specifically binds to a target sequence. In some embodiments, the sequence targeting polypeptide is a catalytically inactive protein, such as dCas9, that lacks endonuclease activity.

Specific: As used herein, the term “specific” refers to an agent having an activity, is understood by those skilled in the art to mean that the agent discriminates between potential target entities or states. For example, an in some embodiments, an agent is said to bind “specifically” to its target or be “site-specific” if it binds preferentially with that target in the presence of one or more competing alternative targets. In some embodiments, specific interaction is dependent upon the presence of a particular structural feature of the target entity (e.g., an epitope, a cleft, a binding motif). It is to be understood that specificity need not be absolute. In some embodiments, specificity may be evaluated relative to that of the binding agent for one or more other potential target entities (e.g., competitors). In some embodiments, specificity is evaluated relative to that of a reference specific binding agent. In some embodiments specificity is evaluated relative to that of a reference non-specific binding agent. In some embodiments, the agent or entity does not detectably bind to the competing alternative target under conditions of binding to its target entity. In some embodiments, the agent binds with higher on-rate, lower off-rate, increased affinity, decreased dissociation, and/or increased stability to its target entity as compared with the competing alternative target(s).

Subject: As used herein, the term “subject” or “test subject” refers to any organism to which a provided compound or composition is administered in accordance with the present disclosure e.g., for experimental, diagnostic, prophylactic, and/or therapeutic purposes. Typical subjects include animals (e.g., mammals such as mice, rats, rabbits, non-human primates, and humans; insects; worms; etc.) and plants. In some embodiments, a subject may be suffering from, and/or susceptible to a disease, disorder, and/or condition.

Substantially: As used herein, the term “substantially” refers to the qualitative condition of exhibiting total or near-total extent or degree of a characteristic or property of interest. One of ordinary skill in the art will understand that biological and chemical phenomena rarely, if ever, go to completion and/or proceed to completeness or achieve or avoid an absolute result. The term “substantially” may therefore be used in some embodiments herein to capture potential lack of completeness inherent in many biological and chemical phenomena.

Target: An agent or entity is considered to “target” another agent or entity, in accordance with the present disclosure, if it binds specifically to the targeted agent or entity under conditions in which they come into contact with one another. In some embodiments, a nucleic acid having a particular sequence targets a nucleic acid of substantially complementary sequence. In some embodiments, target binding is direct binding; in some embodiments, target binding may be indirect binding.

Target gene: As used herein, the term “target gene” means a gene that is targeted for modulation. In some embodiments, the target gene is proximal to a breakpoint and a target anchor sequence, e.g., a cancer-specific target anchor sequence. In some embodiments, the target gene comprises a breakpoint and/or a target anchor sequence, e.g., a cancer-specific target anchor sequence. In some embodiments, the target gene is an oncogene, e.g., a fusion oncogene. In some embodiments, a target gene is part of a targeted genomic complex (e.g., a gene that has at least part of its genomic sequence as part of a target genomic complex, e.g., inside an anchor sequence-mediated conjunction), which genomic complex is inhibited, dissociated, and/or destabilized by one or more disrupting agents as described herein. In some embodiments, a target gene is modulated by a genomic sequence of a target gene being directly contacted by a disrupting agent as described herein. In some embodiments, a target gene is outside of a target genomic complex, for example, a gene that encodes a component of a target genomic complex (e.g., a subunit of a transcription factor). In some embodiments, the target gene encodes a protein. In some embodiments, the target gene encodes a functional RNA.

Targeting moiety: As used herein, the term “targeting moiety” means an agent or entity that specifically interacts (i.e., targets) with a component or set of components, e.g., a component or components that participate in a genomic complex as described herein (e.g., comprising an anchor sequence-mediated conjunction). In some embodiments, a targeting moiety in accordance with the present disclosure targets one or more target component(s) of a genomic complex as described herein. In some embodiments, a targeting moiety targets a genomic complex component that comprises a genomic sequence element (e.g., an anchor sequence element). In some embodiments, a targeting moiety targets a genomic complex component other than a genomic sequence element. In some embodiments, a targeting moiety targets a plurality or combination of genomic complex components, which plurality in some embodiments may include a genomic sequence element. In some aspects, contributions of the present disclosure include the insight that inhibition, dissociation, degradation, and/or modification of one or more genomic complexes, e.g., comprising a target anchor sequence proximal to a target gene (e.g., fusion gene, e.g., fusion oncogene) and/or breakpoint, as described herein, can be achieved by targeting genomic complex component(s), including genomic sequence element(s), with disrupting agents, e.g., site-specific disrupting agents. In some aspects, effective inhibition, dissociation, degradation, and/or modification of one or more genomic complexes, as described herein, can be achieved by targeting complex component(s) comprising genomic sequence element(s). In some embodiments, the present disclosure contemplates that improved (e.g., with respect to, for example, degree of specificity for a particular genomic complex as compared with other genomic complexes that may form or be present in a given system, effectiveness of the inhibition, dissociation, degradation, or modification [e.g., in terms of impact on number of complexes detected in a population]) inhibition, dissociation, degradation, or modification may be achieved by targeting one or more complex components that is not a genomic sequence element and, optionally, may alternatively or additionally include targeting a genomic sequence element, wherein improved inhibition, dissociation, degradation, or modification is relative to that typically achieved through targeting genomic sequence element(s) alone. In some embodiments, a disrupting agent as described herein promotes inhibition, dissociation, degradation, or modification of a target genomic complex. For example, by way of non-limiting example, in some embodiments, a disrupting agent as described herein inhibits, dissociates, degrades (e.g., a component of), and/or modifies (e.g., a component of) an anchor sequence-mediated conjunction by targeting at least one component of a given genomic complex (e.g., comprising the anchor sequence-mediated conjunction). In some embodiments, a disrupting agent as described herein inhibits, dissociates, degrades (e.g., a component of), and/or modifies (e.g., a component of) a particular genomic complex (i.e., a target genomic complex) and does not inhibit, dissociate, degrade (e.g., a component of), and/or modify (e.g., a component of) at least one other particular genomic complex (i.e., a non-target genomic complex) that, for example, may be present in other cells (e.g., in non-target cells) and/or that may be present at a different site in the same cell (i.e., within a target cell). A site-specific disrupting agent as described herein includes a targeting moiety. In some embodiments, a targeting moiety also acts as an effector moiety (e.g. disrupting moiety); in some such embodiments a provided site-specific disrupting agent may lack any effector moiety (e.g. disrupting, modifying, or other effector moiety) separate (or meaningfully distinct) from the targeting moiety.

Therapeutically effective amount: As used herein, the term “therapeutically effective amount” means an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, an effective amount of a substance may vary depending on such factors as desired biological endpoint(s), substance to be delivered, target cell(s) or tissue(s), etc. For example, in some embodiments, an effective amount of compound in a formulation to treat a disease, disorder, and/or condition is an amount that alleviates, ameliorates, relieves, inhibits, prevents, delays onset of, reduces severity of and/or reduces incidence (e.g., frequency, extent, etc.) of one or more symptoms or features of the disease, disorder, and/or condition. In some embodiments, a therapeutically effective amount is administered in a single dose; in some embodiments, multiple unit doses are required to deliver a therapeutically effective amount.

Transcriptional control sequence: As used herein, the term “transcriptional control sequence” as used herein, refers to a nucleic acid sequence that increases or decreases transcription of a gene. An “enhancing sequence” increases the likelihood of gene transcription. A “silencing or repressor sequence” decreases the likelihood of gene transcription.

### DETAILED DESCRIPTION OF CERTAIN EMBODIMENTS

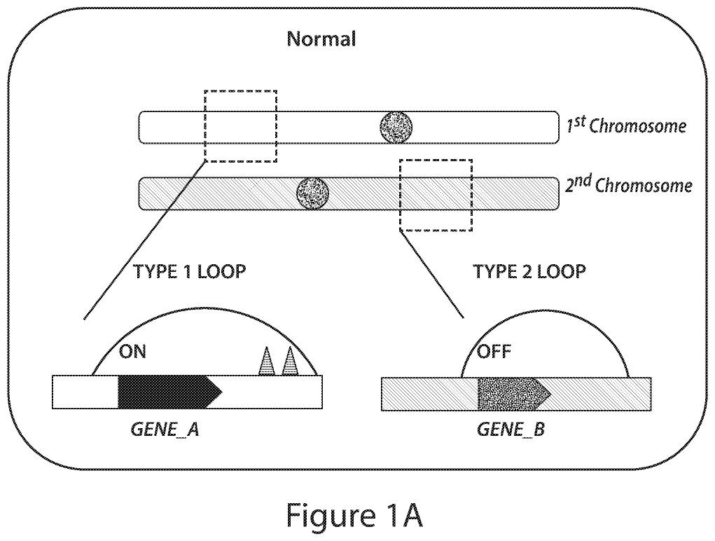

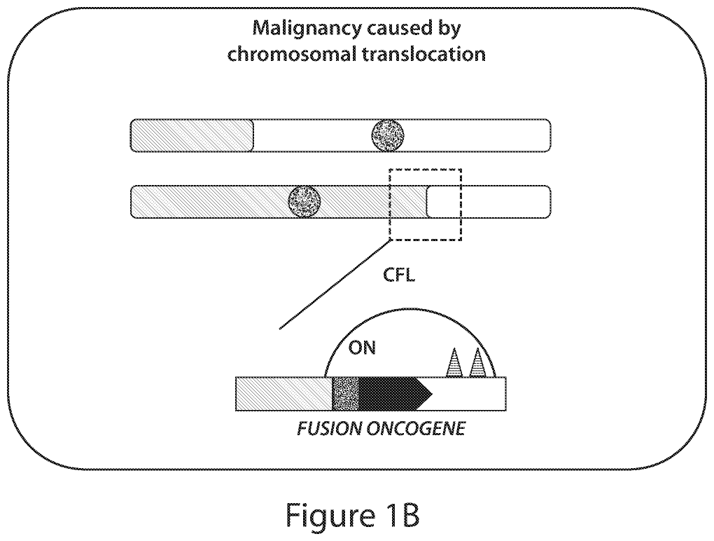

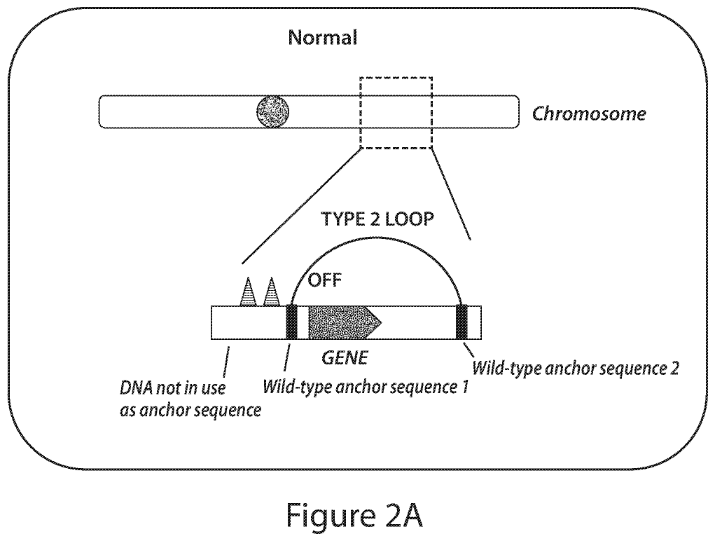

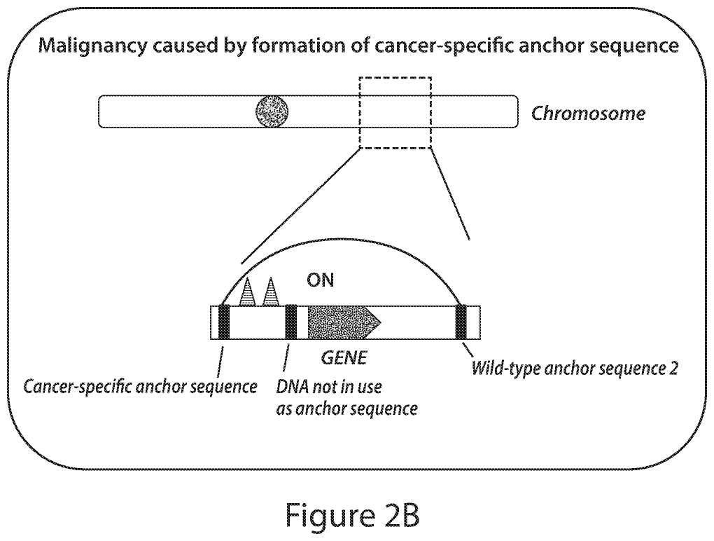

Many diseases are associated with chromosomal rearrangements that create fusion genes proximal to or comprising breakpoints. For example, cancer-associated chromosomal rearrangements, e.g., translocations, are highly recurrent for particular cancer types. These translocations frequently fuse parts of two normally independent genes (FIG. 1A), creating a fusion gene that functions as an oncogene that drives malignant behavior of the tumor cell (FIG. 1B). In addition to the creation of a fusion oncogene, cancer-associated translocations also generate novel genomic complexes, e.g., loops, e.g., Cancer Fusion Loops (CFLs), which are required to maintain the high expression level of the fusion oncogene (FIG. 1B). Because cancer cells are highly dependent upon the expression of the fusion oncogene, CFLs ensure cancer cell growth and viability by providing an epigenetic regulatory landscape that is highly permissive for robust expression of the fusion oncogene. Targeting CFLs and other genomic complexes associated with disease-associated fusion genes represent a novel and therapeutically relevant approach to disrupting the expression of disease associated fusion genes, e.g., fusion oncogenes.

Described herein are experiments directed at identifying target anchor sequences proximal to fusion genes, e.g., fusion oncogenes; targeting the genomic complexes, e.g., CFLs, comprising said target anchor sequences for disruption (e.g., inhibiting their formation and/or destabilizing them) using disrupting agents; and evaluating the effects of disruption on fusion gene expression and other cell (e.g., cancer cell) characteristics (e.g., growth, viability, etc.). The data produced show that techniques known in the art (e.g., ChIP-SEQ) and available data sets can be used to identify anchor sequence candidates near target fusion genes. For the experiments described herein, the target anchor sequences comprised CTCF binding sites and the disrupting agents comprised Cas9 and one or more gRNAs specific for the target anchor sequence (e.g., in these experiments, the disrupting agent comprised a targeting moiety that also served as the effector moiety). Without wishing to be bound by theory, Cas9, when bound to a gRNA specified site, can cleave a CTCF binding site, promote insertions and/or deletion mutations that inhibit binding of CTCF, inhibit the formation of or destabilize a genomic complex, e.g., CFL, at that locus. The data demonstrate that targeting a target anchor sequence with a disrupting agent as described decreases expression of the associated fusion gene (see, e.g., Examples 1 and 2). The data further demonstrate that targeting a target anchor sequence with a disrupting agent as described decreased proliferation and the number of viable cells over time of target cells, e.g., cancer cells (see, e.g., Example 2). As one of skill in the art will readily appreciate, although the experiments described herein utilize Cas9 and gRNAs as disrupting agents, a wide variety of moieties are suitable for use as disrupting agents; a selection of these moieties are described further herein. As one of skill in the art will further appreciate, although the experiments described herein target CTCF binding sites, a number of anchor sequences are known in the art and suitable for use as target anchor sequences in the methods described herein; a selection of these target anchor sequences are described herein. Finally, as one of skill in the art will further appreciate, although the experiments described herein target fusion genes, e.g., fusion oncogenes, associated with two different fusion gene associated diseases, e.g., cancers, a number of other diseases are associated with fusion genes and gross chromosomal rearrangements and known to those in the art. The methods and compositions of the disclosure are also suitable for these further diseases, a selection of which are described herein, and application thereto is explicitly contemplated.

Accordingly, the present disclosure provides, at least in part, technologies for disrupting genomic complexes associated with target genes, wherein the target genes are proximal to or comprise a breakpoint, e.g., produced by a gross chromosomal rearrangement, and wherein the gene and/or breakpoint are proximal to a target anchor sequence. In some embodiments, disrupting these specific genomic complexes comprises contacting a cell that comprises a nucleic acid comprising the gene, breakpoint, and target anchor sequence with a site-specific disrupting agent. In some embodiments, disrupting these genomic complexes decreases the expression of the target gene, modifies the chromatin structure of the nucleic acid, and/or treats cancer in a subject in need thereof.

The disclosure additionally features the recognition that some anchor sequences are specific to cancer cells, and that modifying these anchor sequences can revert the cell to a more non-cancerous phenotype.

### Genomic Complexes

Genomic complexes relevant to the present disclosure include stable structures that comprise a plurality of polypeptide and/or nucleic acid (particularly ribonucleic acid) components and that co-localize two or more genomic sequence elements (e.g., anchor sequences, promoter and/or enhancer elements). In some embodiments, one or more of the genomic sequence elements (e.g., anchor sequences, e.g., target anchor sequences, e.g., target cancer-specific anchor sequence) is proximal to a breakpoint and/or a target gene (e.g., fusion gene, e.g., fusion oncogene). In some embodiments, relevant genomic complexes comprise anchor-sequence-mediated conjunctions (e.g., genomic loops). In some embodiments, genomic sequence elements that are (i.e., in three-dimensional space) in genomic complexes include transcriptional promoter and/or regulatory (e.g., enhancer or repressor) sequences. Alternatively or additionally, in some embodiments, genomic sequence elements that are in genomic complexes include binding sites for one or more of CTCF, YY1, etc.

In some embodiments, a genomic complex (e.g., a cancer-specific genomic complex) described herein is not found in a wild-type cell. In some embodiments, one such genomic complex (e.g., one not normally present in wild-type cells, e.g., non-disease cells, e.g., non-cancer cells) is the target of the methods and compositions described herein. In some embodiments, the genomic complex (e.g., the cancer-specific genomic complex) is generated by a gross chromosomal rearrangement, which fuses together chromosomal regions not normally contiguous with one another (e.g., in wild-type cells, e.g., non-disease cells, e.g. non-cancer cells). he genomic complex may include one or more anchor sequences that are not present in wild-type cells, and/or because it brings together two anchor sequences that are not normally together. More specifically, in some embodiments, the genomic complex may comprise or assemble at a genomic sequence element, e.g., anchor sequence, that does not function as a site for assembly of a genomic complex normally (e.g., in wild-type cells, e.g., non-disease cells, e.g. non-cancer cells), but assembles in a cancer cell. In some embodiments, the genomic complex may be proximal to or comprise genomic sequences (e.g., associated/target gene, e.g., fusion gene) that are not proximal or comprised within the genomic complex normally (e.g., in wildtype cells, e.g., non-disease cells, e.g. non-cancer cells), but are present in a cancer cell. In some embodiments, both may occur, e.g., in the same genomic complex. In some embodiments, the genomic complex brings together at least two anchor sequences and is proximal to or comprises a fusion oncogene (e.g., the expression of which the genomic complex promotes). In some embodiments, the genomic complex comprises a Cancer Fusion Loop (CFL).

In some embodiments, a genomic complex whose incidence is decreased in accordance with the present disclosure comprises, or consists of, one or more components chosen from: a genomic sequence element (e.g., an anchor sequence, e.g., a CTCF binding motif, a YY1 binding motif, etc., that may, in some embodiments, be recognized by a nucleating component), one or more polypeptide components (e.g., one or more nucleating polypeptides, one or more transcriptional machinery proteins, and/or one or more transcriptional regulatory proteins), and/or one or more non-genomic nucleic acid components (e.g., non-coding RNA and/or an mRNA, for example, transcribed from a gene associated with the genomic complex).

In some embodiments, a genomic complex component is part of a genomic complex, wherein the genomic complex brings together two genomic sequence elements that are spaced apart from one another on a chromosome, e.g., via an interaction between and among a plurality of protein and/or other components.

In some embodiments, a genomic sequence element is an anchor sequences to which one or more protein components of the complex binds; thus in some embodiments, a genomic complex comprises an anchor-sequence-mediated conjunction. In some embodiments, a genomic sequence element comprises a CTCF binding motif, a promoter and/or an enhancer. In some embodiments, a genomic sequence element includes at least one or both of a promoter and/or regulatory site (e.g., an enhancer). In some embodiments, complex formation is nucleated at the genomic sequence element(s) and/or by binding of one or more of the protein component(s) to the genomic sequence element(s).

Genomic sequence elements involved in genomic complexes as described herein, may be non-contiguous with one another. In some embodiments with noncontiguous genomic sequence elements (e.g., anchor sequences, promoters, and/or transcriptional regulatory sequences), a first genomic sequence element (e.g., anchor sequence, promoter, or transcriptional regulatory sequence) may be separated from a second genomic sequence element (e.g., anchor sequence, promoter, or transcriptional regulatory sequence) by about 500 bp to about 500 Mb, about 750 bp to about 200 Mb, about 1 kb to about 100 Mb, about 25 kb to about 50 Mb, about 50 kb to about 1 Mb, about 100 kb to about 750 kb, about 150 kb to about 500 kb, or about 175 kb to about 500 kb. In some embodiments, a first genomic sequence element (e.g., anchor sequence, promoter, or transcriptional, regulatory sequence) is separated from a second genomic sequence element (e.g., anchor sequence, promoter, or transcriptional regulatory sequence) by about 500 bp, 600 bp, 700 bp, 800 bp, 900 bp, 1 kb, 5 kb, 10 kb, 15 kb, 20 kb, 25 kb, 30 kb, 35 kb, 40 kb, 45 kb, 50 kb, 55 kb, 60 kb, 65 kb, 70 kb, 75 kb, 80 kb, 85 kb, 90 kb, 95 kb, 100 kb, 125 kb, 150 kb, 175 kb, 200 kb, 225 kb, 250 kb, 275 kb, 300 kb, 350 kb, 400 kb, 500 kb, 600 kb, 700 kb, 800 kb, 900 kb, 1 Mb, 2 Mb, 3 Mb, 4 Mb, 5 Mb, 6 Mb, 7 Mb, 8 Mb, 9 Mb, 10 Mb, 15 Mb, 20 Mb, 25 Mb, 50 Mb, 75 Mb, 100 Mb, 200 Mb, 300 Mb, 400 Mb, 500 Mb, or any size therebetween.

### Anchor Sequence-Mediated Conjunction

In some embodiments, a genomic complex relevant to the present disclosure is or comprises an anchor sequence-mediated conjunction. In some embodiments, an anchor-sequence-mediated conjunction is formed when nucleating polypeptide(s) bind to anchor sequences in the genome and interactions between and among these proteins and, optionally, one or more other components, forms a conjunction in which the anchor sequences are physically co-localized. In many embodiments described herein, one or more genes is associated with an anchor-sequence-mediated conjunction; in such embodiments, the anchor sequence-mediated conjunction typically includes one or more anchor sequences, one or more genes, and one or more transcriptional control sequences, such as an enhancing or silencing sequence. In some embodiments, a transcriptional control sequence is within, partially within, or outside an anchor sequence-mediated conjunction.

In some embodiments, a genomic complex as described herein (e.g., an anchor sequence-mediated conjunction) is or comprises a genomic loop, such as an intra-chromosomal loop. In certain embodiments, genomic complex as described herein (e.g., an anchor sequence-mediated conjunction) comprises a plurality of genomic loops. One or more genomic loops may include a first anchor sequence, a nucleic acid sequence, a transcriptional control sequence, and a second anchor sequence. In some embodiments, at least one genomic loop includes, in order, a first anchor sequence, a transcriptional control sequence, and a second anchor sequence; or a first anchor sequence, a nucleic acid sequence, and a second anchor sequence. In yet some embodiments, either one or both of nucleic acid sequences and transcriptional control sequence is located within a genomic loop. In yet some embodiments, either one or both of nucleic acid sequences and transcriptional control sequence is located outside a genomic loop. In some embodiments, one or more genomic loops comprise a transcriptional control sequence. In some embodiments, genomic complex (e.g., an anchor sequence-mediated conjunction) includes a TATA box, a CAAT box, a GC box, or a CAP site.

In some embodiments, an anchor sequence-mediated conjunction comprises a plurality of genomic loops; in some such embodiments, an anchor sequence-mediated conjunction comprises at least one of an anchor sequence, a nucleic acid sequence, and a transcriptional control sequence in one or more genomic loops.

Types of Loops

In some embodiments, a genomic loop comprises one or more, e.g., 2, 3, 4, 5, or more, genes.

In some embodiments, the present disclosure provides methods of modulating (e.g., decreasing) expression of a target gene in a loop comprising inhibiting, dissociating, degrading, and/or modifying a genomic complex that achieves co-localization of genomic sequences that are outside of, not part of, or comprised within (i) a gene whose expression is modulated (e.g. a target gene); and/or (ii) one or more associated transcriptional control sequences that influence transcription of the gene whose expression is modulated.

In some embodiments, the present disclosure provides methods of modulating (e.g., decreasing) transcription of a target gene comprising inhibiting formation of and/or destabilizing a complex that achieves co-localization of genomic sequences that are non-contiguous with (i) a gene whose expression is modulated; and/or (ii) associated transcriptional control sequences that influence transcription of the gene whose expression is modulated.

In some embodiments, an anchor sequence-mediated conjunction is associated with one or more, e.g., 2, 3, 4, 5, or more, transcriptional control sequences. In some embodiments, a target gene is non-contiguous with one or more transcriptional control sequences. In some embodiments where a gene is non-contiguous with its transcriptional control sequence(s), a gene may be separated from one or more transcriptional control sequences by about 100 bp to about 500 Mb, about 500 bp to about 200 Mb, about 1 kb to about 100 Mb, about 25 kb to about 50 Mb, about 50 kb to about 1 Mb, about 100 kb to about 750 kb, about 150 kb to about 500 kb, or about 175 kb to about 500 kb. In some embodiments, a gene is separated from a transcriptional control sequence by about 100 bp, 300 bp, 500 bp, 600 bp, 700 bp, 800 bp, 900 bp, 1 kb, 5 kb, 10 kb, 15 kb, 20 kb, 25 kb, 30 kb, 35 kb, 40 kb, 45 kb, 50 kb, 55 kb, 60 kb, 65 kb, 70 kb, 75 kb, 80 kb, 85 kb, 90 kb, 95 kb, 100 kb, 125 kb, 150 kb, 175 kb, 200 kb, 225 kb, 250 kb, 275 kb, 300 kb, 350 kb, 400 kb, 500 kb, 600 kb, 700 kb, 800 kb, 900 kb, 1 Mb, 2 Mb, 3 Mb, 4 Mb, 5 Mb, 6 Mb, 7 Mb, 8 Mb, 9 Mb, 10 Mb, 15 Mb, 20 Mb, 25 Mb, 50 Mb, 75 Mb, 100 Mb, 200 Mb, 300 Mb, 400 Mb, 500 Mb, or any size therebetween.

In some embodiments, a particular type of anchor sequence-mediated conjunction (genomic loop) may help to determine how to modulate gene expression, e.g., choice of targeting moiety, by destabilization or inhibiting formation of a genomic loop. For example, in some embodiments, some types of anchor sequence-mediated conjunctions comprise one or more transcription control sequences within an anchor sequence-mediated conjunction. Destabilization or inhibiting formation of such a genomic loop can modulate (e.g., decrease), transcription of a target gene within a genomic loop.

By way of non-limiting example, genomic loops may be categorized by certain structural features and types. As further described herein, in some embodiments, certain types of genomic loops may be formed in particular ways, in order to effect certain structural features (e.g. loop topology). In some embodiments, changes in structural features may alter post-nucleating activities and programs. In some embodiments, changes in structural features may result from changes to proteins, non-coding sequences, etc. that are part of a genomic complex but not part of a gene itself. In some embodiments, changes in non-structural (e.g. functional) features in absence of structural changes, may result from changes to proteins, non-coding sequences, etc.

Type 1

In some embodiments, expression of a target gene is regulated, modulated, or influenced by one or more transcriptional control sequences associated with an anchor sequence-mediated conjunction. In some embodiments, anchor sequence-mediated conjunctions are or comprise one or more associated genes and one or more transcriptional control sequences. For example, a target gene and one or more transcriptional control sequences may be located within, at least partially, an anchor sequence-mediated conjunction, e.g., a Type 1, subtype 1 genomic loop, see, e.g., FIG. 6.

An anchor sequence-mediated conjunction as depicted in FIG. 6 may also be referred to as a “Type 1, EP subtype.” In certain embodiments, teachings of the present disclosure are particularly relevant to Type 1, EP subtype genomic loops.

In some embodiments, a target gene has a defined state of expression, e.g., in its untreated state, e.g., in a diseased state. For example, a target gene may have a high level of expression when an associated anchor sequence-mediated conjunction is present. Changing incidence (e.g., frequency, extent, etc.) of such an associated anchor sequence-mediated conjunction may alter expression of the gene, e.g., decreased transcription due to conformational changes of DNA previously open to transcription within an anchor sequence-mediated conjunction, e.g., decreased transcription due to conformational changes of DNA by removing a target gene from proximity to enhancing sequences.

In some embodiments, both an associated gene and one or more transcriptional control sequences, e.g., enhancing sequences, reside inside an anchor sequence-mediated conjunction. In some embodiments, destabilization or inhibiting formation (e.g. decreasing incidence) of a given genomic complex decreases expression of a given gene.

In some embodiments, a gene associated with an anchor sequence-mediated conjunction is accessible to one or more transcriptional control sequences that reside inside, at least partially, an anchor sequence-mediated conjunction.

In some embodiments, destabilization or inhibiting formation of a genomic complex decreases expression of a gene. Changing incidence of an associated anchor sequence-mediated conjunction may alter expression of the gene.

Type 2

In some embodiments, expression of a target gene is regulated, modulated, or influenced by one or more transcriptional control sequences associated with, but inaccessible due to an anchor sequence-mediated conjunction. Transcriptional control sequences may be separated from a given gene, e.g., reside on the opposite side, at least partially, e.g., inside or outside, of an anchor sequence-mediated conjunction as a gene, e.g., a gene is inaccessible to transcriptional control sequences due to proximity of an anchor sequence-mediated conjunction. In some embodiments, one or more enhancing sequences are separated from a gene by an anchor sequence-mediated conjunction, e.g., a Type 2 genomic loop, see, e.g., FIG. 6.

In some embodiments, a gene is enclosed within an anchor sequence-mediated conjunction (loop), while a transcriptional control sequence (e.g., enhancing sequence) is not enclosed within an anchor sequence-mediated conjunction. This subtype of Type 2 may be referred to as “Type 2, subtype 1” genomic loop (see, e.g. FIG. 6).

In some embodiments, a Type 2 transcriptional control sequence (e.g., enhancing sequence) is enclosed within an anchor sequence-mediated conjunction, while a gene is not enclosed within an anchor sequence-mediated conjunction. This subtype of Type 2 may be referred to as “Type 2, subtype 2” genomic loop (see, e.g. FIG. 6).

In some embodiments, a gene is inaccessible to one or more transcriptional control sequences due to an anchor sequence-mediated conjunction. Changing incidence of such an associated anchor sequence-mediated conjunction may alter expression of the gene.

In some embodiments, a gene is inside and outside an anchor sequence-mediated conjunction and inaccessible to one or more transcriptional control sequences. Changing incidence of such an associated anchor sequence-mediated conjunction may alter expression of the gene.

In some embodiments, a gene is inside an anchor sequence-mediated conjunction and inaccessible to one or more transcriptional control sequences residing outside, at least partially, an anchor sequence-mediated conjunction.

In some embodiments, a gene is outside, at least partially, an anchor sequence-mediated conjunction and inaccessible to one or more transcriptional control sequences residing inside an anchor sequence-mediated conjunction. Changing incidence of such an associated anchor sequence-mediated conjunction may alter expression of the gene.

In some embodiments, a target gene has a defined state of expression, e.g., in its untreated state, e.g., in a diseased state. For example, a target gene may have a moderate to low level of expression. Changing incidence of such an associated anchor sequence-mediated conjunction may alter expression of the gene.

Type 3

In some embodiments, expression of a target gene is regulated, modulated, or influenced by one or more transcriptional control sequences associated with an anchor sequence-mediated conjunction, but not necessarily located on a same side of an anchor sequence-mediated conjunction as each other. For example, an anchor sequence-mediated conjunction is associated with one or more genes and one or more transcriptional control sequences reside inside and outside, at least partially, relative to an anchor sequence-mediated conjunction. In some embodiments, one or more enhancing sequences reside inside an anchor sequence-mediated conjunction and one or more repressor signals, e.g., silencing sequences, reside outside an anchor sequence-mediated conjunction, e.g., a Type 3 genomic loop, see, e.g., FIG. 6.

In some embodiments, a gene is inaccessible to one or more transcriptional control sequences due to an anchor sequence-mediated conjunction. Changing incidence of such an associated anchor sequence-mediated conjunction may alter expression of the gene, e.g., to regulate, modulate, or influence expression the gene.

In some embodiments, a gene is inside an anchor sequence-mediated conjunction and inaccessible to one or more transcriptional control sequences, e.g., silencing/repressor sequences, residing outside an anchor sequence-mediated conjunction. Changing incidence of such an associated anchor sequence-mediated conjunction may alter expression of the gene.

In some embodiments, a gene is inside and outside an anchor sequence-mediated conjunction and inaccessible to one or more transcriptional control sequences, e.g., silencing/repressor sequences, anchor sequence-mediated conjunction residing outside an anchor sequence-mediated conjunction. Changing incidence of such an associated anchor sequence-mediated conjunction may alter expression of the gene. For example, destabilization or inhibiting formation (e.g. decreasing incidence) of a genomic complex decreases expression of a gene.

In some embodiments, a gene is outside an anchor sequence-mediated conjunction and inaccessible to one or more transcriptional control sequences, e.g., silencing/repressor sequences, inside an anchor sequence-mediated conjunction. Changing incidence of such an associated anchor sequence-mediated conjunction may alter expression of the gene. For example, destabilization or inhibiting formation (e.g. decreasing incidence) of an anchor sequence-mediated conjunction decreases expression of a gene.

In some embodiments, a target gene has a defined state of expression, e.g., in its untreated state, e.g., in a diseased state. For example, a target gene may have a high level of expression in its native state when an associated anchor sequence-mediated conjunction is present. Changing incidence of such an associated anchor sequence-mediated conjunction may alter expression of the gene. For example, by destabilizing or inhibiting formation (e.g. decreasing incidence) of a genomic complex, expression of a target gene may be modulated, e.g., decreased transcription due to conformational changes of DNA, e.g., decreased transcription due to conformational changes of DNA previously open to transcription within an anchor sequence-mediated conjunction, e.g., decreased transcription due to conformational changes of DNA bringing repressing or silencing sequences into closer association with a target gene, e.g., decreased transcription due to conformational changes of DNA removing distance between a target gene and silencing or repressing sequences.

Type 4

In some embodiments, expression of a target gene is regulated, modulated, or influenced by one or more transcriptional control sequences associated with an anchor sequence-mediated conjunction, but not necessarily located within an anchor sequence-mediated conjunction. For example, an anchor sequence-mediated conjunction is associated with one or more genes and one or more transcriptional control sequences reside inside and outside, at least partially, an anchor sequence-mediated conjunction, e.g., a Type 4 genomic loop, see, e.g. FIG. 6.

In some embodiments, a gene is inaccessible to one or more transcriptional control sequences due to an anchor sequence-mediated conjunction. Changing incidence of such an associated anchor sequence-mediated conjunction may alter expression of the gene. For example, destabilization or inhibiting formation (e.g. decreasing incidence) of a genomic complex allows a transcriptional control sequence to regulate, modulate, or influence expression of a gene.

In some embodiments, a gene is inside an anchor sequence-mediated conjunction and inaccessible to one or more transcriptional control sequences residing outside an anchor sequence-mediated conjunction. Changing incidence of such an associated anchor sequence-mediated conjunction may alter expression of the gene. Stabilizing (e.g., increasing incidence of) the anchor sequence-mediated conjunction may have an opposite effect.

In some embodiments, a gene is inside and outside an anchor sequence-mediated conjunction and inaccessible to one or more transcriptional control sequences (e.g., an enhancing sequence, e.g., residing outside an anchor sequence-mediated conjunction. Changing incidence of such an associated anchor sequence-mediated conjunction may alter expression of the gene.

In some embodiments, a gene is outside an anchor sequence-mediated conjunction and inaccessible to one or more transcriptional control sequences (e.g., an enhancing sequence) inside an anchor sequence-mediated conjunction. Changing incidence of such an associated anchor sequence-mediated conjunction may alter expression of the gene.

In some embodiments, a target gene has a defined state of expression, e.g., in its untreated state, e.g., in a diseased state. For example, in some embodiments, a target gene may have a high level of expression in its untreated state when an associated anchor sequence-mediated conjunction is present. Changing incidence of such an associated anchor sequence-mediated conjunction may alter expression of the gene. For example, modulating incidence of a genomic complex modulates expression of a target gene, e.g., decreased transcription due to conformational changes to close off DNA to transcription, e.g., decreased transcription due to conformational changes of DNA by creating additional space between enhancing sequences and a target gene.

### Cancer Fusion Loops

Gross chromosomal rearrangements such as translocations, insertions, deletions, and inversions can operably link sequences that are not normally (e.g., in wild-type and/or non-disease cells) contiguous.

In some embodiments, a gross chromosomal rearrangement operably links a first protein encoding nucleic acid sequence and a second protein encoding nucleic acid sequence or fragments thereof, e.g., a first gene and a second gene or fragments thereof, to create a fusion gene. In such an embodiment, the breakpoint produced by the gross chromosomal rearrangement is comprised within the protein encoding sequence of the fusion gene, e.g., between the first protein encoding nucleic acid sequence (e.g., the 5′ protein encoding sequence of the fusion gene) and the second protein encoding nucleic acid sequence (e.g., the 3′protein encoding sequence of the fusion gene). Depending on the gross chromosomal rearrangement (e.g., the genomic loci of the first and second protein encoding nucleic acid sequences, the type of rearrangement), a fusion gene may have transcription, epigenetic, and/or chromosomal topology characteristics similar to the first protein encoding nucleic acid sequence (e.g., the first gene), the second protein encoding nucleic acid sequence (e.g., the second gene), or have the characteristics of neither the first or the second sequence (e.g., first or second gene).

In some embodiments, a gross chromosomal rearrangement operably links a first protein encoding nucleic acid sequence or fragment thereof (e.g., a gene or a fragment thereof) with a second nucleic acid sequence that does not normally (e.g., in wild-type and/or non-disease cells) encode for a protein. In some embodiments, the protein encoding nucleic acid sequence or fragment thereof is situated 5′ (e.g., upstream) of the nucleic acid sequence that does not normally encode for a protein in the fusion gene. In some embodiments, the protein encoding nucleic acid sequence or fragment thereof is situated 3′ (e.g., downstream) of the nucleic acid sequence that does not normally encode for a protein in the fusion gene. In an embodiment, the breakpoint produced by the gross chromosomal rearrangement is directly adjacent to the protein-encoding nucleic acid sequence or fragment thereof. In a further embodiment where the breakpoint is directly adjacent to the protein encoding nucleic acid sequence or fragment thereof, the nucleic acid sequence not normally encoding for a protein contributes one or more amino acid encoding codons to the mRNA transcribed from the fusion gene (e.g., when the fusion gene is transcribed, a portion of the non-encoding sequence is transcribed and subsequently translated along with the protein normally encoded by the protein encoding sequence). In some embodiments, the breakpoint produced by the gross chromosomal rearrangement is proximal to the protein encoding nucleic acid sequence or fragment thereof. In a further embodiment where the breakpoint is proximal to the protein encoding nucleic acid sequence or fragment thereof but not directly adjacent, the nucleic acid sequence not normally encoding for a protein does not contribute any amino acid encoding codons to the mRNA transcribed from the fusion gene.

In some embodiments, the fusion gene is transcribed at a level similar to (e.g., the same as or essentially the same as) the protein encoding nucleic acid sequence. In some embodiments, the fusion gene is transcribed at a higher level (e.g., 10, 20, 30, 40, 50, 60, 70, 80, 90, 100, 125, 150, 175, or 200% higher) than the protein encoding nucleic acid sequence is normally (e.g., in a wildtype and/or non-disease cell) expressed, e.g., when not subjected to the gross chromosomal rearrangement.

In some embodiments, the fusion gene is transcribed at a level similar to (e.g., the same as or essentially the same as) the first protein encoding nucleic acid sequence (e.g., the wild-type gene corresponding to the 5′ sequence in the fusion gene). In some embodiments, the fusion gene is transcribed at a higher level (e.g., 10, 20, 30, 40, 50, 60, 70, 80, 90, 100, 125, 150, 175, or 200% higher) than the first protein encoding nucleic acid sequence is normally (e.g., in a wild-type and/or non-disease cell) expressed, e.g., when not subjected to the gross chromosomal rearrangement.

In some embodiments, the fusion gene is transcribed at a level similar to (e.g., the same as or essentially the same as) the second protein encoding nucleic acid sequence (e.g., the wild-type gene corresponding to the 3′ sequence in the fusion gene). In some embodiments, the fusion gene is transcribed at a higher level (e.g., 10, 20, 30, 40, 50, 60, 70, 80, 90, 100, 125, 150, 175, or 200% higher) than the second protein encoding nucleic acid sequence is normally (e.g., in a wild-type and/or non-disease cell) expressed, e.g., when not subjected to the gross chromosomal rearrangement.

In some embodiments, the fusion gene and/or proximal genomic region are epigenetically dissimilar to the epigenetic makeup of the first and/or second nucleic acid sequences of the fusion gene, e.g., prior to the gross chromosomal rearrangement. In some embodiments, the fusion gene and/or proximal genomic region comprise epigenetic markers for active transcription and/or euchromatin. In some embodiments, the first nucleic acid sequence (e.g., wild-type gene corresponding to the 5′ sequence) prior to the gross chromosomal rearrangement comprised epigenetic markers silencing and/or repressing transcription, e.g., heterochromatin epigenetic markers. In some embodiments, the second nucleic acid sequence (e.g., wild-type gene corresponding to the 3′ sequence) prior to the gross chromosomal rearrangement comprised epigenetic markers silencing and/or repressing transcription, e.g., heterochromatin epigenetic markers. In some embodiments, the fusion gene and/or proximal genomic region comprise epigenetic markers that promote transcription of the fusion gene more strongly than the epigenetic markers present on or proximal to the first nucleic acid sequence (e.g., wild-type gene corresponding to the 5′ sequence) prior to the gross chromosomal rearrangement. In some embodiments, the fusion gene and/or proximal genomic region comprise epigenetic markers that promote transcription of the fusion gene more strongly than the epigenetic markers present on or proximal to the second nucleic acid sequence (e.g., wild-type gene corresponding to the 3′ sequence) prior to the gross chromosomal rearrangement.

In some embodiments, the fusion gene is comprised within a genomic complex. In some embodiments, the fusion gene is comprised within an anchor sequence-mediated conjunction.

In some embodiments, the fusion gene is comprised partially within a genomic complex, e.g., the transcriptional start site of the fusion gene is comprised within the genomic complex. In some embodiments, the fusion gene is comprised partially within an anchor sequence-mediated conjunction, e.g., the transcriptional start site of the fusion gene is comprised within the anchor sequence-mediated conjunction.

In some embodiments, the genomic complex, e.g., comprising an anchor sequence-mediated conjunction, e.g., loop, that the fusion gene is comprised within or partially within comprises one or more genomic sequence elements, e.g., anchor sequences, that were part of a genomic complex, e.g., comprising an anchor sequence-mediated conjunction, e.g., loop, prior to the gross chromosomal rearrangement. In an embodiment, one such genomic sequence element, e.g., anchor sequence, contributes to the genomic complex, e.g., comprising an anchor sequence-mediated conjunction, e.g., loop, comprising or partially comprising the fusion gene. In an embodiment, two (e.g., both) such genomic sequence elements, e.g., anchor sequences, contribute to the genomic complex, e.g., comprising an anchor sequence-mediated conjunction, e.g., loop, comprising or partially comprising the fusion gene.

In some embodiments, the genomic complex, e.g., comprising an anchor sequence-mediated conjunction, e.g., loop, that the fusion gene is comprised within or partially within comprises one or more genomic sequence elements, e.g., anchor sequences, that were not part of a genomic complex, e.g., comprising an anchor sequence-mediated conjunction, e.g., loop, prior to the gross chromosomal rearrangement. In an embodiment, one such genomic sequence element, e.g., anchor sequence, contributes to the genomic complex, e.g., comprising an anchor sequence-mediated conjunction, e.g., loop, comprising or partially comprising the fusion gene. In an embodiment, two (e.g., both) such genomic sequence elements, e.g., anchor sequences, contribute to the genomic complex, e.g., comprising an anchor sequence-mediated conjunction, e.g., loop, comprising or partially comprising the fusion gene.

In some embodiments, a gross chromosomal rearrangement creates a fusion gene the expression of which (e.g., the level of expression) is associated with a disease. In some embodiments, that disease is a cancer. Some diseases, e.g., cancers, depend on expression (e.g., a particular level of expression) of an associated fusion gene for the manifestation of symptoms and/or disease progression in a subject. In some embodiments, fusion oncogenes are comprised within or partially within a genomic complex, e.g., comprised within an anchor sequence-mediated conjunction, e.g., loop. In some embodiments, the expression of a fusion oncogene is dependent upon its associated CFL. Without wishing to be bound by theory, disruption of a CFL (e.g., inhibiting their formation and/or destabilizing them) using a disrupting agent described herein can alter, e.g., decrease, expression of the associated fusion oncogene. In some embodiments, disruption of a CFL (e.g., inhibiting their formation and/or destabilizing them) using a disrupting agent described herein can alter, e.g., decrease, expression of the associated fusion oncogene and treat the associated cancer and/or the symptoms of the associated cancer in a subject having the associated cancer.

### Genomic Sequence Elements

Genomic complexes as described herein, when present, achieve co-localization (in three-dimensional space) of two or more genomic sequence elements. In some embodiments, a relevant genomic sequence element is one to which a component of the genomic complex binds specifically. In some embodiments, a relevant genomic sequence element may be or comprise an anchor sequence, a promoter, a regulatory sequence, an associated gene, or a combination thereof.

### Anchor Sequences

In general, an anchor sequence is a genomic sequence element to which a genomic complex component binds specifically. In some embodiments, binding to an anchor sequence nucleates complex formation.

Each anchor sequence-mediated conjunction comprises one or more anchor sequences, e.g., a plurality. In some embodiments, anchor sequences can be manipulated or altered to form and/or stabilize naturally occurring loops, to form one or more new loops (e.g., to form exogenous loops or to form non-naturally occurring loops with exogenous or altered anchor sequences, see, e.g., FIG. 6), or to inhibit formation of or destabilize naturally occurring or exogenous loops. Such alterations may modulate gene expression by, e.g., changing topological structure of DNA, e.g., by thereby modulating ability of a target gene to interact with gene regulation and control factors (e.g., enhancing and silencing/repressor sequences).

In some embodiments, chromatin structure is modified by substituting, adding or deleting one or more nucleotides within an anchor sequence-mediated conjunction. In some embodiments, chromatin structure is modified by substituting, adding, or deleting one or more nucleotides within an anchor sequence of an anchor sequence-mediated conjunction.

In some embodiments, an anchor sequence comprises a common nucleotide sequence, e.g., a CTCF-binding motif: N(T/C/G)N(G/A/T)CC(A/T/G)(C/G)(C/T/A)AG(G/A)(G/T)GG(C/A/T)(G/A)(C/G)(C/T/A)(G/A/C) (SEQ ID NO:1), where N is any nucleotide.

A CTCF-binding motif may also be in an opposite orientation, e.g., (G/A/C)(C/T/A)(C/G)(G/A)(C/A/T)GG(G/T)(G/A)GA(C/T/A)(C/G)(A/T/G)CC(G/A/T)N(T/C/G)N (SEQ ID NO:2). In some embodiments, an anchor sequence comprises SEQ ID NO:1 or SEQ ID NO:2 or a sequence at least 75%, at least 80%, at least 90%, at least 95%, at least 96%, at least 97%, at least 98%, at least 99% identical to either SEQ ID NO:1 or SEQ ID NO:2.

In some embodiments, an anchor sequence-mediated conjunction comprises at least a first anchor sequence and a second anchor sequence. For example, in some embodiments, a first anchor sequence and a second anchor sequence may each comprise a common nucleotide sequence, e.g., each comprises a CTCF binding motif.

In some embodiments, a first anchor sequence and second anchor sequence comprise different sequences, e.g., a first anchor sequence comprises a CTCF binding motif and a second anchor sequence comprises an anchor sequence other than a CTCF binding motif. In some embodiments, each anchor sequence comprises a common nucleotide sequence and one or more flanking nucleotides on one or both sides of a common nucleotide sequence.

Two CTCF-binding motifs (e.g., contiguous or non-contiguous CTCF binding motifs) that can form a conjunction may be present in a genome in any orientation, e.g., in the same orientation (tandem) either 5′-3′ (left tandem, e.g., the two CTCF-binding motifs that comprise SEQ ID NO:1) or 3′-5′ (right tandem, e.g., the two CTCF-binding motifs comprise SEQ ID NO:2), or convergent orientation, where one CTCF-binding motif comprises SEQ ID NO:1 and another other comprises SEQ ID NO:2. CTCFBSDB 2.0: Database For CTCF binding motifs And Genome Organization (on the world wide web at insulatordb.uthsc.edu/) can be used to identify CTCF binding motifs associated with a target gene.

In some embodiments, an anchor sequence comprises a CTCF binding motif associated with a target gene, wherein the target gene is associated with a disease, disorder and/or condition.

In some embodiments, chromatin structure may be modified by substituting, adding, or deleting one or more nucleotides within at least one anchor sequence, e.g., a nucleating polypeptide binding motif. One or more nucleotides may be specifically targeted, e.g., a targeted alteration, for substitution, addition or deletion within an anchor sequence, e.g., a nucleating polypeptide binding motif.

In some embodiments, an anchor sequence-mediated conjunction may be altered by changing an orientation of at least one common nucleotide sequence, e.g., a nucleating polypeptide binding motif. In some embodiments, an anchor sequence comprises a nucleating polypeptide binding motif, e.g., CTCF binding motif, and a targeting moiety introduces an alteration in at least one nucleating polypeptide binding motif, e.g. altering binding affinity for a nucleating polypeptide.

In some embodiments, an anchor sequence-mediated conjunction may be altered by introducing an exogenous anchor sequence. In some embodiments, addition of a non-naturally occurring or exogenous anchor sequence to destabilize or inhibit formation of a naturally occurring anchor sequence-mediated conjunction, e.g., by inducing a non-naturally occurring loop to form, alters (e.g., decreases) transcription of a nucleic acid sequence.

Promoter Sequences

In some embodiments, a genomic complex as described herein achieves co-localization of genomic sequence elements that include a promoter. Those skilled in the art are aware that a promoter is, typically, a sequence element that initiates transcription of an associated gene. Promoters are typically near the 5′ end of a gene, not far from its transcription start site.

As those of ordinary skill are aware, transcription of protein-coding genes in eukaryotic cells is typically initiated by binding of general transcription factors (e.g., TFIID, TFIIE, TFIIH, etc.) and Mediator to core promoter sequences as a preinitiation complex that directs RNA polymerase II to the transcription start site, and in many instances remains bound to the core promoter sequences even after RNA polymerase escapes and elongation of the primary transcript is initiated.

In many embodiments, a promoter includes a sequence element such as TATA, Inr, DPE, or BRE, but those skilled in the art are well aware that such sequences are not necessarily required to define a promoter.

### Transcriptional Regulatory Sequences

In some embodiments, a genomic complex as described herein achieves co-localization of genomic sequence elements that include one or more transcriptional regulatory sequences. Those skilled in the art are familiar with a variety of positive (e.g., enhancers) or negative (e.g., repressors or silencers) transcriptional regulatory sequence elements that are associated with genes. Typically, when a cognate regulatory protein is bound to such a transcriptional regulatory sequence, transcription from the associated gene(s) is altered (i.e., increased for a positive regulatory sequence; decreased for a negative regulatory sequence.

### Associated Genes

As described herein, in some embodiments, destabilization or inhibiting formation of genomic complexes achieves and/or results in alteration of expression of one or more genes associated with the genomic complex(es) (e.g., a target gene).

In some embodiments, an associated gene is a fusion gene. In some embodiments, a fusion gene comprises a first nucleic acid sequence and a second nucleic acid sequence that are not normally found contiguous with one another in a wild-type cell (e.g., not contiguous with one another based on the Genome Reference Consortium human genome (build 38)). The first nucleic acid sequence can comprise a gene or a portion of a gene. In some embodiments, the second nucleic acid sequence comprises a second gene or portion of a second gene. In some embodiments, the second nucleic acid sequence comprises a sequence that does not normally encode a protein in a wild-type cell. In some embodiments, the second nucleic acid is translated as part of a fusion gene. In some embodiments, the second nucleic acid sequence comprises a regulatory sequence. In some embodiments, the second nucleic acid sequence comprises an intronic sequence. In some embodiments a fusion gene comprises a breakpoint (e.g., created by a gross chromosomal rearrangement). In some embodiments, a fusion gene is proximal to a breakpoint (e.g., created by a gross chromosomal rearrangement). In some embodiments, a fusion gene and/or breakpoint are formed by a gross chromosomal rearrangement (e.g., a translocation, inversion, deletion, duplication, or insertion). The gross chromosomal rearrangement may result in the first and/or second nucleic acid sequence becoming associated with a genomic complex, e.g., comprising an anchor sequence-mediated conjunction. For example, the gross chromosomal rearrangement may result in the first and/or second nucleic acid sequence being inside a genomic complex, e.g., a loop, (e.g., wherein the first and/or second nucleic acid sequence was not inside a genomic complex, e.g., a loop, before the rearrangement). For example, the gross chromosomal rearrangement may result in the first and/or second nucleic acid sequence being outside a genomic complex, e.g., a loop, (e.g., wherein the first and/or second nucleic acid sequence was inside a genomic complex, e.g., a loop, before the rearrangement). The association or non-association with a genomic complex, in some embodiments, may cause the fusion gene to be subject to regulation by transcriptional regulatory sequences (e.g., by being brought into proximity to a transcriptional regulatory sequence). The gross chromosomal rearrangement may result in altered and/or non-native expression of the fusion gene. In some embodiments, the first and/or second nucleic acid sequences of the fusion gene are expressed at a higher level than before the gross chromosomal rearrangement. In some embodiments, the high level of expression of the fusion gene is associated one or more conditions or diseases in a subject, e.g., human subject. In some embodiments, the one or more conditions or diseases include cancer.

In some embodiments, an associated gene is a fusion gene and an oncogene (a fusion oncogene). A fusion oncogene is a fusion gene that is capable of causing or promoting cancer (e.g., causing or promoting a cancerous cell state, e.g., characterized by dysregulated growth, division, and/or invasion) under appropriate physiological and/or cellular conditions. A number of fusion oncogenes are known to those skilled in the art and some fusion oncogenes are known to be associated with particular types of cancers or cell types. In some embodiments, the fusion oncogene is a fusion oncogene listed in Table 1. In some embodiments, the cancer is a cancer of Table 1. In some embodiments, the fusion oncogene is a fusion oncogene listed in Table 1 and the cancer is a cancer from the same row of Table 1.

TABLE 1
Exemplary selected genes associated with translocation mutations
in cancers (e.g., solid tumors and hematologic malignancies)

| Gene | Exemplary Translocation Partners | Exemplary Cancer Types |
|---|---|---|
| ABL1 | BCR, ETV6, NUP214 | CML, ALL, T-ALL |
| ABL2 | ETV6 | AML |
| ACSL3 | ETV1 | prostate |
| AF15Q14 | MLL | AML |
| AF1Q | MLL | ALL |
| AF3p21 | MLL | ALL |
| AF5q31 | MLL | ALL |
| AFF1 | KMT2A | ALL, e.g., pediatric ALL |
| AKAP9 | BRAF | papillary thyroid |
| ALK | NPM1, TPM3, TFG, TPM4, ATIC, | ALCL, lung cancer, e.g., NSCLC, |
| | CLTC, MSN, ALO17, CARS, EML4 | Neuroblastoma |
| ALO17 | ALK | ALCL |
| ARHGEF12 | MLL | AML |
| ARHH | BCL6 | NHL |
| ARNT | ETV6 | AML |
| ASPSCR1 | TFE3 | alveolar soft part sarcoma |
| ATF1 | EWSR1, FUS | malignant melanoma of soft parts, |
| | | angiomatoid fibrous histiocytoma |
| ATIC | ALK | ALCL |
| BCL10 | IGH | MALT |
| BCL11A | IGH | B-CLL |
| BCL11B | TLX3 | T-ALL |
| BCL2 | IGH | NHL, CLL |
| BCL3 | IGH | CLL |
| BCL5 | MYC | CLL |
| BCL6 | IG loci, ZNFN1A1, LCP1, PIM1, | NHL, CLL |
| | TFRC, MHC2TA, NACA, HSPCB, | |
| | HSPCA, HIST1H4I, IL21R, | |
| | POU2AF1, ARHH, EIF4A2, SFRS3 | |
| BCL7A | MYC | BNHL |
| BCL9 | IGH, IGL | B-ALL |
| BCR | ABL1, FGFR1,JAK2 | CML, ALL, AML |
| BIRC3 | MALT1 | MALT |
| BRAF | AKAP9, KIAA1549 | melanoma, colorectal, papillary |
| | | thyroid, borderline ov, Non small- |
| | | cell lung cancer (NSCLC), |
| | | cholangiocarcinoma, pilocytic |
| | | astrocytoma |
| BRD3 | NUT | lethal midline carcinoma of young |
| | | people |
| BRD4 | NUT | lethal midline carcinoma of young |
| | | people |
| BTG1 | MYC | BCLL |
| C12orf9 | LPP | lipoma |
| C15orf21 | ETV1 | prostate |
| CANT1 | ETV4 | prostate |
| CARS | ALK | ALCL |
| CBFA2T1 | MLL, RUNX1 | AML |
| CBFA2T3 | RUNX1 | AML |
| CBFB | MYH11 | AML |
| CBL | MLL | AML, JMML, MDS |
| CCND1 | IGH, FSTL3 | CLL, B-ALL, breast |
| CCND2 | IGL | NHL, CLL |
| CCND3 | IGH | MM |
| CD74 | ROS1 | NSCLC |
| CDH11 | USP6 | aneurysmal bone cysts |
| CDK6 | MLLT10 | ALL |
| CDX2 | ETV6 | AML |
| CEP1 | FGFR1 | MPD, NHL |
| CHCHD7 | PLAG1 | salivary adenoma |
| CHIC2 | ETV6 | AML |
| CHN1 | TAF15 | extraskeletal myxoid |
| | | chondrosarcoma |
| CIC | DUX4 | soft tissue sarcoma |
| CLTC | ALK, TFE3 | ALCL, renal |
| CLTCL1 | | ALCL |
| CMKOR1 | HMGA2 | lipoma |
| COL1A1 | PDGFB, USP6 | dermatofibrosarcoma protuberans, |
| | | aneurysmal bone cyst |
| COX6C | HMGA2 | uterine leiomyoma |
| CREB1 | EWSR1 | clear cell sarcoma, angiomatoid |
| | | fibrous histiocytoma |
| CREB3L2 | FUS | fibromyxoid sarcoma |
| CREBBP | MLL, MORF, RUNXBP2 | AL, AML |
| CRTC3 | MAML2 | salivary gland mucoepidermoid |
| CTNNB1 | PLAG1 | colorectal, cvarian, hepatoblastoma, |
| | | others, pleomorphic salivary |
| | | adenoma |
| D10S170 | RET, PDGFRB | papillary thyroid, CML |
| DDIT3 | FUS | liposarcoma |
| DDX10 | NUP98 | AML* |
| DDX5 | ETV4 | prostate |
| DDX6 | IGH | B-NHL |
| DEK | NUP214 | AML |
| DUX4 | CIC | soft tissue sarcoma |
| EIF4A2 | BCL6 | NHL |
| ELF4 | ERG | AML |
| ELK4 | SLC45A3 | prostate |
| ELKS | RET | papillary thyroid |
| ELL | MLL | AL |
| ELN | PAX5 | B-ALL |
| EML4 | ALK | NSCLC |
| EP300 | MLL, RUNXBP2 | colorectal, breast, pancreatic, AML |
| EPS15 | MLL | ALL |
| ERG | EWSR1, TMPRSS2, ELF4, FUS, | Ewing sarcoma, prostate, AML |
| | HERPUD1 | |
| ETV1 | EWSR1, TMPRSS2, SLC45A3, | Ewing sarcoma, prostate |
| | C15orf21, HNRNPA2B1. ACSL3 | |
| ETV4 | EWSR1, TMPRSS2, DDX5, KLK2, | Ewing sarcoma, Prostate carcinoma |
| | CANT1 | |
| ETV5 | TMPRSS2, SCL45A3 | Prostate |
| ETV6 | NTRK3, RUNX1, PDGFRB, ABL1, | congenital fibrosarcoma, multiple |
| | MN1, ABL2, FACL6, CHIC2, | leukemia and lymphoma, secretory |
| | ARNT, JAK2, EVI1, CDX2, STL, | breast, MDS, ALL |
| | HLXB9, MDS2, PER1, SYK, TTL, | |
| | FGFR3, PAX5 | |
| ETV6 | NTRK3, RUNX1, PDGFRB, ABL1, | congenital fibrosarcoma, multiple |
| | MN1, ABL2, FACL6, CHIC2, | leukemia and lymphoma, secretory |
| | ARNT, JAK2, EVI1, CDX2, STL, | breast, MDS, ALL |
| | HLXB9, MDS2, PER1, SYK, TTL, | |
| | FGFR3, PAX5 | |
| EVI1 | RUNX1, ETV6, PRDM16, RPN1 | AML, CML |
| EWSR1 | FLI1, ERG, ZNF278, NR4A3, FEV, | Ewing sarcoma, desmoplastic small |
| | ATF1, ETV1, ETV4, WT1, ZNF384, | round cell tumor, ALL, clear cell |
| | CREB1, POU5F1, PBX1 | sarcoma, sarcoma, myoepithelioma |
| FACL6 | ETV6 | AML, AEL |
| FCGR2B | | ALL |
| FEV | EWSR1, FUS | Ewing sarcoma |
| FGFR1 | BCR, FOP, ZNF198, CEP1 | MPD, NHL |
| FGFR1OP | FGFR1 | MPD, NHL |
| FGFR3 | IGH, ETV6 | bladder, MM, T-cell lymphoma |
| FIP1L1 | PDGFRA | idiopathic hypereosinophilic |
| | | syndrome |
| FLI1 | EWSR1 | Ewing sarcoma |
| FNBP1 | MLL | AML |
| FOXO1A | PAX3 | alveolar rhabdomyosarcomas |
| FOXO3A | MLL | AL |
| FOXP1 | PAX5 | ALL |
| FSTL3 | CCND1 | B-CLL |
| FUS | DDIT3, ERG, FEV, ATF1, | liposarcoma, AML, Ewing sarcoma, |
| | CREB3L2 | angiomatoid fibrous histiocytoma, |
| | | fibromyxoid sarcoma |
| FVT1 | IGK | B-NHL |
| GAS7 | MLL | AML* |
| GMPS | MLL | AML |
| GOLGA5 | RET | papillary thyroid |
| GPHN | MLL | AL |
| GRAF | MLL | AML, MDS |
| HCMOGT-1 | PDGFRB | JMML |
| HEAB | MLL | AML |
| HEI10 | HMGA2 | uterine leiomyoma |
| HERPUD1 | ERG | prostate |
| HIP1 | PDGFRB | CMML |
| HIST1H4I | BCL6 | NHL |
| HLF | TCF3 | ALL |
| HLXB9 | ETV6 | AML |
| HMGA1 | | microfollicular thyroid adenoma, |
| | | various benign mesenchymal tumors |
| HMGA2 | LHFP, RAD51L1, LPP, HEI10, | lipoma |
| | COX6C, CMKOR1, NFIB | |
| HNRNPA2B1 | ETV1 | prostate |
| HOOK3 | RET | papillary thyroid |
| HOXA11 | NUP98 | CML |
| HOXA13 | NUP98 | AML |
| HOXA9 | NUP98, MSI2 | AML* |
| HOXC11 | NUP98 | AML |
| HOXC13 | NUP98 | AML |
| HOXD11 | NUP98 | AML |
| HOXD13 | NUP98 | AML* |
| HSPCA | BCL6 | NHL |
| HSPCB | BCL6 | NHL |
| IGH | MYC, FGFR3, PAX5, IRTA1, IRF4, | MM, Burkitt lymphoma, NHL, CLL, |
| | CCND1, BCL9, BCL8, BCL6, | B-ALL, MALT, MLCLS |
| | BCL2, BCL3, BCL10, BCL11A. | |
| | LHX4, DDX6, NFKB2, PAFAH1B2, | |
| | PCSK7 | |
| IGK | MYC, FVT1 | Burkitt lymphoma, B-NHL |
| IGL | BCL9, MYC, CCND2 | Burkitt lymphoma |
| IL2 | TNFRSF17 | intestinal T-cell lymphoma |
| IL21R | BCL6 | NHL |
| IRF4 | IGH | MM |
| IRTA1 | IGH | B-NHL |
| ITK | SYK | peripheral T-cell lymphoma |
| JAK2 | ETV6, PCM1, BCR | ALL, AML, MPD, CML |
| JAZF1 | SUZ12 | endometrial stromal tumours |
| KDM5A | NUP98 | AML |
| KLK2 | ETV4 | prostate |
| KTN1 | RET | papillary thyroid |
| LAF4 | MLL, RUNX1 | ALL, T-ALL |
| LASP1 | MLL | AML |
| LCK | TRB | T-ALL |
| LCP1 | BCL6 | NHL |
| LCX | MLL | AML |
| LHFP | HMGA2 | lipoma |
| LIFR | PLAG1 | salivary adenoma |
| LMO1 | TRD | T-ALL |
| LMO2 | TRD | T-ALL |
| LPP | HMGA2, MLL, C12orf9 | lipoma, leukemia |
| LYL1 | TRB | T-ALL |
| MAF | IGH | MM |
| MAFB | IGH | MM |
| MALT1 | BIRC3 | MALT |
| MAML2 | MECT1, CRTC3 | salivary gland mucoepidermoid |
| MDS1 | RUNX1 | MDS, AML |
| MDS2 | ETV6 | MDS |
| MECT1 | MAML2 | salivary gland mucoepidermoid |
| MHC2TA | BCL6 | NHL |
| MKL1 | RBM15 | acute megakaryocytic leukemia |
| MLF1 | NPM1 | AML |
| MLL (also | MLL, MLLT1, MLLT2, MLLT3, | AML, ALL |
| called KMT2A) | MLLT4, MLLT7, MLLT10, | |
| | MLLT6, ELL, EPS15, AF1Q, | |
| | CREBBP, SH3GL1, FNBP1, | |
| | PNUTL1, MSF, GPHN, GMPS, | |
| | SSH3BP1, ARHGEF12, GAS7, | |
| | FOXO3A, LAF4, LCX, SEPT6, | |
| | LPP, CBFA2T1, GRAF, EP300, | |
| | PICALM, HEAB | |
| MLLT1 | MLL | AL |
| MLLT10 | MLL, PICALM, CDK6 | AL |
| MLLT2 | MLL | AL |
| MLLT3 | MLL | ALL |
| MLLT4 | MLL | AL |
| MLLT6 | MLL | AL |
| MLLT7 | MLL | AL |
| MN1 | ETV6 | AML, meningioma |
| MSF | MLL | AML* |
| MSI2 | HOXA9 | CML |
| MSN | ALK | ALCL |
| MTCP1 | TRA | T cell prolymphocytic leukemia |
| MUC1 | IGH | B-NHL |
| MYB | NFIB | adenoid cystic carcinoma |
| MYC | IGK, BCL5, BCL7A , BTG1, TRA, | Burkitt lymphoma, amplified in |
| | IGH | other cancers, B-CLL |
| MYH11 | CBFB | AML |
| MYH9 | ALK | ALCL |
| MYST4 | CREBBP | AML |
| NACA | BCL6 | NHL |
| NCOA1 | PAX3 | alveolar rhadomyosarcoma |
| NCOA2 | RUNXBP2 | AML |
| NCOA4 | RET | papillary thyroid |
| NFIB | MYB, HGMA2 | adenoid cystic carcinoma, lipoma |
| NFKB2 | IGH | B-NHL |
| NIN | PDGFRB | MPD |
| NONO | TFE3 | papillary renal cancer |
| NOTCH1 | TRB | T-ALL |
| NPM1 | ALK, RARA, MLF1 | NHL, APL, AML |
| NR4A3 | EWSR1 | extraskeletal myxoid |
| | | chondrosarcoma |
| NSD1 | NUP98 | AML |
| NTRK1 | TPM3, TPR, TFG | papillary thyroid |
| NTRK3 | ETV6 | congenital fibrosarcoma, Secretory |
| | | breast |
| NUMA1 | RARA | APL |
| NUP214 | DEK, SET, ABL1 | AML, T-ALL |
| NUP98 | HOXA9, NSD1, WHSC1L1, | AML |
| | DDX10, TOP1, HOXD13, PMX1, | |
| | HOXA13, HOXD11, HOXA11, | |
| | RAP1GDS1, HOXC11 | |
| NUT | BRD4, BRD3 | lethal midline carcinoma of young |
| | | people |
| OLIG2 | TRA | T-ALL |
| OMD | USP6 | aneurysmal bone cysts |
| PAFAH1B2 | IGH | MLCLS |
| PAX3 | FOXO1A, NCOA1 | alveolar rhabdomyosarcoma |
| PAX5 | IGH, ETV6, PML, FOXP1, ZNF521, | NHL, ALL, B-ALL |
| | ELN | |
| PAX7 | FOXO1A | alveolar rhabdomyosarcoma |
| PAX8 | PPARG | follicular thyroid |
| PBX1 | TCF3, EWSR1 | pre B-ALL, myoepithelioma |
| PCM1 | RET, JAK2 | papillary thyroid, CML, MPD |
| PCSK7 | IGH | MLCLS |
| PDE4DIP | PDGFRB | MPD |
| PDGFB | COL1A1 | DFSP |
| PDGFRA | FIP1L1 | GIST, idiopathic hypereosinophilic |
| | | syndrome |
| PDGFRB | ETV6, TRIP11, HIP1, RAB5EP, H4, | MPD, AML, CMML, CML |
| | NIN, HCMOGT-1, PDE4DIP | |
| PER1 | ETV6 | AML, CMML |
| PICALM | MLLT10, MLL | TALL, AML, |
| PIM1 | BCL6 | NHL |
| PLAG1 | TCEA1, LIFR, CTNNB1, CHCHD7 | salivary adenoma |
| PML | RARA, PAX5 | APL, ALL |
| PMX1 | NUP98 | AML |
| PNUTL1 | MLL | AML |
| POU2AF1 | BCL6 | NHL |
| POU5F1 | EWSR1 | sarcoma |
| PPARG | PAX8 | follicular thyroid |
| PRCC | TFE3 | papillary renal |
| PRDM16 | EVI1 | MDS, AML |
| PRKAR1A | RET | papillary thyroid |
| PRO1073 | TFEB | renal cell carcinoma (childhood |
| | | epithelioid) |
| PSIP2 | NUP98 | AML |
| RAB5EP | PDGFRB | CMML |
| RAD51L1 | HMGA2 | lipoma, uterine leiomyoma |
| RAF1 | SRGAP3 | pilocytic astrocytoma |
| RANBP17 | TRD | ALL |
| RAP1GDS1 | NUP98 | T-ALL |
| RARA | PML, ZNF145, TIF1, NUMA1, | APL |
| | NPM1 | |
| RBM15 | MKL1 | acute megakaryocytic leukemia |
| RET | H4, PRKAR1A, NCOA4, PCM1, | medullary thyroid, papillary thyroid, |
| | GOLGA5, TRIM33, KTN1, | pheochromocytoma |
| | TRIM27, HOOK3 | |
| ROS1 | GOPC, ROS1 | glioblastoma, NSCLC |
| RPL22 | RUNX1 | AML, CML |
| RPN1 | EVI1 | AML |
| RUNX1 | RPL22, MDS1, EVI1, CBFA2T3, | AML, preB- ALL, T-ALL |
| | CBFA2T1, ETV6, LAF4 | |
| RUNXBP2 | CREBBP, NCOA2, EP300 | AML |
| SEPT6 | MLL | AML |
| SET | NUP214 | AML |
| SFPQ | TFE3 | papillary renal cell |
| SFRS3 | BCL6 | follicular lymphoma |
| SH3GL1 | MLL | AL |
| SIL | TAL1 | T-ALL |
| SLC45A3 | ETV1, ETV5, ELK4, ERG | prostate |
| SRGAP3 | RAF1 | pilocytic astrocytoma |
| SS18 | SSX1, SSX2 | synovial sarcoma |
| SS18L1 | SSX1 | synovial sarcoma |
| SSH3BP1 | MLL | AML |
| SSX1 | SS18 | synovial sarcoma |
| SSX2 | SS18 | synovial sarcoma |
| SSX4 | SS18 | synovial sarcoma |
| STL | ETV6 | B-ALL |
| SUZ12 | JAZF1 | endometrial stromal tumours |
| SYK | ETV6, ITK | MDS, peripheral T-cell lymphoma |
| TAF15 | TEC, CHN1, ZNF384 | extraskeletal myxoid |
| | | chondrosarcomas, ALL |
| TAL1 | TRD, SIL | lymphoblastic leukemia/biphasic |
| TAL2 | TRB | T-ALL |
| TCEA1 | PLAG1 | salivary adenoma |
| TCF12 | TEC | extraskeletal myxoid |
| | | chondrosarcoma |
| TCF3 | PBX1, HLF, TFPT | lung cancer, ALL, e.g., pre B-ALL |
| TCL1A | TRA | T-CLL |
| TCL6 | TRA | T-ALL |
| TFE3 | SFPQ, ASPSCR1, PRCC, NONO, | papillary renal, alveolar soft part |
| | CLTC | sarcoma, renal |
| TFEB | ALPHA | renal (childhood epithelioid) |
| TFG | NTRK1, ALK | papillary thyroid, ALCL, NSCLC |
| TFPT | TCF3 | Lung cancer, ALL, e.g., pre-B ALL |
| TFRC | BCL6 | NHL |
| THRAP3 | USP6 | aneurysmal bone cysts |
| TIF1 | RARA | APL |
| TLX1 | TRB, TRD | T-ALL |
| TLX3 | BCL11B | T-ALL |
| TMPRSS2 | ERG, ETV1, ETV4, ETV5 | prostate |
| TNFRSF17 | IL2 | intestinal T-cell lymphoma |
| TOP1 | NUP98 | AML* |
| TPM3 | NTRK1, ALK | papillary thyroid, ALCL |
| TPM4 | ALK | ALCL |
| TPR | NTRK1 | papillary thyroid |
| TRA | ATL, OLIG2, MYC, TCL1A, TCL6, | T-ALL |
| | MTCP1, TCL6 | |
| TRB | HOX11, LCK, NOTCH1, TAL2, | T-ALL |
| | LYL1 | |
| TRD | TAL1, HOX11, TLX1, LMO1, | T-cell leukemia |
| | LMO2, RANBP17 | |
| TRIM27 | RET | papillary thyroid |
| TRIM33 | RET | papillary thyroid |
| TRIP11 | PDGFRB | AML |
| TTL | ETV6 | ALL |
| USP6 | COL1A1, CDH11, ZNF9, OMD | aneurysmal bone cysts |
| WHSC1 | IGH | MM |
| WHSC1L1 | NUP98 | AML |
| ZNF145 | RARA | APL |
| ZNF198 | FGFR1 | MPD, NHL |
| ZNF278 | EWSR1 | Ewing sarcoma |
| ZNF331 | | follicular thyroid adenoma |
| ZNF384 | EWSR1, TAF15 | ALL |
| ZNF521 | PAX5 | ALL |
| ZNF9 | USP6 | aneurysmal bone cysts |
| ZNFN1A1 | BCL6 | ALL, DLBL |

In some embodiments, the fusion oncogene is chosen from: ACBD6-RRP15, ACSL3_ENST00000357430-ETV1, ACTB-GLI1, AGPAT5-MCPH1, AGTRAP-BRAF, AKAP9_ENST00000356239-BRAF, ARFIP1-FHDC1, ARID1A-MAST2_ENST00000361297, ASPSCR1-TFE3, ATG4C-FBXO38, ATIC-ALK, BBS9-PKD1L1, BCR-ABL1, BCR-JAK2, BRD3-NUTM1, BRD4_ENST00000263377-NUTM1, C2orf44-ALK, CANT1-ETV4, CARS-ALK, CBFA2T3-GLIS2, CCDCl₆-RET, CD74_ENST00000009530-NRG1, CD74_ENST00000009530-ROS1, CDH11-USP6_ENST00000250066, CDKN2D-WDFY2, CEP89-BRAF, CHCHD7-PLAG1, CIC-DUX4L1, CIC-FOXO4, CLCN6-BRAF, CLIP1-ROS1, CLTC-ALK, CLTC-TFE3, CNBP-USP6_ENST00000250066, COL1A1-PDGFB, COL1A1-USP6_ENST00000250066, COL1A2-PLAG1, CRTC1-MAML2, CRTC3-MAML2, CTAGE5-SIP1, CTNNB1-PLAG1, DCTN1-ALK, DDX5_ENST00000540698-ETV4, DHH-RHEBL1, DNAJB1-PRKACA, EIF3E-RSPO2, EIF3K-CYP39A1, EML4-ALK, EPC1-PHF1, ERC1-RET, ERC1-ROS1, ERO1L-FERMT2, ESRP1-RAF1, ETV6-ABL1, ETV6-ITPR2, ETV6-JAK2, ETV6-NTRK3, ETV6-RUNX1, EWSR1-ATF1, EWSR1-CREB1, EWSR1-DDIT3, EWSR1-ERG, EWSR1-ETV1, EWSR1-ETV4, EWSR1-FEV, EWSR1-FLI1, EWSR1-NFATC1, EWSR1-NFATC2, EWSR1-NR4A3, EWSR1-PATZ1, EWSR1-PBX1, EWSR1-POU5F1, EWSR1-SMARCA5, EWSR1-SP3, EWSR1-WT1, EWSR1-YY1, EWSR1-ZNF384, EWSR1-ZNF444_ENST00000337080, EZR-ROS1, FAM131B_ENST00000443739-BRAF, FBXL18-RNF216, FCHSD1-BRAF, FGFR1-ZNF703, FGFR1 ENST00000447712-PLAG1, FGFR1_ENST00000447712-TACC1, FGFR3-BAIAP2L1, FGFR3-TACC3, FN1-ALK, FUS-ATF1, FUS-CREB3L1, FUS-CREB3L2, FUS-DDIT3, FUS-ERG, FUS-FEV, GATM-BRAF, GMDS-PDE8B, GNAI1-BRAF, GOLGAS-RET, GOPC-ROS1, GPBP1L1-MAST2_ENST00000361297, HACL1-RAF1, HAS2-PLAG1, HERPUD1-BRAF, HEY1-NCOA2, HIP1-ALK, HLA-A-ROS1, HMGA2-ALDH2_ENST00000261733, HMGA2-CCNBlIP1, HMGA2-COX6C, HMGA2-EBF1, HMGA2-FHIT_ENST00000476844, HMGA2-LHFP, HMGA2-LPP, HMGA2-NFIB_ENST00000397581, HMGA2-RAD51B, HMGA2-WW1_ENST00000286574, HN1-USH1G, HNRNPA2B1-ETV1, HOOKS-RET, IL6R-ATP8B2, INTS4-GAB2, IRF2BP2-CDX1, JAZF1-PHF1, JAZF1-SUZ12, KIAA1549-BRAF, KIAA1598-ROS1, KIFSB-ALK, KIFSB-RET, KLC1-ALK, KLK2-ETV1, KLK2-ETV4, KMT2A-ABI1, KMT2A-ABI2, KMT2A-ACTN4, KMT2A-AFF1, KMT2A-AFF3, KMT2A-AFF4, KMT2A-ARHGAP26, KMT2A-ARHGEF12, KMT2A-BTBD18, KMT2A-CASCS, KMT2A-CASP8AP2, KMT2A-CBL, KMT2A-CREBBP, KMT2A-CT45A2, KMT2A-DAB2IP, KMT2A-EEFSEC, KMT2A-ELL, KMT2A-EP300, KMT2A-EPS15, KMT2A-FOXO3, KMT2A-FOXO4, KMT2A-FRYL, KMT2A-GAS7, KMT2A-GMPS, KMT2A-GPHN, KMT2A-KIAA0284_ENST00000414716, KMT2A-KIAA1524, KMT2A-LASP1, KMT2A-LPP, KMT2A-MAPRE1, KMT2A-MLLT1, KMT2A-MLLT10, KMT2A-MLLT11, KMT2A-MLLT3, KMT2A-MLLT4_ENST00000392108, KMT2A-MLLT6, KMT2A-MYO1F, KMT2A-NCKIPSD, KMT2A-NRIP3, KMT2A-PDS5A, KMT2A-PICALM, KMT2A-PRRC1, KMT2A-SARNP, KMT2A-SEPT2, KMT2A-SEPT5, KMT2A-SEPT6, KMT2A-SEPT9_ENST00000427177, KMT2A-SH3GL1, KMT2A-SORBS2, KMT2A-TET1, KMT2A-TOP3A, KMT2A-ZFYVE19, KTN1-RET, LIFR_ENST00000263409-PLAG1, LMNA-NTRK1_ENST00000392302, LRIG3-ROS1, LSM14A-BRAF, MARK4-ERCC2, MBOAT2-PRKCE, MBTD1_ENST00000586178-CXorf67_ENST00000342995, MEAF6-PHF1, MKRN1-BRAF, MSN-ALK, MYB_ENST00000341911-NFIB_ENST00000397581, MYO5A-ROS1, NAB2-STAT6, NACC2-NTRK2, NCOA4 ENST00000452682-RET, NDRG1-ERG, NF1-ACCN1, NFIA-EHF, NFIX_ENST00000360105-MAST1_ENST00000251472, NONO-TFE3, NOTCH1_ENST00000277541-GABBR2, NPM1-ALK, NTN1-ACLY, NUP107-LGR5, NUP214-ABL1, NUP98-KDM5A_ENST00000399788, OMD-USP6_ENST00000250066, PAX3-FOXO1, PAX3-NCOA1, PAX3-NCOA2, PAX5-JAK2, PAX7-FOXO1, PAX8-PPARG, PCM1-JAK2, PCM1-RET, PLA2R1-RBMS1, PLXND1-TMCC1, PML-RARA, PPFIBP1-ALK, PPFIBP1-ROS1, PRCC-TFE3, PRKAR1A-RET, PTPRK-RSPO3, PWWP2A-ROS1, QKI-NTRK2, RAF1-DAZL, RANBP2-ALK, RBM14-PACS1, RGS22-SYCP1, RNF130-BRAF, RUNX1-RUNX1T1, SDC4-ROS1, SEC16A_NM_014866.1-NOTCH1_ENST00000277541, SEC31A-ALK, SEC31A-JAK2, SEPT8-AFF4, SET-NUP214, SFPQ-TFE3, SLC22A1-CUTA, SLC26A6-PRKAR2A, SLC34A2-ROS1, SLC45A3-BRAF, SLC45A3-ELK4, SLC45A3-ERG, SLC45A3-ETV1, SLC45A3-ETV5_ENST00000306376, SND1-BRAF, SQSTM1-ALK, SRGAP3-RAF1, SS18-SSX1, SS18-SSX2, SS18-SSX4, SS18L1-SSX1, SSBP2-JAK2, SSH2-SUZ12, STIL-TAL1, STRN-ALK, SUSD1-ROD1, TADA2A_ENST00000394395-MAST1_ENST00000251472, TAF15-NR4A3, TBL1XR1-TP63, TCEA1_ENST00000521604-PLAG1, TCF12-NR4A3, TCF3-PBX1, TECTA-TBCEL, TFG-ALK, TFG-NR4A3, TFG-NTRK1_ENST00000392302, THRAP3-USP6_ENST00000250066, TMPRSS2-ERG, TMPRSS2-ETV1, TMPRSS2-ETV4, TMPRSS2-ETV5_ENST00000306376, TP53-NTRK1_ENST00000392302, TPM3-ALK, TPM3-NTRK1_ENST00000392302, TPM3-ROS1, TPM3_ENST00000368530-ROS1, TPM4-ALK, TRIM24-RET, TRIM27-RET, TRIM33_ENST00000358465-RET, UBE2L3-KRAS, VCL-ALK, VTI1A-TCF7L2, YWHAE_ENST00000264335-FAM22A_ENST00000381707, YWHAE_ENST00000264335-NUTM2B, ZC3H7B-BCOR_ENST00000378444, ZCCHC8-ROS1, ZNF700-MAST1_ENST00000251472, or ZSCAN30-BRAF.

In some embodiments, the gene (e.g., oncogene) or its gene product comprises one or more alterations relative to the corresponding wild-type gene (e.g., proto-oncogene). For instance, the one or more alterations may comprise a mutation or mutations within the gene or gene product, which affects amount or activity of the gene or gene product, as compared to the normal or wild-type gene. The alteration can be in amount, structure, and/or activity in a cancer tissue or cancer cell, as compared to its amount, structure, and/or activity, in a normal or healthy tissue or cell (e.g., a control), and can be associated with a disease state, such as cancer. For example, an alteration can comprise an altered nucleotide sequence (e.g., a mutation), amino acid sequence, chromosomal translocation, intra-chromosomal inversion, copy number, expression level, protein level, protein activity, or methylation status, in a cancer tissue or cancer cell, as compared to a normal, healthy tissue or cell. Exemplary mutations include, but are not limited to, point mutations (e.g., silent, missense, or nonsense), deletions, insertions, inversions, duplications, translocations, and inter- and intra-chromosomal rearrangements. Mutations can be present in the coding or non-coding region of the gene. In certain embodiments, the alteration(s) comprises a rearrangement, e.g., a genomic rearrangement comprising one or more introns or fragments thereof (e.g., one or more rearrangements in the 5′- and/or 3′-UTR).

In some embodiments, an associated gene may be a gene involved in cell development and/or differentiation.

In some embodiments, an associated gene may be a gene involved in one or more diseases, disorders, or conditions, e.g., cancer.

In some embodiments, an associated gene may be fusion gene selected from: CCDCl₆-RET, PAX3-FOXO, BRC-ABL1, EML4-ALK, ETV6-RUNX1, TMPRSS2-ERG, TCF3-PBX1, KMT2A-AFF1, or EWSR1-FLI1.In some embodiments, an associated gene may be a gene that encodes a component of transcription machinery and/or a transcriptional regulator; in some such embodiments, the target gene may encode a polypeptide that itself participates in one or more genomic complexes within the relevant system (e.g., cell, tissue, organism, etc.). In some such embodiments, targeted destabilization or inhibiting formation of the genomic complex with which the gene is associated may modulate expression both of the associated gene and with one or more genes associated with the genomic complexes in which the encoded polypeptide(s) participate. In some embodiments, a gene associated with a genomic complex in accordance with the present invention encodes a transcriptional regulator selected from the group consisting of activators and repressors.

### Polypeptide Components

As described herein, certain polypeptide complex components such as, for example, transcription machinery and/or regulatory factors, may be targeted as a way to modulate genomic complexes containing them, for example, by altering, e.g. structure and/or function, extent of complex formation, etc., as described herein. In some embodiments, disrupting agents for use in the methods described herein target one or more polypeptide components of a genomic complex. In some embodiments, polypeptide components include nucleating polypeptides, components of the transcription machinery, transcription regulators, or any protein listed in Table 2.

Nucleating polypeptides

A nucleating polypeptide may promote formation of an anchor sequence-mediated conjunction. Nucleating polypeptides that may be targeted by disrupting agents as described herein may include, for example, proteins (e.g., CTCF, USF1, YY1, TAF3, ZNF143, etc.) that bind specifically to anchor sequences, or other proteins (e.g., transcription factors, etc.) whose binding to a particular genomic sequence element may initiate formation of a genomic complex as described herein.

A nucleating polypeptide may be, e.g., CTCF, cohesin, USF1, YY1, TATA-box binding protein associated factor 3 (TAF3), ZNF143 binding motif, or another polypeptide that promotes formation of an anchor sequence-mediated conjunction. A nucleating polypeptide may be an endogenous polypeptide or other protein, such as a transcription factor, e.g., autoimmune regulator (AIRE), another factor, e.g., X-inactivation specific transcript (XIST), or an engineered polypeptide that is engineered to recognize a specific DNA sequence of interest, e.g., having a zinc finger, leucine zipper or bHLH domain for sequence recognition. A nucleating polypeptide may modulate DNA interactions within or around the anchor sequence-mediated conjunction. For example, a nucleating polypeptide can recruit other factors to an anchor sequence that alters an anchor sequence-mediated conjunction formation or formation and/or stabilization.

A nucleating polypeptide may also have a dimerization domain for homo- or heterodimerization. One or more nucleating polypeptides, e.g., endogenous and engineered, may interact to promote formation of an anchor sequence-mediated conjunction. In some embodiments, a nucleating polypeptide is engineered to destabilize an anchor sequence-mediated conjunction. In some embodiments, a nucleating polypeptide is engineered to decrease binding of a target sequence, e.g., target sequence binding affinity is decreased.

Nucleating polypeptides and their corresponding anchor sequences may be identified through use of cells that harbor inactivating mutations in CTCF and Chromosome Conformation Capture or 3C-based methods, e.g., Hi-C or high-throughput sequencing, to examine topologically associated domains, e.g., topological interactions between distal DNA regions or loci, in the absence of CTCF. Long-range DNA interactions may also be identified. Additional analyses may include ChIA-PET analysis using a bait, such as Cohesin, YY1 or USF1, ZNF143 binding motif, and MS to identify complexes that are associated with a bait.

In some embodiments, one or more nucleating polypeptides have a binding affinity for an anchor sequence greater than or less than a reference value, e.g., binding affinity for an anchor sequence in absence of an alteration.

In some embodiments, a nucleating polypeptide is modulated, e.g. a binding affinity for an anchor sequence within an anchor sequence-mediated conjunction, to alter its interaction with an anchor sequence-mediated conjunction.

Transcription Machinery

Those skilled in the art are familiar with proteins that participate as part of the transcription machinery involved in transcribing a particular gene (e.g., a protein-coding gene). For example, RNA polymerase (e.g., RNA polymerase II), general transcription factors such as TFIIA, TFIIB, TFIID, TFIIE, TFIIF, and TFIIH, Mediator, certain elongation factors, etc.

Targeting one or more components of transcription machinery involved in a particular genomic complex may alter extent of complex formation and/or may alter expression of one or more genes associated with the complex. For example, in some embodiments, targeting a transcription machinery component may decrease complex level, for example by inhibiting or destabilizing interactions between the targeted component and one or more other components of a genomic complex.

Transcription Regulators

In some embodiments, technologies provided herein may inhibit formation of and/or destabilize a particular genomic complex by targeting one or more transcription regulatory proteins involved or otherwise associated with the complex.

Those skilled in the art are aware of a large variety of transcriptional regulatory proteins (see Table 2), many of which are DNA binding proteins (e.g., containing a DNA binding domain such as a helix-loop-helix motif, ETS, a forkhead, a leucine zipper, a Pit-Oct-Unc domain, and/or a zinc finger as described below), many of which interact with core transcriptional machinery by way of interaction with Mediator. In some embodiments, a transcriptional regulatory protein may be or comprise an activator (e.g., that may bind to an enhancer). In some embodiments, a transcriptional regulatory protein may be or comprise a repressor (e.g., that may bind to a silencer).

In some embodiments, targeting a transcriptional regulator protein may decrease genomic complex formation level, for example by inhibiting and/or destabilizing interactions between the targeted component and one or more other components (e.g., with Mediator).

In some embodiments, a transcriptional regulatory protein is classified by superclass, class, and family.

In some embodiments, a superclass of transcriptional regulatory proteins is or comprises a “Basic Domain.” In some embodiments, within a “Basic Domain” superclass are classes comprising Leucine zipper (bZIP), Helix-loop-helix factors (bHLH), Helix-loop-helix/leucine zipper factors (bHLH-ZIP), NF-1, RF-X, and bHSH.

In some embodiments, a “Leucine zipper (bZIP)” class comprises families AP-1 and AP-1-like (includes c-FOS/c-JUN), CREB, C/EBP-like, bZIP/PAR, Plant G-box binding factors and ZIP only.

In some embodiments, a “Helix-loop-helix factors (bHLH)” class comprises families Ubiquitous (class A) factors, Myogenic transcription factors (MyoD), Achaete-Scute, and Tal/Twist/Atonal/Hen.

In some embodiments, a “Helix-loop-helix/leucine zipper factors (bHLH-ZIP)” class comprises families Ubiquitious bHLH-ZIP (includes USF (USF1, USF2); SREBP), and Cell-cycle controlling factors (c-Myc).

In some embodiments, a “NF-1” class comprises families NF-1 (A, B, C, X).

In some embodiments, a “RF-X” class comprises families RF-X (1, 2, 3, 4, 5, ANK).

In some embodiments, a superclass of transcriptional regulatory proteins is or comprises “Zinc-coordinating DNA-binding domains.” In some embodiments, within a “Zinc-coordinating DNA binding domains” superclass are classes comprising Cys4 zinc finger of nuclear receptor type, Diverse Cys4 zinc fingers, Cys2His2 (C2H2) zinc finger domain, Cys6 cysteine-zinc cluster, and Zinc fingers of alternating composition.

In some embodiments, a “Cys4 zinc finger of nuclear receptor type” class comprises families Steroid hormone receptors and Thyroid hormone receptor-like factors.

In some embodiments, a “Diverse Cys4 zinc fingers” class comprises a GATA-factors family.

In some embodiments, a “Cys2His2 (C2H2) zinc finger domain” class comprises families Ubiquitous factors (includes TFIIIA, Sp1), Developmental/cell cycle regulators (includes Kruppel), and Large factors with NF-6B-like binding properties.

In some embodiments, a superclass of transcriptional regulatory proteins is or comprises “Helix-turn-helix.” In some embodiments, within a “Helix-turn-helix” superclass are classes comprising Homeo domain, Paired box, Fork head/winged helix, Heat Shock Factors, Tryptophan clusters, and TEA (Transcriptional Enhancer factor) domain.

In some embodiments, a “Homeo domain” class comprises families Homeo domain only (includes Ubx), POU domain factors (includes Oct), Homeo domain with LIM region, and Homeo domain plus zinc finger motifs.

In some embodiments, a “Paired box domain” class comprises families Paired box plus homeo domain and Paired box domain only.

In some embodiments, a “Fork head/winged helix” class comprises families Developmental regulators (includes forkhead), Tissue-specific regulators, Cell-cycle controlling factors, and Other regulators.

In some embodiments, a “Head Shock Factors” class comprises an HSF family.

In some embodiments, a “Tryptophan clusters” class comprises families Myb, ETS-type, and Interferon regulatory factors.

In some embodiments, a “TEA domain” class comprises families TEA (TEAD1, TEAD2, TEAD3, TEAD4).

In some embodiments, a superclass of transcriptional regulatory proteins is or comprises “Beta-scaffold factors with minor groove contacts.” In some embodiments, within a “Beta-scaffold factors with minor groove contacts” superclass are classes comprising RHR (Rel homology region), STAT, p53, MADS box, Beta-barrel alpha helix transcription factors, TATA binding proteins, HMG-box, Heterometric CCAAT factors, Grainyhead, Cold-shock domain factors, and Runt.

In some embodiments, a “RHR (Rel homology region)” class comprises families Rel/Ankyrin; NF-kB, Ankyrin only, and NFAT (nuclear factor of activated T-cells) (NFATC1, NFATC2, NFATC3).

In some embodiments, a “STAT” class comprises a STAT family.

In some embodiments, a “p53” class comprises a p53 family.

In some embodiments, a “MADS box” class comprises families Regulators of differentiation (includes Mef2), Responders to external signals (SRF (serum response factor)), and Metabolic regulators (ARG80).

In some embodiments, a “TATA binding proteins” class comprises a TBP family.

In some embodiments, a “HMG-box” class comprises families SOX genes and SRY, TCF-1, HMG2-related (SSRP1), UBF, and MATA.

In some embodiments, a “Heterometric CCAAT factors” class comprises a Heteromeric CCAAT factors family.

In some embodiments, a “Grainyhead” class comprises a Grainyhead family.

In some embodiments, a “Cold-shock domain (CSD) factors” class comprises a CSD family.

In some embodiments, a “Runt class” comprises a Runt family.

In some embodiments, other classes of transcriptional regulatory proteins comprise Copper first proteins, HMGI(Y) and HMGA1, Pocket domain, E1A-like factors, and AP2/EREBP-related factors.

In some embodiments, class “AP2/EREBP-related factors” comprises families “AP2, EREBP, AP2/B3 (ARF, ABI, RAV).

TABLE 2
Exemplary Human Transcription Regulatory Proteins

| # | Entry name | Protein names | Gene names |
|---|---|---|---|
| 1 | APLP2_HUMAN | Amyloid-like protein 2 (APLP-2) (APPH) | APLP2 |
| | | (Amyloid protein homolog) (CDEI box-binding | APPL2 |
| | | protein) (CDEBP) | |
| 2 | A4_HUMAN | Amyloid-beta A4 protein (ABPP) (APPI) (APP) | APP |
| | | (Alzheimer disease amyloid protein) (Amyloid | A4 |
| | | precursor protein) (Amyloid-beta precursor protein) | AD1 |
| | | (Cerebral vascular amyloid peptide) (CVAP) | |
| | | (PreA4) (Protease nexin-II) (PN-II) [Cleaved into: | |
| | | N-APP; Soluble APP-alpha (S-APP-alpha); Soluble | |
| | | APP-beta (S-APP-beta); C99; Amyloid-beta | |
| | | protein 42 (Abeta42) (Beta-APP42); Amyloid-beta | |
| | | protein 40 (Abeta40) (Beta-APP40); C83; P3(42); | |
| | | P3(40); C80; Gamma-secretase C-terminal | |
| | | fragment 59 (Amyloid intracellular domain 59) | |
| | | (AICD-59) (AID(59)) (Gamma-CTF(59)); Gamma- | |
| | | secretase C-terminal fragment 57 (Amyloid | |
| | | intracellular domain 57) (AICD-57) (AID(57)) | |
| | | (Gamma-CTF(57)); Gamma-secretase C-terminal | |
| | | fragment 50 (Amyloid intracellular domain 50) | |
| | | (AICD-50) (AID(50)) (Gamma-CTF(50)); C31] | |
| 3 | ANDR_HUMAN | Androgen receptor (Dihydrotestosterone receptor) | AR |
| | | (Nuclear receptor subfamily 3 group C member 4) | DHTR |
| | | | NR3C4 |
| 4 | AIRE_HUMAN | Autoimmune regulator (Autoimmune | AIRE |
| | | polyendocrinopathy candidiasis ectodermal | APECED |
| | | dystrophy protein) (APECED protein) | |
| 5 | PKCB1_HUMAN | Protein kinase C-binding protein 1 (Cutaneous T- | ZMYND8 |
| | | cell lymphoma-associated antigen se14-3) (CTCL- | KIAA1125 |
| | | associated antigen se14-3) (Rack7) (Zinc finger | PRKCBP1 |
| | | MYND domain-containing protein 8) | RACK7 |
| 6 | HCFC1_HUMAN | Host cell factor 1 (HCF) (HCF-1) (C1 factor) | HCFC1 |
| | | (CFF) (VCAF) (VP16 accessory protein) [Cleaved | HCF1 |
| | | into: HCF N-terminal chain 1; HCF N-terminal | HFC1 |
| | | chain 2; HCF N-terminal chain 3; HCF N-terminal | |
| | | chain 4; HCF N-terminal chain 5; HCF N-terminal | |
| | | chain 6; HCF C-terminal chain 1; HCF C-terminal | |
| | | chain 2; HCF C-terminal chain 3; HCF C-terminal | |
| | | chain 4; HCF C-terminal chain 5; HCF C-terminal | |
| | | chain 6] | |
| 7 | SPT4H_HUMAN | Transcription elongation factor SPT4 (hSPT4) | SUPT4H1 |
| | | (DRB sensitivity-inducing factor 14 kDa subunit) | SPT4H |
| | | (DSIF p14) (DRB sensitivity-inducing factor small | SUPT4H |
| | | subunit) (DSIF small subunit) | |
| 8 | RFXK_HUMAN | DNA-binding protein RFXANK (Ankyrin repeat | RFXANK |
| | | family A protein 1) (Regulatory factor X subunit B) | ANKRA1 |
| | | (RFX-B) (Regulatory factor X-associated ankyrin- | RFXB |
| | | containing protein) | |
| 9 | TF2H3_HUMAN | General transcription factor IIH subunit 3 (Basic | GTF2H3 |
| | | transcription factor 2 34 kDa subunit) (BTF2 p34) | |
| | | (General transcription factor IIH polypeptide 3) | |
| | | (TFIIH basal transcription factor complex p34 | |
| | | subunit) | |
| 10 | CSN5_HUMAN | COP9 signalosome complex subunit 5 (SGN5) | COPS5 |
| | | (Signalosome subunit 5) (EC 3.4.—.—) (Jun | CSN5 |
| | | activation domain-binding protein 1) | JAB1 |
| 11 | SPOP_HUMAN | Speckle-type POZ protein (HIB homolog 1) | SPOP |
| | | (Roadkill homolog 1) | |
| 12 | TF2H2_HUMAN | General transcription factor IIH subunit 2 (Basic | GTF2H2 |
| | | transcription factor 2 44 kDa subunit) (BTF2 p44) | BTF2P44 |
| | | (General transcription factor IIH polypeptide 2) | |
| | | (TFIIH basal transcription factor complex p44 | |
| | | subunit) | |
| 13 | TEAD3_HUMAN | Transcriptional enhancer factor TEF-5 (DTEF-1) | TEAD3 |
| | | (TEA domain family member 3) (TEAD-3) | TEAD5 |
| | | | TEF5 |
| 14 | T2EA_HUMAN | General transcription factor IIE subunit 1 (General | GTF2E1 |
| | | transcription factor IIE 56 kDa subunit) | TF2E1 |
| | | (Transcription initiation factor IIE subunit alpha) | |
| | | (TFIIE-alpha) | |
| 15 | TF2H4_HUMAN | General transcription factor IIH subunit 4 (Basic | GTF2H4 |
| | | transcription factor 2 52 kDa subunit) (BTF2 p52) | |
| | | (General transcription factor IIH polypeptide 4) | |
| | | (TFIIH basal transcription factor complex p52 | |
| | | subunit) | |
| 16 | MYCN_HUMAN | N-myc proto-oncogene protein (Class E basic | MYCN |
| | | helix-loop-helix protein 37) (bHLHe37) | BHLHE37 |
| | | | NMYC |
| 17 | MAVS_HUMAN | Mitochondrial antiviral-signaling protein (MAVS) | MAVS |
| | | (CARD adapter inducing interferon beta) (Cardif) | IPS1 |
| | | (Interferon beta promoter stimulator protein 1) | KIAA1271 |
| | | (IPS-1) (Putative NF-kappa-B-activating protein | VISA |
| | | 031N) (Virus-induced-signaling adapter) (VISA) | |
| 18 | SCMH1_HUMAN | Polycomb protein SCMH1 (Sex comb on midleg | SCMH1 |
| | | homolog 1) | |
| 19 | SCML2_HUMAN | Sex comb on midleg-like protein 2 | SCML2 |
| 20 | SP140_HUMAN | Nuclear body protein SP140 (Lymphoid-restricted | SP140 |
| | | homolog of Sp100) (LYSp100) (Nuclear | LYSP100 |
| | | autoantigen Sp-140) (Speckled 140 kDa) | |
| 21 | SP100_HUMAN | Nuclear autoantigen Sp-100 (Nuclear dot- | SP100 |
| | | associated Sp100 protein) (Speckled 100 kDa) | |
| 22 | GTD2A_HUMAN | General transcription factor II-I repeat domain- | GTF2IRD2 |
| | | containing protein 2A (GTF2I repeat domain- | GTF2IRD2A |
| | | containing protein 2A) (Transcription factor | |
| | | GTF2IRD2-alpha) | |
| 23 | GTD2B_HUMAN | General transcription factor II-I repeat domain- | GTF2IRD2B |
| | | containing protein 2B (GTF2I repeat domain- | |
| | | containing protein 2B) (Transcription factor | |
| | | GTF2IRD2-beta) | |
| 24 | GT2D1_HUMAN | General transcription factor II-I repeat domain- | GTF2IRD1 |
| | | containing protein 1 (GTF2I repeat domain- | CREAM1 |
| | | containing protein 1) (General transcription factor | GTF3 |
| | | III) (MusTRD1/BEN) (Muscle TFII-I repeat | MUSTRD1 |
| | | domain-containing protein 1) (Slow-muscle-fiber | RBAP2 |
| | | enhancer-binding protein) (USE B1-binding | WBSCR11 |
| | | protein) (Williams-Beuren syndrome chromosomal | WBSCR12 |
| | | region 11 protein) (Williams-Beuren syndrome | |
| | | chromosomal region 12 protein) | |
| 25 | GTF2I_HUMAN | General transcription factor II-I (GTFII-I) (TFII-I) | GTF2I |
| | | (Bruton tyrosine kinase-associated protein 135) | BAP135 |
| | | (BAP-135) (BTK-associated protein 135) (SRF- | WBSCR6 |
| | | Phox1-interacting protein) (SPIN) (Williams- | |
| | | Beuren syndrome chromosomal region 6 protein) | |
| 26 | AFF1_HUMAN | AF4/FMR2 family member 1 (ALL1-fused gene | AFF1 |
| | | from chromosome 4 protein) (Protein AF-4) | AF4 |
| | | (Protein FEL) (Proto-oncogene AF4) | FEL |
| | | | MLLT2 |
| | | | PBM1 |
| 27 | PER1_HUMAN | Period circadian protein homolog 1 (hPER1) | PER1 |
| | | (Circadian clock protein PERIOD 1) (Circadian | KIAA0482 |
| | | pacemaker protein Rigui) | PER |
| | | | RIGUI |
| 28 | MED1_HUMAN | Mediator of RNA polymerase II transcription | MED1 |
| | | subunit 1 (Activator-recruited cofactor 205 kDa | ARC205 |
| | | component) (ARC205) (Mediator complex subunit | CRSP1 |
| | | 1) (Peroxisome proliferator-activated receptor- | CRSP200 |
| | | binding protein) (PBP) (PPAR-binding protein) | DRIP205 |
| | | (Thyroid hormone receptor-associated protein | DRIP230 |
| | | complex 220 kDa component) (Trap220) (Thyroid | PBP |
| | | receptor-interacting protein 2) (TR-interacting | PPARBP |
| | | protein 2) (TRIP-2) (Vitamin D receptor- | PPARGBP |
| | | interacting protein complex component DRIP205) | RB18A |
| | | (p53 regulatory protein RB18A) | TRAP220 |
| | | | TRIP2 |
| 29 | BAZ2A_HUMAN | Bromodomain adjacent to zinc finger domain | BAZ2A |
| | | protein 2A (Transcription termination factor I- | KIAA0314 |
| | | interacting protein 5) (TTF-I-interacting protein 5) | TIP5 |
| | | (Tip5) (hWALp3) | |
| 30 | TYB4_HUMAN | Thymosin beta-4 (T beta-4) (Fx) [Cleaved into: | TMSB4X |
| | | Hematopoietic system regulatory peptide | TB4X |
| | | (Seraspenide)] | THYB4 |
| | | | TMSB4 |
| 31 | ANXA3_HUMAN | Annexin A3 (35-alpha calcimedin) (Annexin III) | ANXA3 |
| | | (Annexin-3) (Inositol 1,2-cyclic phosphate 2- | ANX3 |
| | | phosphohydrolase) (Lipocortin III) (Placental | |
| | | anticoagulant protein III) (PAP-III) | |
| 32 | FLNA_HUMAN | Filamin-A (FLN-A) (Actin-binding protein 280) | FLNA |
| | | (ABP-280) (Alpha-filamin) (Endothelial actin- | FLN |
| | | binding protein) (Filamin-1) (Non-muscle filamin) | FLN1 |
| 33 | LIF_HUMAN | Leukemia inhibitory factor (LIF) (Differentiation- | LIF |
| | | stimulating factor) (D factor) (Melanoma-derived | HILDA |
| | | LPL inhibitor) (MLPLI) (Emfilermin) | |
| 34 | MAX_HUMAN | Protein max (Class D basic helix-loop-helix protein | MAX |
| | | 4) (bHLHd4) (Myc-associated factor X) | BHLHD4 |
| 35 | PEBB_HUMAN | Core-binding factor subunit beta (CBF-beta) | CBFB |
| | | (Polyomavirus enhancer-binding protein 2 beta | |
| | | subunit) (PEA2-beta) (PEBP2-beta) (SL3-3 | |
| | | enhancer factor 1 subunit beta) (SL3/AKV core- | |
| | | binding factor beta subunit) | |
| 36 | SRY_HUMAN | Sex-determining region Y protein (Testis- | SRY |
| | | determining factor) | TDF |
| 37 | NC2A_HUMAN | Dr1-associated corepressor (Dr1-associated protein | DRAP1 |
| | | 1) (Negative cofactor 2-alpha) (NC2-alpha) | |
| 38 | THAP1_HUMAN | THAP domain-containing protein 1 | THAP1 |
| 39 | FEV_HUMAN | Protein FEV (Fifth Ewing variant protein) (PC12 | FEV |
| | | ETS domain-containing transcription factor 1) | PET1 |
| | | (PC12 ETS factor 1) (Pet-1) | |
| 40 | DLX3_HUMAN | Homeobox protein DLX-3 | DLX3 |
| 41 | HXB1_HUMAN | Homeobox protein Hox-B1 (Homeobox protein | HOXB1 |
| | | Hox-2I) | HOX2I |
| 42 | PO6F1_HUMAN | POU domain, class 6, transcription factor 1 (Brain- | POU6F1 |
| | | specific homeobox/POU domain protein 5) (Brain- | BRN5 |
| | | 5) (Brn-5) (mPOU homeobox protein) | MPOU |
| | | | TCFB1 |
| 43 | USF1_HUMAN | Upstream stimulatory factor 1 (Class B basic helix- | USF1 |
| | | loop-helix protein 11) (bHLHb11) (Major late | BHLHB11 |
| | | transcription factor 1) | USF |
| 44 | CDX2_HUMAN | Homeobox protein CDX-2 (CDX-3) (Caudal-type | CDX2 |
| | | homeobox protein 2) | CDX3 |
| 45 | PITX2_HUMAN | Pituitary homeobox 2 (ALL1-responsive protein | PITX2 |
| | | ARP1) (Homeobox protein PITX2) (Paired-like | ARP1 |
| | | homeodomain transcription factor 2) (RIEG bicoid- | RGS |
| | | related homeobox transcription factor) (Solurshin) | RIEG |
| | | | RIEG1 |
| 46 | NKX25_HUMAN | Homeobox protein Nkx-2.5 (Cardiac-specific | NKX2-5 |
| | | homeobox) (Homeobox protein CSX) (Homeobox | CSX |
| | | protein NK-2 homolog E) | NKX2.5 |
| | | | NKX2E |
| 47 | TAL1_HUMAN | T-cell acute lymphocytic leukemia protein 1 (TAL- | TAL1 |
| | | I) (Class A basic helix-loop-helix protein 17) | BHLHA17 |
| | | (bHLHa17) (Stem cell protein) (T-cell | SCL |
| | | leukemia/lymphoma protein 5) | TCL5 |
| 48 | SPDEF_HUMAN | SAM pointed domain-containing Ets transcription | SPDEF |
| | | factor (Prostate epithelium-specific Ets | PDEF |
| | | transcription factor) (Prostate-specific Ets) | PSE |
| | | (Prostate-derived Ets factor) | |
| 49 | TBP_HUMAN | TATA-box-binding protein (TATA sequence- | TBP |
| | | binding protein) (TATA-binding factor) (TATA- | GTF2D1 |
| | | box factor) (Transcription initiation factor TFIID | TF2D |
| | | TBP subunit) | TFIID |
| 50 | AIM2_HUMAN | Interferon-inducible protein AIM2 (Absent in | AIM2 |
| | | melanoma 2) | |
| 51 | CEBPB_HUMAN | CCAAT/enhancer-binding protein beta (C/EBP | CEBPB |
| | | beta) (Liver activator protein) (LAP) (Liver- | TCF5 |
| | | enriched inhibitory protein) (LIP) (Nuclear factor | PP9092 |
| | | NF-IL6) (Transcription factor 5) (TCF-5) | |
| 52 | NFYA_HUMAN | Nuclear transcription factor Y subunit alpha | NFYA |
| | | (CAAT box DNA-binding protein subunit A) | |
| | | (Nuclear transcription factor Y subunit A) (NF- | |
| | | YA) | |
| 53 | FOXA3_HUMAN | Hepatocyte nuclear factor 3-gamma (HNF-3- | FOXA3 |
| | | gamma) (HNF-3G) (Fork head-related protein FKH | HNF3G |
| | | H3) (Forkhead box protein A3) (Transcription | TCF3G |
| | | factor 3G) (TCF-3G) | |
| 54 | MAFA_HUMAN | Transcription factor MafA (Pancreatic beta-cell- | MAFA |
| | | specific transcriptional activator) (Transcription | |
| | | factor RIPE3b1) (V-maf musculoaponeurotic | |
| | | fibrosarcoma oncogene homolog A) | |
| 55 | MEF2B_HUMAN | Myocyte-specific enhancer factor 2B (RSRFR2) | MEF2B |
| | | (Serum response factor-like protein 2) | XMEF2 |
| 56 | DMRT1_HUMAN | Doublesex- and mab-3-related transcription factor | DMRT1 |
| | | 1 (DM domain expressed in testis protein 1) | DMT1 |
| 57 | FOS_HUMAN | Proto-oncogene c-Fos (Cellular oncogene fos) | FOS |
| | | (G0/G1 switch regulatory protein 7) | G0S7 |
| 58 | MEIS1_HUMAN | Homeobox protein Meis1 | MEIS1 |
| 59 | PAX5_HUMAN | Paired box protein Pax-5 (B-cell-specific | PAX5 |
| | | transcription factor) (BSAP) | |
| 60 | TBX1_HUMAN | T-box transcription factor TBX1 (T-box protein 1) | TBX1 |
| | | (Testis-specific T-box protein) | |
| 61 | E2F4_HUMAN | Transcription factor E2F4 (E2F-4) | E2F4 |
| 62 | TYY1_HUMAN | Transcriptional repressor protein YY1 (Delta | YY1 |
| | | transcription factor) (INO80 complex subunit S) | INO80S |
| | | (NF-E1) (Yin and yang 1) (YY-1) | |
| 63 | VDR_HUMAN | Vitamin D3 receptor (VDR) (1,25- | VDR |
| | | dihydroxyvitamin D3 receptor) (Nuclear receptor | NR1I1 |
| | | subfamily 1 group I member 1) | |
| 64 | ELK1_HUMAN | ETS domain-containing protein Elk-1 | ELK1 |
| 65 | PBX1_HUMAN | Pre-B-cell leukemia transcription factor 1 | PBX1 |
| | | (Homeobox protein PBX1) (Homeobox protein PRL) | PRL |
| 66 | ELK4_HUMAN | ETS domain-containing protein Elk-4 (Serum | ELK4 |
| | | response factor accessory protein 1) (SAP-1) (SRF | SAP1 |
| | | accessory protein 1) | |
| 67 | FOXP3_HUMAN | Forkhead box protein P3 (Scurfin) [Cleaved into: | FOXP3 |
| | | Forkhead box protein P3, C-terminally processed; | IPEX |
| | | Forkhead box protein P3 41 kDa form] | JM2 |
| 68 | ERR2_HUMAN | Steroid hormone receptor ERR2 (ERR beta-2) | ESRRB |
| | | (Estrogen receptor-like 2) (Estrogen-related | ERRB2 |
| | | receptor beta) (ERR-beta) (Nuclear receptor | ESRL2 |
| | | subfamily 3 group B member 2) | NR3B2 |
| 69 | TEAD4_HUMAN | Transcriptional enhancer factor TEF-3 (TEA | TEAD4 |
| | | domain family member 4) (TEAD-4) | RTEF1 |
| | | (Transcription factor 13-like 1) (Transcription | TCF13L1 |
| | | factor RTEF-1) | TEF3 |
| 70 | GATA3_HUMAN | Trans-acting T-cell-specific transcription factor | GATA3 |
| | | GATA-3 (GATA-binding factor 3) | |
| 71 | TFDP2_HUMAN | Transcription factor Dp-2 (E2F dimerization | TFDP2 |
| | | partner 2) | DP2 |
| 72 | THB_HUMAN | Thyroid hormone receptor beta (Nuclear receptor | THRB |
| | | subfamily 1 group A member 2) (c-erbA-2) (c- | ERBA2 |
| | | erbA-beta) | NR1A2 |
| | | | THR1 |
| 73 | RARA_HUMAN | Retinoic acid receptor alpha (RAR-alpha) (Nuclear | RARA |
| | | receptor subfamily 1 group B member 1) | NR1B1 |
| 74 | RXRA_HUMAN | Retinoic acid receptor RXR-alpha (Nuclear | RXRA |
| | | receptor subfamily 2 group B member 1) (Retinoid | NR2B1 |
| | | X receptor alpha) | |
| 75 | ETS2_HUMAN | Protein C-ets-2 | ETS2 |
| 76 | HNF4A_HUMAN | Hepatocyte nuclear factor 4-alpha (HNF-4-alpha) | HNF4A |
| | | (Nuclear receptor subfamily 2 group A member 1) | HNF4 |
| | | (Transcription factor 14) (TCF-14) (Transcription | NR2A1 |
| | | factor HNF-4) | TCF14 |
| 77 | MEIS2_HUMAN | Homeobox protein Meis2 (Meis1-related protein 1) | MEIS2 |
| | | | MRG1 |
| 78 | PAX3_HUMAN | Paired box protein Pax-3 (HuP2) | PAX3 |
| | | | HUP2 |
| 79 | MECP2_HUMAN | Methyl-CpG-binding protein 2 (MeCp-2 protein) | MECP2 |
| | | (MeCp2) | |
| 80 | SUH_HUMAN | Recombining binding protein suppressor of hairless | RBPJ |
| | | (CBF-1) (J kappa-recombination signal-binding | IGKJRB |
| | | protein) (RBP-J kappa) (RBP-J) (RBP-JK) (Renal | IGKJRB1 |
| | | carcinoma antigen NY-REN-30) | RBPJK |
| | | | RBPSUH |
| 81 | IRF7_HUMAN | Interferon regulatory factor 7 (IRF-7) | IRF7 |
| 82 | PPARG_HUMAN | Peroxisome proliferator-activated receptor gamma | PPARG |
| | | (PPAR-gamma) (Nuclear receptor subfamily 1 | NR1C3 |
| | | group C member 3) | |
| 83 | MEF2A_HUMAN | Myocyte-specific enhancer factor 2A (Serum | MEF2A |
| | | response factor-like protein 1) | MEF2 |
| 84 | SRF_HUMAN | Serum response factor (SRF) | SRF |
| 85 | SOX9_HUMAN | Transcription factor SOX-9 | SOX9 |
| 86 | HSF1_HUMAN | Heat shock factor protein 1 (HSF 1) (Heat shock | HSF1 |
| | | transcription factor 1) (HSTF 1) | HSTF1 |
| 87 | EGR1_HUMAN | Early growth response protein 1 (EGR-1) (AT225) | EGR1 |
| | | (Nerve growth factor-induced protein A) (NGFI-A) | KROX24 |
| | | (Transcription factor ETR103) (Transcription | ZNF225 |
| | | factor Zif268) (Zinc finger protein 225) (Zinc | |
| | | finger protein Krox-24) | |
| 88 | ESR1_HUMAN | Estrogen receptor (ER) (ER-alpha) (Estradiol | ESR1 |
| | | receptor) (Nuclear receptor subfamily 3 group A | ESR |
| | | member 1) | NR3A1 |
| 89 | NR1D1_HUMAN | Nuclear receptor subfamily 1 group D member 1 | NR1D1 |
| | | (Rev-erbA-alpha) (V-erbA-related protein 1) | EAR1 |
| | | (EAR-1) | HREV |
| | | | THRAL |
| 90 | BMAL1_HUMAN | Aryl hydrocarbon receptor nuclear translocator-like | ARNTL |
| | | protein 1 (Basic-helix-loop-helix-PAS protein | BHLHE5 |
| | | MOP3) (Brain and muscle ARNT-like 1) (Class E | BMAL1 |
| | | basic helix-loop-helix protein 5) (bHLHe5) | MOP3 |
| | | (Member of PAS protein 3) (PAS domain- | PASD3 |
| | | containing protein 3) (bHLH-PAS protein JAP3) | |
| 91 | HNF1A_HUMAN | Hepatocyte nuclear factor 1-alpha (HNF-1-alpha) | HNF1A |
| | | (HNF-1A) (Liver-specific transcription factor LF- | TCF1 |
| | | B1) (LFB1) (Transcription factor 1) (TCF-1) | |
| 92 | FUBP1_HUMAN | Far upstream element-binding protein 1 (FBP) | FUBP1 |
| | | (FUSE-binding protein 1) (DNA helicase V) (hDH | |
| | | V) | |
| 93 | FOXO1_HUMAN | Forkhead box protein O1 (Forkhead box protein | FOXO1 |
| | | O1A) (Forkhead in rhabdomyosarcoma) | FKHR |
| | | | FOXO1A |
| 94 | FOXP2_HUMAN | Forkhead box protein P2 (CAG repeat protein 44) | FOXP2 |
| | | (Trinucleotide repeat-containing gene 10 protein) | CAGH44 |
| | | | TNRC10 |
| 95 | PO2F1_HUMAN | POU domain, class 2, transcription factor 1 (NF- | POU2F1 |
| | | A1) (Octamer-binding protein 1) (Oct-1) (Octamer- | OCT1 |
| | | binding transcription factor 1) (OTF-1) | OTF1 |
| 96 | TBX3_HUMAN | T-box transcription factor TBX3 (T-box protein 3) | TBX3 |
| 97 | STAT1_HUMAN | Signal transducer and activator of transcription 1- | STAT1 |
| | | alpha/beta (Transcription factor ISGF-3 | |
| | | components p91/p84) | |
| 98 | FOXM1_HUMAN | Forkhead box protein M1 (Forkhead-related protein | FOXM1 |
| | | FKHL16) (Hepatocyte nuclear factor 3 forkhead | FKHL16 |
| | | homolog 11) (HFH-11) (HNF-3/fork-head homolog | HFH11 |
| | | 11) (M-phase phosphoprotein 2) (MPM-2 reactive | MPP2 |
| | | phosphoprotein 2) (Transcription factor Trident) | WIN |
| | | (Winged-helix factor from INS-1 cells) | |
| 99 | TOP1_HUMAN | DNA topoisomerase 1 (EC 5.99.1.2) (DNA | TOP1 |
| | | topoisomerase I) | |
| 100 | CLOCK_HUMAN | Circadian locomoter output cycles protein kaput | CLOCK |
| | | (hCLOCK) (EC 2.3.1.48) (Class E basic helix- | BHLHE8 |
| | | loop-helix protein 8) (bHLHe8) | KIAA0334 |
| 101 | E2F8_HUMAN | Transcription factor E2F8 (E2F-8) | E2F8 |
| 102 | NFKB2_HUMAN | Nuclear factor NF-kappa-B p100 subunit (DNA- | NFKB2 |
| | | binding factor KBF2) (H2TF1) (Lymphocyte | LYT10 |
| | | translocation chromosome 10 protein) (Nuclear | |
| | | factor of kappa light polypeptide gene enhancer in | |
| | | B-cells 2) (Oncogene Lyt-10) (Lyt10) [Cleaved | |
| | | into: Nuclear factor NF-kappa-B p52 subunit] | |
| 103 | NFAC2_HUMAN | Nuclear factor of activated T-cells, cytoplasmic 2 | NFATC2 |
| | | (NF-ATc2) (NFATc2) (NFAT pre-existing subunit) | NFAT1 |
| | | (NF-ATp) (T-cell transcription factor NFAT1) | NFATP |
| 104 | NFAC1_HUMAN | Nuclear factor of activated T-cells, cytoplasmic 1 | NFATC1 |
| | | (NF-ATc1) (NFATc1) (NFAT transcription | NFAT2 |
| | | complex cytosolic component) (NF-ATc) (NFATc) | NFATC |
| 105 | RFX1_HUMAN | MHC class II regulatory factor RFX1 (Enhancer | RFX1 |
| | | factor C) (EF-C) (Regulatory factor X 1) (RFX) | |
| | | (Transcription factor RFX1) | |
| 106 | GLI1_HUMAN | Zinc finger protein GLI1 (Glioma-associated | GLI1 |
| | | oncogene) (Oncogene GLI) | GLI |
| 107 | SRBP1_HUMAN | Sterol regulatory element-binding protein 1 | SREBF1 |
| | | (SREBP-1) (Class D basic helix-loop-helix protein | BHLHD1 |
| | | 1) (bHLHd1) (Sterol regulatory element-binding | SREBP1 |
| | | transcription factor 1) [Cleaved into: Processed | |
| | | sterol regulatory element-binding protein 1] | |
| 108 | ARI5B_HUMAN | AT-rich interactive domain-containing protein 5B | ARID5B |
| | | (ARID domain-containing protein 5B) (MRFl-like | DESRT |
| | | protein) (Modulator recognition factor 2) (MRF-2) | MRF2 |
| 109 | NFAT5_HUMAN | Nuclear factor of activated T-cells 5 (NF-AT5) (T- | NFAT5 |
| | | cell transcription factor NFAT5) (Tonicity- | KIAA0827 |
| | | responsive enhancer-binding protein) (TonE- | TONEBP |
| | | binding protein) (TonEBP) | |
| 110 | PHF5A_HUMAN | PHD finger-like domain-containing protein 5A | PHF5A |
| | | (PHD finger-like domain protein 5A) (Splicing | |
| | | factor 3B-associated 14 kDa protein) (SF3b14b) | |
| 111 | EDF1_HUMAN | Endothelial differentiation-related factor 1 (EDF-1) | EDF1 |
| | | (Multiprotein-bridging factor 1) (MBF1) | |
| 112 | LMO4_HUMAN | LIM domain transcription factor LMO4 (Breast | LMO4 |
| | | tumor autoantigen) (LIM domain only protein 4) | |
| | | (LMO-4) | |
| 113 | MAD1_HUMAN | Max dimerization protein 1 (Max dimerizer 1) | MXD1 |
| | | (Protein MAD) | MAD |
| 114 | TSN_HUMAN | Translin (EC 3.1.—.—) (Component 3 of promoter of | TSN |
| | | RISC) (C3PO) | |
| 115 | NKX31_HUMAN | Homeobox protein Nkx-3.1 (Homeobox protein | NKX3-1 |
| | | NK-3 homolog A) | NKX3.1 |
| | | | NKX3A |
| 116 | TFAM_HUMAN | Transcription factor A, mitochondrial (mtTFA) | TFAM |
| | | (Mitochondrial transcription factor 1) (MtTF1) | TCF6 |
| | | (Transcription factor 6) (TCF-6) (Transcription | TCF6L2 |
| | | factor 6-like 2) | |
| 117 | BARX1_HUMAN | Homeobox protein BarH-like 1 | BARX1 |
| 118 | GSC_HUMAN | Homeobox protein goosecoid | GSC |
| 119 | HXC9_HUMAN | Homeobox protein Hox-C9 (Homeobox protein | HOXC9 |
| | | Hox-3B) | HOX3B |
| 120 | SNAI1_HUMAN | Zinc finger protein SNAI1 (Protein snail homolog | SNAI1 |
| | | 1) (Protein sna) | SNAH |
| 121 | ELF5_HUMAN | ETS-related transcription factor Elf-5 (E74-like | ELF5 |
| | | factor 5) (Epithelium-restricted ESE-1-related Ets | ESE2 |
| | | factor) (Epithelium-specific Ets transcription factor | |
| | | 2) (ESE-2) | |
| 122 | HHEX_HUMAN | Hematopoietically-expressed homeobox protein | HHEX |
| | | HHEX (Homeobox protein HEX) (Homeobox | HEX |
| | | protein PRH) | PRH |
| | | | PRHX |
| 123 | HES1_HUMAN | Transcription factor HES-1 (Class B basic helix- | HES1 |
| | | loop-helix protein 39) (bHLHb39) (Hairy and | BHLHB39 |
| | | enhancer of split 1) (Hairy homolog) (Hairy-like | HL |
| | | protein) (hHL) | HRY |
| 124 | HXB13_HUMAN | Homeobox protein Hox-B13 | HOXB13 |
| 125 | SIX1_HUMAN | Homeobox protein SIX1 (Sine oculis homeobox | SIX1 |
| | | homolog 1) | |
| 126 | DLX5_HUMAN | Homeobox protein DLX-5 | DLX5 |
| 127 | NANOG_HUMAN | Homeobox protein NANOG (Homeobox | NANOG |
| | | transcription factor Nanog) (hNanog) | |
| 128 | THA11_HUMAN | THAP domain-containing protein 11 | THAP11 |
| | | | HRIHF |
| | | | B2206 |
| 129 | JUN_HUMAN | Transcription factor AP-1 (Activator protein 1) | JUN |
| | | (AP1) (Proto-oncogene c-Jun) (V-jun avian | |
| | | sarcoma virus 17 oncogene homolog) (p39) | |
| 130 | JUND_HUMAN | Transcription factor jun-D | JUND |
| 131 | ZSC16_HUMAN | Zinc finger and SCAN domain-containing protein | ZSCAN16 |
| | | 16 (Zinc finger protein 392) (Zinc finger protein | ZNF392 |
| | | 435) | ZNF435 |
| 132 | PCGF6_HUMAN | Polycomb group RING finger protein 6 (Mel18 and | PCGF6 |
| | | Bmi1-like RING finger) (RING finger protein 134) | MBLR |
| | | | RNF134 |
| 133 | ATF4_HUMAN | Cyclic AMP-dependent transcription factor ATF-4 | ATF4 |
| | | (cAMP-dependent transcription factor ATF-4) | CREB2 |
| | | (Activating transcription factor 4) (Cyclic AMP- | TXREB |
| | | responsive element-binding protein 2) (CREB-2) | |
| | | (cAMP-responsive element-binding protein 2) | |
| | | (DNA-binding protein TAXREB67) (Tax- | |
| | | responsive enhancer element-binding protein 67) | |
| | | (TaxREB67) | |
| 134 | GBX1_HUMAN | Homeobox protein GBX-1 (Gastrulation and brain- | GBX1 |
| | | specific homeobox protein 1) | |
| 135 | ZNF24_HUMAN | Zinc finger protein 24 (Retinoic acid suppression | ZNF24 |
| | | protein A) (RSG-A) (Zinc finger and SCAN | KOX17 |
| | | domain-containing protein 3) (Zinc finger protein | ZNF191 |
| | | 191) (Zinc finger protein KOX17) | ZSCAN3 |
| 136 | NFE2_HUMAN | Transcription factor NF-E2 45 kDa subunit | NFE2 |
| | | (Leucine zipper protein NF-E2) (Nuclear factor, | |
| | | erythroid-derived 2 45 kDa subunit) (p45 NF-E2) | |
| 137 | SNF5_HUMAN | SWI/SNF-related matrix-associated actin- | SMARCB1 |
| | | dependent regulator of chromatin subfamily B | BAF47 |
| | | member 1 (BRG1-associated factor 47) (BAF47) | INI1 |
| | | (Integrase interactor 1 protein) (SNF5 homolog) | SNF5L1 |
| | | (hSNF5) | |
| 138 | HXA13_HUMAN | Homeobox protein Hox-A13 (Homeobox protein | HOXA13 |
| | | Hox-1J) | HOX1J |
| 139 | REQU_HUMAN | Zinc finger protein ubi-d4 (Apoptosis response zinc | DPF2 |
| | | finger protein) (BRG1-associated factor 45D) | BAF45D |
| | | (BAF45D) (D4, zinc and double PHD fingers | REQ |
| | | family 2) (Protein requiem) | UBID4 |
| 140 | P53_HUMAN | Cellular tumor antigen p53 (Antigen NY-CO-13) | TP53 |
| | | (Phosphoprotein p53) (Tumor suppressor p53) | P53 |
| 141 | TGIF1_HUMAN | Homeobox protein TGIF1 (5′-TG-3′-interacting | TGIF1 |
| | | factor 1) | TGIF |
| 142 | ALX4_HUMAN | Homeobox protein aristaless-like 4 | ALX4 |
| | | | KIAA1788 |
| 143 | SOX17_HUMAN | Transcription factor SOX-17 | SOX17 |
| 144 | KLF15_HUMAN | Krueppel-like factor 15 (Kidney-enriched krueppel- | KLF15 |
| | | like factor) | KKLF |
| 145 | HMBX1_HUMAN | Homeobox-containing protein 1 | HMBOX1 |
| 146 | PAX6_HUMAN | Paired box protein Pax-6 (Aniridia type II protein) | PAX6 |
| | | (Oculorhombin) | AN2 |
| 147 | COT1_HUMAN | COUP transcription factor 1 (COUP-TF1) (COUP | NR2F1 |
| | | transcription factor I) (COUP-TF I) (Nuclear | EAR3 |
| | | receptor subfamily 2 group F member 1) (V-erbA- | ERBAL3 |
| | | related protein 3) (EAR-3) | TFCOUP1 |
| 148 | IRF3_HUMAN | Interferon regulatory factor 3 (IRF-3) | IRF3 |
| 149 | PKNX1_HUMAN | Homeobox protein PKNOX1 (Homeobox protein | PKNOX1 |
| | | PREP-1) (PBX/knotted homeobox 1) | PREP1 |
| 150 | MYC_HUMAN | Myc proto-oncogene protein (Class E basic helix- | MYC |
| | | loop-helix protein 39) (bHLHe39) (Proto-oncogene | BHLHE39 |
| | | c-Myc) (Transcription factor p64) | |
| 151 | PPARD_HUMAN | Peroxisome proliferator-activated receptor delta | PPARD |
| | | (PPAR-delta) (NUCI) (Nuclear hormone receptor | NR1C2 |
| | | 1) (NUC1) (Nuclear receptor subfamily 1 group C | PPARB |
| | | member 2) (Peroxisome proliferator-activated | |
| | | receptor beta) (PPAR-beta) | |
| 152 | ETS1_HUMAN | Protein C-ets-1 (p54) | ETS1 |
| | | | EWSR2 |
| 153 | WT1_HUMAN | Wilms tumor protein (WT33) | WT1 |
| 154 | PAX8_HUMAN | Paired box protein Pax-8 | PAX8 |
| 155 | IRF4_HUMAN | Interferon regulatory factor 4 (IRF-4) | IRF4 |
| | | (Lymphocyte-specific interferon regulatory factor) | MUM1 |
| | | (LSIRF) (Multiple myeloma oncogene 1) (NF- | |
| | | EM5) | |
| 156 | FLI1_HUMAN | Friend leukemia integration 1 transcription factor | FLI1 |
| | | (Proto-oncogene Fli-1) (Transcription factor | |
| | | ERGB) | |
| 157 | ETV6_HUMAN | Transcription factor ETV6 (ETS translocation | ETV6 |
| | | variant 6) (ETS-related protein Tel1) (Tel) | TEL |
| | | | TEL1 |
| 158 | RUNX1_HUMAN | Runt-related transcription factor 1 (Acute myeloid | RUNX1 |
| | | leukemia 1 protein) (Core-binding factor subunit | AML1 |
| | | alpha-2) (CBF-alpha-2) (Oncogene AML-1) | CBFA2 |
| | | (Polyomavirus enhancer-binding protein 2 alpha B | |
| | | subunit) (PEA2-alpha B) (PEBP2-alpha B) (SL3-3 | |
| | | enhancer factor 1 alpha B subunit) (SL3/AKV | |
| | | core-binding factor alpha B subunit) | |
| 159 | KLF5_HUMAN | Krueppel-like factor 5 (Basic transcription element- | KLF5 |
| | | binding protein 2) (BTE-binding protein 2) (Colon | BTEB2 |
| | | krueppel-like factor) (GC-box-binding protein 2) | CKLF |
| | | (Intestinal-enriched krueppel-like factor) | IKLF |
| | | (Transcription factor BTEB2) | |
| 160 | NR1H2_HUMAN | Oxysterols receptor LXR-beta (Liver X receptor | NR1H2 |
| | | beta) (Nuclear receptor NER) (Nuclear receptor | LXRB |
| | | subfamily 1 group H member 2) (Ubiquitously- | NER |
| | | expressed nuclear receptor) | UNR |
| 161 | ZIC3_HUMAN | Zinc finger protein ZIC 3 (Zinc finger protein 203) | ZIC3 |
| | | (Zinc finger protein of the cerebellum 3) | ZNF203 |
| 162 | ETV1_HUMAN | ETS translocation variant 1 (Ets-related protein 81) | ETV1 |
| | | | ER81 |
| 163 | PO2F2_HUMAN | POU domain, class 2, transcription factor 2 | POU2F2 |
| | | (Lymphoid-restricted immunoglobulin octamer- | OCT2 |
| | | binding protein NF-A2) (Octamer-binding protein | OTF2 |
| | | 2) (Oct-2) (Octamer-binding transcription factor 2) | |
| | | (OTF-2) | |
| 164 | KLF10_HUMAN | Krueppel-like factor 10 (EGR-alpha) | KLF10 |
| | | (Transforming growth factor-beta-inducible early | TIEG |
| | | growth response protein 1) (TGFB-inducible early | TIEG1 |
| | | growth response protein 1) (TIEG-1) | |
| 165 | ETV4_HUMAN | ETS translocation variant 4 (Adenovirus E1A | ETV4 |
| | | enhancer-binding protein) (E1A-F) (Polyomavirus | E1AF |
| | | enhancer activator 3 homolog) (Protein PEA3) | PEA3 |
| 166 | ERG_HUMAN | Transcriptional regulator ERG (Transforming | ERG |
| | | protein ERG) | |
| 167 | TRI34_HUMAN | Tripartite motif-containing protein 34 (Interferon- | TRIM34 |
| | | responsive finger protein 1) (RING finger protein 21) | IFP1 |
| | | | RNF21 |
| 168 | TRIM5_HUMAN | Tripartite motif-containing protein 5 (EC 2.3.2.27) | TRIM5 |
| | | (RING finger protein 88) (RING-type E3 ubiquitin | RNF88 |
| | | transferase TRIM5) | |
| 169 | TISD_HUMAN | mRNA decay activator protein ZFP36L2 (Butyrate | ZFP36L2 |
| | | response factor 2) (EGF-response factor 2) (ERF-2) | BRF2 |
| | | (TPA-induced sequence lid) (Zinc finger protein | ERF2 |
| | | 36, C3H1 type-like 2) (ZFP36-like 2) | RNF162C |
| | | | TIS11D |
| 170 | RCOR3_HUMAN | REST corepressor 3 | RCOR3 |
| | | | KIAA1343 |
| 171 | IRF5_HUMAN | Interferon regulatory factor 5 (IRF-5) | IRF5 |
| 172 | FOXC2_HUMAN | Forkhead box protein C2 (Forkhead-related protein | FOXC2 |
| | | FKHL14) (Mesenchyme fork head protein 1) | FKHL14 |
| | | (MFH-1 protein) (Transcription factor FKH-14) | MFH1 |
| 173 | TRAF2_HUMAN | TNF receptor-associated factor 2 (EC 2.3.2.27) (E3 | TRAF2 |
| | | ubiquitin-protein ligase TRAF2) (RING-type E3 | TRAP3 |
| | | ubiquitin transferase TRAF2) (Tumor necrosis | |
| | | factor type 2 receptor-associated protein 3) | |
| 174 | ATF2_HUMAN | Cyclic AMP-dependent transcription factor ATF-2 | ATF2 |
| | | (cAMP-dependent transcription factor ATF-2) (EC | CREB2 |
| | | 2.3.1.48) (Activating transcription factor 2) (Cyclic | CREBP1 |
| | | AMP-responsive element-binding protein 2) | |
| | | (CREB-2) (cAMP-responsive element-binding | |
| | | protein 2) (HB16) (Histone acetyltransferase | |
| | | ATF2) (cAMP response element-binding protein | |
| | | CRE-BP1) | |
| 175 | FOXO4_HUMAN | Forkhead box protein O4 (Fork head domain | FOXO4 |
| | | transcription factor AFX1) | AFX |
| | | | AFX1 |
| | | | MLLT7 |
| 176 | INSM1_HUMAN | Insulinoma-associated protein 1 (Zinc finger | INSM1 |
| | | protein IA-1) | IA1 |
| 177 | TBX5_HUMAN | T-box transcription factor TBX5 (T-box protein 5) | TBX5 |
| 178 | TRAF6_HUMAN | TNF receptor-associated factor 6 (EC 2.3.2.27) (E3 | TRAF6 |
| | | ubiquitin-protein ligase TRAF6) (Interleukin-1 | RNF85 |
| | | signal transducer) (RING finger protein 85) | |
| | | (RING-type E3 ubiquitin transferase TRAF6) | |
| 179 | HSF2_HUMAN | Heat shock factor protein 2 (HSF 2) (Heat shock | HSF2 |
| | | transcription factor 2) (HSTF 2) | HSTF2 |
| 180 | NR5A2_HUMAN | Nuclear receptor subfamily 5 group A member 2 | NR5A2 |
| | | (Alpha-1-fetoprotein transcription factor) (B1- | B1F |
| | | binding factor) (hB1F) (CYP7A promoter-binding | CPF |
| | | factor) (Hepatocytic transcription factor) (Liver | FTF |
| | | receptor homolog 1) (LRH-1) | |
| 181 | HOMEZ_HUMAN | Homeobox and leucine zipper protein Homez | HOMEZ |
| | | (Homeodomain leucine zipper-containing factor) | KIAA1443 |
| 182 | TF65_HUMAN | Transcription factor p65 (Nuclear factor NF-kappa- | RELA |
| | | B p65 subunit) (Nuclear factor of kappa light | NFKB3 |
| | | polypeptide gene enhancer in B-cells 3) | |
| 183 | HNF1B_HUMAN | Hepatocyte nuclear factor 1-beta (HNF-1-beta) | HNF1B |
| | | (HNF-1B) (Homeoprotein LFB3) (Transcription | TCF2 |
| | | factor 2) (TCF-2) (Variant hepatic nuclear factor 1) | |
| | | (vHNF1) | |
| 184 | DEAF1_HUMAN | Deformed epidermal autoregulatory factor 1 | DEAF1 |
| | | homolog (Nuclear DEAF-1-related transcriptional | SPN |
| | | regulator) (NUDR) (Suppressin) (Zinc finger | ZMYND5 |
| | | MYND domain-containing protein 5) | |
| 185 | PHF1_HUMAN | PHD finger protein 1 (Protein PHF1) (hPHF1) | PHF1 |
| | | (Polycomb-like protein 1) (hPCll) | PCL1 |
| 186 | IKZF4_HUMAN | Zinc finger protein Eos (Ikaros family zinc finger | IKZF4 |
| | | protein 4) | KIAA1782 |
| | | | ZNFN1A4 |
| 187 | TRI29_HUMAN | Tripartite motif-containing protein 29 (Ataxia | TRIM29 |
| | | telangiectasia group D-associated protein) | ATDC |
| 188 | ARI3A_HUMAN | AT-rich interactive domain-containing protein 3A | ARID3A |
| | | (ARID domain-containing protein 3A) (B-cell | DRIL1 |
| | | regulator of IgH transcription) (Bright) (Dead | DRIL3 |
| | | ringer-like protein 1) (E2F-binding protein 1) | DRX |
| | | | E2FBP1 |
| 189 | COE3_HUMAN | Transcription factor COE3 (Early B-cell factor 3) | EBF3 |
| | | (EBF-3) (Olf-1/EBF-like 2) (O/E-2) (OE-2) | COE3 |
| 190 | MTG8_HUMAN | Protein CBFA2T1 (Cyclin-D-related protein) | RUNX1T1 |
| | | (Eight twenty one protein) (Protein ETO) (Protein | AML1T1 |
| | | MTG8) (Zinc finger MYND domain-containing | CBFA2T1 |
| | | protein 2) | CDR |
| | | | ETO |
| | | | MTG8 |
| | | | ZMYND2 |
| 191 | NF2L2_HUMAN | Nuclear factor erythroid 2-related factor 2 (NF-E2- | NFE2L2 |
| | | related factor 2) (NFE2-related factor 2) (HEBP1) | NRF2 |
| | | (Nuclear factor, erythroid derived 2, like 2) | |
| 192 | P73_HUMAN | Tumor protein p73 (p53-like transcription factor) | TP73 |
| | | (p53-related protein) | P73 |
| 193 | TFE2_HUMAN | Transcription factor E2-alpha (Class B basic helix- | TCF3 |
| | | loop-helix protein 21) (bHLHb21) | BHLHB21 |
| | | (Immunoglobulin enhancer-binding factor | E2A |
| | | E12/E47) (Immunoglobulin transcription factor 1) | ITF1 |
| | | (Kappa-E2-binding factor) (Transcription factor 3) | |
| | | (TCF-3) (Transcription factor ITF-1) | |
| 194 | FOXK2_HUMAN | Forkhead box protein K2 (Cellular transcription | FOXK2 |
| | | factor ILF-1) (FOXK1) (Interleukin enhancer- | ILF |
| | | binding factor 1) | ILF1 |
| 195 | ZMYM5_HUMAN | Zinc finger MYM-type protein 5 (Zinc finger | ZMYM5 |
| | | protein 198-like 1) (Zinc finger protein 237) | ZNF198L1 |
| | | | ZNF237 |
| | | | HSPC050 |
| 196 | KAISO_HUMAN | Transcriptional regulator Kaiso (Zinc finger and | ZBTB33 |
| | | BTB domain-containing protein 33) | KAISO |
| | | | ZNF348 |
| 197 | FOXP1_HUMAN | Forkhead box protein P1 (Mac-1-regulated | FOXP1 |
| | | forkhead) (MFH) | HSPC215 |
| 198 | TAF6_HUMAN | Transcription initiation factor TFIID subunit 6 | TAF6 |
| | | (RNA polymerase II TBP-associated factor subunit | TAF2E |
| | | E) (Transcription initiation factor TFIID 70 kDa | TAFII70 |
| | | subunit) (TAF(II)70) (TAFII-70) (TAFII70) | |
| | | (Transcription initiation factor TFIID 80 kDa | |
| | | subunit) (TAF(II)80) (TAFII-80) (TAFII80) | |
| 199 | P63_HUMAN | Tumor protein 63 (p63) (Chronic ulcerative | TP63 |
| | | stomatitis protein) (CUSP) (Keratinocyte | KET |
| | | transcription factor KET) (Transformation-related | P63 |
| | | protein 63) (TP63) (Tumor protein p73-like) | P73H |
| | | (p73L) (p40) (p51) | P73L |
| | | | TP73L |
| 200 | PATZ1_HUMAN | POZ-, AT hook-, and zinc finger-containing protein | PATZ1 |
| | | 1 (BTB/POZ domain zinc finger transcription | PATZ |
| | | factor) (Protein kinase A RI subunit alpha- | RIAZ |
| | | associated protein) (Zinc finger and BTB domain- | ZBTB19 |
| | | containing protein 19) (Zinc finger protein 278) | ZNF278 |
| | | (Zinc finger sarcoma gene protein) | ZSG |
| 201 | ZBED1_HUMAN | Zinc finger BED domain-containing protein 1 | ZBED1 |
| | | (Putative Ac-like transposable element) (dREF | ALTE |
| | | homolog) | DREF |
| | | | KIAA0785 |
| | | | TRAMP |
| 202 | ZN224_HUMAN | Zinc finger protein 224 (Bone marrow zinc finger | ZNF224 |
| | | 2) (BMZF-2) (Zinc finger protein 233) (Zinc finger | BMZF2 |
| | | protein 255) (Zinc finger protein 27) (Zinc finger | KOX22 |
| | | protein KOX22) | ZNF233 |
| | | | ZNF255 |
| | | | ZNF27 |
| 203 | MTA1_HUMAN | Metastasis-associated protein MTA1 | MTA1 |
| 204 | CTCF_HUMAN | Transcriptional repressor CTCF (11-zinc finger | CTCF |
| | | protein) (CCCTC-binding factor) (CTCFL paralog) | |
| 205 | SATB2_HUMAN | DNA-binding protein SATB2 (Special AT-rich | SATB2 |
| | | sequence-binding protein 2) | KIAA1034 |
| 206 | PROX1_HUMAN | Prospero homeobox protein 1 (Homeobox | PROX1 |
| | | prospero-like protein PROX1) (PROX-1) | |
| 207 | DACH1_HUMAN | Dachshund homolog 1 (Dach1) | DACH1 |
| | | | DACH |
| 208 | SATB1_HUMAN | DNA-binding protein SATB1 (Special AT-rich | SATB1 |
| | | sequence-binding protein 1) | |
| 209 | GCR_HUMAN | Glucocorticoid receptor (GR) (Nuclear receptor | NR3C1 |
| | | subfamily 3 group C member 1) | GRL |
| 210 | IF16_HUMAN | Gamma-interferon-inducible protein 16 (Ifi-16) | IFI16 |
| | | (Interferon-inducible myeloid differentiation | IFNGIP1 |
| | | transcriptional activator) | |
| 211 | SP1_HUMAN | Transcription factor Sp1 | SP1 |
| | | | TSFP1 |
| 212 | ARNT_HUMAN | Aryl hydrocarbon receptor nuclear translocator | ARNT |
| | | (ARNT protein) (Class E basic helix-loop-helix | BHLHE2 |
| | | protein 2) (bHLHe2) (Dioxin receptor, nuclear | |
| | | translocator) (Hypoxia-inducible factor 1-beta) | |
| | | (HIF-1-beta) (HIF 1-beta) | |
| 213 | CDC5L_HUMAN | Cell division cycle 5-like protein (Cdc5-like | CDC5L |
| | | protein) (Pombe cdc5-related protein) | KIAA0432 |
| | | | PCDC5RP |
| 214 | ZBT17_HUMAN | Zinc finger and BTB domain-containing protein 17 | ZBTB17 |
| | | (Myc-interacting zinc finger protein 1) (Miz-1) | MIZ1 |
| | | (Zinc finger protein 151) (Zinc finger protein 60) | ZNF151 |
| | | | ZNF60 |
| 215 | ZHX2_HUMAN | Zinc fingers and homeoboxes protein 2 (Alpha- | ZHX2 |
| | | fetoprotein regulator 1) (AFP regulator 1) | AFR1 |
| | | (Regulator of AFP) (Zinc finger and homeodomain | KIAA0854 |
| | | protein 2) | RAF |
| 216 | ZKSC5_HUMAN | Zinc finger protein with KRAB and SCAN | ZKSCAN5 |
| | | domains 5 (Zinc finger protein 95 homolog) (Zfp- | KIAA1015 |
| | | 95) | ZFP95 |
| 217 | STAT6_HUMAN | Signal transducer and activator of transcription 6 | STAT6 |
| | | (IL-4 Stat) | |
| 218 | ZN484_HUMAN | Zinc finger protein 484 | ZNF484 |
| 219 | ZFP28_HUMAN | Zinc finger protein 28 homolog (Zfp-28) | ZFP28 |
| | | (Krueppel-like zinc finger factor X6) | KIAA1431 |
| 220 | ZHX1_HUMAN | Zinc fingers and homeoboxes protein 1 | ZHX1 |
| 221 | PRGR_HUMAN | Progesterone receptor (PR) (Nuclear receptor | PGR |
| | | subfamily 3 group C member 3) | NR3C3 |
| 222 | ZN268_HUMAN | Zinc finger protein 268 (Zinc finger protein HZF3) | ZNF268 |
| 223 | ZHX3_HUMAN | Zinc fingers and homeoboxes protein 3 (Triple | ZHX3 |
| | | homeobox protein 1) (Zinc finger and | KIAA0395 |
| | | homeodomain protein 3) | TIX1 |
| 224 | NFKB1_HUMAN | Nuclear factor NF-kappa-B p105 subunit (DNA- | NFKB1 |
| | | binding factor KBF1) (EBP-1) (Nuclear factor of | |
| | | kappa light polypeptide gene enhancer in B-cells 1) | |
| | | [Cleaved into: Nuclear factor NF-kappa-B p50 | |
| | | subunit] | |
| 225 | MCR_HUMAN | Mineralocorticoid receptor (MR) (Nuclear receptor | NR3C2 |
| | | subfamily 3 group C member 2) | MCR |
| | | | MLR |
| 226 | HLTF_HUMAN | Helicase-like transcription factor (EC 2.3.2.27) (EC | HLTF |
| | | 3.6.4.—) (DNA-binding protein/plasminogen | HIP116A |
| | | activator inhibitor 1 regulator) (HIP116) (RING | RNF80 |
| | | finger protein 80) (RING-type E3 ubiquitin | SMARCA3 |
| | | transferase HLTF) (SWI/SNF-related matrix- | SNF2L3 |
| | | associated actin-dependent regulator of chromatin | ZBU1 |
| | | subfamily A member 3) (Sucrose nonfermenting | |
| | | protein 2-like 3) | |
| 227 | PHF20_HUMAN | PHD finger protein 20 (Glioma-expressed antigen | PHF20 |
| | | 2) (Hepatocellular carcinoma-associated antigen | C20orf104 |
| | | 58) (Novel zinc finger protein) (Transcription | GLEA2 |
| | | factor TZP) | HCA58 |
| | | | NZF |
| | | | TZP |
| 228 | PARP1_HUMAN | Poly [ADP-ribose] polymerase 1 (PARP-1) (EC | PARP1 |
| | | 2.4.2.30) (ADP-ribosyltransferase diphtheria toxin- | ADPRT |
| | | like 1) (ARTD1) (NAD(+) ADP-ribosyltransferase | PPOL |
| | | 1) (ADPRT 1) (Poly[ADP-ribose] synthase 1) | |
| 229 | ST18_HUMAN | Suppression of tumorigenicity 18 protein (Zinc | ST18 |
| | | finger protein 387) | KIAA0535 |
| | | | ZNF387 |
| 230 | ZN217_HUMAN | Zinc finger protein 217 | ZNF217 |
| | | | ZABC1 |
| 231 | SRBP2_HUMAN | Sterol regulatory element-binding protein 2 | SREBF2 |
| | | (SREBP-2) (Class D basic helix-loop-helix protein | BHLHD2 |
| | | 2) (bHLHd2) (Sterol regulatory element-binding | SREBP2 |
| | | transcription factor 2) [Cleaved into: Processed | |
| | | sterol regulatory element-binding protein 2] | |
| 232 | TAF2_HUMAN | Transcription initiation factor TFIID subunit 2 (150 | TAF2 |
| | | kDa cofactor of initiator function) (RNA | CIF150 |
| | | polymerase II TBP-associated factor subunit B) | TAF2B |
| | | (TBP-associated factor 150 kDa) (Transcription | |
| | | initiation factor TFIID 150 kDa subunit) | |
| | | (TAF(II)150) (TAFII-150) (TAFII150) | |
| 233 | ARI4A_HUMAN | AT-rich interactive domain-containing protein 4A | ARID4A |
| | | (ARID domain-containing protein 4A) | RBBP1 |
| | | (Retinoblastoma-binding protein 1) (RBBP-1) | RBP1 |
| 234 | CUX2_HUMAN | Homeobox protein cut-like 2 (Homeobox protein | CUX2 |
| | | cux-2) | CUTL2 |
| | | | KIAA0293 |
| 235 | ARI1A_HUMAN | AT-rich interactive domain-containing protein 1A | ARID1A |
| | | (ARID domain-containing protein 1A) (B120) | BAF250 |
| | | (BRG1-associated factor 250) (BAF250) (BRG1- | BAF250A |
| | | associated factor 250a) (BAF250A) (Osa homolog | C1orf4 |
| | | 1) (hOSA1) (SWI-like protein) (SWI/SNF complex | OSA1 |
| | | protein p270) (SWI/SNF-related, matrix- | SMARCF1 |
| | | associated, actin-dependent regulator of chromatin | |
| | | subfamily F member 1) (hELD) | |
| 236 | PA2GX_HUMAN | Group 10 secretory phospholipase A2 (EC 3.1.1.4) | PLA2G10 |
| | | (Group X secretory phospholipase A2) (GX | |
| | | sPLA2) (sPLA2-X) (Phosphatidylcholine 2- | |
| | | acylhydrolase 10) | |
| 237 | PARK7_HUMAN | Protein/nucleic acid deglycase DJ-1 (EC 3.1.2.—) | PARK7 |
| | | (EC 3.5.1.—) (EC 3.5.1.124) (Maillard deglycase) | |
| | | (Oncogene DJ1) (Parkinson disease protein 7) | |
| | | (Parkinsonism-associated deglycase) (Protein DJ-1) | |
| | | (DJ-1) | |
| 238 | RNF4_HUMAN | E3 ubiquitin-protein ligase RNF4 (EC 2.3.2.27) | RNF4 |
| | | (RING finger protein 4) (RING-type E3 ubiquitin | SNURF |
| | | transferase RNF4) (Small nuclear ring finger | RES4-26 |
| | | protein) (Protein SNURF) | |
| 239 | CNOT7_HUMAN | CCR4-NOT transcription complex subunit 7 (EC | CNOT7 |
| | | 3.1.13.4) (BTG1-binding factor 1) (CCR4- | CAF1 |
| | | associated factor 1) (CAF-1) (Caf1a) | |
| 240 | HMOX1_HUMAN | Heme oxygenase 1 (HO-1) (EC 1.14.14.18) | HMOX1 |
| | | | HO |
| | | | HO1 |
| 241 | RNF41_HUMAN | E3 ubiquitin-protein ligase NRDP1 (EC 2.3.2.27) | RNF41 |
| | | (RING finger protein 41) (RING-type E3 ubiquitin | FLRF |
| | | transferase NRDP1) | NRDP1 |
| | | | SBBI03 |
| 242 | PLS1_HUMAN | Phospholipid scramblase 1 (PL scramblase 1) | PLSCR1 |
| | | (Ca(2+)-dependent phospholipid scramblase 1) | |
| | | (Erythrocyte phospholipid scramblase) | |
| | | (MmTRA1b) | |
| 243 | RING2_HUMAN | E3 ubiquitin-protein ligase RING2 (EC 2.3.2.27) | RNF2 |
| | | (Huntingtin-interacting protein 2-interacting | BAP1 |
| | | protein 3) (HIP2-interacting protein 3) (Protein | DING |
| | | DinG) (RING finger protein 1B) (RING1b) (RING | HIPI3 |
| | | finger protein 2) (RING finger protein BAP-1) | RING1B |
| | | (RING-type E3 ubiquitin transferase RING2) | |
| 244 | PEX14_HUMAN | Peroxisomal membrane protein PEX14 (PTS1 | PEX14 |
| | | receptor-docking protein) (Peroxin-14) | |
| | | (Peroxisomal membrane anchor protein PEX14) | |
| 245 | PTEN_HUMAN | Phosphatidylinositol 3,4,5-trisphosphate 3- | PTEN |
| | | phosphatase and dual-specificity protein | MMAC1 |
| | | phosphatase PTEN (EC 3.1.3.16) (EC 3.1.3.48) | TEP1 |
| | | (EC 3.1.3.67) (Mutated in multiple advanced | |
| | | cancers 1) (Phosphatase and tensin homolog) | |
| 246 | TDG_HUMAN | G/T mismatch-specific thymine DNA glycosylase | TDG |
| | | (EC 3.2.2.29) (Thymine-DNA glycosylase) | |
| | | (hTDG) | |
| 247 | TRI31_HUMAN | E3 ubiquitin-protein ligase TRIM31 (EC 2.3.2.27) | TRIM31 |
| | | (RING-type E3 ubiquitin transferase TRIM31) | C6orf13 |
| | | (Tripartite motif-containing protein 31) | RNF |
| 248 | ENOA_HUMAN | Alpha-enolase (EC 4.2.1.11) (2-phospho-D- | ENO1 |
| | | glycerate hydro-lyase) (C-myc promoter-binding | ENO1L1 |
| | | protein) (Enolase 1) (MBP-1) (MPB-1) (Non- | MBPB1 |
| | | neural enolase) (NNE) (Phosphopyruvate | MPB1 |
| | | hydratase) (Plasminogen-binding protein) | |
| 249 | RUVB2_HUMAN | RuvB-like 2 (EC 3.6.4.12) (48 kDa TATA box- | RUVBL2 |
| | | binding protein-interacting protein) (48 kDa TBP- | INO80J |
| | | interacting protein) (51 kDa erythrocyte cytosolic | TIP48 |
| | | protein) (ECP-51) (INO80 complex subunit J) | TIP49B |
| | | (Repressing pontin 52) (Reptin 52) (TIP49b) | CGI-46 |
| | | (TIP60-associated protein 54-beta) (TAP54-beta) | |
| 250 | PRKN_HUMAN | E3 ubiquitin-protein ligase parkin (Parkin) (EC | PRKN |
| | | 2.3.2.—) (Parkin RBR E3 ubiquitin-protein ligase) | PARK2 |
| | | (Parkinson juvenile disease protein 2) (Parkinson | |
| | | disease protein 2) | |
| 251 | RO52_HUMAN | E3 ubiquitin-protein ligase TRIM21 (EC 2.3.2.27) | TRIM21 |
| | | (52 kDa Ro protein) (52 kDa ribonucleoprotein | RNF81 |
| | | autoantigen Ro/SS-A) (RING finger protein 81) | RO52 |
| | | (RING-type E3 ubiquitin transferase TRIM21) | SSA1 |
| | | (Ro(SS-A)) (Sjoegren syndrome type A antigen) | |
| | | (SS-A) (Tripartite motif-containing protein 21) | |
| 252 | SYSC_HUMAN | Serine-tRNA ligase, cytoplasmic (EC 6.1.1.11) | SARS |
| | | (Seryl-tRNA synthetase) (SerRS) (Seryl- | SERS |
| | | tRNA(Ser/Sec) synthetase) | |
| 253 | FBW1A_HUMAN | F-box/WD repeat-containing protein 1A | BTRC |
| | | (E3RSIkappaB) (Epididymis tissue protein Li 2a) | BTRCP |
| | | (F-box and WD repeats protein beta-TrCP) | FBW1A |
| | | (pIkappaBalpha-E3 receptor subunit) | FBXW1A |
| 254 | XRCC6_HUMAN | X-ray repair cross-complementing protein 6 (EC | XRCC6 |
| | | 3.6.4.—) (EC 4.2.99.—) (5′-deoxyribose-5-phosphate | G22P1 |
| | | lyase Ku70) (5′-dRP lyase Ku70) (70 kDa subunit | |
| | | of Ku antigen) (ATP-dependent DNA helicase 2 | |
| | | subunit 1) (ATP-dependent DNA helicase II 70 | |
| | | kDa subunit) (CTC box-binding factor 75 kDa | |
| | | subunit) (CTC75) (CTCBF) (DNA repair protein | |
| | | XRCC6) (Lupus Ku autoantigen protein p70) | |
| | | (Ku70) (Thyroid-lupus autoantigen) (TLAA) (X- | |
| | | ray repair complementing defective repair in | |
| | | Chinese hamster cells 6) | |
| 255 | PIAS2_HUMAN | E3 SUMO-protein ligase PIAS2 (EC 6.3.2.—) | PIAS2 |
| | | (Androgen receptor-interacting protein 3) (ARIP3) | PIASX |
| | | (DAB2-interacting protein) (DIP) (Msx-interacting | |
| | | zinc finger protein) (Miz1) (PIAS-NY protein) | |
| | | (Protein inhibitor of activated STAT x) (Protein | |
| | | inhibitor of activated STAT2) | |
| 256 | KEAP1_HUMAN | Kelch-like ECH-associated protein 1 (Cytosolic | KEAP1 |
| | | inhibitor of Nrf2) (INrf2) (Kelch-like protein 19) | INRF2 |
| | | | KIAA0132 |
| | | | KLHL19 |
| 257 | TRI25_HUMAN | E3 ubiquitin/ISG15 ligase TRIM25 (EC 6.3.2.n3) | TRIM25 |
| | | (Estrogen-responsive finger protein) (RING finger | EFP |
| | | protein 147) (RING-type E3 ubiquitin transferase) | RNF147 |
| | | (EC 2.3.2.27) (RING-type E3 ubiquitin transferase | ZNF147 |
| | | TRIM25) (Tripartite motif-containing protein 25) | |
| | | (Ubiquitin/ISG15-conjugating enzyme TRIM25) | |
| | | (Zinc finger protein 147) | |
| 258 | ANM5_HUMAN | Protein arginine N-methyltransferase 5 (EC | PRMT5 |
| | | 2.1.1.320) (72 kDa ICln-binding protein) (Histone- | HRMT1L5 |
| | | arginine N-methyltransferase PRMT5) (Jak- | IBP72 |
| | | binding protein 1) (Shk1 kinase-binding protein 1 | JBP1 |
| | | homolog) (SKB1 homolog) (SKB1Hs) [Cleaved | SKB1 |
| | | into: Protein arginine N-methyltransferase 5, N- | |
| | | terminally processed] | |
| 259 | TRI32_HUMAN | E3 ubiquitin-protein ligase TRIM32 (EC 2.3.2.27) | TRIM32 |
| | | (72 kDa Tat-interacting protein) (RING-type E3 | HT2A |
| | | ubiquitin transferase TRIM32) (Tripartite motif- | |
| | | containing protein 32) (Zinc finger protein HT2A) | |
| 260 | XRCC5_HUMAN | X-ray repair cross-complementing protein 5 (EC | XRCC5 |
| | | 3.6.4.—) (86 kDa subunit of Ku antigen) (ATP- | G22P2 |
| | | dependent DNA helicase 2 subunit 2) (ATP- | |
| | | dependent DNA helicase II 80 kDa subunit) (CTC | |
| | | box-binding factor 85 kDa subunit) (CTC85) | |
| | | (CTCBF) (DNA repair protein XRCC5) (Ku80) | |
| | | (Ku86) (Lupus Ku autoantigen protein p86) | |
| | | (Nuclear factor IV) (Thyroid-lupus autoantigen) | |
| | | (TLAA) (X-ray repair complementing defective | |
| | | repair in Chinese hamster cells 5 (double-strand- | |
| | | break rejoining)) | |
| 261 | TRIM1_HUMAN | Probable E3 ubiquitin-protein ligase MID2 (EC | MID2 |
| | | 2.3.2.27) (Midin-2) (Midline defect 2) (Midline-2) | FXY2 |
| | | (RING finger protein 60) (RING-type E3 ubiquitin | RNF60 |
| | | transferase MID2) (Tripartite motif-containing | TRIM1 |
| | | protein 1) | |
| 262 | SIR1_HUMAN | NAD-dependent protein deacetylase sirtuin-1 | SIRT1 |
| | | (hSIRT1) (EC 3.5.1.—) (Regulatory protein SIR2 | SIR2L1 |
| | | homolog 1) (SIR2-like protein 1) (hSIR2) [Cleaved | |
| | | into: SirtT1 75 kDa fragment (75SirT1)] | |
| 263 | UHRF1_HUMAN | E3 ubiquitin-protein ligase UHRF1 (EC 2.3.2.27) | UHRF1 |
| | | (Inverted CCAAT box-binding protein of 90 kDa) | ICBP90 |
| | | (Nuclear protein 95) (Nuclear zinc finger protein | NP95 |
| | | Np95) (HuNp95) (hNp95) (RING finger protein | RNF106 |
| | | 106) (RING-type E3 ubiquitin transferase UHRF1) | |
| | | (Transcription factor ICBP90) (Ubiquitin-like PHD | |
| | | and RING finger domain-containing protein 1) | |
| | | (hUHRF1) (Ubiquitin-like-containing PHD and | |
| | | RING finger domains protein 1) | |
| 264 | KDM1A_HUMAN | Lysine-specific histone demethylase 1A (EC 1.—.—.—) | KDM1A |
| | | (BRAF35-HDAC complex protein BHC110) | AOF2 |
| | | (Flavin-containing amine oxidase domain- | KDM1 |
| | | containing protein 2) | KIAA0601 |
| | | | LSD1 |
| 265 | WWP2_HUMAN | NEDD4-like E3 ubiquitin-protein ligase WWP2 | WWP2 |
| | | (EC 2.3.2.26) (Atrophin-1-interacting protein 2) | |
| | | (AIP2) (HECT-type E3 ubiquitin transferase | |
| | | WWP2) (WW domain-containing protein 2) | |
| 266 | SRRM1_HUMAN | Serine/arginine repetitive matrix protein 1 (SR- | SRRM1 |
| | | related nuclear matrix protein of 160 kDa) | SRM160 |
| | | (SRm160) (Ser/Arg-related nuclear matrix protein) | |
| 267 | CBL_HUMAN | E3 ubiquitin-protein ligase CBL (EC 2.3.2.27) | CBL |
| | | (Casitas B-lineage lymphoma proto-oncogene) | CBL2 |
| | | (Proto-oncogene c-Cbl) (RING finger protein 55) | RNF55 |
| | | (RING-type E3 ubiquitin transferase CBL) (Signal | |
| | | transduction protein CBL) | |
| 268 | DDX58_HUMAN | Probable ATP-dependent RNA helicase DDX58 | DDX58 |
| | | (EC 3.6.4.13) (DEAD box protein 58) (RIG-I-like | |
| | | receptor 1) (RLR-1) (Retinoic acid-inducible gene | |
| | | 1 protein) (RIG-1) (Retinoic acid-inducible gene I | |
| | | protein) (RIG-I) | |
| 269 | TRI37_HUMAN | E3 ubiquitin-protein ligase TRIM37 (EC 2.3.2.27) | TRIM37 |
| | | (Mulibrey nanism protein) (RING-type E3 | KIAA0898 |
| | | ubiquitin transferase TRIM37) (Tripartite motif- | MUL |
| | | containing protein 37) | POB1 |
| 270 | AFF4_HUMAN | AF4/FMR2 family member 4 (ALL1-fused gene | AFF4 |
| | | from chromosome 5q31 protein) (Protein AF-5q31) | AF5Q31 |
| | | (Major CDK9 elongation factor-associated protein) | MCEF |
| | | | HSPC092 |
| 271 | DHX9_HUMAN | ATP-dependent RNA helicase A (EC 3.6.4.13) | DHX9 |
| | | (DEAH box protein 9) (DExH-box helicase 9) | DDX9 |
| | | (Leukophysin) (LKP) (Nuclear DNA helicase II) | LKP |
| | | (NDH II) (RNA helicase A) | NDH2 |
| 272 | KDM5B_HUMAN | Lysine-specific demethylase 5B (EC 1.14.11.—) | KDM5B |
| | | (Cancer/testis antigen 31) (CT31) (Histone | JARID1B |
| | | demethylase JARID1B) (Jumonji/ARID domain- | PLU1 |
| | | containing protein IB) (PLU-1) (Retinoblastoma- | RBBP2H1 |
| | | binding protein 2 homolog 1) (RBP2-H1) | |
| 273 | KDM5A_HUMAN | Lysine-specific demethylase 5A (EC 1.14.11.—) | KDM5A |
| | | (Histone demethylase JARID1A) (Jumonji/ARID | JARID1A |
| | | domain-containing protein 1A) (Retinoblastoma- | RBBP2 |
| | | binding protein 2) (RBBP-2) | RBP2 |
| 274 | H33_HUMAN | Histone H3.3 | H3F3A |
| | | | H3.3A |
| | | | H3F3 |
| | | | PP781; |
| | | | H3F3B |
| | | | H3.3B |
| 275 | H2AY_HUMAN | Core histone macro-H2A.1 (Histone macroH2A1) | H2AFY |
| | | (mH2A1) (Histone H2A.y) (H2A/y) | MACR |
| | | (Medulloblastoma antigen MU-MB-50.205) | OH2A1 |
| 276 | TAF3_HUMAN | Transcription initiation factor TFIID subunit 3 (140 | TAF3 |
| | | kDa TATA box-binding protein-associated factor) | |
| | | (TBP-associated factor 3) (Transcription initiation | |
| | | factor TFIID 140 kDa subunit) (TAF(II)140) | |
| | | (TAF140) (TAFII-140) (TAFII140) | |
| 277 | EED_HUMAN | Polycomb protein EED (hEED) (Embryonic | EED |
| | | ectoderm development protein) (WD protein | |
| | | associating with integrin cytoplasmic tails 1) | |
| | | (WAIT-1) | |
| 278 | TAD2A_HUMAN | Transcriptional adapter 2-alpha (Transcriptional | TADA2A |
| | | adapter 2-like) (ADA2-like protein) | TADA2L |
| | | | KL04P |
| 279 | HDAC1_HUMAN | Histone deacetylase 1 (HD1) (EC 3.5.1.98) | HDAC1 |
| | | | RPD3L1 |
| 280 | HDAC2_HUMAN | Histone deacetylase 2 (HD2) (EC 3.5.1.98) | HDAC2 |
| 281 | KAT7_HUMAN | Histone acetyltransferase KAT7 (EC 2.3.1.48) | KAT7 |
| | | (Histone acetyltransferase binding to ORC1) | HBO1 |
| | | (Lysine acetyltransferase 7) (MOZ, YBF2/SAS3, | HBOa |
| | | SAS2 and TIP60 protein 2) (MYST-2) | MYST2 |
| 282 | MEN1_HUMAN | Menin | MEN1 |
| | | | SCG2 |
| 283 | EZH2_HUMAN | Histone-lysine N-methyltransferase EZH2 (EC | EZH2 |
| | | 2.1.1.43) (ENX-1) (Enhancer of zeste homolog 2) | KMT6 |
| | | (Lysine N-methyltransferase 6) | |
| 284 | KAT2B_HUMAN | Histone acetyltransferase KAT2B (EC 2.3.1.48) | KAT2B |
| | | (Histone acetyltransferase PCAF) (Histone | PCAF |
| | | acetylase PCAF) (Lysine acetyltransferase 2B) | |
| | | (P300/CBP-associated factor) (P/CAF) | |
| 285 | HDAC4_HUMAN | Histone deacetylase 4 (HD4) (EC 3.5.1.98) | HDAC4 |
| | | | KIAA0288 |
| 286 | HDAC5_HUMAN | Histone deacetylase 5 (HD5) (EC 3.5.1.98) | HDAC5 |
| | | (Antigen NY-CO-9) | KIAA0600 |
| 287 | JARD2_HUMAN | Protein Jumonji (Jumonji/ARID domain-containing | JARID2 |
| | | protein 2) | JMJ |
| 288 | EP300_HUMAN | Histone acetyltransferase p300 (p300 HAT) (EC | EP300 |
| | | 2.3.1.48) (E1A-associated protein p300) | P300 |
| 289 | CBP_HUMAN | CREB-binding protein (EC 2.3.1.48) | CREBBP |
| | | | CBP |
| 290 | NSD1_HUMAN | Histone-lysine N-methyltransferase, H3 lysine-36 | NSD1 |
| | | and H4 lysine-20 specific (EC 2.1.1.43) (Androgen | ARA267 |
| | | receptor coactivator 267 kDa protein) (Androgen | KMT3B |
| | | receptor-associated protein of 267 kDa) (H3-K36- | |
| | | HMTase) (H4-K20-HMTase) (Lysine N- | |
| | | methyltransferase 3B) (Nuclear receptor-binding | |
| | | SET domain-containing protein 1) (NR-binding | |
| | | SET domain-containing protein) | |
| 291 | KMT2B_HUMAN | Histone-lysine N-methyltransferase 2B (Lysine N- | KMT2B |
| | | methyltransferase 2B) (EC 2.1.1.43) | HRX2 |
| | | (Myeloid/lymphoid or mixed-lineage leukemia | KIAA0304 |
| | | protein 4) (Trithorax homolog 2) (WW domain- | MLL2 |
| | | binding protein 7) (WBP-7) | MLL4 |
| | | | TRX2 |
| | | | WBP7 |
| 292 | KMT2A_HUMAN | Histone-lysine N-methyltransferase 2A (Lysine N- | KMT2A |
| | | methyltransferase 2A) (EC 2.1.1.43) (ALL-1) | ALL1 |
| | | (CXXC-type zinc finger protein 7) | CXXC7 |
| | | (Myeloid/lymphoid or mixed-lineage leukemia) | HRX |
| | | (Myeloid/lymphoid or mixed-lineage leukemia | HTRX |
| | | protein 1) (Trithorax-like protein) (Zinc finger | MLL |
| | | protein HRX) [Cleaved into: MLL cleavage | MLL1 |
| | | product N320 (N-terminal cleavage product of 320 | TRX1 |
| | | kDa) (p320); MLL cleavage product C180 (C- | |
| | | terminal cleavage product of 180 kDa) (p180)] | |
| 293 | NDKA_HUMAN | Nucleoside diphosphate kinase A (NDK A) (NDP | NME1 |
| | | kinase A) (EC 2.7.4.6) (Granzyme A-activated | NDPKA |
| | | DNase) (GAAD) (Metastasis inhibition factor | NM23 |
| | | nm23) (NM23-H1) (Tumor metastatic process- | |
| | | associated protein) | |
| 294 | NDKB_HUMAN | Nucleoside diphosphate kinase B (NDK B) (NDP | NME2 |
| | | kinase B) (EC 2.7.4.6) (C-myc purine-binding | NM23B |
| | | transcription factor PUF) (Histidine protein kinase | |
| | | NDKB) (EC 2.7.13.3) (nm23-H2) | |
| 295 | MK01_HUMAN | Mitogen-activated protein kinase 1 (MAP kinase 1) | MAPK1 |
| | | (MAPK 1) (EC 2.7.11.24) (ERT1) (Extracellular | ERK2 |
| | | signal-regulated kinase 2) (ERK-2) (MAP kinase | PRKM1 |
| | | isoform p42) (p42-MAPK) (Mitogen-activated | PRKM2 |
| | | protein kinase 2) (MAP kinase 2) (MAPK 2) | |
| 296 | MK14_HUMAN | Mitogen-activated protein kinase 14 (MAP kinase | MAPK14 |
| | | 14) (MAPK 14) (EC 2.7.11.24) (Cytokine | CSBP |
| | | suppressive anti-inflammatory drug-binding | CSBP1 |
| | | protein) (CSAID-binding protein) (CSBP) (MAP | CSBP2 |
| | | kinase MXI2) (MAX-interacting protein 2) | CSPB1 |
| | | (Mitogen-activated protein kinase p38 alpha) | MXI2 |
| | | (MAP kinase p38 alpha) (Stress-activated protein | SAPK2A |
| | | kinase 2a) (SAPK2a) | |
| 297 | MK11_HUMAN | Mitogen-activated protein kinase 11 (MAP kinase | MAPK11 |
| | | 11) (MAPK 11) (EC 2.7.11.24) (Mitogen-activated | PRKM11 |
| | | protein kinase p38 beta) (MAP kinase p38 beta) | SAPK2 |
| | | (p38b) (Stress-activated protein kinase 2b) | SAPK2B |
| | | (SAPK2b) (p38-2) | |
| 298 | CDK9_HUMAN | Cyclin-dependent kinase 9 (EC 2.7.11.22) (EC | CDK9 |
| | | 2.7.11.23) (C-2K) (Cell division cycle 2-like | CDC2L4 |
| | | protein kinase 4) (Cell division protein kinase 9) | TAK |
| | | (Serine/threonine-protein kinase PITALRE) (Tat- | |
| | | associated kinase complex catalytic subunit) | |
| 299 | MK03_HUMAN | Mitogen-activated protein kinase 3 (MAP kinase 3) | MAPK3 |
| | | (MAPK 3) (EC 2.7.11.24) (ERT2) (Extracellular | ERK1 |
| | | signal-regulated kinase 1) (ERK-1) (Insulin- | PRKM3 |
| | | stimulated MAP2 kinase) (MAP kinase isoform | |
| | | p44) (p44-MAPK) (Microtubule-associated protein | |
| | | 2 kinase) (p44-ERK1) | |
| 300 | PIM1_HUMAN | Serine/threonine-protein kinase pim-1 (EC | PIM1 |
| | | 2.7.11.1) | |
| 301 | MK09_HUMAN | Mitogen-activated protein kinase 9 (MAP kinase 9) | MAPK9 |
| | | (MAPK 9) (EC 2.7.11.24) (JNK-55) (Stress- | JNK2 |
| | | activated protein kinase 1a) (SAPK1a) (Stress- | PRKM9 |
| | | activated protein kinase JNK2) (c-Jun N-terminal | SAPK1A |
| | | kinase 2) | |
| 302 | MK08_HUMAN | Mitogen-activated protein kinase 8 (MAP kinase 8) | MAPK8 |
| | | (MAPK 8) (EC 2.7.11.24) (JNK-46) (Stress- | JNK1 |
| | | activated protein kinase 1c) (SAPK1c) (Stress- | PRKM8 |
| | | activated protein kinase JNK1) (c-Jun N-terminal | SAPK1 |
| | | kinase 1) | SAPK1C |
| 303 | SGK1_HUMAN | Serine/threonine-protein kinase Sgk1 (EC 2.7.11.1) | SGK1 |
| | | (Serum/glucocorticoid-regulated kinase 1) | SGK |
| 304 | MK10_HUMAN | Mitogen-activated protein kinase 10 (MAP kinase | MAPK10 |
| | | 10) (MAPK 10) (EC 2.7.11.24) (MAP kinase p49 | JNK3 |
| | | 3F12) (Stress-activated protein kinase 1b) | JNK3A |
| | | (SAPK1b) (Stress-activated protein kinase JNK3) | PRKM10 |
| | | (c-Jun N-terminal kinase 3) | SAPK1B |
| 305 | AKT1_HUMAN | RAC-alpha serine/threonine-protein kinase (EC | AKT1 |
| | | 2.7.11.1) (Protein kinase B) (PKB) (Protein kinase | PKB |
| | | B alpha) (PKB alpha) (Proto-oncogene c-Akt) | RAC |
| | | (RAC-PK-alpha) | |
| 306 | STK3_HUMAN | Serine/threonine-protein kinase 3 (EC 2.7.11.1) | STK3 |
| | | (Mammalian STE20-like protein kinase 2) (MST- | KRS1 |
| | | 2) (STE20-like kinase MST2) (Serine/threonine- | MST2 |
| | | protein kinase Krs-1) [Cleaved into: | |
| | | Serine/threonine-protein kinase 3 36 kDa subunit | |
| | | (MST2/N); Serine/threonine-protein kinase 3 | |
| | | 20 kDa subunit (MST2/C)] | |
| 307 | PP2BA_HUMAN | Serine/threonine-protein phosphatase 2B catalytic | PPP3CA |
| | | subunit alpha isoform (EC 3.1.3.16) (CAM-PRP | CALNA |
| | | catalytic subunit) (Calmodulin-dependent | CNA |
| | | calcineurin A subunit alpha isoform) | |
| 308 | HCK_HUMAN | Tyrosine-protein kinase HCK (EC 2.7.10.2) | HCK |
| | | (Hematopoietic cell kinase) (Hemopoietic cell | |
| | | kinase) (p59-HCK/p60-HCK) (p59Hck) (p61Hck) | |
| 309 | TXK_HUMAN | Tyrosine-protein kinase TXK (EC 2.7.10.2) | TXK |
| | | (Protein-tyrosine kinase 4) (Resting lymphocyte | PTK4 |
| | | kinase) | RLK |
| 310 | KSYK_HUMAN | Tyrosine-protein kinase SYK (EC 2.7.10.2) (Spleen | SYK |
| | | tyrosine kinase) (p72-Syk) | |
| 311 | DDR2_HUMAN | Discoidin domain-containing receptor 2 (Discoidin | DDR2 |
| | | domain receptor 2) (EC 2.7.10.1) (CD167 antigen- | NTRKR3 |
| | | like family member B) (Discoidin domain- | TKT |
| | | containing receptor tyrosine kinase 2) | TYRO10 |
| | | (Neurotrophic tyrosine kinase, receptor-related 3) | |
| | | (Receptor protein-tyrosine kinase TKT) (Tyrosine- | |
| | | protein kinase TYRO10) (CD antigen CD167b) | |
| 312 | KPCD2_HUMAN | Serine/threonine-protein kinase D2 (EC 2.7.11.13) | PRKD2 |
| | | (nPKC-D2) | PKD2 |
| | | | HSPC187 |
| 313 | M3K10_HUMAN | Mitogen-activated protein kinase kinase kinase 10 | MAP3K10 |
| | | (EC 2.7.11.25) (Mixed lineage kinase 2) (Protein | MLK2 |
| | | kinase MST) | MST |
| 314 | KIT_HUMAN | Mast/stem cell growth factor receptor Kit (SCFR) | KIT |
| | | (EC 2.7.10.1) (Piebald trait protein) (PBT) (Proto- | SCFR |
| | | oncogene c-Kit) (Tyrosine-protein kinase Kit) | |
| | | (p145 c-kit) (v-kit Hardy-Zuckerman 4 feline | |
| | | sarcoma viral oncogene homolog) (CD antigen | |
| | | CD117) | |
| 315 | JAK2_HUMAN | Tyrosine-protein kinase JAK2 (EC 2.7.10.2) (Janus | JAK2 |
| | | kinase 2) (JAK-2) | |
| 316 | MTPN_HUMAN | Myotrophin (Protein V-1) | MTPN |
| 317 | NFYB_HUMAN | Nuclear transcription factor Y subunit beta (CAAT | NFYB |
| | | box DNA-binding protein subunit B) (Nuclear | HAP3 |
| | | transcription factor Y subunit B) (NF-YB) | |
| 318 | EDN1_HUMAN | Endothelin-1 (Preproendothelin-1) (PPET1) | EDN1 |
| | | [Cleaved into: Endothelin-1 (ET-1); Big | |
| | | endothelin-1] | |
| 319 | PRIO_HUMAN | Major prion protein (PrP) (ASCR) (PrP27-30) | PRNP |
| | | (PrP33-35C) (CD antigen CD230) | ALTPRP |
| | | | PRIP |
| | | | PRP |
| 320 | NR0B2_HUMAN | Nuclear receptor subfamily 0 group B member 2 | NR0B2 |
| | | (Orphan nuclear receptor SHP) (Small heterodimer | SHP |
| | | partner) | |
| 321 | CEBPE_HUMAN | CCAAT/enhancer-binding protein epsilon (C/EBP | CEBPE |
| | | epsilon) | |
| 322 | HEY1_HUMAN | Hairy/enhancer-of-split related with YRPW motif | HEY1 |
| | | protein 1 (Cardiovascular helix-loop-helix factor 2) | BHLHB31 |
| | | (CHF-2) (Class B basic helix-loop-helix protein 31) | CHF2 |
| | | (bHLHb31) (HES-related repressor protein 1) | HERP2 |
| | | (Hairy and enhancer of split-related protein 1) | HESR1 |
| | | (HESR-1) (Hairy-related transcription factor 1) | HRT1 |
| | | (HRT-1) (hHRT1) | |
| 323 | TISB_HUMAN | mRNA decay activator protein ZFP36L1 (Butyrate | ZFP36L1 |
| | | response factor 1) (EGF-response factor 1) (ERF-1) | BERG36 |
| | | (TPA-induced sequence 11b) (Zinc finger protein | BRF1 |
| | | 36, C3H1 type-like 1) (ZFP36-like 1) | ERF1 |
| | | | RNF162B |
| | | | TIS11B |
| 324 | CREB1_HUMAN | Cyclic AMP-responsive element-binding protein 1 | CREB1 |
| | | (CREB-1) (cAMP-responsive element-binding | |
| | | protein 1) | |
| 325 | E2F5_HUMAN | Transcription factor E2F5 (E2F-5) | E2F5 |
| 326 | NODAL_HUMAN | Nodal homolog | NODAL |
| 327 | NR1I3_HUMAN | Nuclear receptor subfamily 1 group I member 3 | NR1I3 |
| | | (Constitutive activator of retinoid response) | CAR |
| | | (Constitutive active response) (Constitutive | |
| | | androstane receptor) (CAR) (Orphan nuclear | |
| | | receptor MB67) | |
| 328 | KLF1_HUMAN | Krueppel-like factor 1 (Erythroid krueppel-like | KLF1 |
| | | transcription factor) (EKLF) | EKLF |
| 329 | ELF3_HUMAN | ETS-related transcription factor Elf-3 (E74-like | ELF3 |
| | | factor 3) (Epithelial-restricted with serine box) | ERT |
| | | (Epithelium-restricted Ets protein ESX) | ESX |
| | | (Epithelium-specific Ets transcription factor 1) | JEN |
| | | (ESE-1) | |
| 330 | ZN174_HUMAN | Zinc finger protein 174 (AW-1) (Zinc finger and | ZNF174 |
| | | SCAN domain-containing protein 8) | ZSCAN8 |
| 331 | HNF4G_HUMAN | Hepatocyte nuclear factor 4-gamma (HNF-4- | HNF4G |
| | | gamma) (Nuclear receptor subfamily 2 group A | NR2A2 |
| | | member 2) | |
| 332 | NR2E3_HUMAN | Photoreceptor-specific nuclear receptor (Nuclear | NR2E3 |
| | | receptor subfamily 2 group E member 3) (Retina- | PNR |
| | | specific nuclear receptor) | RNR |
| 333 | TFDP1_HUMAN | Transcription factor Dp-1 (DRTF1-polypeptide 1) | TFDP1 |
| | | (DRTF1) (E2F dimerization partner 1) | DP1 |
| 334 | LDB1_HUMAN | LIM domain-binding protein 1 (LDB-1) (Carboxyl- | LDB1 |
| | | terminal LIM domain-binding protein 2) (CLIM-2) | CLIM2 |
| | | (LIM domain-binding factor CLIM2) (hLdb1) | |
| | | (Nuclear LIM interactor) | |
| 335 | COT2_HUMAN | COUP transcription factor 2 (COUP-TF2) | NR2F2 |
| | | (Apolipoprotein A-I regulatory protein 1) (ARP-1) | ARP1 |
| | | (COUP transcription factor II) (COUP-TF II) | TFCOUP2 |
| | | (Nuclear receptor subfamily 2 group F member 2) | |
| 336 | ERR1_HUMAN | Steroid hormone receptor ERR1 (Estrogen | ESRRA |
| | | receptor-like 1) (Estrogen-related receptor alpha) | ERR1 |
| | | (ERR-alpha) (Nuclear receptor subfamily 3 group | ESRL1 |
| | | B member 1) | NR3B1 |
| 337 | EGLN1_HUMAN | Egl nine homolog 1 (EC 1.14.11.29) (Hypoxia- | EGLN1 |
| | | inducible factor prolyl hydroxylase 2) (HIF-PH2) | C1orf12 |
| | | (HIF-prolyl hydroxylase 2) (HPH-2) (Prolyl | PNAS-118 |
| | | hydroxylase domain-containing protein 2) (PHD2) | PNAS-137 |
| | | (SM-20) | |
| 338 | NR1I2_HUMAN | Nuclear receptor subfamily 1 group I member 2 | NR1I2 |
| | | (Orphan nuclear receptor PAR1) (Orphan nuclear | PXR |
| | | receptor PXR) (Pregnane X receptor) (Steroid and | |
| | | xenobiotic receptor) (SXR) | |
| 339 | E2F1_HUMAN | Transcription factor E2F1 (E2F-1) (PBR3) | E2F1 |
| | | (Retinoblastoma-associated protein 1) (RBAP-1) | RBBP3 |
| | | (Retinoblastoma-binding protein 3) (RBBP-3) | |
| | | (pRB-binding protein E2F-1) | |
| 340 | E2F2_HUMAN | Transcription factor E2F2 (E2F-2) | E2F2 |
| 341 | GATA4_HUMAN | Transcription factor GATA-4 (GATA-binding | GATA4 |
| | | factor 4) | |
| 342 | NR1H3_HUMAN | Oxysterols receptor LXR-alpha (Liver X receptor | NR1H3 |
| | | alpha) (Nuclear receptor subfamily 1 group H | LXRA |
| | | member 3) | |
| 343 | RARG_HUMAN | Retinoic acid receptor gamma (RAR-gamma) | RARG |
| | | (Nuclear receptor subfamily 1 group B member 3) | NR1B3 |
| 344 | NFYC_HUMAN | Nuclear transcription factor Y subunit gamma | NFYC |
| | | (CAAT box DNA-binding protein subunit C) | |
| | | (Nuclear transcription factor Y subunit C) (NF-YC) | |
| | | (Transactivator HSM-1/2) | |
| 345 | STF1_HUMAN | Steroidogenic factor 1 (SF-1) (STF-1) (hSF-1) | NR5A1 |
| | | (Adrenal 4-binding protein) (Fushi tarazu factor | AD4BP |
| | | homolog 1) (Nuclear receptor subfamily 5 group A | FTZF1 |
| | | member 1) (Steroid hormone receptor Ad4BP) | SF1 |
| 346 | PPARA_HUMAN | Peroxisome proliferator-activated receptor alpha | PPARA |
| | | (PPAR-alpha) (Nuclear receptor subfamily 1 group | NR1C1 |
| | | C member 1) | PPAR |
| 347 | NR0B1_HUMAN | Nuclear receptor subfamily 0 group B member 1 | NR0B1 |
| | | (DSS-AHC critical region on the X chromosome | AHC |
| | | protein 1) (Nuclear receptor DAX-1) | DAX1 |
| 348 | NR1H4_HUMAN | Bile acid receptor (Farnesoid X-activated receptor) | NR1H4 |
| | | (Farnesol receptor HRR-1) (Nuclear receptor | BAR |
| | | subfamily 1 group H member 4) (Retinoid X | FXR |
| | | receptor-interacting protein 14) (RXR-interacting | HRR1 |
| | | protein 14) | RIP14 |
| 349 | THA_HUMAN | Thyroid hormone receptor alpha (Nuclear receptor | THRA |
| | | subfamily 1 group A member 1) (V-erbA-related | EAR7 |
| | | protein 7) (EAR-7) (c-erbA-1) (c-erbA-alpha) | ERBA1 |
| | | | NR1A1 |
| | | | THRA1 |
| | | | THRA2 |
| 350 | ETV5_HUMAN | ETS translocation variant 5 (Ets-related protein | ETV5 |
| | | ERM) | ERM |
| 351 | KLF11_HUMAN | Krueppel-like factor 11 (Transforming growth | KLF11 |
| | | factor-beta-inducible early growth response protein | FKLF |
| | | 2) (TGFB-inducible early growth response protein | TIEG2 |
| | | 2) (TIEG-2) | |
| 352 | RORG_HUMAN | Nuclear receptor ROR-gamma (Nuclear receptor | RORC |
| | | RZR-gamma) (Nuclear receptor subfamily 1 group | NR1F3 |
| | | F member 3) (RAR-related orphan receptor C) | RORG |
| | | (Retinoid-related orphan receptor-gamma) | RZRG |
| 353 | RORA_HUMAN | Nuclear receptor ROR-alpha (Nuclear receptor | RORA |
| | | RZR-alpha) (Nuclear receptor subfamily 1 group F | NR1F1 |
| | | member 1) (RAR-related orphan receptor A) | RZRA |
| | | (Retinoid-related orphan receptor-alpha) | |
| 354 | MITF_HUMAN | Microphthalmia-associated transcription factor | MITF |
| | | (Class E basic helix-loop-helix protein 32) | BHLHE32 |
| | | (bHLHe32) | |
| 355 | ESR2_HUMAN | Estrogen receptor beta (ER-beta) (Nuclear receptor | ESR2 |
| | | subfamily 3 group A member 2) | ESTRB |
| | | | NR3A2 |
| 356 | ZGPAT_HUMAN | Zinc finger CCCH-type with G patch domain- | ZGPAT |
| | | containing protein (G patch domain-containing | GPATC6 |
| | | protein 6) (Zinc finger CCCH domain-containing | GPATCH6 |
| | | protein 9) (Zinc finger and G patch domain- | KIAA1847 |
| | | containing protein) | ZC3H9 |
| | | | ZC3HDC9 |
| | | | ZIP |
| 357 | RXRB_HUMAN | Retinoic acid receptor RXR-beta (Nuclear receptor | RXRB |
| | | subfamily 2 group B member 2) (Retinoid X | NR2B2 |
| | | receptor beta) | |
| 358 | NR1D2_HUMAN | Nuclear receptor subfamily 1 group D member 2 | NR1D2 |
| | | (Orphan nuclear hormone receptor BD73) (Rev-erb | |
| | | alpha-related receptor) (RVR) (Rev-erb-beta) (V- | |
| | | erbA-related protein 1-related) (EAR-1R) | |
| 359 | ZBT7A_HUMAN | Zinc finger and BTB domain-containing protein 7A | ZBTB7A |
| | | (Factor binding IST protein 1) (FBI-1) (Factor that | FBI1 |
| | | binds to inducer of short transcripts protein 1) | LRF |
| | | (HIV-1 1st-binding protein 1) | ZBTB7 |
| | | (Leukemia/lymphoma-related factor) (POZ and | ZNF857A |
| | | Krueppel erythroid myeloid ontogenic factor) | |
| | | (POK erythroid myeloid ontogenic factor) | |
| | | (Pokemon) (TTF-I-interacting peptide 21) (TIP21) | |
| | | (Zinc finger protein 857A) | |
| 360 | NR2C2_HUMAN | Nuclear receptor subfamily 2 group C member 2 | NR2C2 |
| | | (Orphan nuclear receptor TAK1) (Orphan nuclear | TAK1 |
| | | receptor TR4) (Testicular receptor 4) | TR4 |
| 361 | NR4A1_HUMAN | Nuclear receptor subfamily 4 group A member 1 | NR4A1 |
| | | (Early response protein NAK1) (Nuclear hormone | GFRP1 |
| | | receptor NUR/77) (Nur77) (Orphan nuclear | HMR |
| | | receptor HMR) (Orphan nuclear receptor TR3) | NAK1 |
| | | (ST-59) (Testicular receptor 3) | |
| 362 | NR4A2_HUMAN | Nuclear receptor subfamily 4 group A member 2 | NR4A2 |
| | | (Immediate-early response protein NOT) (Orphan | NOT |
| | | nuclear receptor NURR1) (Transcriptionally- | NURR1 |
| | | inducible nuclear receptor) | TINUR |
| 363 | MYNN_HUMAN | Myoneurin (Zinc finger and BTB domain- | MYNN |
| | | containing protein 31) | OSZF |
| | | | ZBTB31 |
| | | | SBBIZ1 |
| 364 | RFX5_HUMAN | DNA-binding protein RFX5 (Regulatory factor X 5) | RFX5 |
| 365 | P66A_HUMAN | Transcriptional repressor p66-alpha (Hp66alpha) | GATAD2A |
| | | (GATA zinc finger domain-containing protein 2A) | |
| 366 | BMAL2_HUMAN | Aryl hydrocarbon receptor nuclear translocator-like | ARNTL2 |
| | | protein 2 (Basic-helix-loop-helix-PAS protein | BHLHE6 |
| | | MOP9) (Brain and muscle ARNT-like 2) (CYCLE- | BMAL2 |
| | | like factor) (CLIF) (Class E basic helix-loop-helix | CLIF |
| | | protein 6) (bHLHe6) (Member of PAS protein 9) | MOP9 |
| | | (PAS domain-containing protein 9) | PASD9 |
| 367 | ITF2_HUMAN | Transcription factor 4 (TCF-4) (Class B basic | TCF4 |
| | | helix-loop-helix protein 19) (bHLHb19) | BHLHB19 |
| | | (Immunoglobulin transcription factor 2) (ITF-2) | ITF2 |
| | | (SL3-3 enhancer factor 2) (SEF-2) | SEF2 |
| 368 | ZBT16_HUMAN | Zinc finger and BTB domain-containing protein 16 | ZBTB16 |
| | | (Promyelocytic leukemia zinc finger protein) (Zinc | PLZF |
| | | finger protein 145) (Zinc finger protein PLZF) | ZNF145 |
| 369 | HTF4_HUMAN | Transcription factor 12 (TCF-12) (Class B basic | TCF12 |
| | | helix-loop-helix protein 20) (bHLHb20) (DNA- | BHLHB20 |
| | | binding protein HTF4) (E-box-binding protein) | HEB |
| | | (Transcription factor HTF-4) | HTF4 |
| 370 | TZAP_HUMAN | Telomere zinc finger-associated protein (TZAP) | ZBTB48 |
| | | (Krueppel-related zinc finger protein 3) (hKR3) | HKR3 |
| | | (Zinc finger and BTB domain-containing protein | TZAP |
| | | 48) (Zinc finger protein 855) | ZNF855 |
| 371 | MZF1_HUMAN | Myeloid zinc finger 1 (MZF-1) (Zinc finger and | MZF1 |
| | | SCAN domain-containing protein 6) (Zinc finger | MZF |
| | | protein 42) | ZNF42 |
| | | | ZSCAN6 |
| 372 | BACH1_HUMAN | Transcription regulator protein BACH1 (BTB and | BACH1 |
| | | CNC homolog 1) (HA2303) | |
| 373 | ZN483_HUMAN | Zinc finger protein 483 (Zinc finger protein with | ZNF483 |
| | | KRAB and SCAN domains 16) | KIAA1962 |
| | | | ZKSCAN16 |
| 374 | LMBL1_HUMAN | Lethal(3)malignant brain tumor-like protein 1 (H- | L3MBTL1 |
| | | l(3)mbt) (H-l(3)mbt protein) (L(3)mbt-like) | KIAA0681 |
| | | (L(3)mbt protein homolog) (L3MBTL1) | L3MBT |
| | | | L3MBTL |
| 375 | DMTF1_HUMAN | Cyclin-D-binding Myb-like transcription factor 1 | DMTF1 |
| | | (hDMTF1) (Cyclin-D-interacting Myb-like protein | DMP1 |
| | | 1) (hDMP1) | |
| 376 | UBF1_HUMAN | Nucleolar transcription factor 1 (Autoantigen | UBTF |
| | | NOR-90) (Upstream-binding factor 1) (UBF-1) | UBF |
| | | | UBF1 |
| 377 | STAT3_HUMAN | Signal transducer and activator of transcription 3 | STAT3 |
| | | (Acute-phase response factor) | APRF |
| 378 | LMBL3_HUMAN | Lethal(3)malignant brain tumor-like protein 3 (H- | L3MBTL3 |
| | | 1(3)mbt-like protein 3) (L(3)mbt-like protein 3) | KIAA1798 |
| | | (MBT-1) | MBT1 |
| 379 | STRN3_HUMAN | Striatin-3 (Cell cycle autoantigen SG2NA) (S/G2 | STRN3 |
| | | antigen) | GS2NA |
| | | | SG2NA |
| 380 | PRGC1_HUMAN | Peroxisome proliferator-activated receptor gamma | PPARGC1A |
| | | coactivator 1-alpha (PGC-1-alpha) (PPAR-gamma | LEM6 |
| | | coactivator 1-alpha) (PPARGC-1-alpha) (Ligand | PGC1 |
| | | effect modulator 6) | PGC1A |
| | | | PPARGC1 |
| 381 | PRDM4_HUMAN | PR domain zinc finger protein 4 (EC 2.1.1.—) (PR | PRDM4 |
| | | domain-containing protein 4) | PFM1 |
| 382 | LRRF1_HUMAN | Leucine-rich repeat flightless-interacting protein 1 | LRRFIP1 |
| | | (LRR FLII-interacting protein 1) (GC-binding | GCF2 |
| | | factor 2) (TAR RNA-interacting protein) | TRIP |
| 383 | PRDM1_HUMAN | PR domain zinc finger protein 1 (EC 2.1.1.—) | PRDM1 |
| | | (BLIMP-1) (Beta-interferon gene positive | BLIMP1 |
| | | regulatory domain I-binding factor) (PR domain- | |
| | | containing protein 1) (Positive regulatory domain I- | |
| | | binding factor 1) (PRDI-BF1) (PRDI-binding | |
| | | factor 1) | |
| 384 | HIF1A_HUMAN | Hypoxia-inducible factor 1-alpha (HIF-l-alpha) | HIF1A |
| | | (HIFl-alpha) (ARNT-interacting protein) (Basic- | BHLHE78 |
| | | helix-loop-helix-PAS protein MOP1) (Class E | MOP1 |
| | | basic helix-loop-helix protein 78) (bHLHe78) | PASD8 |
| | | (Member of PAS protein 1) (PAS domain- | |
| | | containing protein 8) | |
| 385 | BACH2_HUMAN | Transcription regulator protein BACH2 (BTB and | BACH2 |
| | | CNC homolog 2) | |
| 386 | STAT2_HUMAN | Signal transducer and activator of transcription 2 | STAT2 |
| | | (pH3) | |
| 387 | EPAS1_HUMAN | Endothelial PAS domain-containing protein 1 | EPAS1 |
| | | (EPAS-1) (Basic-helix-loop-helix-PAS protein | BHLHE73 |
| | | MOP2) (Class E basic helix-loop-helix protein 73) | HIF2A |
| | | (bHLHe73) (HIF-1-alpha-like factor) (HLF) | MOP2 |
| | | (Hypoxia-inducible factor 2-alpha) (HIF-2-alpha) | PASD2 |
| | | (HIF2-alpha) (Member of PAS protein 2) (PAS | |
| | | domain-containing protein 2) | |
| 388 | NFAC4_HUMAN | Nuclear factor of activated T-cells, cytoplasmic 4 | NFATC4 |
| | | (NF-ATc4) (NFATc4) (T-cell transcription factor | NFAT3 |
| | | NFAT3) (NF-AT3) | |
| 389 | PHF12_HUMAN | PHD finger protein 12 (PHD factor 1) (Pf1) | PHF12 |
| | | | KIAA1523 |
| 390 | FOG1_HUMAN | Zinc finger protein ZFPM1 (Friend of GAT A | ZFPM1 |
| | | protein 1) (FOG-1) (Friend of GATA 1) (Zinc finger | FOG1 |
| | | protein 89A) (Zinc finger protein multitype 1) | ZFN89A |
| 391 | PRGC2_HUMAN | Peroxisome proliferator-activated receptor gamma | PPARGC1B |
| | | coactivator 1-beta (PGC-1-beta) (PPAR-gamma | PERC |
| | | coactivator 1-beta) (PPARGC-1-beta) (PGC-1- | PGC1 |
| | | related estrogen receptor alpha coactivator) | PGC1B |
| | | | PPARGC1 |
| 392 | AF10_HUMAN | Protein AF-10 (ALL1-fused gene from | MLLT10 |
| | | chromosome 10 protein) | AF10 |
| 393 | NFAC3_HUMAN | Nuclear factor of activated T-cells, cytoplasmic 3 | NFATC3 |
| | | (NF-ATc3) (NFATc3) (NFATx) (T-cell | NFAT4 |
| | | transcription factor NFAT4) (NF-AT4) | |
| 394 | REST_HUMAN | RE1-silencing transcription factor (Neural- | REST |
| | | restrictive silencer factor) (X2 box repressor) | NRSF |
| | | | XBR |
| 395 | ZEB1_HUMAN | Zinc finger E-box-binding homeobox 1 (NIL-2-A | ZEB1 |
| | | zinc finger protein) (Negative regulator of IL2) | AREB6 |
| | | (Transcription factor 8) (TCF-8) | TCF8 |
| 396 | UBN1_HUMAN | Ubinuclein-1 (HIRA-binding protein) (Protein | UBN1 |
| | | VT4) (Ubiquitously expressed nuclear protein) | |
| 397 | RFC1_HUMAN | Replication factor C subunit 1 (Activator 1 140 | RFC1 |
| | | kDa subunit) (A1 140 kDa subunit) (Activator 1 | RFC140 |
| | | large subunit) (Activator 1 subunit 1) (DNA- | |
| | | binding protein PO-GA) (Replication factor C 140 | |
| | | kDa subunit) (RF-C 140 kDa subunit) (RFC140) | |
| | | (Replication factor C large subunit) | |
| 398 | NRIP1_HUMAN | Nuclear receptor-interacting protein 1 (Nuclear | NRIP1 |
| | | factor RIP140) (Receptor-interacting protein 140) | |
| 399 | MUC1_HUMAN | Mucin-1 (MUC-1) (Breast carcinoma-associated | MUC1 |
| | | antigen DF3) (Cancer antigen 15-3) (CA 15-3) | PUM |
| | | (Carcinoma-associated mucin) (Episialin) | |
| | | (H23AG) (Krebs von den Lungen-6) (KL-6) | |
| | | (PEMT) (Peanut-reactive urinary mucin) (PUM) | |
| | | (Polymorphic epithelial mucin) (PEM) (Tumor- | |
| | | associated epithelial membrane antigen) (EMA) | |
| | | (Tumor-associated mucin) (CD antigen CD227) | |
| | | [Cleaved into: Mucin-1 subunit alpha (MUC1-NT) | |
| | | (MUC1-alpha); Mucin-1 subunit beta (MUC 1-beta) | |
| | | (MUC1-CT)] | |
| 400 | PRD16_HUMAN | PR domain zinc finger protein 16 (PR domain- | PRDM16 |
| | | containing protein 16) (Transcription factor MEL1) | KIAA1675 |
| | | (MDS 1/EVI1-like gene 1) | MEL1 |
| | | | PFM13 |
| 401 | RFX7_HUMAN | DNA-binding protein RFX7 (Regulatory factor X 7) | RFX7 |
| | | (Regulatory factor X domain-containing protein 2) | RFXDC2 |
| 402 | NCOA2_HUMAN | Nuclear receptor coactivator 2 (NCoA-2) (Class E | NCOA2 |
| | | basic helix-loop-helix protein 75) (bHLHe75) | BHLHE75 |
| | | (Transcriptional intermediary factor 2) (hTIF2) | SRC2 |
| | | | TIF2 |
| 403 | RHG35_HUMAN | Rho GTPase-activating protein 35 (Glucocorticoid | ARHGAP35 |
| | | receptor DNA-binding factor 1) (Glucocorticoid | GRF1 |
| | | receptor repression factor 1) (GRF-1) (Rho GAP | GRLF1 |
| | | p190A) (p190-A) | KIAA1722 |
| | | | P190A |
| | | | p190AR |
| | | | HOGAP |
| 404 | GLI3_HUMAN | Transcriptional activator GLI3 (GLI3 form of 190 | GLI3 |
| | | kDa) (GLI3-190) (GLI3 full-length protein) | |
| | | (GLI3FL) [Cleaved into: Transcriptional repressor | |
| | | GLI3R (GLI3 C-terminally truncated form) (GLI3 | |
| | | form of 83 kDa) (GLI3-83)] | |
| 405 | PEG3_HUMAN | Paternally-expressed gene 3 protein (Zinc finger | PEG3 |
| | | and SCAN domain-containing protein 24) | KIAA0287 |
| | | | ZSCAN24 |
| 406 | PRDM2_HUMAN | PR domain zinc finger protein 2 (EC 2.1.1.43) | PRDM2 |
| | | (GATA-3-binding protein G3B) (Lysine N- | KMT8 |
| | | methyltransferase 8) (MTB-ZF) (MTE-binding | RIZ |
| | | protein) (PR domain-containing protein 2) | |
| | | (Retinoblastoma protein-interacting zinc finger | |
| | | protein) (Zinc finger protein RIZ) | |
| 407 | TP53B_HUMAN | TP53-binding protein 1 (53BP1) (p53-binding | TP53BP1 |
| | | protein 1) (p53BP1) | |
| 408 | ZEP1_HUMAN | Zinc finger protein 40 (Cirhin interaction protein) | HIVEP1 |
| | | (CIRIP) (Gate keeper of apoptosis-activating | ZNF40 |
| | | protein) (GAAP) (Human immunodeficiency virus | |
| | | type I enhancer-binding protein 1) (HIV-EP1) | |
| | | (Major histocompatibility complex-binding protein | |
| | | 1) (MBP-1) (Positive regulatory domain II-binding | |
| | | factor 1) (PRDII-BF1) | |
| 409 | BPTF_HUMAN | Nucleosome-remodeling factor subunit BPTF | BPTF |
| | | (Bromodomain and PHD finger-containing | FAC1 |
| | | transcription factor) (Fetal Alz-50 clone 1 protein) | FALZ |
| | | (Fetal Alzheimer antigen) | |
| 410 | ZFHX3_HUMAN | Zinc finger homeobox protein 3 (AT motif-binding | ZFHX3 |
| | | factor 1) (AT-binding transcription factor 1) | ATBF1 |
| | | (Alpha-fetoprotein enhancer-binding protein) (Zinc | |
| | | finger homeodomain protein 3) (ZFH-3) | |
| 411 | SNPC5_HUMAN | snRNA-activating protein complex subunit 5 | SNAPC5 |
| | | (SNAPc subunit 5) (Small nuclear RNA-activating | SNAP19 |
| | | complex polypeptide 5) (snRNA-activating protein | |
| | | complex 19 kDa subunit) (SNAPc 19 kDa subunit) | |
| 412 | TAL2_HUMAN | T-cell acute lymphocytic leukemia protein 2 (TAL- | TAL2 |
| | | 2) (Class A basic helix-loop-helix protein 19) | BHLHA19 |
| | | (bHLHa19) | |
| 413 | HMGA2_HUMAN | High mobility group protein HMGI-C (High | HMGA2 |
| | | mobility group AT-hook protein 2) | HMGIC |
| 414 | CRBL2_HUMAN | cAMP-responsive element-binding protein-like 2 | CREBL2 |
| 415 | PFD1_HUMAN | Prefoldin subunit 1 | PFDN1 |
| | | | PFD1 |
| 416 | BATF_HUMAN | Basic leucine zipper transcriptional factor ATF-like | BATF |
| | | (B-cell-activating transcription factor) (B-ATF) | |
| | | (SF-HT-activated gene 2 protein) (SFA-2) | |
| 417 | BEX1_HUMAN | Protein BEX1 (Brain-expressed X-linked protein 1) | BEX1 |
| 418 | BATF3_HUMAN | Basic leucine zipper transcriptional factor ATF-like | BATF3 |
| | | 3 (B-ATF-3) (21 kDa small nuclear factor isolated | SNFT |
| | | from T-cells) (Jun dimerization protein p21SNFT) | |
| 419 | T22D3_HUMAN | TSC22 domain family protein 3 (DSIP- | TSC22D3 |
| | | immunoreactive peptide) (Protein DIP) (hDIP) | DSIPI |
| | | (Delta sleep-inducing peptide immunoreactor) | GILZ |
| | | (Glucocorticoid-induced leucine zipper protein) | |
| | | (GILZ) (TSC-22-like protein) (TSC-22-related | |
| | | protein) (TSC-22R) | |
| 420 | HEN2_HUMAN | Helix-loop-helix protein 2 (HEN-2) (Class A basic | NHLH2 |
| | | helix-loop-helix protein 34) (bHLHa34) (Nescient | BHLHA34 |
| | | helix loop helix 2) (NSCL-2) | HEN2 |
| | | | KIAA0490 |
| 421 | CYTL1_HUMAN | Cytokine-like protein 1 (Protein C17) | CYTL1 |
| | | | C4orf4 |
| | | | UNQ1942/ |
| | | | PRO4425 |
| 422 | ZN818_HUMAN | Putative zinc finger protein 818 | ZNF818P |
| | | | ZNF818 |
| 423 | RGCC_HUMAN | Regulator of cell cycle RGCC (Response gene to | RGCC |
| | | complement 32 protein) (RGC-32) | C13orf15 |
| | | | RGC32 |
| 424 | CEBPG_HUMAN | CCAAT/enhancer-binding protein gamma (C/EBP | CEBPG |
| | | gamma) | |
| 425 | CHCH2_HUMAN | Coiled-coil-helix-coiled-coil-helix domain- | CHCHD2 |
| | | containing protein 2 (Aging-associated gene 10 | C7orf17 |
| | | protein) (HCV NS2 trans-regulated protein) | AAG10 |
| | | (NS2TP) | |
| 426 | ID1_HUMAN | DNA-binding protein inhibitor ID-1 (Class B basic | ID1 |
| | | helix-loop-helix protein 24) (bHLHb24) (Inhibitor | BHLHB24 |
| | | of DNA binding 1) (Inhibitor of differentiation 1) | ID |
| 427 | MAFK_HUMAN | Transcription factor MafK (Erythroid transcription | MAFK |
| | | factor NF-E2 p18 subunit) | |
| 428 | TCAL1_HUMAN | Transcription elongation factor A protein-like 1 | TCEAL1 |
| | | (TCEA-like protein 1) (Nuclear phosphoprotein | SIIR |
| | | p21/SIIR) (Transcription elongation factor S-II | |
| | | protein-like 1) | |
| 429 | LITAF_HUMAN | Lipopolysaccharide-induced tumor necrosis factor- | LITAF |
| | | alpha factor (LPS-induced TNF-alpha factor) | PIG7 |
| | | (Small integral membrane protein of lysosome/late | SIMPLE |
| | | endosome) (p53-induced gene 7 protein) | |
| 430 | ZNF56_HUMAN | Putative zinc finger protein 56 (Putative zinc finger | ZNF56 |
| | | protein 742) | ZNF742 |
| 431 | MAFG_HUMAN | Transcription factor MafG (V-maf | MAFG |
| | | musculoaponeurotic fibrosarcoma oncogene | |
| | | homolog G) (hMAF) | |
| 432 | JDP2_HUMAN | Jun dimerization protein 2 | JDP2 |
| 433 | MAFF_HUMAN | Transcription factor MafF (U-Maf) (V-maf | MAFF |
| | | musculoaponeurotic fibrosarcoma oncogene | |
| | | homolog F) | |
| 434 | FER3L_HUMAN | Fer3-like protein (Basic helix-loop-helix protein N- | FERD3L |
| | | twist) (Class A basic helix-loop-helix protein 31) | BHLHA31 |
| | | (bHLHa31) (Nephew of atonal 3) (Neuronal twist) | NATO3 |
| | | | NTWIST |
| 435 | HES5_HUMAN | Transcription factor HES-5 (Class B basic helix- | HES5 |
| | | loop-helix protein 38) (bHLHb38) (Hairy and | BHLHB38 |
| | | enhancer of split 5) | |
| 436 | DDIT3_HUMAN | DNA damage-inducible transcript 3 protein (DDIT- | DDIT3 |
| | | 3) (C/EBP zeta) (C/EBP-homologous protein) | CHOP |
| | | (CHOP) (C/EBP-homologous protein 10) (CHOP- | CHOP10 |
| | | 10) (CCAAT/enhancer-binding protein | GADD153 |
| | | homologous protein) (Growth arrest and DNA | |
| | | damage-inducible protein GADD153) | |
| 437 | MDS1_HUMAN | MDS1 and EVI1 complex locus protein MDS1 | MECOM |
| | | (Myelodysplasia syndrome 1 protein) | MDS1 |
| | | (Myelodysplasia syndrome-associated protein 1) | |
| 438 | ASCL4_HUMAN | Achaete-scute homolog 4 (ASH-4) (hASH4) | ASCL4 |
| | | (Achaete-scute-like protein 4) (Class A basic helix- | BHLHA44 |
| | | loop-helix protein 44) (bHLHa44) | HASH4 |
| 439 | HES2_HUMAN | Transcription factor HES-2 (Class B basic helix- | HES2 |
| | | loop-helix protein 40) (bHLHb40) (Hairy and | BHLHB40 |
| | | enhancer of split 2) | |
| 440 | DLX6_HUMAN | Homeobox protein DLX-6 | DLX6 |
| 441 | CNBP_HUMAN | Cellular nucleic acid-binding protein (CNBP) (Zinc | CNBP |
| | | finger protein 9) | RNF163 |
| | | | ZNF9 |
| 442 | SCND1_HUMAN | SCAN domain-containing protein 1 | SCAND1 |
| | | | SDP1 |
| 443 | TCF21_HUMAN | Transcription factor 21 (TCF-21) (Capsulin) (Class | TCF21 |
| | | A basic helix-loop-helix protein 23) (bHLHa23) | BHLHA23 |
| | | (Epicardin) (Podocyte-expressed 1) (Pod-1) | POD1 |
| 444 | ASCL3_HUMAN | Achaete-scute homolog 3 (ASH-3) (hASH3) (Class | ASCL3 |
| | | A basic helix-loop-helix protein 42) (bHLHa42) | BHLHA42 |
| | | (bHLH transcriptional regulator Sgn-1) | HASH3 |
| | | | SGN1 |
| 445 | ATF3_HUMAN | Cyclic AMP-dependent transcription factor ATF-3 | ATF3 |
| | | (cAMP-dependent transcription factor ATF-3) | |
| | | (Activating transcription factor 3) | |
| 446 | MSRB2_HUMAN | Methionine-R-sulfoxide reductase B2, | MSRB2 |
| | | mitochondrial (MsrB2) (EC 1.8.4.—) | CBS-1 |
| | | | MSRB |
| | | | CGI-131 |
| 447 | RHXF1_HUMAN | Rhox homeobox family member 1 (Ovary-, testis- | RHOXF1 |
| | | and epididymis-expressed gene protein) (Paired- | OTEX |
| | | like homeobox protein PEPP-1) | PEPP1 |
| 448 | TBPL1_HUMAN | TATA box-binding protein-like protein 1 (TBP- | TBPL1 |
| | | like protein 1) (21 kDa TBP-like protein) (Second | TLF |
| | | TBP of unique DNA protein) (STUD) (TATA box- | TLP |
| | | binding protein-related factor 2) (TBP-related | TLP21 |
| | | factor 2) (TBP-like factor) (TBP-related protein) | TRF2 |
| | | | TRP |
| 449 | HES3_HUMAN | Transcription factor HES-3 (Class B basic helix- | HES3 |
| | | loop-helix protein 43) (bHLHb43) (Hairy and | BHLHB43 |
| | | enhancer of split 3) | |
| 450 | DPRX_HUMAN | Divergent paired-related homeobox | DPRX |
| 451 | DMRTC_HUMAN | Doublesex- and mab-3-related transcription factor | DMRTC1; |
| | | C1 | DMRTC1B |
| 452 | ASCL2_HUMAN | Achaete-scute homolog 2 (ASH-2) (hASH2) (Class | ASCL2 |
| | | A basic helix-loop-helix protein 45) (bHLHa45) | BHLHA45 |
| | | (Mash2) | HASH2 |
| 453 | CTTE1_HUMAN | Cbp/p300-interacting transactivator 1 (Melanocyte- | CITED1 |
| | | specific protein 1) | MSG1 |
| 454 | ZN525_HUMAN | Zinc finger protein 525 | ZNF525 |
| | | | KIAA1979 |
| 455 | TCF15_HUMAN | Transcription factor 15 (TCF-15) (Class A basic | TCF15 |
| | | helix-loop-helix protein 40) (bHLHa40) (Paraxis) | BHLHA40 |
| | | (Protein bHLH-EC2) | BHLHEC2 |
| 456 | SCX_HUMAN | Basic helix-loop-helix transcription factor scleraxis | SCX |
| | | (Class A basic helix-loop-helix protein 41) | BHLHA41 |
| | | (bHLHa41) (Class A basic helix-loop-helix protein | BHLHA48 |
| | | 48) (bHLHa48) | SCXA |
| | | | SCXB |
| 457 | PTTG1_HUMAN | Securin (Esp1-associated protein) (Pituitary tumor- | PTTG1 |
| | | transforming gene 1 protein) (Tumor-transforming | EAP1 |
| | | protein 1) (hPTTG) | PTTG |
| | | | TUTR1 |
| 458 | GSC2_HUMAN | Homeobox protein goosecoid-2 (GSC-2) | GSC2 |
| | | (Homeobox protein goosecoid-like) (GSC-L) | GSCL |
| 459 | MAD3_HUMAN | Max dimerization protein 3 (Max dimerizer 3) | MXD3 |
| | | (Class C basic helix-loop-helix protein 13) | BHLHC13 |
| | | (bHLHc13) (Max-associated protein 3) (Max- | MAD3 |
| | | interacting transcriptional repressor MAD3) (Myx) | |
| 460 | MUSC_HUMAN | Musculin (Activated B-cell factor 1) (ABF-1) | MSC |
| | | (Class A basic helix-loop-helix protein 22) | ABF1 |
| | | (bHLHa22) | BHLHA22 |
| 461 | ZN137_HUMAN | Putative zinc finger protein 137 (Zinc finger | ZNF137P |
| | | protein 137 pseudogene) | ZNF137 |
| 462 | HMGB2_HUMAN | High mobility group protein B2 (High mobility | HMGB2 |
| | | group protein 2) (HMG-2) | HMG2 |
| 463 | NGN3_HUMAN | Neurogenin-3 (NGN-3) (Class A basic helix-loop- | NEUROG3 |
| | | helix protein 7) (bHLHa7) (Protein atonal homolog | ATOH5 |
| | | 5) | BHLHA7 |
| | | | NGN3 |
| 464 | OVOL3_HUMAN | Putative transcription factor ovo-like protein 3 | OVOL3 |
| 465 | HAND1_HUMAN | Heart- and neural crest derivatives-expressed | HAND1 |
| | | protein 1 (Class A basic helix-loop-helix protein | BHLHA27 |
| | | 27) (bHLHa27) (Extraembryonic tissues, heart, | EHAND |
| | | autonomic nervous system and neural crest | |
| | | derivatives-expressed protein 1) (eHAND) | |
| 466 | HAND2_HUMAN | Heart- and neural crest derivatives-expressed | HAND2 |
| | | protein 2 (Class A basic helix-loop-helix protein | BHLHA26 |
| | | 26) (bHLHa26) (Deciduum, heart, autonomic | DHAND |
| | | nervous system and neural crest derivatives- | |
| | | expressed protein 2) (dHAND) | |
| 467 | HXB7_HUMAN | Homeobox protein Hox-B7 (Homeobox protein | HOXB7 |
| | | HHO.C1) (Homeobox protein Hox-2C) | HOX2C |
| 468 | FIGLA_HUMAN | Factor in the germline alpha (FIGalpha) (Class C | FIGLA |
| | | basic helix-loop-helix protein 8) (bHLHc8) | BHLHC8 |
| | | (Folliculogenesis-specific basic helix-loop-helix | |
| | | protein) (Transcription factor FIGa) | |
| 469 | HXC5_HUMAN | Homeobox protein Hox-C5 (Homeobox protein | HOXC5 |
| | | CP1) (Homeobox protein Hox-3D) | HOX3D |
| 470 | IER2_HUMAN | Immediate early response gene 2 protein (Protein | IER2 |
| | | ETR101) | ETR101 |
| | | | PIP92 |
| 471 | HXB6_HUMAN | Homeobox protein Hox-B6 (Homeobox protein | HOXB6 |
| | | Hox-2.2) (Homeobox protein Hox-2B) (Homeobox | HOX2B |
| | | protein Hu-2) | |
| 472 | MYOG_HUMAN | Myogenin (Class C basic helix-loop-helix protein | MYOG |
| | | 3) (bHLHc3) (Myogenic factor 4) (Myf-4) | BHLHC3 |
| | | | MYF4 |
| 473 | HES6_HUMAN | Transcription cofactor HES-6 (C-HAIRY1) (Class | HES6 |
| | | B basic helix-loop-helix protein 41) (bHLHb41) | BHLHB41 |
| | | (Hairy and enhancer of split 6) | |
| 474 | HES7_HUMAN | Transcription factor HES-7 (hHes7) (Class B basic | HES7 |
| | | helix-loop-helix protein 37) (bHLHb37) (Hairy and | BHLHB37 |
| | | enhancer of split 7) (bHLH factor Hes7) | |
| 475 | PROP1_HUMAN | Homeobox protein prophet of Pit-1 (PROP-1) | PROP1 |
| | | (Pituitary-specific homeodomain factor) | |
| 476 | YETS4_HUMAN | YEATS domain-containing protein 4 (Glioma- | YEATS4 |
| | | amplified sequence 41) (Gas41) (NuMA-binding | GAS41 |
| | | protein 1) (NuBI-1) (NuBI1) | |
| 477 | MXI1_HUMAN | Max-interacting protein 1 (Max interactor 1) (Class | MXI1 |
| | | C basic helix-loop-helix protein 11) (bHLHc11) | BHLHC11 |
| 478 | HXA7_HUMAN | Homeobox protein Hox-A7 (Homeobox protein | HOXA7 |
| | | Hox 1.1) (Homeobox protein Hox-1A) | HOX1A |
| 479 | MIXL1_HUMAN | Homeobox protein MIXL1 (Homeodomain protein | MIXL1 |
| | | MIX) (hMix) (MIX1 homeobox-like protein 1) | MIXL |
| | | (Mix.l homeobox-like protein) | |
| 480 | BSH_HUMAN | Brain-specific homeobox protein homolog | BSX |
| | | | BSX1 |
| 481 | HXA6_HUMAN | Homeobox protein Hox-A6 (Homeobox protein | HOXA6 |
| | | Hox-1B) | HOX1B |
| 482 | SOX15_HUMAN | Protein SOX-15 (Protein SOX-12) (Protein SOX- | SOX15 |
| | | 20) | SOX12 |
| | | | SOX20 |
| | | | SOX26 |
| | | | SOX27 |
| 483 | HXC6_HUMAN | Homeobox protein Hox-C6 (Homeobox protein | HOXC6 |
| | | CP25) (Homeobox protein HHO.C8) (Homeobox | HOX3C |
| | | protein Hox-3C) | |
| 484 | ASCL1_HUMAN | Achaete-scute homolog 1 (ASH-1) (hASH1) (Class | ASCL1 |
| | | A basic helix-loop-helix protein 46) (bHLHa46) | ASH1 |
| | | | BHLHA46 |
| | | | HASH1 |
| 485 | TGIF2_HUMAN | Homeobox protein TGIF2 (5′-TG-3′-interacting | TGIF2 |
| | | factor 2) (TGF-beta-induced transcription factor 2) | |
| | | (TGFB-induced factor 2) | |
| 486 | NRL_HUMAN | Neural retina-specific leucine zipper protein (NRL) | NRL |
| | | | D14S46E |
| 487 | NGN1_HUMAN | Neurogenin-1 (NGN-1) (Class A basic helix-loop- | NEUROG1 |
| | | helix protein 6) (bHLHa6) (Neurogenic basic- | BHLHA6 |
| | | helix-loop-helix protein) (Neurogenic | NEUROD3 |
| | | differentiation factor 3) (NeuroD3) | NGN |
| | | | NGN1 |
| 488 | NKX28_HUMAN | Homeobox protein Nkx-2.8 (Homeobox protein | NKX2-8 |
| | | NK-2 homolog H) | NKX-2.8 |
| | | | NKX2G |
| | | | NKX2H |
| 489 | DLX4_HUMAN | Homeobox protein DLX-4 (Beta protein 1) | DLX4 |
| | | (Homeobox protein DLX-7) (Homeobox protein | BP1 |
| | | DLX-8) | DLX7 |
| | | | DLX8 |
| | | | DLX9 |
| 490 | SOX14_HUMAN | Transcription factor SOX-14 (Protein SOX-28) | SOX14 |
| | | | SOX28 |
| 491 | HELT_HUMAN | Hairy and enhancer of split-related protein HELT | HELT |
| | | (HES/HEY-like transcription factor) | |
| 492 | HXC8_HUMAN | Homeobox protein Hox-C8 (Homeobox protein | HOXC8 |
| | | Hox-3A) | HOX3A |
| 493 | MYF6_HUMAN | Myogenic factor 6 (Myf-6) (Class C basic helix- | MYF6 |
| | | loop-helix protein 4) (bHLHc4) (Muscle-specific | BHLHC4 |
| | | regulatory factor 4) | MRF4 |
| 494 | HXB8_HUMAN | Homeobox protein Hox-B8 (Homeobox protein | HOXB8 |
| | | Hox-2.4) (Homeobox protein Hox-2D) | HOX2D |
| 495 | NUCKS_HUMAN | Nuclear ubiquitous casein and cyclin-dependent | NUCKS1 |
| | | kinase substrate 1 (P1) | NUCKS |
| | | | JC7 |
| 496 | KLF9_HUMAN | Krueppel-like factor 9 (Basic transcription element- | KLF9 |
| | | binding protein 1) (BTE-binding protein 1) (GC- | BTEB |
| | | box-binding protein 1) (Transcription factor | BTEB1 |
| | | BTEB1) | |
| 497 | ISX_HUMAN | Intestine-specific homeobox (RAX-like homeobox) | ISX |
| | | | RAXLX |
| 498 | PRRX1_HUMAN | Paired mesoderm homeobox protein 1 (Homeobox | PRRX1 |
| | | protein PHOX1) (Paired-related homeobox protein | PMX1 |
| | | 1) (PRX-1) | |
| 499 | SPIC_HUMAN | Transcription factor Spi-C | SPIC |
| 500 | HXB9_HUMAN | Homeobox protein Hox-B9 (Homeobox protein | HOXB9 |
| | | Hox-2.5) (Homeobox protein Hox-2E) | HOX2E |
| 501 | HXB4_HUMAN | Homeobox protein Hox-B4 (Homeobox protein | HOXB4 |
| | | Hox-2.6) (Homeobox protein Hox-2F) | HOX2F |
| 502 | RCAN1_HUMAN | Calcipressin-1 (Adapt78) (Down syndrome critical | RCAN1 |
| | | region protein 1) (Myocyte-enriched calcineurin- | ADAPT78 |
| | | interacting protein 1) (MCIP1) (Regulator of | CSP1 |
| | | calcineurin 1) | DSC1 |
| | | | DSCR1 |
| 503 | EMX2_HUMAN | Homeobox protein EMX2 (Empty spiracles | EMX2 |
| | | homolog 2) (Empty spiracles-like protein 2) | |
| 504 | KLF16_HUMAN | Krueppel-like factor 16 (Basic transcription | KLF16 |
| | | element-binding protein 4) (BTE-binding protein 4) | BTEB4 |
| | | (Novel Sp1-like zinc finger transcription factor 2) | NSLP2 |
| | | (Transcription factor BTEB4) (Transcription factor | |
| | | NSLP2) | |
| 505 | PRRX2_HUMAN | Paired mesoderm homeobox protein 2 (Paired- | PRRX2 |
| | | related homeobox protein 2) (PRX-2) | PMX2 |
| | | | PRX2 |
| 506 | MEOX1_HUMAN | Homeobox protein MOX-1 (Mesenchyme | MEOX1 |
| | | homeobox 1) | MOX1 |
| 507 | DLX1_HUMAN | Homeobox protein DLX-1 | DLX1 |
| 508 | HXD4_HUMAN | Homeobox protein Hox-D4 (Homeobox protein | HOXD4 |
| | | HHO.C13) (Homeobox protein Hox-4B) | HOX4B |
| | | (Homeobox protein Hox-5.1) | |
| 509 | MYF5_HUMAN | Myogenic factor 5 (Myf-5) (Class C basic helix- | MYF5 |
| | | loop-helix protein 2) (bHLHc2) | BHLHC2 |
| 510 | EMX1_HUMAN | Homeobox protein EMX1 (Empty spiracles | EMX1 |
| | | homolog 1) (Empty spiracles-like protein 1) | |
| 511 | EAF2_HUMAN | ELL-associated factor 2 (Testosterone-regulated | EAF2 |
| | | apoptosis inducer and tumor suppressor protein) | TRAITS |
| | | | BM-040 |
| 512 | XBP1_HUMAN | X-box-binding protein 1 (XBP-1) (Tax-responsive | XBP1 |
| | | element-binding protein 5) (TREB-5) [Cleaved | TREB5 |
| | | into: X-box-binding protein 1, cytoplasmic form; | XBP2 |
| | | X-box-binding protein 1, luminal form] | |
| 513 | ZN664_HUMAN | Zinc finger protein 664 (Zinc finger protein 176) | ZNF664 |
| | | (Zinc finger protein from organ of Corti) | ZFOC1 |
| | | | ZNF176 |
| 514 | SPIB_HUMAN | Transcription factor Spi-B | SPIB |
| 515 | ZN138_HUMAN | Zinc finger protein 138 | ZNF138 |
| 516 | DRGX_HUMAN | Dorsal root ganglia homeobox protein (Paired- | DRGX |
| | | related homeobox protein-like 1) | PRRXL1 |
| 517 | GSX1_HUMAN | GS homeobox 1 (Homeobox protein GSH-1) | GSX1 |
| | | | GSH1 |
| 518 | HXC4_HUMAN | Homeobox protein Hox-C4 (Homeobox protein | HOXC4 |
| | | CP19) (Homeobox protein Hox-3E) | HOX3E |
| 519 | NKX63_HUMAN | Homeobox protein Nkx-6.3 | NKX6-3 |
| 520 | MSX2_HUMAN | Homeobox protein MSX-2 (Homeobox protein | MSX2 |
| | | Hox-8) | HOX8 |
| 521 | OVOL1_HUMAN | Putative transcription factor Ovo-like 1 (hOvo1) | OVOL1 |
| 522 | MESP1_HUMAN | Mesoderm posterior protein 1 (Class C basic helix- | MESP1 |
| | | loop-helix protein 5) (bHLHc5) | BHLHC5 |
| 523 | SNAI2_HUMAN | Zinc finger protein SNAI2 (Neural crest | SNAI2 |
| | | transcription factor Slug) (Protein snail homolog 2) | SLUG |
| | | | SLUGH |
| 524 | CEBPD_HUMAN | CCAAT/enhancer-binding protein delta (C/EBP | CEBPD |
| | | delta) (Nuclear factor NF-IL6-beta) (NF-IL6-beta) | |
| 525 | GATD1_HUMAN | GATA zinc finger domain-containing protein 1 | GATAD1 |
| | | (Ocular development-associated gene protein) | ODAG |
| 526 | HXB5_HUMAN | Homeobox protein Hox-B5 (Homeobox protein | HOXB5 |
| | | HHO.C10) (Homeobox protein Hox-2A) | HOX2A |
| | | (Homeobox protein Hu-1) | |
| 527 | HXA5_HUMAN | Homeobox protein Hox-A5 (Homeobox protein | HOXA5 |
| | | Hox-1C) | HOX1C |
| 528 | HXD12_HUMAN | Homeobox protein Hox-D12 (Homeobox protein | HOXD12 |
| | | Hox-4H) | HOX4H |
| 529 | NFAM1_HUMAN | NFAT activation molecule 1 (Calcineurin/NFAT- | NFAM1 |
| | | activating ITAM-containing protein) (NFAT- | CNAIP |
| | | activating protein with ITAM motif 1) | |
| 530 | SPI1_HUMAN | Transcription factor PU.1 (31 kDa-transforming | SPI1 |
| | | protein) | |
| 531 | ATF1_HUMAN | Cyclic AMP-dependent transcription factor ATF-1 | ATF1 |
| | | (cAMP-dependent transcription factor ATF-1) | |
| | | (Activating transcription factor 1) (Protein | |
| | | TREB36) | |
| 532 | FOSL1_HUMAN | Fos-related antigen 1 (FRA-1) | FOSL1 |
| | | | FRA1 |
| 533 | ZGLP1_HUMAN | GATA-type zinc finger protein 1 (GATA-like | ZGLP1 |
| | | protein 1) (GLP-1) | GLP1 |
| 534 | ZN501_HUMAN | Zinc finger protein 501 (Zinc finger protein 52) | ZNF501 |
| | | | ZNF52 |
| 535 | HXA9_HUMAN | Homeobox protein Hox-A9 (Homeobox protein | HOXA9 |
| | | Hox-1G) | HOX1G |
| 536 | NGN2_HUMAN | Neurogenin-2 (NGN-2) (Class A basic helix-loop- | NEUROG2 |
| | | helix protein 8) (bHLHa8) (Protein atonal homolog | ATOH4 |
| | | 4) | BHLHA8 |
| | | | NGN2 |
| 537 | HMX2_HUMAN | Homeobox protein HMX2 (Homeobox protein H6 | HMX2 |
| | | family member 2) | |
| 538 | NKX22_HUMAN | Homeobox protein Nkx-2.2 (Homeobox protein | NKX2-2 |
| | | NK-2 homolog B) | NKX2.2 |
| | | | NKX2B |
| 539 | BATF2_HUMAN | Basic leucine zipper transcriptional factor ATF-like | BATF2 |
| | | 2 (B-ATF-2) (Suppressor of AP-1 regulated by | |
| | | IFN) (SARI) | |
| 540 | OVOL2_HUMAN | Transcription factor Ovo-like 2 (hOvo2) (Zinc | OVOL2 |
| | | finger protein 339) | ZNF339 |
| 541 | SOX21_HUMAN | Transcription factor SOX-21 (SOX-A) | SOX21 |
| | | | SOX25 |
| | | | SOXA |
| 542 | NKX62_HUMAN | Homeobox protein Nkx-6.2 (Homeobox protein | NKX6-2 |
| | | NK-6 homolog B) | GTX |
| | | | NKX6B |
| 543 | ASCL5_HUMAN | Achaete-scute homolog 5 (ASH-5) (hASH5) (Class | ASCL5 |
| | | A basic helix-loop-helix protein 47) (bHLHa47) | BHLHA47 |
| 544 | BARX2_HUMAN | Homeobox protein BarH-like 2 | BARX2 |
| 545 | LYL1_HUMAN | Protein lyl-1 (Class A basic helix-loop-helix | LYL1 |
| | | protein 18) (bHLHa18) (Lymphoblastic leukemia- | BHLHA18 |
| | | derived sequence 1) | |
| 546 | E2F6_HUMAN | Transcription factor E2F6 (E2F-6) | E2F6 |
| 547 | LBX1_HUMAN | Transcription factor LBX1 (Ladybird homeobox | LBX1 |
| | | protein homolog 1) | LBX1H |
| 548 | ATF5_HUMAN | Cyclic AMP-dependent transcription factor ATF-5 | ATF5 |
| | | (cAMP-dependent transcription factor ATF-5) | ATFX |
| | | (Activating transcription factor 5) (Transcription | |
| | | factor ATFx) | |
| 549 | HXC12_HUMAN | Homeobox protein Hox-C12 (Homeobox protein | HOXC12 |
| | | Hox-3F) | HOC3F |
| | | | HOX3F |
| 550 | KLF6_HUMAN | Krueppel-like factor 6 (B-cell-derived protein 1) | KLF6 |
| | | (Core promoter element-binding protein) (GC-rich | BCD1 |
| | | sites-binding factor GBF) (Proto-oncogene BCD1) | COPEB |
| | | (Suppressor of tumorigenicity 12 protein) | CPBP |
| | | (Transcription factor Zf9) | ST12 |
| 551 | PDX1_HUMAN | Pancreas/duodenum homeobox protein 1 (PDX-1) | PDX1 |
| | | (Glucose-sensitive factor) (GSF) (Insulin promoter | IPF1 |
| | | factor 1) (IPF-1) (Insulin upstream factor 1) (IUF- | STF1 |
| | | 1) (Islet/duodenum homeobox-1) (IDX-1) | |
| | | (Somatostatin-transactivating factor 1) (STF-1) | |
| 552 | PHX2A_HUMAN | Paired mesoderm homeobox protein 2A (ARIX1 | PHOX2A |
| | | homeodomain protein) (Aristaless homeobox | ARIX |
| | | protein homolog) (Paired-like homeobox 2A) | PMX2A |
| 553 | KLF13_HUMAN | Krueppel-like factor 13 (Basic transcription | KLF13 |
| | | element-binding protein 3) (BTE-binding protein 3) | BTEB3 |
| | | (Novel Sp1-like zinc finger transcription factor 1) | NSLP1 |
| | | (RANTES factor of late activated T-lymphocytes | |
| | | 1) (RFLAT-1) (Transcription factor BTEB3) | |
| | | (Transcription factor NSLP1) | |
| 554 | RHXF2_HUMAN | Rhox homeobox family member 2 (Paired-like | RHOXF2 |
| | | homeobox protein PEPP-2) (Testis homeobox gene 1) | PEPP2 |
| | | | THG1 |
| 555 | RHF2B_HUMAN | Rhox homeobox family member 2B | RHOXF2B |
| 556 | OTX2_HUMAN | Homeobox protein OTX2 (Orthodenticle homolog 2) | OTX2 |
| 557 | HXD8_HUMAN | Homeobox protein Hox-D8 (Homeobox protein | HOXD8 |
| | | Hox-4E) (Homeobox protein Hox-5.4) | HOX4E |
| 558 | VAX2_HUMAN | Ventral anterior homeobox 2 | VAX2 |
| 559 | SIX2_HUMAN | Homeobox protein SIX2 (Sine oculis homeobox | SIX2 |
| | | homolog 2) | |
| 560 | PIT1_HUMAN | Pituitary-specific positive transcription factor 1 | POU1F1 |
| | | (PIT-1) (Growth hormone factor 1) (GHF-1) | GHF1 |
| | | | PIT1 |
| 561 | ZC3H8_HUMAN | Zinc finger CCCH domain-containing protein 8 | ZC3H8 |
| | | | ZC3HDC8 |
| 562 | CNOT8_HUMAN | CCR4-NOT transcription complex subunit 8 (EC | CNOT8 |
| | | 3.1.13.4) (CAF1-like protein) (CALIFp) (CAF2) | CALIF |
| | | (CCR4-associated factor 8) (Caf1b) | POP2 |
| 563 | FOXR1_HUMAN | Forkhead box protein R1 (Forkhead box protein | FOXR1 |
| | | N5) | FOXN5 |
| | | | DLNB13 |
| 564 | SHOX_HUMAN | Short stature homeobox protein (Pseudoautosomal | SHOX |
| | | homeobox-containing osteogenic protein) (Short | PHOG |
| | | stature homeobox-containing protein) | |
| 565 | OZF_HUMAN | Zinc finger protein OZF (Only zinc finger protein) | ZNF146 |
| | | (Zinc finger protein 146) | OZF |
| 566 | SNAI3_HUMAN | Zinc finger protein SNAI3 (Protein snail homolog | SNAI3 |
| | | 3) (Zinc finger protein 293) | ZNF293 |
| 567 | CC033_HUMAN | Protein C3orf33 (Protein AC3-33) | C3orf33 |
| | | | MSTP052 |
| 568 | HLF_HUMAN | Hepatic leukemia factor | HLF |
| 569 | MLX_HUMAN | Max-like protein X (Class D basic helix-loop-helix | MLX |
| | | protein 13) (bHLHd13) (Max-like bHLHZip | BHLHD13 |
| | | protein) (Protein BigMax) (Transcription factor- | TCFL4 |
| | | like protein 4) | |
| 570 | CRX_HUMAN | Cone-rod homeobox protein | CRX |
| | | | CORD2 |
| 571 | PHB2_HUMAN | Prohibitin-2 (B-cell receptor-associated protein | PHB2 |
| | | BAP37) (D-prohibitin) (Repressor of estrogen | BAP |
| | | receptor activity) | REA |
| 572 | EHF_HUMAN | ETS homologous factor (hEHF) (ETS domain- | EHF |
| | | containing transcription factor) (Epithelium- | ESE3 |
| | | specific Ets transcription factor 3) (ESE-3) | ESE3B |
| | | | ESEJ |
| 573 | NKX26_HUMAN | Homeobox protein Nkx-2.6 (Homeobox protein | NKX2-6 |
| | | NK-2 homolog F) | NKX2F |
| 574 | KLF7_HUMAN | Krueppel-like factor 7 (Ubiquitous krueppel-like | KLF7 |
| | | factor) | UKLF |
| 575 | PITX3_HUMAN | Pituitary homeobox 3 (Homeobox protein PITX3) | PITX3 |
| | | (Paired-like homeodomain transcription factor 3) | PTX3 |
| 576 | MSX1_HUMAN | Homeobox protein MSX-1 (Homeobox protein | MSX1 |
| | | Hox-7) (Msh homeobox 1-like protein) | HOX7 |
| 577 | TEF_HUMAN | Thyrotroph embryonic factor | TEF |
| | | | KIAA1655 |
| 578 | GSX2_HUMAN | GS homeobox 2 (Genetic-screened homeobox 2) | GSX2 |
| | | (Homeobox protein GSH-2) | GSH2 |
| 579 | HXC11_HUMAN | Homeobox protein Hox-C11 (Homeobox protein | HOXC11 |
| | | Hox-3H) | HOX3H |
| 580 | MEOX2_HUMAN | Homeobox protein MOX-2 (Growth arrest-specific | MEOX2 |
| | | homeobox) (Mesenchyme homeobox 2) | GAX |
| | | | MOX2 |
| 581 | SCND2_HUMAN | Putative SCAN domain-containing protein | SCAND2P |
| | | SCAND2P (SCAN domain-containing protein 2 | SCAND2 |
| | | pseudogene) | |
| 582 | NKX12_HUMAN | NK1 transcription factor-related protein 2 | NKX1-2 |
| | | (Homeobox protein SAX-1) (NKX-1.1) | C10orf121 |
| | | | NKX1.1 |
| 583 | ZFP42_HUMAN | Zinc finger protein 42 homolog (Zfp-42) (Reduced | ZFP42 |
| | | expression protein 1) (REX-1) (hREX-1) (Zinc | REX1 |
| | | finger protein 754) | ZNF754 |
| 584 | FOXR2_HUMAN | Forkhead box protein R2 (Forkhead box protein | FOXR2 |
| | | N6) | FOXN6 |
| 585 | PLPP3_HUMAN | Phospholipid phosphatase 3 (EC 3.1.3.4) (Lipid | PLPP3 |
| | | phosphate phosphohydrolase 3) (PAP2-beta) | LPP3 |
| | | (Phosphatidate phosphohydrolase type 2b) | PPAP2B |
| | | (Phosphatidic acid phosphatase 2b) (PAP-2b) | |
| | | (PAP2b) (Vascular endothelial growth factor and | |
| | | type I collagen-inducible protein) (VCIP) | |
| 586 | PURB_HUMAN | Transcriptional activator protein Pur-beta (Purine- | PURB |
| | | rich element-binding protein B) | |
| 587 | HXA11_HUMAN | Homeobox protein Hox-A11 (Homeobox protein | HOXA11 |
| | | Hox-1I) | HOX1I |
| 588 | DDRGK_HUMAN | DDRGK domain-containing protein 1 (Dashurin) | DDRGK1 |
| | | (UFM1-binding and PCI domain-containing protein | C20orf116 |
| | | 1) | UFBP1 |
| 589 | PHX2B_HUMAN | Paired mesoderm homeobox protein 2B | PHOX2B |
| | | (Neuroblastoma Phox) (NBPhox) (PHOX2B | PMX2B |
| | | homeodomain protein) (Paired-like homeobox 2B) | |
| 590 | PITX1_HUMAN | Pituitary homeobox 1 (Hindlimb-expressed | PITX1 |
| | | homeobox protein backfoot) (Homeobox protein | BFT |
| | | PΓΓχ1) (Paired-like homeodomain transcription | PTX1 |
| | | factor 1) | |
| 591 | ARGFX_HUMAN | Arginine-fifty homeobox | ARGFX |
| 592 | SOX12_HUMAN | Transcription factor SOX-12 (Protein SOX-22) | SOX12 |
| | | | SOX22 |
| 593 | SFRP5_HUMAN | Secreted frizzled-related protein 5 (sFRP-5) | SFRP5 |
| | | (Frizzled-related protein 1b) (FRP-1b) (Secreted | FRP1B |
| | | apoptosis-related protein 3) (SARP-3) | SARP3 |
| 594 | FOXI2_HUMAN | Forkhead box protein 12 | FOXI2 |
| 595 | ANKR1_HUMAN | Ankyrin repeat domain-containing protein 1 | ANKRD1 |
| | | (Cardiac ankyrin repeat protein) (Cytokine- | C193 |
| | | inducible gene C-193 protein) (Cytokine-inducible | CARP |
| | | nuclear protein) | HA1A2 |
| 596 | FOXE3_HUMAN | Forkhead box protein E3 (Forkhead-related protein | FOXE3 |
| | | FKHL12) (Forkhead-related transcription factor 8) | FKHL12 |
| | | (FREAC-8) | FREAC8 |
| 597 | HXA4_HUMAN | Homeobox protein Hox-A4 (Homeobox protein | HOXA4 |
| | | Hox-1.4) (Homeobox protein Hox-1D) | HOX1D |
| 598 | MYOD1_HUMAN | Myoblast determination protein 1 (Class C basic | MYOD1 |
| | | helix-loop-helix protein 1) (bHLHc1) (Myogenic | BHLHC1 |
| | | factor 3) (Myf-3) | MYF3 |
| | | | MYOD |
| 599 | ATOH8_HUMAN | Protein atonal homolog 8 (Class A basic helix- | ATOH8 |
| | | loop-helix protein 21) (bHLHa21) (Helix-loop- | ATH6 |
| | | helix protein hATH-6) (hATH6) | BHLHA21 |
| 600 | CXXC5_HUMAN | CXXC-type zinc finger protein 5 (CF5) (Putative | CXXC5 |
| | | MAPK-activating protein PM08) (Putative NF- | HSPC195 |
| | | kappa-B-activating protein 102) (Retinoid- | TCCCIA00297 |
| | | inducible nuclear factor) (RINF) | |
| 601 | PURA_HUMAN | Transcriptional activator protein Pur-alpha (Purine- | PURA |
| | | rich single-stranded DNA-binding protein alpha) | PUR1 |
| 602 | KLF14_HUMAN | Krueppel-like factor 14 (Basic transcription | KLF14 |
| | | element-binding protein 5) (BTE-binding protein 5) | BTEB5 |
| | | (Transcription factor BTEB5) | |
| 603 | OLIG2_HUMAN | Oligodendrocyte transcription factor 2 (Oligo2) | OLIG2 |
| | | (Class B basic helix-loop-helix protein 1) | BHLHB1 |
| | | (bHLHb1) (Class E basic helix-loop-helix protein | BHLHE19 |
| | | 19) (bHLHe19) (Protein kinase C-binding protein | PRKCBP2 |
| | | 2) (Protein kinase C-binding protein RACK17) | RACK17 |
| 604 | MAFB_HUMAN | Transcription factor MafB (Maf-B) (V-maf | MAFB |
| | | musculoaponeurotic fibrosarcoma oncogene | KRML |
| | | homolog B) | |
| 605 | FOXB1_HUMAN | Forkhead box protein B1 (Transcription factor | FOXB1 |
| | | FKH-5) | FKH5 |
| 606 | IRF1_HUMAN | Interferon regulatory factor 1 (IRF-1) | IRF1 |
| 607 | ALX1_HUMAN | ALX homeobox protein 1 (Cartilage homeoprotein | ALX1 |
| | | 1) (CART-1) | CART1 |
| 608 | FOSL2_HUMAN | Fos-related antigen 2 (FRA-2) | FOSL2 |
| | | | FRA2 |
| 609 | ZNF73_HUMAN | Zinc finger protein 73 (Zinc finger protein 186) | ZNF73 |
| | | (hZNF2) | ZNF186 |
| 610 | ZN444_HUMAN | Zinc finger protein 444 (Endothelial zinc finger | ZNF444 |
| | | protein 2) (EZF-2) (Zinc finger and SCAN domain- | EZF2 |
| | | containing protein 17) | ZSCAN17 |
| 611 | HEYL_HUMAN | Hairy/enhancer-of-split related with YRPW motif- | HEYL |
| | | like protein (hHeyL) (Class B basic helix-loop- | BHLHB33 |
| | | helix protein 33) (bHLHb33) (Hairy-related | HRT3 |
| | | transcription factor 3) (HRT-3) (hHRT3) | |
| 612 | DLX2_HUMAN | Homeobox protein DLX-2 | DLX2 |
| 613 | HXD1_HUMAN | Homeobox protein Hox-D1 (Homeobox protein | HOXD1 |
| | | Hox-GG) | HOX4 |
| | | | HOX4G |
| 614 | PTF1A_HUMAN | Pancreas transcription factor 1 subunit alpha (Class | PTF1A |
| | | A basic helix-loop-helix protein 29) (bHLHa29) | BHLHA29 |
| | | (Pancreas-specific transcription factor 1a) (bHLH | PTF1P48 |
| | | transcription factor p48) (p48 DNA-binding | |
| | | subunit of transcription factor PTF1) (PTF1-p48) | |
| 615 | PO5F2_HUMAN | POU domain, class 5, transcription factor 2 (Sperm | POU5F2 |
| | | 1 POU domain transcription factor) (SPRM-1) | SPRM1 |
| 616 | SOLH1_HUMAN | Spermatogenesis- and oogenesis-specific basic | SOHLH1 |
| | | helix-loop-helix-containing protein 1 | C9orf157 |
| | | | NOHLH |
| | | | TEB2 |
| 617 | SCML1_HUMAN | Sex comb on midleg-like protein 1 | SCML1 |
| 618 | FOXS1_HUMAN | Forkhead box protein S1 (Forkhead-like 18 protein) | FOXS1 |
| | | (Forkhead-related transcription factor 10) (FREAC- | FKHL18 |
| | | 10) | FREAC10 |
| 619 | HXC13_HUMAN | Homeobox protein Hox-Cl3 (Homeobox protein | HOXC13 |
| | | Hox-3G) | HOX3G |
| 620 | ZN660_HUMAN | Zinc finger protein 660 | ZNF660 |
| 621 | SIX3_HUMAN | Homeobox protein SIX3 (Sine oculis homeobox | SIX3 |
| | | homolog 3) | |
| 622 | HSFX3_HUMAN | Heat shock transcription factor, X-linked member 3 | HSFX3 |
| 623 | HSFX4_HUMAN | Heat shock transcription factor, X-linked member 4 | HSFX4 |
| 624 | HME2_HUMAN | Homeobox protein engrailed-2 (Homeobox protein | EN2 |
| | | en-2) (Hu-En-2) | |
| 625 | SNPC2_HUMAN | snRNA-activating protein complex subunit 2 | SNAPC2 |
| | | (SNAPc subunit 2) (Proximal sequence element- | SNAP45 |
| | | binding transcription factor subunit delta) (PSE- | |
| | | binding factor subunit delta) (PTF subunit delta) | |
| | | (Small nuclear RNA-activating complex | |
| | | polypeptide 2) (snRNA-activating protein complex | |
| | | 45 kDa subunit) (SNAPc 45 kDa subunit) | |
| 626 | VAX1_HUMAN | Ventral anterior homeobox 1 | VAX1 |
| 627 | HXA1_HUMAN | Homeobox protein Hox-A1 (Homeobox protein | HOXA1 |
| | | Hox-1F) | HOX1F |
| 628 | ZN396_HUMAN | Zinc finger protein 396 (Zinc finger and SCAN | ZNF396 |
| | | domain-containing protein 14) | ZSCAN14 |
| 629 | HEY2_HUMAN | Hairy/enhancer-of-split related with YRPW motif | HEY2 |
| | | protein 2 (Cardiovascular helix-loop-helix factor 1) | BHLHB32 |
| | | (hCHF1) (Class B basic helix-loop-helix protein | CHF1 |
| | | 32) (bHLHb32) (HES-related repressor protein 2) | GRL |
| | | (Hairy and enhancer of split-related protein 2) | HERP |
| | | (HESR-2) (Hairy-related transcription factor 2) | HERP1 |
| | | (HRT-2) (hHRT2) (Protein gridlock homolog) | HRT2 |
| 630 | NDF6_HUMAN | Neurogenic differentiation factor 6 (NeuroD6) | NEUROD6 |
| | | (Class A basic helix-loop-helix protein 2) | ATOH2 |
| | | (bHLHa2) (Protein atonal homolog 2) | BHLHA2 |
| | | | My051 |
| 631 | HXD11_HUMAN | Homeobox protein Hox-D11 (Homeobox protein | HOXD11 |
| | | Hox-4F) | HOX4F |
| 632 | PO4F3_HUMAN | POU domain, class 4, transcription factor 3 (Brain- | POU4F3 |
| | | specific homeobox/POU domain protein 3C) | BRN3C |
| | | (Brain-3C) (Brn-3C) | |
| 633 | FOSB_HUMAN | Protein fosB (G0/G1 switch regulatory protein 3) | FOSB |
| | | | G0S3 |
| 634 | TFAP4_HUMAN | Transcription factor AP-4 (Activating enhancer- | TFAP4 |
| | | binding protein 4) (Class C basic helix-loop-helix | BHLHC41 |
| | | protein 41) (bHLHc41) | |
| 635 | HXD10_HUMAN | Homeobox protein Hox-D10 (Homeobox protein | HOXD10 |
| | | Hox-4D) (Homeobox protein Hox-4E) | HOX4D |
| | | | HOX4E |
| 636 | PAX9_HUMAN | Paired box protein Pax-9 | PAX9 |
| 637 | ETV7_HUMAN | Transcription factor ETV7 (ETS translocation | ETV7 |
| | | variant 7) (ETS-related protein Tel2) (Tel-related | TEL2 |
| | | Ets factor) (Transcription factor Tel-2) | TELB |
| | | | TREF |
| 638 | DMRTB_HUMAN | Doublesex- and mab-3-related transcription factor | DMRTB1 |
| | | B1 | |
| 639 | ETV2_HUMAN | ETS translocation variant 2 (Ets-related protein 71) | ETV2 |
| | | | ER71 |
| | | | ETSRP71 |
| 640 | HXC10_HUMAN | Homeobox protein Hox-C10 (Homeobox protein | HOXC10 |
| | | Hox-3I) | HOX3I |
| 641 | ALX3_HUMAN | Homeobox protein aristaless-like 3 (Proline-rich | ALX3 |
| | | transcription factor ALX3) | |
| 642 | DBX1_HUMAN | Homeobox protein DBX1 (Developing brain | DBX1 |
| | | homeobox protein 1) | |
| 643 | HXD13_HUMAN | Homeobox protein Hox-D13 (Homeobox protein | HOXD13 |
| | | Hox-4I) | HOX4I |
| 644 | PCGF2_HUMAN | Polycomb group RING finger protein 2 (DNA- | PCGF2 |
| | | binding protein Mel-18) (RING finger protein 110) | MEL18 |
| | | (Zinc finger protein 144) | RNF110 |
| | | | ZNF144 |
| 645 | FANK1_HUMAN | Fibronectin type 3 and ankyrin repeat domains | FANK1 |
| | | protein 1 | HSD13 |
| | | | UNQ6504/ |
| | | | PRO21382 |
| 646 | FOXL1_HUMAN | Forkhead box protein L1 (Forkhead-related protein | FOXL1 |
| | | FKHL11) (Forkhead-related transcription factor 7) | FKHL11 |
| | | (FREAC-7) | FREAC7 |
| 647 | KLF3_HUMAN | Krueppel-like factor 3 (Basic krueppel-like factor) | KLF3 |
| | | (CACCC-box-binding protein BKLF) (TEF-2) | BKLF |
| 648 | TCF19_HUMAN | Transcription factor 19 (TCF-19) (Transcription | TCF19 |
| | | factor SC1) | SC1 |
| 649 | SFRP4_HUMAN | Secreted frizzled-related protein 4 (sFRP-4) | SFRP4 |
| | | (Frizzled protein, human endometrium) (FrpHE) | FRPHE |
| 650 | USF2_HUMAN | Upstream stimulatory factor 2 (Class B basic helix- | USF2 |
| | | loop-helix protein 12) (bHLHb12) (FOS-interacting | BHLHB12 |
| | | protein) (FIP) (Major late transcription factor 2) | |
| | | (Upstream transcription factor 2) | |
| 651 | HM20A_HUMAN | High mobility group protein 20A (HMG box- | HMG20A |
| | | containing protein 20A) (HMG domain-containing | HMGX1 |
| | | protein 1) (HMG domain-containing protein | HMGXB1 |
| | | HMGX1) | |
| 652 | TFEC_HUMAN | Transcription factor EC (TFE-C) (Class E basic | TFEC |
| | | helix-loop-helix protein 34) (bHLHe34) | BHLHE34 |
| | | (Transcription factor EC-like) (hTFEC-L) | TCFEC |
| | | | TFECL |
| 653 | JUNB_HUMAN | Transcription factor jun-B | JUNB |
| 654 | GBX2_HUMAN | Homeobox protein GBX-2 (Gastrulation and brain- | GBX2 |
| | | specific homeobox protein 2) | |
| 655 | SCRT1_HUMAN | Transcriptional repressor scratch 1 (Scratch | SCRT1 |
| | | homolog 1 zinc finger protein) (SCRT) (Scratch 1) | |
| | | (hScrt) | |
| 656 | ISL1_HUMAN | Insulin gene enhancer protein ISL-1 (Islet-1) | ISL1 |
| 657 | IRF2_HUMAN | Interferon regulatory factor 2 (IRF-2) | IRF2 |
| 658 | ZN367_HUMAN | Zinc finger protein 367 (C2H2 zinc finger protein | ZNF367 |
| | | ZFF29) | ZFF29 |
| 659 | HXD9_HUMAN | Homeobox protein Hox-D9 (Homeobox protein | HOXD9 |
| | | Hox-4C) (Homeobox protein Hox-5.2) | HOX4C |
| 660 | WNT3A_HUMAN | Protein Wnt-3a | WNT3A |
| 661 | ZHANG_HUMAN | CREB/ATF bZIP transcription factor (Host cell | CREBZF |
| | | factor-binding transcription factor Zhangfei) (HCF- | ZF |
| | | binding transcription factor Zhangfei) | |
| 662 | NKX24_HUMAN | Homeobox protein Nkx-2.4 (Homeobox protein | NKX2-4 |
| | | NK-2 homolog D) | NKX2D |
| 663 | ATOH1_HUMAN | Protein atonal homolog 1 (Class A basic helix- | ATOH1 |
| | | loop-helix protein 14) (bHLHa14) (Helix-loop- | ATH1 |
| | | helix protein hATH-1) (hATH1) | BHLHA14 |
| 664 | KLF2_HUMAN | Krueppel-like factor 2 (Lung krueppel-like factor) | KLF2 |
| | | | LKLF |
| 665 | ZN781_HUMAN | Zinc finger protein 781 | ZNF781 |
| 666 | HXB2_HUMAN | Homeobox protein Hox-B2 (Homeobox protein | HOXB2 |
| | | Hox-2.8) (Homeobox protein Hox-2H) (K8) | HOX2H |
| 667 | LHX8_HUMAN | LIM/homeobox protein Lhx8 (LIM homeobox | LHX8 |
| | | protein 8) | |
| 668 | NDF1_HUMAN | Neurogenic differentiation factor 1 (NeuroD) | NEUROD1 |
| | | (NeuroD1) (Class A basic helix-loop-helix protein | BHLHA3 |
| | | 3) (bHLHa3) | NEUROD |
| 669 | HMX3_HUMAN | Homeobox protein HMX3 (Homeobox protein H6 | HMX3 |
| | | family member 3) (Homeobox protein Nkx-5.1) | NKX-5.1 |
| | | | NKX5-1 |
| 670 | CEBPA_HUMAN | CCAAT/enhancer-binding protein alpha (C/EBP | CEBPA |
| | | alpha) | CEBP |
| 671 | MYCP1_HUMAN | Putative myc-like protein MYCLP1 (Protein L- | MYCLP1 |
| | | Myc-2) (V-myc myelocytomatosis viral oncogene | MYCL1P1 |
| | | homolog pseudogene 1) | MYCL2 |
| 672 | ZN391_HUMAN | Zinc finger protein 391 | ZNF391 |
| 673 | ISL2_HUMAN | Insulin gene enhancer protein ISL-2 (Islet-2) | ISL2 |
| 674 | KLF8_HUMAN | Krueppel-like factor 8 (Basic krueppel-like factor | KLF8 |
| | | 3) (Zinc finger protein 741) | BKLF3 |
| | | | ZNF741 |
| 675 | P5F1B_HUMAN | Putative POU domain, class 5, transcription factor | POU5F1B |
| | | 1B (Oct4-pg1) (Octamer-binding protein 3-like) | OCT4PG1 |
| | | (Octamer-binding transcription factor 3-like) | OTF3C |
| | | | OTF3P1 |
| | | | POU5F1P1 |
| | | | POU5FLC20 |
| | | | POU5FLC8 |
| 676 | ANKR2_HUMAN | Ankyrin repeat domain-containing protein 2 | ANKRD2 |
| | | (Skeletal muscle ankyrin repeat protein) (hArpp) | ARPP |
| 677 | PO5F1_HUMAN | POU domain, class 5, transcription factor 1 | POU5F1 |
| | | (Octamer-binding protein 3) (Oct-3) (Octamer- | OCT3 |
| | | binding protein 4) (Oct-4) (Octamer-binding | OCT4 |
| | | transcription factor 3) (OTF-3) | OTF3 |
| 678 | WNT2_HUMAN | Protein Wnt-2 (Int-1-like protein 1) (Int-1-related | WNT2 |
| | | protein) (IRP) | INT1L1 |
| | | | IRP |
| 679 | CREM_HUMAN | cAMP-responsive element modulator (Inducible | CREM |
| | | cAMP early repressor) (ICER) | |
| 680 | ETV3L_HUMAN | ETS translocation variant 3-like protein | ETV3L |
| 681 | PO3F4_HUMAN | POU domain, class 3, transcription factor 4 (Brain- | POU3F4 |
| | | specific homeobox/POU domain protein 4) (Brain- | BRN4 |
| | | 4) (Brn-4) (Octamer-binding protein 9) (Oct-9) | OTF9 |
| | | (Octamer-binding transcription factor 9) (OTF-9) | |
| 682 | LHX6_HUMAN | LIM/homeobox protein Lhx6 (LIM homeobox | LHX6 |
| | | protein 6) (LIM/homeobox protein Lhx6.1) | LHX6.1 |
| 683 | NKX23_HUMAN | Homeobox protein Nkx-2.3 (Homeobox protein | NKX2-3 |
| | | NK-2 homolog C) | NKX23 |
| | | | NKX2C |
| 684 | MYCL_HUMAN | Protein L-Myc (Class E basic helix-loop-helix | MYCL |
| | | protein 38) (bHLHe38) (Protein L-Myc-1) (V-myc | BHLHE38 |
| | | myelocytomatosis viral oncogene homolog) | LMYC |
| | | | MYCL1 |
| 685 | FOXH1_HUMAN | Forkhead box protein H1 (Forkhead activin signal | FOXH1 |
| | | transducer 1) (Fast-1) (hFAST-1) (Forkhead activin | FAST1 |
| | | signal transducer 2) (Fast-2) | FAST2 |
| 686 | VSX1_HUMAN | Visual system homeobox 1 (Homeodomain protein | VSX1 |
| | | RINX) (Retinal inner nuclear layer homeobox | RINX |
| | | protein) (Transcription factor VSX1) | |
| 687 | DMRTD_HUMAN | Doublesex- and mab-3-related transcription factor | DMRTC2 |
| | | C2 | |
| 688 | NKX61_HUMAN | Homeobox protein Nkx-6.1 (Homeobox protein | NKX6-1 |
| | | NK-6 homolog A) | NKX6A |
| 689 | SNPC1_HUMAN | snRNA-activating protein complex subunit 1 | SNAPC1 |
| | | (SNAPc subunit 1) (Proximal sequence element- | SNAP43 |
| | | binding transcription factor subunit gamma) (PSE- | |
| | | binding factor subunit gamma) (PTF subunit | |
| | | gamma) (Small nuclear RNA-activating complex | |
| | | polypeptide 1) (snRNA-activating protein complex | |
| | | 43 kDa subunit) (SNAPc 43 kDa subunit) | |
| 690 | WNT1_HUMAN | Proto-oncogene Wnt-1 (Proto-oncogene Int-1 | WNT1 |
| | | homolog) | INTI |
| 691 | NKX21_HUMAN | Homeobox protein Nkx-2.1 (Homeobox protein | NKX2-1 |
| | | NK-2 homolog A) (Thyroid nuclear factor 1) | NKX2A |
| | | (Thyroid transcription factor 1) (TTF-1) (Thyroid- | TITF1 |
| | | specific enhancer-binding protein) (T/EBP) | TTF1 |
| 692 | TYY2_HUMAN | Transcription factor YY2 (Yin and yang 2) (YY-2) | YY2 |
| | | (Zinc finger protein 631) | ZNF631 |
| 693 | YBOX3_HUMAN | Y-box-binding protein 3 (Cold shock domain- | YBX3 |
| | | containing protein A) (DNA-binding protein A) | CSDA |
| | | (Single-strand DNA-binding protein NF-GMB) | DBPA |
| 694 | FOXE1_HUMAN | Forkhead box protein E1 (Forkhead box protein | FOXE1 |
| | | E2) (Forkhead-related protein FKHL15) (HFKH4) | FKHL15 |
| | | (HNF-3/fork head-like protein 5) (HFKL5) | FOXE2 |
| | | (Thyroid transcription factor 2) (TTF-2) | TITF2 |
| | | | TTF2 |
| 695 | MAF_HUMAN | Transcription factor Maf (Proto-oncogene c-Maf) | MAF |
| | | (V-maf musculoaponeurotic fibrosarcoma | |
| | | oncogene homolog) | |
| 696 | PBX4_HUMAN | Pre-B-cell leukemia transcription factor 4 | PBX4 |
| | | (Homeobox protein PBX4) | |
| 697 | ZN696_HUMAN | Zinc finger protein 696 | ZNF696 |
| 698 | TBPL2_HUMAN | TATA box-binding protein-like protein 2 (TBP- | TBPL2 |
| | | like protein 2) (TATA box-binding protein-related | TBP2 |
| | | factor 3) (TBP-related factor 3) | TRF3 |
| 699 | FOXL2_HUMAN | Forkhead box protein L2 | FOXL2 |
| 700 | HXA2_HUMAN | Homeobox protein Hox-A2 (Homeobox protein | HOXA2 |
| | | Hox-1K) | HOX1K |
| 701 | SP6_HUMAN | Transcription factor Sp6 (Krueppel-like factor 14) | SP6 |
| | | | KLF14 |
| 702 | GLI4_HUMAN | Zinc finger protein GLI4 (Krueppel-related zinc | GLI4 |
| | | finger protein 4) (Protein HKR4) | HKR4 |
| 703 | BTBD8_HUMAN | BTB/POZ domain-containing protein 8 | BTBD8 |
| 704 | FOXI1_HUMAN | Forkhead box protein I1 (Forkhead-related protein | FOXI1 |
| | | FKHL10) (Forkhead-related transcription factor 6) | FKHL10 |
| | | (FREAC-6) (Hepatocyte nuclear factor 3 forkhead | FREAC6 |
| | | homolog 3) (HFH-3) (HNF-3/fork-head homolog 3) | |
| 705 | FOXF1_HUMAN | Forkhead box protein F1 (Forkhead-related | FOXF1 |
| | | activator 1) (FREAC-1) (Forkhead-related protein | FKHL5 |
| | | FKHL5) (Forkhead-related transcription factor 1) | FREAC1 |
| 706 | ZN883_HUMAN | Zinc finger protein 883 | ZNF883 |
| 707 | WNT5A_HUMAN | Protein Wnt-5a | WNT5A |
| 708 | ABRA_HUMAN | Actin-binding Rho-activating protein (Striated | ABRA |
| | | muscle activator of Rho-dependent signaling) | |
| | | (STARS) | |
| 709 | BHE22_HUMAN | Class E basic helix-loop-helix protein 22 | BHLHE22 |
| | | (bHLHe22) (Class B basic helix-loop-helix protein | BHLHB5 |
| | | 5) (bHLHb5) (Trinucleotide repeat-containing gene | TNRC20 |
| | | 20 protein) | |
| 710 | DMBX1_HUMAN | Diencephalon/mesencephalon homeobox protein 1 | DMBX1 |
| | | (Orthodenticle homolog 3) (Paired-like homeobox | MBX |
| | | protein DMBX1) | OTX3 |
| | | | PAXB |
| 711 | LMX1A_HUMAN | LIM homeobox transcription factor 1-alpha | LMX1A |
| | | (LIM/homeobox protein 1.1) (LMX-1.1) | |
| | | (LIM/homeobox protein LMX1A) | |
| 712 | NDF2_HUMAN | Neurogenic differentiation factor 2 (NeuroD2) | NEUROD2 |
| | | (Class A basic helix-loop-helix protein 1) | BHLHA1 |
| | | (bHLHa1) (NeuroD-related factor) (NDRF) | NDRF |
| 713 | SAV1_HUMAN | Protein Salvador homolog 1 (45 kDa WW domain | SAV1 |
| | | protein) (hWW45) | WW45 |
| 714 | TCF7_HUMAN | Transcription factor 7 (TCF-7) (T-cell-specific | TCF7 |
| | | transcription factor 1) (T-cell factor 1) (TCF-1) | TCF1 |
| 715 | SOX18_HUMAN | Transcription factor SOX-18 | SOX18 |
| 716 | TBX10_HUMAN | T-box transcription factor TBX10 (T-box protein | TBX10 |
| | | 10) | TBX7 |
| 717 | EGR3_HUMAN | Early growth response protein 3 (EGR-3) (Zinc | EGR3 |
| | | finger protein pilot) | PILOT |
| 718 | S2A4R_HUMAN | SLC2A4 regulator (GLUT4 enhancer factor) (GEF) | SLC2A4RG |
| | | (Huntington disease gene regulatory region-binding | HDBP1 |
| | | protein 1) (HDBP-1) | |
| 719 | SOX7_HUMAN | Transcription factor SOX-7 | SOX7 |
| 720 | KLF17_HUMAN | Krueppel-like factor 17 (Zinc finger protein 393) | KLF17 |
| | | | ZNF393 |
| 721 | WN10B_HUMAN | Protein Wnt-10b (Protein Wnt-12) | WNT10B |
| | | | WNT12 |
| 722 | ZSC23_HUMAN | Zinc finger and SCAN domain-containing protein | ZSCAN23 |
| | | 23 (Zinc finger protein 390) (Zinc finger protein | ZNF390 |
| | | 453) | ZNF453 |
| 723 | ZN121_HUMAN | Zinc finger protein 121 (Zinc finger protein 20) | ZNF121 |
| | | | ZNF20 |
| 724 | SOX1_HUMAN | Transcription factor SOX-1 | SOX1 |
| 725 | IRF9_HUMAN | Interferon regulatory factor 9 (IRF-9) (IFN-alpha- | IRF9 |
| | | responsive transcription factor subunit) (ISGF3 p48 | ISGF3G |
| | | subunit) (Interferon-stimulated gene factor 3 | |
| | | gamma) (ISGF-3 gamma) (Transcriptional | |
| | | regulator ISGF3 subunit gamma) | |
| 726 | ZSC9_HUMAN | Zinc finger and SCAN domain-containing protein 9 | ZSCAN9 |
| | | (Cell proliferation-inducing gene 12 protein) | ZNF193 |
| | | (PRD51) (Zinc finger protein 193) | PIG12 |
| 727 | CREB3_HUMAN | Cyclic AMP-responsive element-binding protein 3 | CREB3 |
| | | (CREB-3) (cAMP-responsive element-binding | LZIP |
| | | protein 3) (Leucine zipper protein) (Luman) | |
| | | (Transcription factor LZIP-alpha) [Cleaved into: | |
| | | Processed cyclic AMP-responsive element-binding | |
| | | protein 3 (N-terminal Luman) (Transcriptionally | |
| | | active form)] | |
| 728 | CR3L4_HUMAN | Cyclic AMP-responsive element-binding protein 3- | CREB3L4 |
| | | like protein 4 (cAMP-responsive element-binding | AIBZIP |
| | | protein 3-like protein 4) (Androgen-induced basic | CREB4 |
| | | leucine zipper protein) (AlbZIP) (Attaching to | JAL |
| | | CRE-like 1) (ATCE1) (Cyclic AMP-responsive | |
| | | element-binding protein 4) (CREB-4) (cAMP- | |
| | | responsive element-binding protein 4) (Transcript | |
| | | induced in spermiogenesis protein 40) (Tisp40) | |
| | | (hJAL) [Cleaved into: Processed cyclic AMP- | |
| | | responsive element-binding protein 3-like protein 4] | |
| 729 | GABP1_HUMAN | GA-binding protein subunit beta-1 (GABP subunit | GABPB1 |
| | | beta-1) (GABPB-1) (GABP subunit beta-2) | E4TF1B |
| | | (GABPB-2) (Nuclear respiratory factor 2) | GABPB |
| | | (Transcription factor E4TF1-47) (Transcription | GABPB2 |
| | | factor E4TF1-53) | |
| 730 | T22D4_HUMAN | TSC22 domain family protein 4 (TSC22-related- | TSC22D4 |
| | | inducible leucine zipper protein 2) (Tsc-22-like | THG1 |
| | | protein THG-1) | TILZ2 |
| 731 | LHX3_HUMAN | LIM/homeobox protein Lhx3 (LIM homeobox | LHX3 |
| | | protein 3) | |
| 732 | MESP2_HUMAN | Mesoderm posterior protein 2 (Class C basic helix- | MESP2 |
| | | loop-helix protein 6) (bHLHc6) | BHLHC6 |
| | | | SCDO2 |
| 733 | GATA5_HUMAN | Transcription factor GATA-5 (GATA-binding | GATA5 |
| | | factor 5) | |
| 734 | SP5_HUMAN | Transcription factor Sp5 | SP5 |
| 735 | LEF1_HUMAN | Lymphoid enhancer-binding factor 1 (LEF-1) (T | LEF1 |
| | | cell-specific transcription factor 1-alpha) (TCF1- | |
| | | alpha) | |
| 736 | MYPOP_HUMAN | Myb-related transcription factor, partner of profilin | MYPOP |
| | | (Myb-related protein p42POP) (Partner of profilin) | P42POP |
| 737 | GO45_HUMAN | Golgin-45 (Basic leucine zipper nuclear factor 1) | BLZF1 |
| | | (JEM-1) (p45 basic leucine-zipper nuclear factor) | JEM1 |
| 738 | ZN514_HUMAN | Zinc finger protein 514 | ZNF514 |
| 739 | HSFY1_HUMAN | Heat shock transcription factor, Y-linked (Heat | HSFY1 |
| | | shock transcription factor 2-like protein) (HSF2- | HSF2L |
| | | like) | HSFY; |
| | | | HSFY2 |
| | | | HSF2L |
| | | | HSFY |
| 740 | MNX1_HUMAN | Motor neuron and pancreas homeobox protein 1 | MNX1 |
| | | (Homeobox protein HB9) | HLXB9 |
| 741 | KLF12_HUMAN | Krueppel-like factor 12 (Transcriptional repressor | KLF12 |
| | | AP-2rep) | AP2REP |
| | | | HSPC122 |
| 742 | LMX1B_HUMAN | LIM homeobox transcription factor 1-beta | LMX1B |
| | | (LIM/homeobox protein 1.2) (LMX-1.2) | |
| | | (LIM/homeobox protein LMX1B) | |
| 743 | ZN322_HUMAN | Zinc finger protein 322 (Zinc finger protein 322A) | ZNF322 |
| | | (Zinc finger protein 388) (Zinc finger protein 489) | ZNF322A |
| | | | ZNF388 |
| | | | ZNF489 |
| 744 | FOXQ1_HUMAN | Forkhead box protein Q1 (HNF-3/forkhead-like | FOXQ1 |
| | | protein 1) (HFH-1) (Hepatocyte nuclear factor 3 | HFH1 |
| | | forkhead homolog 1) | |
| 745 | NR2F6_HUMAN | Nuclear receptor subfamily 2 group F member 6 | NR2F6 |
| | | (V-erbA-related protein 2) (EAR-2) | EAR2 |
| | | | ERBAL2 |
| 746 | TFDP3_HUMAN | Transcription factor Dp family member 3 | TFDP3 |
| | | (Cancer/testis antigen 30) (CT30) (Hepatocellular | DP4 |
| | | carcinoma-associated antigen 661) | HCA661 |
| 747 | GLMP_HUMAN | Glycosylated lysosomal membrane protein | GLMP |
| | | (Lysosomal protein NCU-G1) | C1orf85 |
| | | | PSEC0030 |
| | | | UNQ2553/ |
| | | | PRO6182 |
| 748 | LHX1_HUMAN | LIM/homeobox protein Lhx1 (LIM homeobox | LHX1 |
| | | protein 1) (Homeobox protein Lim-1) (hLim-1) | LIM-1 |
| | | | LIM1 |
| 749 | LHX2_HUMAN | LIM/homeobox protein Lhx2 (Homeobox protein | LHX2 |
| | | LH-2) (LIM homeobox protein 2) | LH2 |
| 750 | ZSC31_HUMAN | Zinc finger and SCAN domain-containing protein | ZSCAN31 |
| | | 31 (Zinc finger protein 323) | ZNF310P |
| | | | ZNF323 |
| 751 | ELK3_HUMAN | ETS domain-containing protein Elk-3 (ETS-related | ELK3 |
| | | protein ERP) (ETS-related protein NET) (Serum | NET |
| | | response factor accessory protein 2) (SAP-2) (SRF | SAP2 |
| | | accessory protein 2) | |
| 752 | EVX1_HUMAN | Homeobox even-skipped homolog protein 1 (EVX- | EVX1 |
| | | 1) | |
| 753 | ZFP1_HUMAN | Zinc finger protein 1 homolog (Zfp-1) (Zinc finger | ZFP1 |
| | | protein 475) | ZNF475 |
| 754 | FX4L1_HUMAN | Forkhead box protein D4-like 1 (FOXD4-like 1) | FOXD4L1 |
| 755 | ZSCA1_HUMAN | Zinc finger and SCAN domain-containing protein 1 | ZSCAN1 |
| 756 | PO4F2_HUMAN | POU domain, class 4, transcription factor 2 (Brain- | POU4F2 |
| | | specific homeobox/POU domain protein 3B) | BRN3B |
| | | (Brain-3B) (Brn-3B) | |
| 757 | HXA10_HUMAN | Homeobox protein Hox-A10 (Homeobox protein | HOXA10 |
| | | Hox-1.8) (Homeobox protein Hox-1H) (PL) | HOX1H |
| 758 | SIGIR_HUMAN | Single Ig IL-1-related receptor (Single Ig IL-1R- | SIGIRR |
| | | related molecule) (Single immunoglobulin domain- | UNQ301/ |
| | | containing IL1R-related protein) (Toll/interleukin-1 | PRO342 |
| | | receptor 8) (TIR8) | |
| 759 | NKX11_HUMAN | NK1 transcription factor-related protein 1 | NKX1-1 |
| | | (Homeobox protein 153) (HPX-153) (Homeobox | HPX153 |
| | | protein SAX-2) (NKX-1.1) | |
| 760 | BHE40_HUMAN | Class E basic helix-loop-helix protein 40 | BHLHE40 |
| | | (bHLHe40) (Class B basic helix-loop-helix protein | BHLHB2 |
| | | 2) (bHLHb2) (Differentially expressed in | DEC1 |
| | | chondrocytes protein 1) (DEC1) (Enhancer-of-split | SHARP2 |
| | | and hairy-related protein 2) (SHARP-2) | STRA13 |
| | | (Stimulated by retinoic acid gene 13 protein) | |
| 761 | ZN260_HUMAN | Zinc finger protein 260 (Zfp-260) | ZNF260 |
| | | | ZFP260 |
| 762 | ZN821_HUMAN | Zinc finger protein 821 | ZNF821 |
| 763 | GATA1_HUMAN | Erythroid transcription factor (Eryf1) (GATA- | GATA1 |
| | | binding factor 1) (GATA-1) (GF-1) (NF-E1 DNA- | ERYF1 |
| | | binding protein) | GF1 |
| 764 | RUNX3_HUMAN | Runt-related transcription factor 3 (Acute myeloid | RUNX3 |
| | | leukemia 2 protein) (Core-binding factor subunit | AML2 |
| | | alpha-3) (CBF-alpha-3) (Oncogene AML-2) | CBFA3 |
| | | (Polyomavirus enhancer-binding protein 2 alpha C | PEBP2A3 |
| | | subunit) (PEA2-alpha C) (PEBP2-alpha C) (SL3-3 | |
| | | enhancer factor 1 alpha C subunit) (SL3/AKV | |
| | | core-binding factor alpha C subunit) | |
| 765 | FX4L4_HUMAN | Forkhead box protein D4-like 4 (FOXD4-like 4) | FOXD4L4 |
| | | (Forkhead box protein D4-like 2) (Forkhead box | FOXD4B |
| | | protein D4B) (Myeloid factor-gamma) | FOXD4L2 |
| 766 | FX4L5_HUMAN | Forkhead box protein D4-like 5 (FOXD4-like 5) | FOXD4L5 |
| 767 | FX4L3_HUMAN | Forkhead box protein D4-like 3 (FOXD4-like 3) | FOXD4L3 |
| 768 | FX4L6_HUMAN | Forkhead box protein D4-like 6 (FOXD4-like 6) | FOXD4L6 |
| 769 | PAX2_HUMAN | Paired box protein Pax-2 | PAX2 |
| 770 | ZN232_HUMAN | Zinc finger protein 232 (Zinc finger and SCAN | ZNF232 |
| | | domain-containing protein 11) | ZSCAN11 |
| 771 | PO4F1_HUMAN | POU domain, class 4, transcription factor 1 (Brain- | POU4F1 |
| | | specific homeobox/POU domain protein 3A) | BRN3A |
| | | (Brain-3A) (Brn-3A) (Homeobox/POU domain | RDC1 |
| | | protein RDC-1) (Oct-T1) | |
| 772 | FOXI3_HUMAN | Forkhead box protein I3 | FOXI3 |
| 773 | NFIB_HUMAN | Nuclear factor 1 B-type (NF1-B) (Nuclear factor | NFIB |
| | | 1/B) (CCAAT-box-binding transcription factor) | |
| | | (CTF) (Nuclear factor I/B) (NF-I/B) (NFI-B) | |
| | | (TGGCA-binding protein) | |
| 774 | TAD2B_HUMAN | Transcriptional adapter 2-beta (ADA2-like protein | TADA2B |
| | | beta) (ADA2-beta) | ADA2B |
| 775 | FOXJ1_HUMAN | Forkhead box protein J1 (Forkhead-related protein | FOXJ1 |
| | | FKHL13) (Hepatocyte nuclear factor 3 forkhead | FKHL13 |
| | | homolog 4) (HFH-4) | HFH4 |
| 776 | REXO4_HUMAN | RNA exonuclease 4 (EC 3.1.—.—) (Exonuclease | REXO4 |
| | | XPMC2) (Prevents mitotic catastrophe 2 protein | PMC2 |
| | | homolog) (hPMC2) | XPMC2H |
| 777 | GFI1_HUMAN | Zinc finger protein Gfi-1 (Growth factor | GFI1 |
| | | independent protein 1) (Zinc finger protein 163) | ZNF163 |
| 778 | HSFX1_HUMAN | Heat shock transcription factor, X-linked | HSFX1 |
| | | | LW-1; |
| | | | HSFX2 |
| 779 | SOLH2_HUMAN | Spermatogenesis- and oogenesis-specific basic | SOHLH2 |
| | | helix-loop-helix-containing protein 2 | TEB1 |
| 780 | ZN789_HUMAN | Zinc finger protein 789 | ZNF789 |
| 781 | IRF8_HUMAN | Interferon regulatory factor 8 (IRF-8) (Interferon | IRF8 |
| | | consensus sequence-binding protein) (H-ICSBP) | ICSBP1 |
| | | (ICSBP) | |
| 782 | ZN75C_HUMAN | Putative zinc finger protein 75C (Zinc finger | ZNF75CP |
| | | protein 75C pseudogene) | ZNF75C |
| 783 | ZNF2_HUMAN | Zinc finger protein 2 (Zinc finger protein 2.2) (Zinc | ZNF2 |
| | | finger protein 661) | ZNF661 |
| 784 | ZN134_HUMAN | Zinc finger protein 134 | ZNF134 |
| 785 | Z355P_HUMAN | Putative zinc finger protein 355P (Zinc finger | ZNF355P |
| | | protein ZnFP01) | ZNF834 |
| | | | PRED65 |
| 786 | ZN275_HUMAN | Zinc finger protein 275 | ZNF275 |
| 787 | PBX2_HUMAN | Pre-B-cell leukemia transcription factor 2 | PBX2 |
| | | (Homeobox protein PBX2) (Protein G17) | G17 |
| 788 | SPZ1_HUMAN | Spermatogenic leucine zipper protein 1 (Testis- | SPZ1 |
| | | specific protein 1) (Testis-specific protein NYD- | TSP1 |
| | | TSP1) | |
| 789 | FOXN2_HUMAN | Forkhead box protein N2 (Human T-cell leukemia | FOXN2 |
| | | virus enhancer factor) | HTLF |
| 790 | HXB3_HUMAN | Homeobox protein Hox-B3 (Homeobox protein | HOXB3 |
| | | Hox-2.7) (Homeobox protein Hox-2G) | HOX2G |
| 791 | NOCT_HUMAN | Nocturnin (EC 3.1.13.4) (Carbon catabolite | NOCT |
| | | repression 4-like protein) (Circadian deadenylase | CCR4 |
| | | NOC) | CCRN4L |
| | | | NOC |
| 792 | SP7_HUMAN | Transcription factor Sp7 (Zinc finger protein | SP7 |
| | | osterix) | OSX |
| 793 | FOXB2_HUMAN | Forkhead box protein B2 | FOXB2 |
| 794 | HXD3_HUMAN | Homeobox protein Hox-D3 (Homeobox protein | HOXD3 |
| | | Hox-4A) | HOX1D |
| | | | HOX4A |
| 795 | TADA3_HUMAN | Transcriptional adapter 3 (ADA3 homolog) | TADA3 |
| | | (hADA3) (STAF54) (Transcriptional adapter 3- | ADA3 |
| | | like) (ADA3-like protein) | TADA3L |
| 796 | ZN829_HUMAN | Zinc finger protein 829 | ZNF829 |
| 797 | ZSCA4_HUMAN | Zinc finger and SCAN domain-containing protein 4 | ZSCAN4 |
| | | (Zinc finger protein 494) | ZNF494 |
| 798 | PBX3_HUMAN | Pre-B-cell leukemia transcription factor 3 | PBX3 |
| | | (Homeobox protein PBX3) | |
| 799 | BRAC_HUMAN | Brachyury protein (Protein T) | T |
| 800 | ZBT25_HUMAN | Zinc finger and BTB domain-containing protein 25 | ZBTB25 |
| | | (Zinc finger protein 46) (Zinc finger protein KUP) | C14orf51 |
| | | | KUP |
| | | | ZNF46 |
| 801 | GCM1_HUMAN | Chorion-specific transcription factor GCMa | GCM1 |
| | | (hGCMa) (GCM motif protein 1) (Glial cells | GCMA |
| | | missing homolog 1) | |
| 802 | PO2F3_HUMAN | POU domain, class 2, transcription factor 3 | POU2F3 |
| | | (Octamer-binding protein 11) (Oct-11) (Octamer- | OTF11 |
| | | binding transcription factor 11) (OTF-11) | PLA1 |
| | | (Transcription factor PLA-1) (Transcription factor | |
| | | Skn-1) | |
| 803 | TBX6_HUMAN | T-box transcription factor TBX6 (T-box protein 6) | TBX6 |
| 804 | AP2A_HUMAN | Transcription factor AP-2-alpha (AP2-alpha) (AP-2 | TFAP2A |
| | | transcription factor) (Activating enhancer-binding | AP2TF |
| | | protein 2-alpha) (Activator protein 2) (AP-2) | TFAP2 |
| 805 | ZN154_HUMAN | Zinc finger protein 154 | ZNF154 |
| | | | KIAA2003 |
| 806 | ZN641_HUMAN | Zinc finger protein 641 | ZNF641 |
| 807 | FOXD4_HUMAN | Forkhead box protein D4 (Forkhead-related protein | FOXD4 |
| | | FKHL9) (Forkhead-related transcription factor 5) | FKHL9 |
| | | (FREAC-5) (Myeloid factor-alpha) | FOXD4A |
| | | | FREAC5 |
| 808 | ZN621_HUMAN | Zinc finger protein 621 | ZNF621 |
| 809 | SOX11_HUMAN | Transcription factor SOX-11 | SOX11 |
| 810 | AP2E_HUMAN | Transcription factor AP-2-epsilon (AP2-epsilon) | TFAP2E |
| | | (Activating enhancer-binding protein 2-epsilon) | |
| 811 | TRI14_HUMAN | Tripartite motif-containing protein 14 | TRIM14 |
| | | | KIAA0129 |
| 812 | HXA3_HUMAN | Homeobox protein Hox-A3 (Homeobox protein | HOXA3 |
| | | Hox-1E) | HOX1E |
| 813 | PO3F2_HUMAN | POU domain, class 3, transcription factor 2 (Brain- | POU3F2 |
| | | specific homeobox/POU domain protein 2) (Brain- | BRN2 |
| | | 2) (Brn-2) (Nervous system-specific octamer- | OCT7 |
| | | binding transcription factor N-Oct-3) (Octamer- | OTF7 |
| | | binding protein 7) (Oct-7) (Octamer-binding | |
| | | transcription factor 7) (OTF-7) | |
| 814 | FOXF2_HUMAN | Forkhead box protein F2 (Forkhead-related | FOXF2 |
| | | activator 2) (FREAC-2) (Forkhead-related protein | FKHL6 |
| | | FKHL6) (Forkhead-related transcription factor 2) | FREAC2 |
| 815 | SOX3_HUMAN | Transcription factor SOX-3 | SOX3 |
| 816 | SOX8_HUMAN | Transcription factor SOX-8 | SOX8 |
| 817 | ZNF3_HUMAN | Zinc finger protein 3 (Zinc finger protein HF.12) | ZNF3 |
| | | (Zinc finger protein HZF3.1) (Zinc finger protein | KOX25 |
| | | KOX25) | |
| 818 | TBX20_HUMAN | T-box transcription factor TBX20 (T-box protein | TBX20 |
| | | 20) | |
| 819 | ZIC1_HUMAN | Zinc finger protein ZIC 1 (Zinc finger protein 201) | ZIC1 |
| | | (Zinc finger protein of the cerebellum 1) | ZIC |
| | | | ZNF201 |
| 820 | GABP2_HUMAN | GA-binding protein subunit beta-2 (GABP subunit | GABPB2 |
| | | beta-2) (GABPB-2) | |
| 821 | TBX19_HUMAN | T-box transcription factor TBX19 (T-box protein | TBX19 |
| | | 19) (T-box factor, pituitary) | TPIT |
| 822 | ZBT14_HUMAN | Zinc finger and BTB domain-containing protein 14 | ZBTB14 |
| | | (Zinc finger protein 161 homolog) (Zfp-161) (Zinc | ZFP161 |
| | | finger protein 478) (Zinc finger protein 5 homolog) | ZNF478 |
| | | (ZF5) (Zfp-5) (hZF5) | |
| 823 | CIR1_HUMAN | Corepressor interacting with RBPJ 1 (CBF1- | CIR1 |
| | | interacting corepressor) (Recepin) | CIR |
| 824 | AP2C_HUMAN | Transcription factor AP-2 gamma (AP2-gamma) | TFAP2C |
| | | (Activating enhancer-binding protein 2 gamma) | |
| | | (Transcription factor ERF-1) | |
| 825 | ZN277_HUMAN | Zinc finger protein 277 (Nuclear receptor- | ZNF277 |
| | | interacting factor 4) | NRIF4 |
| | | | ZNF277P |
| 826 | ZN446_HUMAN | Zinc finger protein 446 (Zinc finger protein with | ZNF446 |
| | | KRAB and SCAN domains 20) | ZKSCAN20 |
| 827 | PO3F1_HUMAN | POU domain, class 3, transcription factor 1 | POU3F1 |
| | | (Octamer-binding protein 6) (Oct-6) (Octamer- | OCT6 |
| | | binding transcription factor 6) (OTF-6) (POU | OTF6 |
| | | domain transcription factor SCIP) | |
| 828 | AP2D_HUMAN | Transcription factor AP-2-delta (AP2-delta) | TFAP2D |
| | | (Activating enhancer-binding protein 2-delta) | TFAP2BL1 |
| | | (Transcription factor AP-2-beta-like 1) | |
| 829 | ZN672_HUMAN | Zinc finger protein 672 | ZNF672 |
| 830 | GABPA_HUMAN | GA-binding protein alpha chain (GABP subunit | GABPA |
| | | alpha) (Nuclear respiratory factor 2 subunit alpha) | E4TF1A |
| | | (Transcription factor E4TF1-60) | |
| 831 | FOXA2_HUMAN | Hepatocyte nuclear factor 3-beta (HNF-3-beta) | FOXA2 |
| | | (HNF-3B) (Forkhead box protein A2) | HNF3B |
| | | (Transcription factor 3B) (TCF-3B) | TCF3B |
| 832 | ZN140_HUMAN | Zinc finger protein 140 | ZNF140 |
| 833 | ZNF19_HUMAN | Zinc finger protein 19 (Zinc finger protein KOX12) | ZNF19 |
| | | | KOX12 |
| 834 | ZN239_HUMAN | Zinc finger protein 239 (Zinc finger protein HOK- | ZNF239 |
| | | 2) (Zinc finger protein MOK-2) | HOK2 |
| | | | MOK2 |
| 835 | ZN213_HUMAN | Zinc finger protein 213 (Putative transcription | ZNF213 |
| | | factor CR53) (Zinc finger protein with KRAB and | ZKSCAN21 |
| | | SCAN domains 21) | |
| 836 | AP2B_HUMAN | Transcription factor AP-2-beta (AP2-beta) | TFAP2B |
| | | (Activating enhancer-binding protein 2-beta) | |
| 837 | CR3L3_HUMAN | Cyclic AMP-responsive element-binding protein 3- | CREB3L3 |
| | | like protein 3 (cAMP-responsive element-binding | CREBH |
| | | protein 3-like protein 3) (Transcription factor | HYST1481 |
| | | CREB-H) [Cleaved into: Processed cyclic AMP- | |
| | | responsive element-binding protein 3-like protein 3] | |
| 838 | ZFP2_HUMAN | Zinc finger protein 2 homolog (Zfp-2) (Zinc finger | ZFP2 |
| | | protein 751) | ZNF751 |
| 839 | NFIL3_HUMAN | Nuclear factor interleukin-3-regulated protein (E4 | NFIL3 |
| | | promoter-binding protein 4) (Interleukin-3 | E4BP4 |
| | | promoter transcriptional activator) (Interleukin-3- | IL3BP1 |
| | | binding protein 1) (Transcriptional activator NF- | |
| | | IL3A) | |
| 840 | TRI38_HUMAN | E3 ubiquitin-protein ligase TRIM38 (EC 2.3.2.27) | TRIM38 |
| | | (RING finger protein 15) (RING-type E3 ubiquitin | RNF15 |
| | | transferase TRIM38) (Tripartite motif-containing | RORET |
| | | protein 38) (Zinc finger protein RoRet) | |
| 841 | FOXD1_HUMAN | Forkhead box protein D1 (Forkhead-related protein | FOXD1 |
| | | FKHL8) (Forkhead-related transcription factor 4) | FKHL8 |
| | | (FREAC-4) | FREAC4 |
| 842 | HNF6_HUMAN | Hepatocyte nuclear factor 6 (HNF-6) (One cut | ONECUT1 |
| | | domain family member 1) (One cut homeobox 1) | HNF6 |
| | | | HNF6A |
| 843 | SMAD5_HUMAN | Mothers against decapentaplegic homolog 5 (MAD | SMAD5 |
| | | homolog 5) (Mothers against DPP homolog 5) | MADH5 |
| | | (JV5-1) (SMAD family member 5) (SMAD 5) | |
| | | (Smad5) (hSmad5) | |
| 844 | E2F3_HUMAN | Transcription factor E2F3 (E2F-3) | E2F3 |
| | | | KIAA0075 |
| 845 | TRI15_HUMAN | Tripartite motif-containing protein 15 (RING finger | TRIM15 |
| | | protein 93) (Zinc finger protein 178) (Zinc finger | RNF93 |
| | | protein B7) | ZNF178 |
| | | | ZNFB7 |
| 846 | SOX10_HUMAN | Transcription factor SOX-10 | SOX10 |
| 847 | IRF6_HUMAN | Interferon regulatory factor 6 (IRF-6) | IRF6 |
| 848 | SMAD9_HUMAN | Mothers against decapentaplegic homolog 9 (MAD | SMAD9 |
| | | homolog 9) (Mothers against DPP homolog 9) | MADH6 |
| | | (Madh6) (SMAD family member 9) (SMAD 9) | MADH9 |
| | | (Smad9) | SMAD8 |
| 849 | RORB_HUMAN | Nuclear receptor ROR-beta (Nuclear receptor | RORB |
| | | RZR-beta) (Nuclear receptor subfamily 1 group F | NR1F2 |
| | | member 2) (Retinoid-related orphan receptor-beta) | RZRB |
| 850 | ZN436_HUMAN | Zinc finger protein 436 | ZNF436 |
| | | | KIAA1710 |
| 851 | DMRT3_HUMAN | Doublesex- and mab-3-related transcription factor 3 | DMRT3 |
| | | | DMRTA3 |
| 852 | FOXA1_HUMAN | Hepatocyte nuclear factor 3-alpha (HNF-3-alpha) | FOXA1 |
| | | (HNF-3A) (Forkhead box protein A1) | HNF3A |
| | | (Transcription factor 3A) (TCF-3A) | TCF3A |
| 853 | ZIM3_HUMAN | Zinc finger imprinted 3 (Zinc finger protein 657) | ZIM3 |
| | | | ZNF657 |
| 854 | MEF2C_HUMAN | Myocyte-specific enhancer factor 2C | MEF2C |
| 855 | GPBP1_HUMAN | Vasculin (GC-rich promoter-binding protein 1) | GPBP1 |
| | | (Vascular wall-linked protein) | GPBP |
| | | | SSH6 |
| 856 | SOX4_HUMAN | Transcription factor SOX-4 | SOX4 |
| 857 | GPBL1_HUMAN | Vasculin-like protein 1 (GC-rich promoter-binding | GPBP1L1 |
| | | protein 1-like 1) | SP192 |
| 858 | TRI62_HUMAN | E3 ubiquitin-protein ligase TRIM62 (EC 2.3.2.27) | TRIM62 |
| | | (RING-type E3 ubiquitin transferase TRIM62) | |
| | | (Tripartite motif-containing protein 62) | |
| 859 | ZN383_HUMAN | Zinc finger protein 383 | ZNF383 |
| | | | HSD17 |
| 860 | EGR2_HUMAN | E3 SUMO-protein ligase EGR2 (EC 6.3.2.—) | EGR2 |
| | | (AT591) (Early growth response protein 2) (EGR- | KROX20 |
| | | 2) (Zinc finger protein Krox-20) | |
| 861 | TFEB_HUMAN | Transcription factor EB (Class E basic helix-loop- | TFEB |
| | | helix protein 35) (bHLHe35) | BHLHE35 |
| 862 | MAZ_HUMAN | Myc-associated zinc finger protein (MAZI) (Pur-1) | MAZ |
| | | (Purine-binding transcription factor) (Serum | ZNF801 |
| | | amyloid A-activating factor-1) (SAF-1) | |
| | | (Transcription factor Zif87) (ZF87) (Zinc finger | |
| | | protein 801) | |
| 863 | ZN207_HUMAN | BUB3-interacting and GLEBS motif-containing | ZNF207 |
| | | protein ZNF207 (BuGZ) (hBuGZ) (Zinc finger | BUGZ |
| | | protein 207) | |
| 864 | FOXD3_HUMAN | Forkhead box protein D3 (HNF3/FH transcription | FOXD3 |
| | | factor genesis) | HFH2 |
| 865 | ZSC26_HUMAN | Zinc finger and SCAN domain-containing protein | ZSCAN26 |
| | | 26 (Protein SRE-ZBP) (Zinc finger protein 187) | ZNF187 |
| 866 | ZN302_HUMAN | Zinc finger protein 302 (Zinc finger protein 135- | ZNF302 |
| | | like) (Zinc finger protein 140-like) (Zinc finger | ZNF135L |
| | | protein 327) | ZNF140L |
| | | | ZNF327 |
| 867 | TF2L1_HUMAN | Transcription factor CP2-like protein 1 (CP2- | TFCP2L1 |
| | | related transcriptional repressor 1) (CRTR-1) | CRTR1 |
| | | (Transcription factor LBP-9) | LBP9 |
| 868 | GATA2_HUMAN | Endothelial transcription factor GATA-2 (GATA- | GATA2 |
| | | binding protein 2) | |
| 869 | NR6A1_HUMAN | Nuclear receptor subfamily 6 group A member 1 | NR6A1 |
| | | (Germ cell nuclear factor) (GCNF) (hGCNF) | GCNF |
| | | (Retinoid receptor-related testis-specific receptor) | |
| | | (RTR) (hRTR) | |
| 870 | ZN500_HUMAN | Zinc finger protein 500 (Zinc finger protein with | ZNF500 |
| | | KRAB and SCAN domains 18) | KIAA0557 |
| | | | ZKSCAN18 |
| 871 | BHE41_HUMAN | Class E basic helix-loop-helix protein 41 | BHLHE41 |
| | | (bHLHe41) (Class B basic helix-loop-helix protein | BHLHB3 |
| | | 3) (bHLHb3) (Differentially expressed in | DEC2 |
| | | chondrocytes protein 2) (hDEC2) (Enhancer-of- | SHARP1 |
| | | split and hairy-related protein 1) (SHARP-1) | |
| 872 | ZN117_HUMAN | Zinc finger protein 117 (Provirus-linked krueppel) | ZNF117 |
| | | (h-PLK) (Zinc finger protein HPF9) | |
| 873 | ZN774_HUMAN | Zinc finger protein 774 | ZNF774 |
| 874 | SP9_HUMAN | Transcription factor Sp9 | SP9 |
| 875 | ZN165_HUMAN | Zinc finger protein 165 (Cancer/testis antigen 53) | ZNF165 |
| | | (CT53) (LD65) (Zinc finger and SCAN domain- | ZPF165 |
| | | containing protein 7) | ZSCAN7 |
| 876 | ZN577_HUMAN | Zinc finger protein 577 | ZNF577 |
| 877 | ZN639_HUMAN | Zinc finger protein 639 (Zinc finger protein | ZNF639 |
| | | ANC_2H01) (Zinc finger protein ZASC1) | ZASC1 |
| 878 | HLX_HUMAN | H2.0-like homeobox protein (Homeobox protein | HLX |
| | | HB24) (Homeobox protein HLX1) | HLX1 |
| 879 | ZN345_HUMAN | Zinc finger protein 345 (Zinc finger protein | ZNF345 |
| | | HZF10) | |
| 880 | ZNF71_HUMAN | Endothelial zinc finger protein induced by tumor | ZNF71 |
| | | necrosis factor alpha (Zinc finger protein 71) | EZFIT |
| 881 | FOXG1_HUMAN | Forkhead box protein G1 (Brain factor 1) (BF-1) | FOXG1 |
| | | (BF1) (Brain factor 2) (BF-2) (BF2) (hBF-2) | FKH2 |
| | | (Forkhead box protein G1A) (Forkhead box protein | FKHL1 |
| | | G1B) (Forkhead box protein G1C) (Forkhead- | FKHL2 |
| | | related protein FKHL1) (HFK1) (Forkhead-related | FKHL3 |
| | | protein FKHL2) (HFK2) (Forkhead-related protein | FKHL4 |
| | | FKHL3) (HFK3) | FOXG1A |
| | | | FOXG1B |
| | | | FOXG1C |
| 882 | FOXN3_HUMAN | Forkhead box protein N3 (Checkpoint suppressor 1) | FOXN3 |
| | | | C14orf116 |
| | | | CHES1 |
| 883 | SP8_HUMAN | Transcription factor Sp8 (Specificity protein 8) | SP8 |
| 884 | ZSC22_HUMAN | Zinc finger and SCAN domain-containing protein | ZSCAN22 |
| | | 22 (Krueppel-related zinc finger protein 2) (Protein | HKR2 |
| | | HKR2) (Zinc finger protein 50) | ZNF50 |
| 885 | ZN655_HUMAN | Zinc finger protein 655 (Vav-interacting Krueppel- | ZNF655 |
| | | like protein) | VIK |
| 886 | FOXO6_HUMAN | Forkhead box protein O6 | FOXO6 |
| 887 | HSF4_HUMAN | Heat shock factor protein 4 (HSF 4) (hHSF4) (Heat | HSF4 |
| | | shock transcription factor 4) (HSTF 4) | |
| 888 | ATF7_HUMAN | Cyclic AMP-dependent transcription factor ATF-7 | ATF7 |
| | | (cAMP-dependent transcription factor ATF-7) | ATFA |
| | | (Activating transcription factor 7) (Transcription | |
| | | factor ATF-A) | |
| 889 | ONEC3_HUMAN | One cut domain family member 3 (One cut | ONECUT3 |
| | | homeobox 3) (Transcription factor ONECUT-3) | |
| | | (OC-3) | |
| 890 | ZSC30_HUMAN | Zinc finger and SCAN domain-containing protein | ZSCAN30 |
| | | 30 (ZNF-WYM) (Zinc finger protein 397 opposite | ZNF397OS |
| | | strand) (Zinc finger protein 397OS) | |
| 891 | FOXD2_HUMAN | Forkhead box protein D2 (Forkhead-related protein | FOXD2 |
| | | FKHL17) (Forkhead-related transcription factor 9) | FKHL17 |
| | | (FREAC-9) | FREAC9 |
| 892 | ZSA5B_HUMAN | Zinc finger and SCAN domain-containing protein 5B | ZSCAN5B |
| 893 | SMAD6_HUMAN | Mothers against decapentaplegic homolog 6 (MAD | SMAD6 |
| | | homolog 6) (Mothers against DPP homolog 6) | MADH6 |
| | | (SMAD family member 6) (SMAD 6) (Smad6) | |
| | | (hSMAD6) | |
| 894 | ZSA5C_HUMAN | Putative zinc finger and SCAN domain-containing | ZSCAN5C |
| | | protein 5C (Zinc finger and SCAN domain- | ZSCAN5CP |
| | | containing protein 5C pseudogene) | |
| 895 | SGK3_HUMAN | Serine/threonine-protein kinase Sgk3 (EC 2.7.11.1) | SGK3 |
| | | (Cytokine-independent survival kinase) | CISK |
| | | (Serum/glucocorticoid-regulated kinase 3) | SGKL |
| | | (Serum/glucocorticoid-regulated kinase-like) | |
| 896 | ZSA5A_HUMAN | Zinc finger and SCAN domain-containing protein | ZSCAN5A |
| | | 5A (Zinc finger protein 495) | ZNF495 |
| | | | ZSCAN5 |
| 897 | PLAL2_HUMAN | Zinc finger protein PLAGL2 (Pleiomorphic | PLAGL2 |
| | | adenoma-like protein 2) | KIAA0198 |
| 898 | ZSA5D_HUMAN | Putative zinc finger and SCAN domain-containing | ZSCAN5DP |
| | | protein 5D (Zinc finger and SCAN domain- | ZSCAN5D |
| | | containing protein 5D pseudogene) | |
| 899 | 2A5B_HUMAN | Serine/threonine-protein phosphatase 2A 56 kDa | PPP2R5B |
| | | regulatory subunit beta isoform (PP2A B subunit | |
| | | isoform B′-beta) (PP2A B subunit isoform B56- | |
| | | beta) (PP2A B subunit isoform PR61-beta) (PP2A | |
| | | B subunit isoform R5-beta) | |
| 900 | TRI22_HUMAN | E3 ubiquitin-protein ligase TRIM22 (EC 2.3.2.27) | TRIM22 |
| | | (50 kDa-stimulated trans-acting factor) (RING | RNF94 |
| | | finger protein 94) (RING-type E3 ubiquitin | STAF50 |
| | | transferase TRIM22) (Staf-50) (Tripartite motif- | |
| | | containing protein 22) | |
| 901 | PO3F3_HUMAN | POU domain, class 3, transcription factor 3 (Brain- | POU3F3 |
| | | specific homeobox/POU domain protein 1) (Brain- | BRN1 |
| | | 1) (Brn-1) (Octamer-binding protein 8) (Oct-8) | OTF8 |
| | | (Octamer-binding transcription factor 8) (OTF-8) | |
| 902 | TCFL5_HUMAN | Transcription factor-like 5 protein (Cha | TCFL5 |
| | | transcription factor) (HPV-16 E2-binding protein | CHA |
| | | 1) (E2BP-1) | E2BP1 |
| 903 | ZN888_HUMAN | Zinc finger protein 888 | ZNF888 |
| 904 | PLAG1_HUMAN | Zinc finger protein PLAG1 (Pleiomorphic adenoma | PLAG1 |
| | | gene 1 protein) | |
| 905 | IRX3_HUMAN | Iroquois-class homeodomain protein IRX-3 | IRX3 |
| | | (Homeodomain protein IRXB1) (Iroquois | IRXB1 |
| | | homeobox protein 3) | |
| 906 | TFCP2_HUMAN | Alpha-globin transcription factor CP2 (SAA3 | TFCP2 |
| | | enhancer factor) (Transcription factor LSF) | LSF |
| | | | SEF |
| 907 | NFIX_HUMAN | Nuclear factor 1 X-type (NF1-X) (Nuclear factor | NFIX |
| | | 1/X) (CCAAT-box-binding transcription factor) | |
| | | (CTF) (Nuclear factor I/X) (NF-I/X) (NFI-X) | |
| | | (TGGCA-binding protein) | |
| 908 | ZFP3_HUMAN | Zinc finger protein 3 homolog (Zfp-3) (Zinc finger | ZFP3 |
| | | protein 752) | ZNF752 |
| 909 | NRF1_HUMAN | Nuclear respiratory factor 1 (NRF-1) (Alpha | NRF1 |
| | | palindromic-binding protein) (Alpha-pal) | |
| 910 | DMRTA_HUMAN | Doublesex- and mab-3-related transcription factor | DMRTA1 |
| | | A1 | DMO |
| 911 | ONEC2_HUMAN | One cut domain family member 2 (Hepatocyte | ONECUT2 |
| | | nuclear factor 6-beta) (HNF-6-beta) (One cut | HNF6B |
| | | homeobox 2) (Transcription factor ONECUT-2) | |
| | | (OC-2) | |
| 912 | PAX7_HUMAN | Paired box protein Pax-7 (HuP1) | PAX7 |
| | | | HUP1 |
| 913 | ZN649_HUMAN | Zinc finger protein 649 | ZNF649 |
| 914 | GCM2_HUMAN | Chorion-specific transcription factor GCMb | GCM2 |
| | | (hGCMb) (GCM motif protein 2) (Glial cells | GCMB |
| | | missing homolog 2) | |
| 915 | ZN157_HUMAN | Zinc finger protein 157 (Zinc finger protein | ZNF157 |
| | | HZF22) | |
| 916 | CREB5_HUMAN | Cyclic AMP-responsive element-binding protein 5 | CREB5 |
| | | (CREB-5) (cAMP-responsive element-binding | CREBPA |
| | | protein 5) (CRE-BPa) | |
| 917 | NFIC_HUMAN | Nuclear factor 1 C-type (NF1-C) (Nuclear factor | NFIC |
| | | 1/C) (CCAAT-box-binding transcription factor) | NFI |
| | | (CTF) (Nuclear factor I/C) (NF-I/C) (NFI-C) | |
| | | (TGGCA-binding protein) | |
| 918 | NFIA_HUMAN | Nuclear factor 1 A-type (NF1-A) (Nuclear factor | NFIA |
| | | 1/A) (CCAAT-box-binding transcription factor) | KIAA1439 |
| | | (CTF) (Nuclear factor I/A) (NF-I/A) (NFI-A) | |
| | | (TGGCA-binding protein) | |
| 919 | ZN320_HUMAN | Zinc finger protein 320 | ZNF320 |
| 920 | IKZF3_HUMAN | Zinc finger protein Aiolos (Ikaros family zinc | IKZF3 |
| | | finger protein 3) | ZNFN1A3 |
| 921 | ZSC18_HUMAN | Zinc finger and SCAN domain-containing protein | ZSCAN18 |
| | | 18 (Zinc finger protein 447) | ZNF447 |
| 922 | ZN75D_HUMAN | Zinc finger protein 75D (Zinc finger protein 75) | ZNF75D |
| | | (Zinc finger protein 82) | ZNF75 |
| | | | ZNF82 |
| 923 | ETV3_HUMAN | ETS translocation variant 3 (ETS domain | ETV3 |
| | | transcriptional repressor PE1) (PE-1) (Mitogenic | METS |
| | | Ets transcriptional suppressor) | PE1 |
| 924 | KLF4_HUMAN | Krueppel-like factor 4 (Epithelial zinc finger | KLF4 |
| | | protein EZF) (Gut-enriched krueppel-like factor) | EZF |
| | | | GKLF |
| 925 | ZN395_HUMAN | Zinc finger protein 395 (HD-regulating factor 2) | ZNF395 |
| | | (HDRF-2) (Huntington disease gene regulatory | HDBP2 |
| | | region-binding protein 2) (HD gene regulatory | PBF |
| | | region-binding protein 2) (HDBP-2) | |
| | | (Papillomavirus regulatory factor 1) (PRF-1) | |
| | | (Papillomavirus-binding factor) | |
| 926 | TRI27_HUMAN | Zinc finger protein RFP (EC 2.3.2.27) (RING | TRIM27 |
| | | finger protein 76) (RING-type E3 ubiquitin | RFP |
| | | transferase TRIM27) (Ret finger protein) | RNF76 |
| | | (Tripartite motif-containing protein 27) | |
| 927 | ZNF83_HUMAN | Zinc finger protein 83 (Zinc finger protein 816B) | ZNF83 |
| | | (Zinc finger protein HPF1) | ZNF816B |
| 928 | FOXN4_HUMAN | Forkhead box protein N4 | FOXN4 |
| 929 | HINFP_HUMAN | Histone H4 transcription factor (Histone nuclear | HINFP |
| | | factor P) (HiNF-P) (MBD2-interacting zinc finger | MIZF |
| | | protein) (Methyl-CpG-binding protein 2-interacting | ZNF743 |
| | | zinc finger protein) | |
| 930 | RBPJL_HUMAN | Recombining binding protein suppressor of | RBPJL |
| | | hairless-like protein (Transcription factor RBP-L) | RBPL |
| | | | RBPSUHL |
| 931 | ZN215_HUMAN | Zinc finger protein 215 (BWSCR2-associated zinc | ZNF215 |
| | | finger protein 2) (BAZ-2) (Zinc finger protein with | BAZ2 |
| | | KRAB and SCAN domains 11) | ZKSCAN11 |
| 932 | ZN449_HUMAN | Zinc finger protein 449 (Zinc finger and SCAN | ZNF449 |
| | | domain-containing protein 19) | ZSCAN19 |
| 933 | CR3L1_HUMAN | Cyclic AMP-responsive element-binding protein 3- | CREB3L1 |
| | | like protein 1 (cAMP-responsive element-binding | OASIS |
| | | protein 3-like protein 1) (Old astrocyte specifically- | PSEC0238 |
| | | induced substance) (OASIS) [Cleaved into: | |
| | | Processed cyclic AMP-responsive element-binding | |
| | | protein 3-like protein 1] | |
| 934 | IKZF1_HUMAN | DNA-binding protein Ikaros (Ikaros family zinc | IKZF1 |
| | | finger protein 1) (Lymphoid transcription factor | IK1 |
| | | LyF-1) | IKAROS |
| | | | LYF1 |
| | | | ZNFN1A1 |
| 935 | MBTP2_HUMAN | Membrane-bound transcription factor site-2 | MBTPS2 |
| | | protease (EC 3.4.24.85) (Endopeptidase S2P) | S2P |
| | | (Sterol regulatory element-binding proteins | |
| | | intramembrane protease) (SREBPs intramembrane | |
| | | protease) | |
| 936 | ZFP30_HUMAN | Zinc finger protein 30 homolog (Zfp-30) (Zinc | ZFP30 |
| | | finger protein 745) | KIAA0961 |
| | | | ZNF745 |
| 937 | CR3L2_HUMAN | Cyclic AMP-responsive element-binding protein 3- | CREB3L2 |
| | | like protein 2 (cAMP-responsive element-binding | BBF2H7 |
| | | protein 3-like protein 2) (BBF2 human homolog on | |
| | | chromosome 7) [Cleaved into: Processed cyclic | |
| | | AMP-responsive element-binding protein 3-like | |
| | | protein 2] | |
| 938 | TBX22_HUMAN | T-box transcription factor TBX22 (T-box protein 22) | TBX22 |
| | | | TBOX22 |
| 939 | MEF2D_HUMAN | Myocyte-specific enhancer factor 2D | MEF2D |
| 940 | RUNX2_HUMAN | Runt-related transcription factor 2 (Acute myeloid | RUNX2 |
| | | leukemia 3 protein) (Core-binding factor subunit | AML3 |
| | | alpha-1) (CBF-alpha-1) (Oncogene AML-3) | CBFA1 |
| | | (Osteoblast-specific transcription factor 2) (OSF-2) | OSF2 |
| | | (Polyomavirus enhancer-binding protein 2 alpha A | PEBP2A |
| | | subunit) (PEA2-alpha A) (PEBP2-alpha A) (SL3-3 | |
| | | enhancer factor 1 alpha A subunit) (SL3/AKV | |
| | | core-binding factor alpha A subunit) | |
| 941 | VEZF1_HUMAN | Vascular endothelial zinc finger 1 (Putative | VEZF1 |
| | | transcription factor DB1) (Zinc finger protein 161) | DB1 |
| | | | ZNF161 |
| 942 | Z286A_HUMAN | Zinc finger protein 286A | ZNF286A |
| | | | KIAA1874 |
| | | | ZNF286 |
| 943 | Z286B_HUMAN | Putative zinc finger protein 286B | ZNF286B |
| | | | ZNF286C |
| | | | ZNF286L |
| 944 | ZBT18_HUMAN | Zinc finger and BTB domain-containing protein 18 | ZBTB18 |
| | | (58 kDa repressor protein) (Transcriptional | RP58 |
| | | repressor RP58) (Translin-associated zinc finger | TAZ1 |
| | | protein 1) (TAZ-1) (Zinc finger protein 238) (Zinc | ZNF238 |
| | | finger protein C2H2-171) | |
| 945 | ZN454_HUMAN | Zinc finger protein 454 | ZNF454 |
| 946 | ZN468_HUMAN | Zinc finger protein 468 | ZNF468 |
| 947 | RCOR2_HUMAN | REST corepressor 2 | RCOR2 |
| 948 | ZN765_HUMAN | Zinc finger protein 765 | ZNF765 |
| 949 | GLIS2_HUMAN | Zinc finger protein GLIS2 (GLI-similar 2) | GLIS2 |
| | | (Neuronal Krueppel-like protein) | NKL |
| 950 | ZN678_HUMAN | Zinc finger protein 678 | ZNF678 |
| 951 | IKZF2_HUMAN | Zinc finger protein Helios (Ikaros family zinc | IKZF2 |
| | | finger protein 2) | HELIOS |
| | | | ZNFN1A2 |
| 952 | ZIM2_HUMAN | Zinc finger imprinted 2 (Zinc finger protein 656) | ZIM2 |
| | | | ZNF656 |
| 953 | ZNF35_HUMAN | Zinc finger protein 35 (Zinc finger protein HF.10) | ZNF35 |
| 954 | ZN490_HUMAN | Zinc finger protein 490 | ZNF490 |
| | | | KIAA1198 |
| 955 | ZN572_HUMAN | Zinc finger protein 572 | ZNF572 |
| 956 | GMEB2_HUMAN | Glucocorticoid modulatory element-binding protein | GMEB2 |
| | | 2 (GMEB-2) (DNA-binding protein p79PIF) | KIAA1269 |
| | | (Parvovirus initiation factor p79) (PIF p79) | |
| 957 | UNC4_HUMAN | Homeobox protein unc-4 homolog (Homeobox | UNCX |
| | | protein Uncx4.1) | UNCX4.1 |
| 958 | ZN701_HUMAN | Zinc finger protein 701 | ZNF701 |
| 959 | ZFP82_HUMAN | Zinc finger protein 82 homolog (Zfp-82) (Zinc | ZFP82 |
| | | finger protein 545) | KIAA1948 |
| | | | ZNF545 |
| 960 | ZIC2_HUMAN | Zinc finger protein ZIC 2 (Zinc finger protein of | ZIC2 |
| | | the cerebellum 2) | |
| 961 | ZFP14_HUMAN | Zinc finger protein 14 homolog (Zfp-14) (Zinc | ZFP14 |
| | | finger protein 531) | KIAA1559 |
| | | | ZNF531 |
| 962 | ZNF26_HUMAN | Zinc finger protein 26 (Zinc finger protein KOX20) | ZNF26 |
| | | | KOX20 |
| 963 | ZN397_HUMAN | Zinc finger protein 397 (Zinc finger and SCAN | ZNF397 |
| | | domain-containing protein 15) (Zinc finger protein | ZNF47 |
| | | 47) | ZSCAN15 |
| 964 | ZF69B_HUMAN | Zinc finger protein ZFP69B (Zinc finger protein | ZFP69B |
| | | 643) | ZNF643 |
| 965 | TBX21_HUMAN | T-box transcription factor TBX21 (T-box protein | TBX21 |
| | | 21) (T-cell-specific T-box transcription factor T- | TBET |
| | | bet) (Transcription factor TBLYM) | TBLYM |
| 966 | ZN623_HUMAN | Zinc finger protein 623 | ZNF623 |
| | | | KIAA0628 |
| 967 | ZN835_HUMAN | Zinc finger protein 835 | ZNF835 |
| 968 | ZN155_HUMAN | Zinc finger protein 155 | ZNF155 |
| 969 | ZKSC3_HUMAN | Zinc finger protein with KRAB and SCAN | ZKSCAN3 |
| | | domains 3 (Zinc finger and SCAN domain- | ZFP47 |
| | | containing protein 13) (Zinc finger protein 306) | ZNF306 |
| | | (Zinc finger protein 309) (Zinc finger protein 47 | ZNF309 |
| | | homolog) (Zf47) (Zfp-47) | ZSCAN13 |
| 970 | TRI26_HUMAN | Tripartite motif-containing protein 26 (Acid finger | TRIM26 |
| | | protein) (AFP) (RING finger protein 95) (Zinc | RNF95 |
| | | finger protein 173) | ZNF173 |
| 971 | ZBT7B_HUMAN | Zinc finger and BTB domain-containing protein 7B | ZBTB7B |
| | | (Krueppel-related zinc finger protein cKrox) | ZBTB15 |
| | | (hcKrox) (T-helper-inducing POZ/Krueppel-like | ZFP67 |
| | | factor) (Zinc finger and BTB domain-containing | ZNF857B |
| | | protein 15) (Zinc finger protein 67 homolog) (Zfp- | |
| | | 67) (Zinc finger protein 857B) (Zinc finger protein | |
| | | Th-POK) | |
| 972 | ZN565_HUMAN | Zinc finger protein 565 | ZNF565 |
| 973 | UBIP1_HUMAN | Upstream-binding protein 1 (Transcription factor | UBP1 |
| | | LBP-1) | LBP1 |
| 974 | DMTA2_HUMAN | Doublesex- and mab-3-related transcription factor | DMRTA2 |
| | | A2 (Doublesex- and mab-3-related transcription | DMRT5 |
| | | factor 5) | |
| 975 | CSRN2_HUMAN | Cysteine/serine-rich nuclear protein 2 (CSRNP-2) | CSRNP2 |
| | | (Protein FAM130A1) (TGF-beta-induced apoptosis | C12orf22 |
| | | protein 12) (TAIP-12) | FAM130A1 |
| | | | TAIP12 |
| 976 | ZSC25_HUMAN | Zinc finger and SCAN domain-containing protein | ZSCAN25 |
| | | 25 (Zinc finger protein 498) | ZNF498 |
| 977 | TBX4_HUMAN | T-box transcription factor TBX4 (T-box protein 4) | TBX4 |
| 978 | ZKSC4_HUMAN | Zinc finger protein with KRAB and SCAN | ZKSCAN4 |
| | | domains 4 (P373c6.1) (Zinc finger protein 307) | ZNF307 |
| | | (Zinc finger protein 427) | ZNF427 |
| 979 | ERF_HUMAN | ETS domain-containing transcription factor ERF | ERF |
| | | (Ets2 repressor factor) (PE-2) | |
| 980 | ZNF18_HUMAN | Zinc finger protein 18 (Heart development-specific | ZNF18 |
| | | gene 1 protein) (Zinc finger protein 535) (Zinc | HDSG1 |
| | | finger protein KOX11) (Zinc finger protein with | KOX11 |
| | | KRAB and SCAN domains 6) | ZKSCAN6 |
| | | | ZNF535 |
| 981 | ZN382_HUMAN | Zinc finger protein 382 (KRAB/zinc finger | ZNF382 |
| | | suppressor protein 1) (KS1) (Multiple zinc finger | |
| | | and krueppel-associated box protein KS1) | |
| 982 | TRIM8_HUMAN | Probable E3 ubiquitin-protein ligase TRIM8 (EC | TRIM8 |
| | | 2.3.2.27) (Glioblastoma-expressed RING finger | GERP |
| | | protein) (RING finger protein 27) (RING-type E3 | RNF27 |
| | | ubiquitin transferase TRIM8) (Tripartite motif- | |
| | | containing protein 8) | |
| 983 | FOXC1_HUMAN | Forkhead box protein C1 (Forkhead-related protein | FOXC1 |
| | | FKHL7) (Forkhead-related transcription factor 3) | FKHL7 |
| | | (FREAC-3) | FREAC3 |
| 984 | ZN564_HUMAN | Zinc finger protein 564 | ZNF564 |
| 985 | Z354C_HUMAN | Zinc finger protein 354C (Kidney, ischemia, and | ZNF354C |
| | | developmentally-regulated protein 3) (hKID3) | KID3 |
| 986 | TRAF5_HUMAN | TNF receptor-associated factor 5 (RING finger | TRAF5 |
| | | protein 84) | RNF84 |
| 987 | AATF_HUMAN | Protein AATF (Apoptosis-antagonizing | AATF |
| | | transcription factor) (Rb-binding protein Che-1) | CHE1 |
| | | | DED |
| | | | HSPC277 |
| 988 | ZN250_HUMAN | Zinc finger protein 250 (Zinc finger protein 647) | ZNF250 |
| | | | ZNF647 |
| 989 | ARI3B_HUMAN | AT-rich interactive domain-containing protein 3B | ARID3B |
| | | (ARID domain-containing protein 3B) (Bright and | BDP |
| | | dead ringer protein) (Bright-like protein) | DRIL2 |
| 990 | DMRT2_HUMAN | Doublesex- and mab-3-related transcription factor | DMRT2 |
| | | 2 (Doublesex-like 2 protein) (DSXL-2) | DSXL2 |
| 991 | ZN37A_HUMAN | Zinc finger protein 37A (Zinc finger protein | ZNF37A |
| | | KOX21) | KOX21 |
| | | | ZNF37 |
| 992 | ZN394_HUMAN | Zinc finger protein 394 (Zinc finger protein with | ZNF394 |
| | | KRAB and SCAN domains 14) | ZKSCAN14 |
| 993 | ARX_HUMAN | Homeobox protein ARX (Aristaless-related | ARX |
| | | homeobox) | |
| 994 | ZN461_HUMAN | Zinc finger protein 461 (Gonadotropin-inducible | ZNF461 |
| | | ovary transcription repressor 1) (GIOT-1) | GIOT1 |
| 995 | ZN879_HUMAN | Zinc finger protein 879 | ZNF879 |
| 996 | ZKSC1_HUMAN | Zinc finger protein with KRAB and SCAN | ZKSCAN1 |
| | | domains 1 (Zinc finger protein 139) (Zinc finger | KOX18 |
| | | protein 36) (Zinc finger protein KOX18) | ZNF139 |
| | | | ZNF36 |
| 997 | FZD2_HUMAN | Frizzled-2 (Fz-2) (hFz2) (FzE2) | FZD2 |
| 998 | ZN358_HUMAN | Zinc finger protein 358 | ZNF358 |
| 999 | PRD14_HUMAN | PR domain zinc finger protein 14 (EC 2.1.1.—) (PR | PRDM14 |
| | | domain-containing protein 14) | |
| 1000 | ZN181_HUMAN | Zinc finger protein 181 (HHZ181) | ZNF181 |
| 1001 | F200A_HUMAN | Protein FAM200A | FAM200A |
| | | | C7orf38 |
| 1002 | FOXJ2_HUMAN | Forkhead box protein J2 (Fork head homologous X) | FOXJ2 |
| | | | FHX |
| 1003 | COE2_HUMAN | Transcription factor COE2 (Early B-cell factor 2) | EBF2 |
| | | (EBF-2) | COE2 |
| 1004 | TFE3_HUMAN | Transcription factor E3 (Class E basic helix-loop- | TFE3 |
| | | helix protein 33) (bHLHe33) | BHLHE33 |
| 1005 | ZN431_HUMAN | Zinc finger protein 431 | ZNF431 |
| | | | KIAA1969 |
| 1006 | ZN880_HUMAN | Zinc finger protein 880 | ZNF880 |
| 1007 | ZKSC8_HUMAN | Zinc finger protein with KRAB and SCAN | ZKSCAN8 |
| | | domains 8 (LD5-1) (Zinc finger protein 192) | ZNF192 |
| 1008 | RELB_HUMAN | Transcription factor RelB (I-Rel) | RELB |
| 1009 | PINK1_HUMAN | Serine/threonine-protein kinase PINK1, | PINK1 |
| | | mitochondrial (EC 2.7.11.1) (BRPK) (PTEN- | |
| | | induced putative kinase protein 1) | |
| 1010 | MNT_HUMAN | Max-binding protein MNT (Class D basic helix- | MNT |
| | | loop-helix protein 3) (bHLHd3) (Myc antagonist | BHLHD3 |
| | | MNT) (Protein ROX) | ROX |
| 1011 | ZN677_HUMAN | Zinc finger protein 677 | ZNF677 |
| 1012 | CSRN3_HUMAN | Cysteine/serine-rich nuclear protein 3 (CSRNP-3) | CSRNP3 |
| | | (Protein FAM130A2) (TGF-beta-induced apoptosis | FAM130A2 |
| | | protein 2) (TAIP-2) | TAIP2 |
| 1013 | CRY1_HUMAN | Cryptochrome-1 | CRY1 |
| | | | PHLL1 |
| 1014 | RFX8_HUMAN | DNA-binding protein RFX8 (Regulatory factor X 8) | RFX8 |
| 1015 | ZNF92_HUMAN | Zinc finger protein 92 (Zinc finger protein HTF12) | ZNF92 |
| 1016 | NACC2_HUMAN | Nucleus accumbens-associated protein 2 (NAC-2) | NACC2 |
| | | (BTB/POZ domain-containing protein 14A) | BTBD14A |
| | | (Repressor with BTB domain and BEN domain) | NAC2 |
| | | | RBB |
| 1017 | S6OS1_HUMAN | Protein SIX6OS1 (Six6 opposite strand transcript 1) | SIX6OS1 |
| | | | C14orf39 |
| 1018 | ZN496_HUMAN | Zinc finger protein 496 (Zinc finger protein with | ZNF496 |
| | | KRAB and SCAN domains 17) | ZKSCAN17 |
| 1019 | TAF1B_HUMAN | TATA box-binding protein-associated factor RNA | TAF1B |
| | | polymerase I subunit B (RNA polymerase I- | |
| | | specific TBP-associated factor 63 kDa) (TAFI63) | |
| | | (TATA box-binding protein-associated factor 1B) | |
| | | (TBP-associated factor 1B) (Transcription initiation | |
| | | factor SL1/TIF-IB subunit B) | |
| 1020 | TF7L1_HUMAN | Transcription factor 7-like 1 (HMG box | TCF7L1 |
| | | transcription factor 3) (TCF-3) | TCF3 |
| 1021 | CSRN1_HUMAN | Cysteine/serine-rich nuclear protein 1 (CSRNP-1) | CSRNP1 |
| | | (Axin-1 up-regulated gene 1 protein) (Protein | AXUD1 |
| | | URAX1) (TGF-beta-induced apoptosis protein 3) | TAIP3 |
| | | (TAIP-3) | |
| 1022 | EGR4_HUMAN | Early growth response protein 4 (EGR-4) (AT133) | EGR4 |
| 1023 | TAF5L_HUMAN | TAF5-like RNA polymerase II p300/CBP- | TAF5L |
| | | associated factor-associated factor 65 kDa subunit | PAF65B |
| | | 5L (PCAF-associated factor 65 beta) (PAF65-beta) | |
| 1024 | NPAS1_HUMAN | Neuronal PAS domain-containing protein 1 | NPAS1 |
| | | (Neuronal PAS1) (Basic-helix-loop-helix-PAS | BHLHE11 |
| | | protein MOP5) (Class E basic helix-loop-helix | MOP5 |
| | | protein 11) (bHLHe11) (Member of PAS protein 5) | PASD5 |
| | | (PAS domain-containing protein 5) | |
| 1025 | ZN578_HUMAN | Zinc finger protein 578 | ZNF578 |
| 1026 | CRY2_HUMAN | Cryptochrome-2 | CRY2 |
| | | | KIAA0658 |
| 1027 | ELF2_HUMAN | ETS-related transcription factor Elf-2 (E74-like | ELF2 |
| | | factor 2) (New ETS-related factor) | NERF |
| 1028 | MTF2_HUMAN | Metal-response element-binding transcription | MTF2 |
| | | factor 2 (Metal regulatory transcription factor 2) | PCL2 |
| | | (Metal-response element DNA-binding protein | |
| | | M96) (Polycomb-like protein 2) (hPCl2) | |
| 1029 | P66B_HUMAN | Transcriptional repressor p66-beta (GATA zinc | GATAD2B |
| | | finger domain-containing protein 2B) (p66/p68) | KIAA1150 |
| 1030 | ZN284_HUMAN | Zinc finger protein 284 | ZNF284 |
| | | | ZNF284L |
| 1031 | ARI5A_HUMAN | AT-rich interactive domain-containing protein 5A | ARID5A |
| | | (ARID domain-containing protein 5A) (Modulator | MRF1 |
| | | recognition factor 1) (MRF-1) | |
| 1032 | MTA3_HUMAN | Metastasis-associated protein MTA3 | MTA3 |
| | | | KIAA1266 |
| 1033 | ZBED8_HUMAN | Protein ZBED8 (Transposon-derived Buster3 | ZBED8 |
| | | transposase-like protein) (Zinc finger BED domain- | Buster3 |
| | | containing protein 8) | C5orf54 |
| 1034 | GATA6_HUMAN | Transcription factor GATA-6 (GATA-binding | GATA6 |
| | | factor 6) | |
| 1035 | ZN317_HUMAN | Zinc finger protein 317 | ZNF317 |
| | | | KIAA1588 |
| 1036 | ZNF85_HUMAN | Zinc finger protein 85 (Zinc finger protein HPF4) | ZNF85 |
| | | (Zinc finger protein HTF1) | |
| 1037 | HSF5_HUMAN | Heat shock factor protein 5 (HSF 5) (Heat shock | HSF5 |
| | | transcription factor 5) (HSTF 5) | HSTF5 |
| 1038 | LZTS1_HUMAN | Leucine zipper putative tumor suppressor 1 | LZTS1 |
| | | (F37/esophageal cancer-related gene-coding | FEZ1 |
| | | leucine-zipper motif) (Fez1) | |
| 1039 | DACH2_HUMAN | Dachshund homolog 2 (Dach2) | DACH2 |
| 1040 | MYEF2_HUMAN | Myelin expression factor 2 (MEF-2) (MyEF-2) | MYEF2 |
| | | (MST156) | KIAA1341 |
| 1041 | ZN543_HUMAN | Zinc finger protein 543 | ZNF543 |
| 1042 | ZNF90_HUMAN | Zinc finger protein 90 (Zinc finger protein HTF9) | ZNF90 |
| 1043 | TBX15_HUMAN | T-box transcription factor TBX15 (T-box protein | TBX15 |
| | | 15) (T-box transcription factor TBX14) (T-box | TBX14 |
| | | protein 14) | |
| 1044 | NR2C1_HUMAN | Nuclear receptor subfamily 2 group C member 1 | NR2C1 |
| | | (Orphan nuclear receptor TR2) (Testicular receptor 2) | TR2 |
| 1045 | ZN415_HUMAN | Zinc finger protein 415 | ZNF415 |
| 1046 | MTG8R_HUMAN | Protein CBFA2T2 (ETO homologous on | CBFA2T2 |
| | | chromosome 20) (MTG8-like protein) (MTG8- | EHT |
| | | related protein 1) (Myeloid translocation-related | MTGR1 |
| | | protein 1) (p85) | |
| 1047 | ZSC12_HUMAN | Zinc finger and SCAN domain-containing protein 12 | ZSCAN12 |
| | | (Zinc finger protein 305) (Zinc finger protein 96) | KIAA0426 |
| | | | ZNF305 |
| | | | ZNF96 |
| 1048 | ZN300_HUMAN | Zinc finger protein 300 | ZNF300 |
| 1049 | Z354A_HUMAN | Zinc finger protein 354A (Transcription factor 17) | ZNF354A |
| | | (TCF-17) (Zinc finger protein eZNF) | EZNF |
| | | | HKL1 |
| | | | TCF17 |
| 1050 | EPMIP_HUMAN | EPM2A-interacting protein 1 (Laforin-interacting | EPM2AIP1 |
| | | protein) | KIAA0766 |
| | | | My007 |
| 1051 | TBX18_HUMAN | T-box transcription factor TBX18 (T-box protein 18) | TBX18 |
| 1052 | ZN571_HUMAN | Zinc finger protein 571 | ZNF571 |
| | | | HSPC059 |
| 1053 | Z354B_HUMAN | Zinc finger protein 354B | ZNF354B |
| 1054 | SP2_HUMAN | Transcription factor Sp2 | SP2 |
| | | | KIAA0048 |
| 1055 | ZSCA2_HUMAN | Zinc finger and SCAN domain-containing protein 2 | ZSCAN2 |
| | | (Zinc finger protein 29 homolog) (Zfp-29) (Zinc | ZFP29 |
| | | finger protein 854) | ZNF854 |
| 1056 | ZN221_HUMAN | Zinc finger protein 221 | ZNF221 |
| 1057 | ZN613_HUMAN | Zinc finger protein 613 | ZNF613 |
| 1058 | ZN813_HUMAN | Zinc finger protein 813 | ZNF813 |
| 1059 | GRHL1_HUMAN | Grainyhead-like protein 1 homolog (Mammalian | GRHL1 |
| | | grainyhead) (NH32) (Transcription factor CP2-like | LBP32 |
| | | 2) (Transcription factor LBP-32) | MGR |
| | | | TFCP2L2 |
| 1060 | ELF1_HUMAN | ETS-related transcription factor Elf-1 (E74-like | ELF1 |
| | | factor 1) | |
| 1061 | REL_HUMAN | Proto-oncogene c-Rel | REL |
| 1062 | ZN668_HUMAN | Zinc finger protein 668 | ZNF668 |
| 1063 | ZNF93_HUMAN | Zinc finger protein 93 (Zinc finger protein 505) | ZNF93 |
| | | (Zinc finger protein HTF34) | ZNF505 |
| 1064 | KLHL6_HUMAN | Kelch-like protein 6 | KLHL6 |
| 1065 | FOXJ3_HUMAN | Forkhead box protein J3 | FOXJ3 |
| | | | KIAA1041 |
| 1066 | TAF6L_HUMAN | TAF6-like RNA polymerase II p300/CBP- | TAF6L |
| | | associated factor-associated factor 65 kDa subunit | PAF65A |
| | | 6L (PCAF-associated factor 65-alpha) (PAF65- | |
| | | alpha) | |
| 1067 | SOX13_HUMAN | Transcription factor SOX-13 (Islet cell antigen 12) | SOX13 |
| | | (SRY (Sex determining region Y)-box 13) (Type 1 | |
| | | diabetes autoantigen ICA12) | |
| 1068 | ZN728_HUMAN | Zinc finger protein 728 | ZNF728 |
| 1069 | LMBL4_HUMAN | Lethal(3)malignant brain tumor-like protein 4 (H- | L3MBTL4 |
| | | l(3)mbt-like protein 4) (L(3)mbt-like protein 4) | |
| | | (L3mbt-like 4) | |
| 1070 | ZN131_HUMAN | Zinc finger protein 131 | ZNF131 |
| 1071 | ZNF30_HUMAN | Zinc finger protein 30 (Zinc finger protein KOX28) | ZNF30 |
| | | | KOX28 |
| 1072 | RNF12_HUMAN | E3 ubiquitin-protein ligase RLIM (EC 2.3.2.27) | RLIM |
| | | (LIM domain-interacting RING finger protein) | RNF12 |
| | | (RING finger LIM domain-binding protein) (R- | |
| | | LIM) (RING finger protein 12) (RING-type E3 | |
| | | ubiquitin transferase RLIM) (Renal carcinoma | |
| | | antigen NY-REN-43) | |
| 1073 | GRHL2_HUMAN | Grainyhead-like protein 2 homolog (Brother of | GRHL2 |
| | | mammalian grainyhead) (Transcription factor CP2- | BOM |
| | | like 3) | TFCP2L3 |
| 1074 | GRHL3_HUMAN | Grainyhead-like protein 3 homolog (Sister of | GRHL3 |
| | | mammalian grainyhead) (Transcription factor CP2- | SOM |
| | | like 4) | TFCP2L4 |
| 1075 | NR4A3_HUMAN | Nuclear receptor subfamily 4 group A member 3 | NR4A3 |
| | | (Mitogen-induced nuclear orphan receptor) | CHN |
| | | (Neuron-derived orphan receptor 1) (Nuclear | CSMF |
| | | hormone receptor NOR-1) | MINOR |
| | | | NOR1 |
| | | | TEC |
| 1076 | ZN189_HUMAN | Zinc finger protein 189 | ZNF189 |
| 1077 | ZN471_HUMAN | Zinc finger protein 471 (EZFIT-related protein 1) | ZNF471 |
| | | | ERP1 |
| | | | KIAA1396 |
| 1078 | ZN256_HUMAN | Zinc finger protein 256 (Bone marrow zinc finger | ZNF256 |
| | | 3) (BMZF-3) | BMZF3 |
| 1079 | ZN528_HUMAN | Zinc finger protein 528 | ZNF528 |
| | | | KIAA1827 |
| 1080 | PRDM5_HUMAN | PR domain zinc finger protein 5 (EC 2.1.1.—) (PR | PRDM5 |
| | | domain-containing protein 5) | PFM2 |
| 1081 | ZFP37_HUMAN | Zinc finger protein 37 homolog (Zfp-37) | ZFP37 |
| 1082 | ZN860_HUMAN | Zinc finger protein 860 | ZNF860 |
| 1083 | ZN790_HUMAN | Zinc finger protein 790 | ZNF790 |
| 1084 | ZFP90_HUMAN | Zinc finger protein 90 homolog (Zfp-90) (Zinc | ZFP90 |
| | | finger protein 756) | KIAA1954 |
| | | | ZNF756 |
| 1085 | ZN143_HUMAN | Zinc finger protein 143 (SPH-binding factor) | ZNF143 |
| | | (Selenocysteine tRNA gene transcription-activating | SBF |
| | | factor) (hStaf) | STAF |
| 1086 | CRERF_HUMAN | CREB3 regulatory factor (Luman recruitment | CREBRF |
| | | factor) (LRF) | C5orf41 |
| 1087 | ZN182_HUMAN | Zinc finger protein 182 (Zinc finger protein 21) | ZNF182 |
| | | (Zinc finger protein KOX14) | KOX14 |
| | | | ZNF21 |
| 1088 | MYB_HUMAN | Transcriptional activator Myb (Proto-oncogene c- | MYB |
| | | Myb) | |
| 1089 | ZN605_HUMAN | Zinc finger protein 605 | ZNF605 |
| 1090 | Z780A_HUMAN | Zinc finger protein 780A | ZNF780A |
| 1091 | ZN699_HUMAN | Zinc finger protein 699 (Hangover homolog) | ZNF699 |
| 1092 | ZNF23_HUMAN | Zinc finger protein 23 (Zinc finger protein 359) | ZNF23 |
| | | (Zinc finger protein 612) (Zinc finger protein | KOX16 |
| | | KOX16) | ZNF359 |
| | | | ZNF612 |
| 1093 | ZN568_HUMAN | Zinc finger protein 568 | ZNF568 |
| 1094 | ZNF74_HUMAN | Zinc finger protein 74 (Zinc finger protein 520) | ZNF74 |
| | | (hZNF7) | ZNF520 |
| 1095 | ZN746_HUMAN | Zinc finger protein 746 (Parkin-interacting | ZNF746 |
| | | substrate) (PARIS) | PARIS |
| 1096 | ZN681_HUMAN | Zinc finger protein 681 | ZNF681 |
| 1097 | ZN493_HUMAN | Zinc finger protein 493 | ZNF493 |
| 1098 | FZD1_HUMAN | Frizzled-1 (Fz-1) (hFz1) (FzE1) | FZD1 |
| 1099 | ZN567_HUMAN | Zinc finger protein 567 | ZNF567 |
| 1100 | FOXN1_HUMAN | Forkhead box protein N1 (Winged-helix | FOXN1 |
| | | transcription factor nude) | RONU |
| | | | WHN |
| 1101 | ZN202_HUMAN | Zinc finger protein 202 (Zinc finger protein with | ZNF202 |
| | | KRAB and SCAN domains 10) | ZKSCAN10 |
| 1102 | ZN595_HUMAN | Zinc finger protein 595 | ZNF595 |
| 1103 | RRN3_HUMAN | RNA polymerase I-specific transcription initiation | RRN3 |
| | | factor RRN3 (Transcription initiation factor IA) | TIFIA |
| | | (TIF-IA) | |
| 1104 | ZN816_HUMAN | Zinc finger protein 816 | ZNF816 |
| | | | ZNF816A |
| 1105 | ZN432_HUMAN | Zinc finger protein 432 | ZNF432 |
| | | | KIAA0798 |
| 1106 | ZN274_HUMAN | Neurotrophin receptor-interacting factor homolog | ZNF274 |
| | | (Zinc finger protein 274) (Zinc finger protein | ZKSCAN19 |
| | | HFB101) (Zinc finger protein with KRAB and | SP2114 |
| | | SCAN domains 19) (Zinc finger protein zfp2) (Zf2) | |
| 1107 | MTG16_HUMAN | Protein CBFA2T3 (MTG8-related protein 2) | CBFA2T3 |
| | | (Myeloid translocation gene on chromosome 16 | MTG16 |
| | | protein) (hMTG16) (Zinc finger MYND domain- | MTGR2 |
| | | containing protein 4) | ZMYND4 |
| 1108 | ZN133_HUMAN | Zinc finger protein 133 (Zinc finger protein 150) | ZNF133 |
| | | | ZNF150 |
| 1109 | F200B_HUMAN | Protein FAM200B | FAM200B |
| | | | C4orf53 |
| 1110 | ZN630_HUMAN | Zinc finger protein 630 | ZNF630 |
| 1111 | ZN135_HUMAN | Zinc finger protein 135 (Zinc finger protein 61) | ZNF135 |
| | | (Zinc finger protein 78-like 1) | ZNF61 |
| | | | ZNF78L1 |
| 1112 | ZN254_HUMAN | Zinc finger protein 254 (Bone marrow zinc finger | ZNF254 |
| | | 5) (BMZF-5) (Hematopoietic cell-derived zinc | BMZF5 |
| | | finger protein 1) (HD-ZNF1) (Zinc finger protein | ZNF539 |
| | | 539) (Zinc finger protein 91-like) | ZNF91L |
| 1113 | ZN540_HUMAN | Zinc finger protein 540 | ZNF540 |
| | | | Nbla10512 |
| 1114 | ZNF81_HUMAN | Zinc finger protein 81 (HFZ20) | ZNF81 |
| 1115 | ELF4_HUMAN | ETS-related transcription factor Elf-4 (E74-like | ELF4 |
| | | factor 4) (Myeloid Elf-1-like factor) | ELFR |
| | | | MEF |
| 1116 | CTCFL_HUMAN | Transcriptional repressor CTCFL (Brother of the | CTCFL |
| | | regulator of imprinted sites) (CCCTC-binding | BORIS |
| | | factor) (CTCF paralog) (CTCF-like protein) | |
| | | (Cancer/testis antigen 27) (CT27) (Zinc finger | |
| | | protein CTCF-T) | |
| 1117 | ZN573_HUMAN | Zinc finger protein 573 | ZNF573 |
| 1118 | ZN311_HUMAN | Zinc finger protein 311 (Zinc finger protein zfp-31) | ZNF311 |
| | | | ZFP31 |
| 1119 | SIM2_HUMAN | Single-minded homolog 2 (Class E basic helix- | SIM2 |
| | | loop-helix protein 15) (bHLHe15) | BHLHE15 |
| 1120 | MTA2_HUMAN | Metastasis-associated protein MTA2 (Metastasis- | MTA2 |
| | | associated 1-like 1) (MTA1-L1 protein) (p53 target | MTA1L1 |
| | | protein in deacetylase complex) | PID |
| 1121 | ATF6A_HUMAN | Cyclic AMP-dependent transcription factor ATF-6 | ATF6 |
| | | alpha (cAMP-dependent transcription factor ATF-6 | |
| | | alpha) (Activating transcription factor 6 alpha) | |
| | | (ATF6-alpha) [Cleaved into: Processed cyclic | |
| | | AMP-dependent transcription factor ATF-6 alpha] | |
| 1122 | ZN233_HUMAN | Zinc finger protein 233 | ZNF233 |
| 1123 | ZN251_HUMAN | Zinc finger protein 251 | ZNF251 |
| 1124 | ZN429_HUMAN | Zinc finger protein 429 | ZNF429 |
| 1125 | ZN534_HUMAN | Zinc finger protein 534 (KRAB domain only | ZNF534 |
| | | protein 3) | KRBO3 |
| 1126 | TCF25_HUMAN | Transcription factor 25 (TCF-25) (Nuclear | TCF25 |
| | | localized protein 1) | KIAA1049 |
| | | | NULP1 |
| | | | FKSG26 |
| 1127 | ZN283_HUMAN | Zinc finger protein 283 (Zinc finger protein | ZNF283 |
| | | HZF19) | |
| 1128 | FOXP4_HUMAN | Forkhead box protein P4 (Fork head-related | FOXP4 |
| | | protein-like A) | FKHLA |
| 1129 | ZN334_HUMAN | Zinc finger protein 334 | ZNF334 |
| 1130 | TBR1_HUMAN | T-box brain protein 1 (T-brain-1) (TBR-1) (TES- | TBR1 |
| | | 56) | |
| 1131 | ZNF16_HUMAN | Zinc finger protein 16 (Zinc finger protein KOX9) | ZNF16 |
| | | | HZF1 |
| | | | KOX9 |
| 1132 | ZNF45_HUMAN | Zinc finger protein 45 (BRC1744) (Zinc finger | ZNF45 |
| | | protein 13) (Zinc finger protein KOX5) | KOX5 |
| | | | ZNF13 |
| 1133 | ZN263_HUMAN | Zinc finger protein 263 (Zinc finger protein | ZNF263 |
| | | FPM315) (Zinc finger protein with KRAB and | FPM315 |
| | | SCAN domains 12) | ZKSCAN12 |
| 1134 | EOMES_HUMAN | Eomesodermin homolog (T-box brain protein 2) | EOMES |
| | | (T-brain-2) (TBR-2) | TBR2 |
| 1135 | ZNF7_HUMAN | Zinc finger protein 7 (Zinc finger protein HF.16) | ZNF7 |
| | | (Zinc finger protein KOX4) | KOX4 |
| 1136 | ZN420_HUMAN | Zinc finger protein 420 | ZNF420 |
| 1137 | NKRF_HUMAN | NF-kappa-B-repressing factor (NFkB-repressing | NKRF |
| | | factor) (Protein ITBA4) (Transcription factor NRF) | ITBA4 |
| | | | NRF |
| 1138 | NOBOX_HUMAN | Homeobox protein NOBOX | NOBOX |
| 1139 | PO6F2_HUMAN | POU domain, class 6, transcription factor 2 | POU6F2 |
| | | (Retina-derived POU domain factor 1) (RPF-1) | RPF1 |
| 1140 | ZN770_HUMAN | Zinc finger protein 770 | ZNF770 |
| 1141 | ZBED5_HUMAN | Zinc finger BED domain-containing protein 5 | ZBED5 |
| | | (Transposon-derived Buster1 transposase-like | Buster1 |
| | | protein) | |
| 1142 | NF2L3_HUMAN | Nuclear factor erythroid 2-related factor 3 (NF-E2- | NFE2L3 |
| | | related factor 3) (NFE2-related factor 3) (Nuclear | NRF3 |
| | | factor, erythroid derived 2, like 3) | |
| 1143 | ZN607_HUMAN | Zinc finger protein 607 | ZNF607 |
| 1144 | ZSC32_HUMAN | Zinc finger and SCAN domain-containing protein | ZSCAN32 |
| | | 32 (Human cervical cancer suppressor gene 5 | ZNF434 |
| | | protein) (HCCS-5) (Zinc finger protein 434) | HCCS5 |
| 1145 | ZNF12_HUMAN | Zinc finger protein 12 (Gonadotropin-inducible | ZNF12 |
| | | ovary transcription repressor 3) (GIOT-3) (Zinc | GIOT3 |
| | | finger protein 325) (Zinc finger protein KOX3) | KOX3 |
| | | | ZNF325 |
| 1146 | ZN782_HUMAN | Zinc finger protein 782 | ZNF782 |
| 1147 | MYBB_HUMAN | Myb-related protein B (B-Myb) (Myb-like protein 2) | MYBL2 |
| | | | BMYB |
| 1148 | ZN234_HUMAN | Zinc finger protein 234 (Zinc finger protein 269) | ZNF234 |
| | | (Zinc finger protein HZF4) | ZNF269 |
| 1149 | ATF6B_HUMAN | Cyclic AMP-dependent transcription factor ATF-6 | ATF6B |
| | | beta (cAMP-dependent transcription factor ATF-6 | CREBL1 |
| | | beta) (Activating transcription factor 6 beta) | G13 |
| | | (ATF6-beta) (Protein G13) (cAMP response | |
| | | element-binding protein-related protein) (Creb-rp) | |
| | | (cAMP-responsive element-binding protein-like 1) | |
| | | [Cleaved into: Processed cyclic AMP-dependent | |
| | | transcription factor ATF-6 beta] | |
| 1150 | ZN611_HUMAN | Zinc finger protein 611 | ZNF611 |
| 1151 | FZD6_HUMAN | Frizzled-6 (Fz-6) (hFz6) | FZD6 |
| 1152 | ZN132_HUMAN | Zinc finger protein 132 | ZNF132 |
| 1153 | ZN225_HUMAN | Zinc finger protein 225 | ZNF225 |
| 1154 | DEND_HUMAN | Dendrin | DDN |
| | | | KIAA0749 |
| 1155 | GZF1_HUMAN | GDNF-inducible zinc finger protein 1 (Zinc finger | GZF1 |
| | | and BTB domain-containing protein 23) (Zinc | ZBTB23 |
| | | finger protein 336) | ZNF336 |
| 1156 | ZN175_HUMAN | Zinc finger protein 175 (Zinc finger protein | ZNF175 |
| | | OTK18) | |
| 1157 | TBX2_HUMAN | T-box transcription factor TBX2 (T-box protein 2) | TBX2 |
| 1158 | ZN544_HUMAN | Zinc finger protein 544 | ZNF544 |
| 1159 | ZN840_HUMAN | Putative zinc finger protein 840 (Zinc finger | ZNF840P |
| | | protein 840 pseudogene) | C20orf157 |
| | | | ZNF840 |
| 1160 | K1958_HUMAN | Uncharacterized protein KIAA1958 | KIAA1958 |
| 1161 | ARNT2_HUMAN | Aryl hydrocarbon receptor nuclear translocator 2 | ARNT2 |
| | | (ARNT protein 2) (Class E basic helix-loop-helix | BHLHE1 |
| | | protein 1) (bHLHe1) | KIAA0307 |
| 1162 | ZN470_HUMAN | Zinc finger protein 470 (Chondrogenesis zinc | ZNF470 |
| | | finger protein 1) (CZF-1) | CZF1 |
| 1163 | ZNF28_HUMAN | Zinc finger protein 28 (Zinc finger protein KOX24) | ZNF28 |
| | | | KOX24 |
| 1164 | ZN219_HUMAN | Zinc finger protein 219 | ZNF219 |
| 1165 | ZN600_HUMAN | Zinc finger protein 600 | ZNF600 |
| 1166 | RFX2_HUMAN | DNA-binding protein RFX2 (Regulatory factor X 2) | RFX2 |
| 1167 | ZN750_HUMAN | Zinc finger protein 750 | ZNF750 |
| 1168 | CARTF_HUMAN | Calcium-responsive transcription factor | CARF |
| | | (Amyotrophic lateral sclerosis 2 chromosomal | ALS2CR8 |
| | | region candidate gene 8 protein) (Calcium-response | |
| | | factor) (CaRF) (Testis development protein NYD- | |
| | | SP24) | |
| 1169 | ZSC10_HUMAN | Zinc finger and SCAN domain-containing protein | ZSCAN10 |
| | | 10 (Zinc finger protein 206) | ZNF206 |
| 1170 | ZN615_HUMAN | Zinc finger protein 615 | ZNF615 |
| 1171 | FOXK1_HUMAN | Forkhead box protein K1 (Myocyte nuclear factor) | FOXK1 |
| | | (MNF) | MNF |
| 1172 | HIC1_HUMAN | Hypermethylated in cancer 1 protein (Hic-1) (Zinc | HIC1 |
| | | finger and BTB domain-containing protein 29) | ZBTB29 |
| 1173 | RFX4_HUMAN | Transcription factor RFX4 (Regulatory factor X 4) | RFX4 |
| | | (Testis development protein NYD-SP10) | |
| 1174 | ZN235_HUMAN | Zinc finger protein 235 (Zinc finger protein 270) | ZNF235 |
| | | (Zinc finger protein 93 homolog) (Zfp-93) (Zinc | ZFP93 |
| | | finger protein HZF6) | ZNF270 |
| 1175 | ZN726_HUMAN | Zinc finger protein 726 | ZNF726 |
| 1176 | SIX5_HUMAN | Homeobox protein SIX5 (DM locus-associated | SIX5 |
| | | homeodomain protein) (Sine oculis homeobox | DMAHP |
| | | homolog 5) | |
| 1177 | ZBT20_HUMAN | Zinc finger and BTB domain-containing protein 20 | ZBTB20 |
| | | (Dendritic-derived BTB/POZ zinc finger protein) | DPZF |
| | | (Zinc finger protein 288) | ZNF288 |
| 1178 | ZN267_HUMAN | Zinc finger protein 267 (Zinc finger protein HZF2) | ZNF267 |
| 1179 | ZN761_HUMAN | Zinc finger protein 761 | ZNF761 |
| | | | KIAA2033 |
| 1180 | STAT4_HUMAN | Signal transducer and activator of transcription 4 | STAT4 |
| 1181 | RFX3_HUMAN | Transcription factor RFX3 (Regulatory factor X 3) | RFX3 |
| 1182 | MYBA_HUMAN | Myb-related protein A (A-Myb) (Myb-like protein 1) | MYBL1 |
| | | | AMYB |
| 1183 | MTF1_HUMAN | Metal regulatory transcription factor 1 (MRE- | MTF1 |
| | | binding transcription factor) (Transcription factor | |
| | | MTF-1) | |
| 1184 | SOX30_HUMAN | Transcription factor SOX-30 | SOX30 |
| 1185 | ZN287_HUMAN | Zinc finger protein 287 (Zinc finger protein with | ZNF287 |
| | | KRAB and SCAN domains 13) | ZKSCAN13 |
| 1186 | ZKSC7_HUMAN | Zinc finger protein with KRAB and SCAN | ZKSCAN7 |
| | | domains 7 (Zinc finger protein 167) (Zinc finger | ZNF167 |
| | | protein 448) (Zinc finger protein 64) | ZNF448 |
| | | | ZNF64 |
| 1187 | P52K_HUMAN | 52 kDa repressor of the inhibitor of the protein | THAP12 |
| | | kinase (p52rIPK) (58 kDa interferon-induced | DAP4 |
| | | protein kinase-interacting protein) (p58IPK- | P52RIPK |
| | | interacting protein) (Death-associated protein 4) | PRKRIR |
| | | (THAP domain-containing protein 0) (THAP | THAP0 |
| | | domain-containing protein 12) | |
| 1188 | ZN711_HUMAN | Zinc finger protein 711 (Zinc finger protein 6) | ZNF711 |
| | | | CMPX1 |
| | | | ZNF6 |
| 1189 | PHTF1_HUMAN | Putative homeodomain transcription factor 1 | PHTF1 |
| | | | PHTF |
| 1190 | SOX5_HUMAN | Transcription factor SOX-5 | SOX5 |
| 1191 | S26A3_HUMAN | Chloride anion exchanger (Down-regulated in | SLC26A3 |
| | | adenoma) (Protein DRA) (Solute carrier family 26 | DRA |
| | | member 3) | |
| 1192 | ZBT49_HUMAN | Zinc finger and BTB domain-containing protein 49 | ZBTB49 |
| | | (Zinc finger protein 509) | ZNF509 |
| 1193 | SIM1_HUMAN | Single-minded homolog 1 (Class E basic helix- | SIM1 |
| | | loop-helix protein 14) (bHLHe14) | BHLHE14 |
| 1194 | Z585A_HUMAN | Zinc finger protein 585A | ZNF585A |
| 1195 | Z585B_HUMAN | Zinc finger protein 585B (zinc finger protein 41- | ZNF585B |
| | | like protein) | |
| 1196 | NF2L1_HUMAN | Nuclear factor erythroid 2-related factor 1 (NF-E2- | NFE2L1 |
| | | related factor 1) (NFE2-related factor 1) (Locus | HBZ17 |
| | | control region-factor 1) (Nuclear factor, erythroid | NRF1 |
| | | derived 2, like 1) (Transcription factor 11) (TCF- | TCF11 |
| | | 11) (Transcription factor HBZ17) (Transcription | |
| | | factor LCR-F1) | |
| 1197 | QRIC1_HUMAN | Glutamine-rich protein 1 | QRICH1 |
| 1198 | ZN33B_HUMAN | Zinc finger protein 33B (Zinc finger protein 11B) | ZNF33B |
| | | (Zinc finger protein KOX2) | KOX2 |
| | | | ZNF11B |
| 1199 | T22D2_HUMAN | TSC22 domain family protein 2 (TSC22-related- | TSC22D2 |
| | | inducible leucine zipper protein 4) | KIAA0669 |
| | | | TILZ4 |
| 1200 | GCFC2_HUMAN | GC-rich sequence DNA-binding factor 2 (GC-rich | GCFC2 |
| | | sequence DNA-binding factor) (Transcription | C2orf3 |
| | | factor 9) (TCF-9) | GCF |
| | | | TCF9 |
| 1201 | SIX4_HUMAN | Homeobox protein SIX4 (Sine oculis homeobox | SIX4 |
| | | homolog 4) | |
| 1202 | SP3_HUMAN | Transcription factor Sp3 (SPR-2) | SP3 |
| 1203 | ZN616_HUMAN | Zinc finger protein 616 | ZNF616 |
| 1204 | E4F1_HUMAN | Transcription factor E4F1 (EC 2.3.2.27) (E4F | E4F1 |
| | | transcription factor 1) (Putative E3 ubiquitin- | E4F |
| | | protein ligase E4F1) (RING-type E3 ubiquitin | |
| | | transferase E4F1) (Transcription factor E4F) | |
| | | (p120E4F) (p50E4F) | |
| 1205 | SP4_HUMAN | Transcription factor Sp4 (SPR-1) | SP4 |
| 1206 | STA5B_HUMAN | Signal transducer and activator of transcription 5B | STAT5B |
| 1207 | ZN606_HUMAN | Zinc finger protein 606 (Zinc finger protein 328) | ZNF606 |
| | | | KIAA1852 |
| | | | ZNF328 |
| 1208 | STA5A_HUMAN | Signal transducer and activator of transcription 5A | STAT5A |
| | | | STAT5 |
| 1209 | ZN148_HUMAN | Zinc finger protein 148 (Transcription factor ZBP- | ZNF148 |
| | | 89) (Zinc finger DNA-binding protein 89) | ZBP89 |
| 1210 | ZN227_HUMAN | Zinc finger protein 227 | ZNF227 |
| 1211 | ZXDA_HUMAN | Zinc finger X-linked protein ZXDA | ZXDA |
| 1212 | NPAS4_HUMAN | Neuronal PAS domain-containing protein 4 | NPAS4 |
| | | (Neuronal PAS4) (Class E basic helix-loop-helix | BHLHE79 |
| | | protein 79) (bHLHe79) (HLH-PAS transcription | NXF |
| | | factor NXF) (PAS domain-containing protein 10) | PASD10 |
| 1213 | ZN226_HUMAN | Zinc finger protein 226 | ZNF226 |
| 1214 | ZN841_HUMAN | Zinc finger protein 841 | ZNF841 |
| 1215 | PGBD1_HUMAN | PiggyBac transposable element-derived protein 1 | PGBD1 |
| | | (Cerebral protein 4) | hucep-4 |
| 1216 | ZNF43_HUMAN | Zinc finger protein 43 (Zinc finger protein 39) | ZNF43 |
| | | (Zinc finger protein HTF6) (Zinc finger protein | KOX27 |
| | | KOX27) | ZNF39 |
| | | | ZNF39L1 |
| 1217 | ZN33A_HUMAN | Zinc finger protein 33A (Zinc finger and ZAK- | ZNF33A |
| | | as sociated protein with KRAB domain) (ZZaPK) | KIAA0065 |
| | | (Zinc finger protein 11A) (Zinc finger protein | KOX31 |
| | | KOX31) | ZNF11 |
| | | | ZNF11A |
| | | | ZNF33 |
| 1218 | ZNF41_HUMAN | Zinc finger protein 41 | ZNF41 |
| 1219 | NPAS2_HUMAN | Neuronal PAS domain-containing protein 2 | NPAS2 |
| | | (Neuronal PAS2) (Basic-helix-loop-helix-PAS | BHLHE9 |
| | | protein MOP4) (Class E basic helix-loop-helix | MOP4 |
| | | protein 9) (bHLHe9) (Member of PAS protein 4) | PASD4 |
| | | (PAS domain-containing protein 4) | |
| 1220 | ZN229_HUMAN | Zinc finger protein 229 | ZNF229 |
| 1221 | SOX6_HUMAN | Transcription factor SOX-6 | SOX6 |
| 1222 | ZN438_HUMAN | Zinc finger protein 438 | ZNF438 |
| 1223 | Z780B_HUMAN | Zinc finger protein 780B (Zinc finger protein 779) | ZNF780B |
| | | | ZNF779 |
| 1224 | BC11A_HUMAN | B-cell lymphoma/leukemia 11A (BCL-11A) (B- | BCL11A |
| | | cell CLL/lymphoma 11A) (COUP-TF-interacting | CTIP1 |
| | | protein 1) (Ecotropic viral integration site 9 protein | EVI9 |
| | | homolog) (EVI-9) (Zinc finger protein 856) | KIAA1809 |
| | | | ZNF856 |
| 1225 | ZN546_HUMAN | Zinc finger protein 546 (Zinc finger protein 49) | ZNF546 |
| | | | ZNF49 |
| 1226 | LZTR1_HUMAN | Leucine-zipper-like transcriptional regulator 1 | LZTR1 |
| | | (LZTR-1) | TCFL2 |
| 1227 | AHR_HUMAN | Aryl hydrocarbon receptor (Ah receptor) (AhR) | AHR |
| | | (Class E basic helix-loop-helix protein 76) | BHLHE76 |
| | | (bHLHe76) | |
| 1228 | MLXPL_HUMAN | Carbohydrate-responsive element-binding protein | MLXIPL |
| | | (ChREBP) (Class D basic helix-loop-helix protein | BHLHD14 |
| | | 14) (bHLHd14) (MLX interactor) (MLX- | MIO |
| | | interacting protein-like) (WS basic-helix-loop-helix | WBSCR14 |
| | | leucine zipper protein) (WS-bHLH) (Williams- | |
| | | Beuren syndrome chromosomal region 14 protein) | |
| 1229 | MACC1_HUMAN | Metastasis-associated in colon cancer protein 1 | MACC1 |
| | | (SH3 domain-containing protein 7a5) | |
| 1230 | ZSC29_HUMAN | Zinc finger and SCAN domain-containing protein | ZSCAN29 |
| | | 29 (Zinc finger protein 690) | ZNF690 |
| 1231 | ZN341_HUMAN | Zinc finger protein 341 | ZNF341 |
| 1232 | C2D1B_HUMAN | Coiled-coil and C2 domain-containing protein 1B | CC2D1B |
| | | (Five prime repressor element under dual | KIAA1836 |
| | | repression-binding protein 2) (FRE under dual | |
| | | repression-binding protein 2) (Freud-2) | |
| 1233 | ZXDC_HUMAN | Zinc finger protein ZXDC (ZXD-like zinc finger | ZXDC |
| | | protein) | ZXDL |
| 1234 | TAF4B_HUMAN | Transcription initiation factor TFIID subunit 4B | TAF4B |
| | | (Transcription initiation factor TFIID 105 kDa | TAF2C2 |
| | | subunit) (TAF(II)105) (TAFII-105) (TAFII105) | TAFII105 |
| 1235 | ZN624_HUMAN | Zinc finger protein 624 | ZNF624 |
| | | | KIAA1349 |
| 1236 | TAF1C_HUMAN | TATA box-binding protein-associated factor RNA | TAF1C |
| | | polymerase I subunit C (RNA polymerase I- | |
| | | specific TBP-associated factor 110 kDa) | |
| | | (TAFI110) (TATA box-binding protein-associated | |
| | | factor 1C) (TBP-associated factor 1C) (Transcription | |
| | | initiation factor SL1/TIF-IB subunit C) | |
| 1237 | ZN629_HUMAN | Zinc finger protein 629 (Zinc finger protein 65) | ZNF629 |
| | | | KIAA0326 |
| | | | ZNF65 |
| 1238 | WFS1_HUMAN | Wolframin | WFS1 |
| 1239 | ZN281_HUMAN | Zinc finger protein 281 (GC-box-binding zinc | ZNF281 |
| | | finger protein 1) (Transcription factor ZBP-99) | GZP1 |
| | | (Zinc finger DNA-binding protein 99) | ZBP99 |
| 1240 | ZN808_HUMAN | Zinc finger protein 808 | ZNF808 |
| 1241 | ZN717_HUMAN | Zinc finger protein 717 (Krueppel-like factor X17) | ZNF717 |
| 1242 | TTF1_HUMAN | Transcription termination factor 1 (TTF-1) (RNA | TTF1 |
| | | polymerase I termination factor) (Transcription | |
| | | termination factor I) (TTF-I) | |
| 1243 | MRFL_HUMAN | Myelin regulatory factor-like protein | MYRFL |
| | | | C12orf15 |
| | | | C12orf28 |
| 1244 | E2F7_HUMAN | Transcription factor E2F7 (E2F-7) | E2F7 |
| 1245 | ZN112_HUMAN | Zinc finger protein 112 (Zfp-112) (Zinc finger | ZNF112 |
| | | protein 228) | ZFP112 |
| | | | ZNF228 |
| 1246 | SAFB1_HUMAN | Scaffold attachment factor B1 (SAF-B) (SAF-B1) | SAFB |
| | | (HSP27 estrogen response element-TATA box- | HAP |
| | | binding protein) (HSP27 ERE-TATA-binding | HET |
| | | protein) | SAFB1 |
| 1247 | PAXB1_HUMAN | PAX3- and PAX7-binding protein 1 (GC-rich | PAXBP1 |
| | | sequence DNA-binding factor 1) | C21orf66 |
| | | | GCFC |
| | | | GCFC1 |
| 1248 | MLXIP_HUMAN | MLX-interacting protein (Class E basic helix-loop- | MLXIP |
| | | helix protein 36) (bHLHe36) (Transcriptional | BHLHE36 |
| | | activator MondoA) | KIAA0867 |
| | | | MIR |
| | | | MONDOA |
| 1249 | RFX6_HUMAN | DNA-binding protein RFX6 (Regulatory factor X 6) | RFX6 |
| | | (Regulatory factor X domain-containing protein 1) | RFXDC1 |
| 1250 | NPAS3_HUMAN | Neuronal PAS domain-containing protein 3 | NPAS3 |
| | | (Neuronal PAS3) (Basic-helix-loop-helix-PAS | BHLHE12 |
| | | protein MOP6) (Class E basic helix-loop-helix | MOP6 |
| | | protein 12) (bHLHe12) (Member of PAS protein 6) | PASD6 |
| | | (PAS domain-containing protein 6) | |
| 1251 | ZN836_HUMAN | Zinc finger protein 836 | ZNF836 |
| 1252 | MYCD_HUMAN | Myocardin | MYOCD |
| | | | MYCD |
| 1253 | C2D1A_HUMAN | Coiled-coil and C2 domain-containing protein 1A | CC2D1A |
| | | (Akt kinase-interacting protein 1) (Five prime | AKI1 |
| | | repressor element under dual repression-binding | |
| | | protein 1) (FRE under dual repression-binding | |
| | | protein 1) (Freud-1) (Putative NF-kappa-B- | |
| | | activating protein 023N) | |
| 1254 | SAFB2_HUMAN | Scaffold attachment factor B2 (SAF-B2) | SAFB2 |
| | | | KIAA0138 |
| 1255 | TR150_HUMAN | Thyroid hormone receptor-associated protein 3 | THRAP3 |
| | | (Thyroid hormone receptor-associated protein | TRAP150 |
| | | complex 150 kDa component) (Trap 150) | |
| 1256 | ZKSC2_HUMAN | Zinc finger protein with KRAB and SCAN | ZKSCAN2 |
| | | domains 2 (Zinc finger protein 694) | ZNF694 |
| 1257 | ZBED6_HUMAN | Zinc finger BED domain-containing protein 6 | ZBED6 |
| 1258 | JMY_HUMAN | Junction-mediating and -regulatory protein | JMY |
| 1259 | STOX1_HUMAN | Storkhead-box protein 1 (Winged-helix domain- | STOX1 |
| | | containing protein) | C10orf24 |
| 1260 | BNC1_HUMAN | Zinc finger protein basonuclin-1 | BNC1 |
| | | | BNC |
| 1261 | SALL2_HUMAN | Sal-like protein 2 (Zinc finger protein 795) (Zinc | SALL2 |
| | | finger protein SALL2) (Zinc finger protein Spalt-2) | KIAA0360 |
| | | (Sal-2) (hSal2) | SAL2 |
| | | | ZNF795 |
| 1262 | ZBTB4_HUMAN | Zinc finger and BTB domain-containing protein 4 | ZBTB4 |
| | | (KAISO-like zinc finger protein 1) (KAISO-L1) | KIAA1538 |
| 1263 | ZN197_HUMAN | Zinc finger protein 197 (Zinc finger protein with | ZNF197 |
| | | KRAB and SCAN domains 9) (ZnF20) (pVHL- | ZKSCAN9 |
| | | associated KRAB domain-containing protein) | ZNF166 |
| 1264 | ZN445_HUMAN | Zinc finger protein 445 (Zinc finger protein 168) | ZNF445 |
| | | (Zinc finger protein with KRAB and SCAN | ZKSCAN15 |
| | | domains 15) | ZNF168 |
| 1265 | ZSC20_HUMAN | Zinc finger and SCAN domain-containing protein | ZSCAN20 |
| | | 20 (Zinc finger protein 31) (Zinc finger protein | KOX29 |
| | | 360) (Zinc finger protein KOX29) | ZNF31 |
| | | | ZNF360 |
| 1266 | EMSA1_HUMAN | ELM2 and SANT domain-containing protein 1 | ELMSAN1 |
| | | (MIDEAS) | C14orf117 |
| | | | C14orf43 |
| 1267 | EVI1_HUMAN | MDS1 and EVI1 complex locus protein EVI1 | MECOM |
| | | (Ecotropic virus integration site 1 protein homolog) | EVI1 |
| | | (EVI-1) | |
| 1268 | SALL4_HUMAN | Sal-like protein 4 (Zinc finger protein 797) (Zinc | SALL4 |
| | | finger protein SALL4) | ZNF797 |
| 1269 | CEBPZ_HUMAN | CCAAT/enhancer-binding protein zeta (CCAAT- | CEBPZ |
| | | box-binding transcription factor) (CBF) (CCAAT- | CBF2 |
| | | binding factor) | |
| 1270 | ZN628_HUMAN | Zinc finger protein 628 | ZNF628 |
| 1271 | ZN658_HUMAN | Zinc finger protein 658 | ZNF658 |
| 1272 | T22D1_HUMAN | TSC22 domain family protein 1 (Cerebral protein | TSC22D1 |
| | | 2) (Regulatory protein TSC-22) (TGFB-stimulated | KIAA1994 |
| | | clone 22 homolog) (Transforming growth factor | TGFB1I4 |
| | | beta-1-induced transcript 4 protein) | TSC22 |
| | | | hucep-2 |
| 1273 | Z518B_HUMAN | Zinc finger protein 518B | ZNF518B |
| | | | KIAA1729 |
| 1274 | CAN15_HUMAN | Calpain-15 (EC 3.4.22.—) (Small optic lobes | CAPN15 |
| | | homolog) | SOLH |
| 1275 | NFX1_HUMAN | Transcriptional repressor NF-X1 (EC 6.3.2.—) | NFX1 |
| | | (Nuclear transcription factor, X box-binding | NFX2 |
| | | protein 1) | |
| 1276 | MYT1_HUMAN | Myelin transcription factor 1 (MyT1) (Myelin | MYT1 |
| | | transcription factor I) (MyTI) (PLPB1) (Proteolipid | KIAA0835 |
| | | protein-binding protein) | KIAA1050 |
| | | | MTF1 |
| | | | MYTI |
| | | | PLPB1 |
| 1277 | ZMYM1_HUMAN | Zinc finger MYM-type protein 1 | ZMYM1 |
| 1278 | MYRF_HUMAN | Myelin regulatory factor (EC 3.4.—.—) (Myelin gene | MYRF |
| | | regulatory factor) [Cleaved into: Myelin regulatory | C11orf9 |
| | | factor, N-terminal; Myelin regulatory factor, C- | KIAA0954 |
| | | terminal] | MRF |
| 1279 | FOG2_HUMAN | Zinc finger protein ZFPM2 (Friend of GATA | ZFPM2 |
| | | protein 2) (FOG-2) (Friend of GATA 2) (hFOG-2) | FOG2 |
| | | (Zinc finger protein 89B) (Zinc finger protein | ZNF89B |
| | | multitype 2) | |
| 1280 | AEBP1_HUMAN | Adipocyte enhancer-binding protein 1 (AE-binding | AEBP1 |
| | | protein 1) (Aortic carboxypeptidase-like protein) | ACLP |
| 1281 | ZN516_HUMAN | Zinc finger protein 516 | ZNF516 |
| | | | KIAA0222 |
| 1282 | ZBED4_HUMAN | Zinc finger BED domain-containing protein 4 | ZBED4 |
| | | | KIAA0637 |
| 1283 | MYT1L_HUMAN | Myelin transcription factor 1-like protein (MyT1- | MYT1L |
| | | L) (MyT1L) | KIAA1106 |
| 1284 | HAIR_HUMAN | Lysine-specific demethylase hairless (EC 1.14.11.—) | HR |
| 1285 | ZNF91_HUMAN | Zinc finger protein 91 (Zinc finger protein HPF7) | ZNF91 |
| | | (Zinc finger protein HTF10) | |
| 1286 | ZBT38_HUMAN | Zinc finger and BTB domain-containing protein 38 | ZBTB38 |
| 1287 | HIPK2_HUMAN | Homeodomain-interacting protein kinase 2 | HIPK2 |
| | | (hHIPk2) (EC 2.7.11.1) | |
| 1288 | TREF1_HUMAN | Transcriptional-regulating factor 1 (Breast cancer | TRERF1 |
| | | anti-estrogen resistance 2) (Transcriptional- | BCAR2 |
| | | regulating protein 132) (Zinc finger protein rapa) | RAPA |
| | | (Zinc finger transcription factor TReP-132) | TREP132 |
| 1289 | CMTA2_HUMAN | Calmodulin-binding transcription activator 2 | CAMTA2 |
| | | | KIAA0909 |
| 1290 | BRD8_HUMAN | Bromodomain-containing protein 8 (Skeletal | BRD8 |
| | | muscle abundant protein) (Skeletal muscle | SMAP |
| | | abundant protein 2) (Thyroid hormone receptor | SMAP2 |
| | | coactivating protein of 120 kDa) (TrCP120) (p120) | |
| 1291 | PER2_HUMAN | Period circadian protein homolog 2 (hPER2) | PER2 |
| | | (Circadian clock protein PERIOD 2) | KIAA0347 |
| 1292 | TRPS1_HUMAN | Zinc finger transcription factor Trps1 (Tricho- | TRPS1 |
| | | rhino-phalangeal syndrome type I protein) (Zinc | |
| | | finger protein GC79) | |
| 1293 | SALL3_HUMAN | Sal-like protein 3 (Zinc finger protein 796) (Zinc | SALL3 |
| | | finger protein SALL3) (hSALL3) | ZNF796 |
| 1294 | STK36_HUMAN | Serine/threonine-protein kinase 36 (EC 2.7.11.1) | STK36 |
| | | (Fused homolog) | KIAA1278 |
| 1295 | KDM3A_HUMAN | Lysine-specific demethylase 3A (EC 1.14.11.—) | KDM3A |
| | | (JmjC domain-containing histone demethylation | JHDM2A |
| | | protein 2A) (Jumonji domain-containing protein 1A) | JMJD1 |
| | | | JMJD1A |
| | | | KIAA0742 |
| | | | TSGA |
| 1296 | SALL1_HUMAN | Sal-like protein 1 (Spalt-like transcription factor 1) | SALL1 |
| | | (Zinc finger protein 794) (Zinc finger protein | SAL1 |
| | | SALL1) (Zinc finger protein Spalt-1) (HSal1) (Sal-1) | ZNF794 |
| 1297 | SCND3_HUMAN | SCAN domain-containing protein 3 (Transposon- | ZBED9 |
| | | derived Buster4 transposase-like protein) (Zinc | Buster4 |
| | | finger BED domain-containing protein 9) | KIAA1925 |
| | | | SCAND3 |
| | | | ZNF305P2 |
| | | | ZNF452 |
| 1298 | ZMYM6_HUMAN | Zinc finger MYM-type protein 6 (Transposon- | ZMYM6 |
| | | derived Buster2 transposase-like protein) (Zinc | Buster2 |
| | | finger protein 258) | KIAA1353 |
| | | | ZNF258 |
| 1299 | MBB1A_HUMAN | Myb-binding protein 1A | MYBBP1A |
| | | | P160 |
| 1300 | ZN335_HUMAN | Zinc finger protein 335 (NRC-interacting factor 1) | ZNF335 |
| | | (NIF-1) | |
| 1301 | ZN541_HUMAN | Zinc finger protein 541 | ZNF541 |
| 1302 | ZMYM3_HUMAN | Zinc finger MYM-type protein 3 (Zinc finger | ZMYM3 |
| | | protein 261) | DXS6673E |
| | | | KIAA0385 |
| | | | ZNF261 |
| 1303 | ZMYM2_HUMAN | Zinc finger MYM-type protein 2 (Fused in | ZMYM2 |
| | | myeloproliferative disorders protein) (Rearranged | FIM |
| | | in atypical myeloproliferative disorder protein) | RAMP |
| | | (Zinc finger protein 198) | ZNF198 |
| 1304 | AN30A_HUMAN | Ankyrin repeat domain-containing protein 30A | ANKRD30A |
| | | (Serologically defined breast cancer antigen NY- | |
| | | BR-1) | |
| 1305 | AKNA_HUMAN | AT-hook-containing transcription factor | AKNA |
| | | | KIAA1968 |
| 1306 | PTC1_HUMAN | Protein patched homolog 1 (PTC) (PTC1) | PTCH1 |
| | | | PTCH |
| 1307 | FACD2_HUMAN | Fanconi anemia group D2 protein (Protein FACD2) | FANCD2 |
| | | | FACD |
| 1308 | FANCA_HUMAN | Fanconi anemia group A protein (Protein FACA) | FANCA |
| | | | FAA |
| | | | FACA |
| | | | FANCH |
| 1309 | SNPC4_HUMAN | snRNA-activating protein complex subunit 4 | SNAPC4 |
| | | (SNAPc subunit 4) (Proximal sequence element- | SNAP190 |
| | | binding transcription factor subunit alpha) (PSE- | |
| | | binding factor subunit alpha) (PTF subunit alpha) | |
| | | (snRNA-activating protein complex 190 kDa | |
| | | subunit) (SNAPc 190 kDa subunit) | |
| 1310 | Z518A_HUMAN | Zinc finger protein 518A | ZNF518A |
| | | | KIAA0335 |
| | | | ZNF518 |
| 1311 | ZMYM4_HUMAN | Zinc finger MYM-type protein 4 (Zinc finger | ZMYM4 |
| | | protein 262) | KIAA0425 |
| | | | ZNF262 |
| 1312 | GLI2_HUMAN | Zinc finger protein GLI2 (GLI family zinc finger | GLI2 |
| | | protein 2) (Tax helper protein) | THP |
| 1313 | ARHG5_HUMAN | Rho guanine nucleotide exchange factor 5 | ARHGEF5 |
| | | (Ephexin-3) (Guanine nucleotide regulatory protein | TIM |
| | | TIM) (Oncogene TIM) (Transforming | |
| | | immortalized mammary oncogene) (p60 TIM) | |
| 1314 | LRP5_HUMAN | Low-density lipoprotein receptor-related protein 5 | LRP5 |
| | | (LRP-5) | LR3 |
| | | | LRP7 |
| 1315 | PPRC1_HUMAN | Peroxisome proliferator-activated receptor gamma | PPRC1 |
| | | coactivator-related protein 1 (PGC-1-related | KIAA0595 |
| | | coactivator) (PRC) | |
| 1316 | RREB1_HUMAN | Ras-responsive element-binding protein 1 (RREB- | RREB1 |
| | | 1) (Finger protein in nuclear bodies) (Raf- | FINB |
| | | responsive zinc finger protein LZ321) (Zinc finger | |
| | | motif enhancer-binding protein 1) (Zep-1) | |
| 1317 | SPT6H_HUMAN | Transcription elongation factor SPT6 (hSPT6) | SUPT6H |
| | | (Histone chaperone suppressor of Ty6) (Tat- | KIAA0162 |
| | | cotransactivator 2 protein) (Tat-CT2 protein) | SPT6H |
| 1318 | BTAF1_HUMAN | TATA-binding protein-associated factor 172 (EC | BTAF1 |
| | | 3.6.4.—) (ATP-dependent helicase BTAF1) (B- | TAF172 |
| | | TFIID transcription factor-associated 170 kDa | |
| | | subunit) (TAF(II)170) (TBP-associated factor 172) | |
| | | (TAF-172) | |
| 1319 | NLRC5_HUMAN | Protein NLRC5 (Caterpiller protein 16.1) | NLRC5 |
| | | (CLR16.1) (Nucleotide-binding oligomerization | NOD27 |
| | | domain protein 27) (Nucleotide-binding | NOD4 |
| | | oligomerization domain protein 4) | |
| 1320 | RAI1_HUMAN | Retinoic acid-induced protein 1 | RAI1 |
| | | | KIAA1820 |
| 1321 | ZNFX1_HUMAN | NFX1-type zinc finger-containing protein 1 | ZNFX1 |
| | | | KIAA1404 |
| 1322 | TCF20_HUMAN | Transcription factor 20 (TCF-20) (Nuclear factor | TCF20 |
| | | SPBP) (Protein AR1) (Stromelysin-1 PDGF- | KIAA0292 |
| | | responsive element-binding protein) (SPRE- | SPBP |
| | | binding protein) | |
| 1323 | TF3C1_HUMAN | General transcription factor 3C polypeptide 1 | GTF3C1 |
| | | (TF3C-alpha) (TFIIIC box B-binding subunit) | |
| | | (Transcription factor IIIC 220 kDa subunit) | |
| | | (TFIIIC 220 kDa subunit) (TFIIIC220) | |
| | | (Transcription factor IIIC subunit alpha) | |
| 1324 | MED12_HUMAN | Mediator of RNA polymerase II transcription | MED12 |
| | | subunit 12 (Activator-recruited cofactor 240 kDa | ARC240 |
| | | component) (ARC240) (CAG repeat protein 45) | CAGH45 |
| | | (Mediator complex subunit 12) (OPA-containing | HOPA |
| | | protein) (Thyroid hormone receptor-associated | KIAA0192 |
| | | protein complex 230 kDa component) (Trap230) | TNRC11 |
| | | (Trinucleotide repeat-containing gene 11 protein) | TRAP230 |
| 1325 | ELYS_HUMAN | Protein ELYS (Embryonic large molecule derived | AHCTF1 |
| | | from yolk sac) (Protein MEL-28) (Putative AT- | ELYS |
| | | hook-containing transcription factor 1) | TMBS62 |
| | | | MSTP108 |
| 1326 | ZEP3_HUMAN | Transcription factor HIVEP3 (Human | HIVEP3 |
| | | immunodeficiency virus type I enhancer-binding | KBP1 |
| | | protein 3) (Kappa-B and V(D)J recombination | KIAA1555 |
| | | signal sequences-binding protein) (Kappa-binding | KRC |
| | | protein 1) (KBP-1) (Zinc finger protein ZAS3) | ZAS3 |
| 1327 | ZEP2_HUMAN | Transcription factor HIVEP2 (Human | HIVEP2 |
| | | immunodeficiency virus type I enhancer-binding | |
| | | protein 2) (HIV-EP2) (MHC-binding protein 2) | |
| | | (MBP-2) | |
| 1328 | SETX_HUMAN | Probable helicase senataxin (EC 3.6.4.—) | SETX |
| | | (Amyotrophic lateral sclerosis 4 protein) (SEN1 | ALS4 |
| | | homolog) (Senataxin) | KIAA0625 |
| | | | SCAR1 |
| 1329 | MGAP_HUMAN | MAX gene-associated protein (MAX dimerization | MGA |
| | | protein 5) | KIAA0518 |
| | | | MAD5 |
| 1330 | GOGB1_HUMAN | Golgin subfamily B member 1 (372 kDa Golgi | GOLGB1 |
| | | complex-associated protein) (GCP372) (Giantin) | |
| | | (Macrogolgin) | |
| 1331 | ASC_HUMAN | Apoptosis-associated speck-like protein containing | PYCARD |
| | | a CARD (hASC) (Caspase recruitment domain- | ASC |
| | | containing protein 5) (PYD and CARD domain- | CARD5 |
| | | containing protein) (Target of methylation-induced | TMS1 |
| | | silencing 1) | |
| 1332 | BCL2_HUMAN | Apoptosis regulator Bcl-2 | BCL2 |
| 1333 | ID3_HUMAN | DNA-binding protein inhibitor ID-3 (Class B basic | ID3 |
| | | helix-loop-helix protein 25) (bHLHb25) (Helix- | 1R21 |
| | | loop-helix protein HEIR-1) (ID-like protein | BHLHB25 |
| | | inhibitor HLH 1R21) (Inhibitor of DNA binding 3) | HEIR1 |
| | | (Inhibitor of differentiation 3) | |
| 1334 | ID2_HUMAN | DNA-binding protein inhibitor ID-2 (Class B basic | ID2 |
| | | helix-loop-helix protein 26) (bHLHb26) (Inhibitor | BHLHB26 |
| | | of DNA binding 2) (Inhibitor of differentiation 2) | |
| 1335 | PHB_HUMAN | Prohibitin | PHB |
| 1336 | LN28A_HUMAN | Protein lin-28 homolog A (Lin-28A) (Zinc finger | LIN28A |
| | | CCHC domain-containing protein 1) | CSDD1 |
| | | | LIN28 |
| | | | ZCCHC1 |
| 1337 | HNRPD_HUMAN | Heterogeneous nuclear ribonucleoprotein D0 | HNRNPD |
| | | (hnRNP D0) (AU-rich element RNA-binding | AUF1 |
| | | protein 1) | HNRPD |
| 1338 | TADBP_HUMAN | TAR DNA-binding protein 43 (TDP-43) | TARDBP |
| | | | TDP43 |
| 1339 | HNRPK_HUMAN | Heterogeneous nuclear ribonucleoprotein K | HNRNPK |
| | | (hnRNP K) (Transformation up-regulated nuclear | HNRPK |
| | | protein) (TUNP) | |
| 1340 | G3BP1_HUMAN | Ras GTPase-activating protein-binding protein 1 | G3BP1 |
| | | (G3BP-1) (EC 3.6.4.12) (EC 3.6.4.13) (ATP- | G3BP |
| | | dependent DNA helicase VIII) (hDH VIII) (GAP | |
| | | SH3 domain-binding protein 1) | |
| 1341 | NONO_HUMAN | Non-POU domain-containing octamer-binding | NONO |
| | | protein (NonO protein) (54 kDa nuclear RNA- and | NRB54 |
| | | DNA-binding protein) (55 kDa nuclear protein) | |
| | | (DNA-binding p52/p100 complex, 52 kDa subunit) | |
| | | (NMT55) (p54(nrb)) (p54nrb) | |
| 1342 | FOXO3_HUMAN | Forkhead box protein O3 (AF6q21 protein) | FOXO3 |
| | | (Forkhead in rhabdomyosarcoma-like 1) | FKHRL1 |
| | | | FOXO3A |
| 1343 | CPEB3_HUMAN | Cytoplasmic polyadenylation element-binding | CPEB3 |
| | | protein 3 (CPE-BP3) (CPE-binding protein 3) | KIAA0940 |
| | | (hCPEB-3) | |
| 1344 | AGO1_HUMAN | Protein argonaute-1 (Argonautel) (hAgo1) | AGO1 |
| | | (Argonaute RISC catalytic component 1) | EIF2C1 |
| | | (Eukaryotic translation initiation factor 2C 1) (eIF- | |
| | | 2C 1) (eIF2C 1) (Putative RNA-binding protein | |
| | | Q99) | |
| 1345 | SUMO1_HUMAN | Small ubiquitin-related modifier 1 (SUMO-1) | SUMO1 |
| | | (GAP-modifying protein 1) (GMP1) (SMT3 | SMT3C |
| | | homolog 3) (Sentrin) (Ubiquitin-homology domain | SMT3H 3 |
| | | protein PIC1) (Ubiquitin-like protein SMT3C) | UBL1 |
| | | (Smt3C) (Ubiquitin-like protein UBL1) | OK/SW-cl.43 |
| 1346 | XCL1_HUMAN | Lymphotactin (ATAC) (C motif chemokine 1) | XCL1 |
| | | (Cytokine SCM-1) (Lymphotaxin) (SCM-1-alpha) | LTN |
| | | (Small-inducible cytokine C1) (XC chemokine | SCYC1 |
| | | ligand 1) | |
| 1347 | NDP_HUMAN | Norrin (Norrie disease protein) (X-linked exudative | NDP |
| | | vitreoretinopathy 2 protein) | EVR2 |
| 1348 | UBC9_HUMAN | SUMO-conjugating enzyme UBC9 (EC 2.3.2.—) | UBE2I |
| | | (RING-type E3 SUMO transferase UBC9) | UBC9 |
| | | (SUMO-protein ligase) (Ubiquitin carrier protein 9) | UBCE9 |
| | | (Ubiquitin carrier protein I) (Ubiquitin-conjugating | |
| | | enzyme E2 I) (Ubiquitin-protein ligase I) (p18) | |
| 1349 | TNFL4_HUMAN | Tumor necrosis factor ligand superfamily member | TNFSF4 |
| | | 4 (Glycoprotein Gp34) (OX40 ligand) (OX40L) | TXGP1 |
| | | (TAX transcriptionally-activated glycoprotein 1) | |
| | | (CD antigen CD252) | |
| 1350 | TNFA_HUMAN | Tumor necrosis factor (Cachectin) (TNF-alpha) | TNF |
| | | (Tumor necrosis factor ligand superfamily member | TNFA |
| | | 2) (TNF-a) [Cleaved into: Tumor necrosis factor, | TNFSF2 |
| | | membrane form (N-terminal fragment) (NTF); | |
| | | Intracellular domain 1 (ICD1); Intracellular domain | |
| | | 2 (ICD2); C-domain 1; C-domain 2; Tumor | |
| | | necrosis factor, soluble form] | |
| 1351 | TNR4_HUMAN | Tumor necrosis factor receptor superfamily | TNFRSF4 |
| | | member 4 (ACT35 antigen) (OX40L receptor) | TXGP1L |
| | | (TAX transcriptionally-activated glycoprotein 1 | |
| | | receptor) (CD antigen CD134) | |
| 1352 | TNF11_HUMAN | Tumor necrosis factor ligand superfamily member | TNFSF11 |
| | | 11 (Osteoclast differentiation factor) (ODF) | OPGL |
| | | (Osteoprotegerin ligand) (OPGL) (Receptor | RANKL |
| | | activator of nuclear factor kappa-B ligand) | TRANCE |
| | | (RANKL) (TNF-related activation-induced | |
| | | cytokine) (TRANCE) (CD antigen CD254) | |
| | | [Cleaved into: Tumor necrosis factor ligand | |
| | | superfamily member 11, membrane form; Tumor | |
| | | necrosis factor ligand superfamily member 11, | |
| | | soluble form] | |
| 1353 | NECD_HUMAN | Necdin | NDN |
| 1354 | TRIB1_HUMAN | Tribbles homolog 1 (TRB-1) (G-protein-coupled | TRIB1 |
| | | receptor-induced gene 2 protein) (GIG-2) (SKIP1) | C8FW |
| | | | GIG2 |
| | | | TRB1 |
| 1355 | BMR1A_HUMAN | Bone morphogenetic protein receptor type-1A | BMPR1A |
| | | (BMP type-1A receptor) (BMPR-1A) (EC | ACVRLK3 |
| | | 2.7.11.30) (Activin receptor-like kinase 3) (ALK-3) | ALK3 |
| | | (Serine/threonine-protein kinase receptor R5) | |
| | | (SKR5) (CD antigen CD292) | |
| 1356 | FZD4_HUMAN | Frizzled-4 (Fz-4) (hFz4) (FzE4) (CD antigen | FZD4 |
| | | CD344) | |
| 1357 | ZNT9_HUMAN | Zinc transporter 9 (ZnT-9) (Human embryonic lung | SLC30A9 |
| | | protein) (HuEL) (Solute carrier family 30 member 9) | C4orf1 |
| | | | HUEL |
| 1358 | TNR11_HUMAN | Tumor necrosis factor receptor superfamily | TNFRSF11A |
| | | member 11A (Osteoclast differentiation factor | RANK |
| | | receptor) (ODFR) (Receptor activator of NF-KB) | |
| | | (CD antigen CD265) | |
| 1359 | TF7L2_HUMAN | Transcription factor 7-like 2 (HMG box | TCF7L2 |
| | | transcription factor 4) (T-cell-specific transcription | TCF4 |
| | | factor 4) (T-cell factor 4) (TCF-4) (hTCF-4) | |
| 1360 | DVL2_HUMAN | Segment polarity protein dishevelled homolog | DVL2 |
| | | DVL-2 (Dishevelled-2) (DSH homolog 2) | |
| 1361 | CTNB1_HUMAN | Catenin beta-1 (Beta-catenin) | CTNNB1 |
| | | | CTNNB |
| | | | OK/SW-cl.35 |
| | | | PRO2286 |
| 1362 | NLRP3_HUMAN | NACHT, LRR and PYD domains-containing | NLRP3 |
| | | protein 3 (Angiotensin/vasopressin receptor | C1orf7 |
| | | AII/AVP-like) (Caterpiller protein 1.1) (CLR1.1) | CIAS1 |
| | | (Cold-induced autoinflammatory syndrome 1 | NALP3 |
| | | protein) (Cryopyrin) (PYRIN-containing APAF1- | PYPAF1 |
| | | like protein 1) | |
| 1363 | LRP6_HUMAN | Low-density lipoprotein receptor-related protein 6 | LRP6 |
| | | (LRP-6) | |
| 1364 | NOTC1_HUMAN | Neurogenic locus notch homolog protein 1 (Notch | NOTCH1 |
| | | 1) (hN1) (Translocation-associated notch protein | TAN1 |
| | | TAN-1) [Cleaved into: Notch 1 extracellular | |
| | | truncation (NEXT); Notch 1 intracellular domain | |
| | | (NICD)] | |
| 1365 | PCBP1_HUMAN | Poly(rC)-binding protein 1 (Alpha-CP1) | PCBP1 |
| | | (Heterogeneous nuclear ribonucleoprotein E1) | |
| | | (hnRNP E1) (Nucleic acid-binding protein | |
| | | SUB2.3) | |
| 1366 | BUD31_HUMAN | Protein BUD31 homolog (Protein EDG-2) (Protein | BUD31 |
| | | G10 homolog) | EDG2 |
| 1367 | YBOX1_HUMAN | Nuclease-sensitive element-binding protein 1 | YBX1 |
| | | (CCAAT-binding transcription factor I subunit A) | NSEP1 |
| | | (CBF-A) (DNA-binding protein B) (DBPB) | YB1 |
| | | (Enhancer factor I subunit A) (EFI-A) (Y-box | |
| | | transcription factor) (Y-box-binding protein 1) | |
| | | (YB-1) | |
| 1368 | ZRAB2_HUMAN | Zinc finger Ran-binding domain-containing protein 2 | ZRANB2 |
| | | (Zinc finger protein 265) (Zinc finger, splicing) | ZIS |
| | | | ZNF265 |
| 1369 | SFPQ_HUMAN | Splicing factor, proline- and glutamine-rich (100 | SFPQ |
| | | kDa DNA-pairing protein) (hPOMp100) (DNA- | PSF |
| | | binding p52/p100 complex, 100 kDa subunit) | |
| | | (Polypyrimidine tract-binding protein-associated- | |
| | | splicing factor) (PSF) (PTB-associated-splicing | |
| | | factor) | |
| 1370 | CNBP1_HUMAN | Beta-catenin-interacting protein 1 (Inhibitor of | CTNNBIP1 |
| | | beta-catenin and Tcf-4) | ICAT |
| 1371 | HMGA1_HUMAN | High mobility group protein HMG-I/HMG-Y | HMGA1 |
| | | (HMG-I(Y)) (High mobility group AT-hook | HMGIY |
| | | protein 1) (High mobility group protein A1) (High | |
| | | mobility group protein R) | |
| 1372 | TCP4_HUMAN | Activated RNA polymerase II transcriptional | SUB1 |
| | | coactivator p15 (Positive cofactor 4) (PC4) (SUB1 | PC4 |
| | | homolog) (p14) | RPO2TC1 |
| 1373 | IL5_HUMAN | Interleukin-5 (IL-5) (B-cell differentiation factor I) | IL5 |
| | | (Eosinophil differentiation factor) (T-cell replacing | |
| | | factor) (TRF) | |
| 1374 | IL4_HUMAN | Interleukin-4 (IL-4) (B-cell stimulatory factor 1) | IL4 |
| | | (BSF-1) (Binetrakin) (Lymphocyte stimulatory | |
| | | factor 1) (Pitrakinra) | |
| 1375 | RBTN2_HUMAN | Rhombotin-2 (Cysteine-rich protein TTG-2) (LIM | LMO2 |
| | | domain only protein 2) (LMO-2) (T-cell | RBTN2 |
| | | translocation protein 2) | RBTNL1 |
| | | | RHOM2 |
| | | | TTG2 |
| 1376 | IL10_HUMAN | Interleukin-10 (IL-10) (Cytokine synthesis | IL10 |
| | | inhibitory factor) (CSIF) | |
| 1377 | TWST1_HUMAN | Twist-related protein 1 (Class A basic helix-loop- | TWIST1 |
| | | helix protein 38) (bHLHa38) (H-twist) | BHLHA38 |
| | | | TWIST |
| 1378 | MD2L2_HUMAN | Mitotic spindle assembly checkpoint protein | MAD2L2 |
| | | MAD2B (Mitotic arrest deficient 2-like protein 2) | MAD2B |
| | | (MAD2-like protein 2) (REV7 homolog) (hREV7) | REV7 |
| 1379 | IL6_HUMAN | Interleukin-6 (IL-6) (B-cell stimulatory factor 2) | IL6 |
| | | (BSF-2) (CTL differentiation factor) (CDF) | IFNB2 |
| | | (Hybridoma growth factor) (Interferon beta-2) | |
| | | (IFN-beta-2) | |
| 1380 | HMGB1_HUMAN | High mobility group protein B1 (High mobility | HMGB1 |
| | | group protein 1) (HMG-1) | HMG1 |
| 1381 | OBF1_HUMAN | POU domain class 2-associating factor 1 (B-cell- | POU2AF1 |
| | | specific coactivator OBF-1) (BOB-1) (OCA-B) | OBF1 |
| | | (OCT-binding factor 1) | |
| 1382 | IL1B_HUMAN | Interleukin-1 beta (IL-1 beta) (Catabolin) | IL1B |
| | | | IL1F2 |
| 1383 | CITE2_HUMAN | Cbp/p300-interacting transactivator 2 (MSG- | CITED2 |
| | | related protein 1) (MRG-1) (P35srj) | MRG1 |
| 1384 | RFXAP_HUMAN | Regulatory factor X-as sociated protein (RFX- | RFXAP |
| | | associated protein) (RFX DNA-binding complex | |
| | | 36 kDa subunit) | |
| 1385 | TSNAX_HUMAN | Translin-associated protein X (Translin-associated | TSNAX |
| | | factor X) | TRAX |
| 1386 | SOX2_HUMAN | Transcription factor SOX-2 | SOX2 |
| 1387 | PA2G4_HUMAN | Proliferation-associated protein 2G4 (Cell cycle | PA2G4 |
| | | protein p38-2G4 homolog) (hG4-1) (ErbB3- | EBP1 |
| | | binding protein 1) | |
| 1388 | SMAD3_HUMAN | Mothers against decapentaplegic homolog 3 (MAD | SMAD3 |
| | | homolog 3) (Mad3) (Mothers against DPP homolog | MADH3 |
| | | 3) (hMAD-3) (JV15-2) (SMAD family member 3) | |
| | | (SMAD 3) (Smad3) (hSMAD3) | |
| 1389 | SMAD7_HUMAN | Mothers against decapentaplegic homolog 7 (MAD | SMAD7 |
| | | homolog 7) (Mothers against DPP homolog 7) | MADH7 |
| | | (Mothers against decapentaplegic homolog 8) | MADH8 |
| | | (MAD homolog 8) (Mothers against DPP homolog | |
| | | 8) (SMAD family member 7) (SMAD 7) (Smad7) | |
| | | (hSMAD7) | |
| 1390 | TEAD1_HUMAN | Transcriptional enhancer factor TEF-1 (NTEF-1) | TEAD1 |
| | | (Protein GT-IIC) (TEA domain family member 1) | TCF13 |
| | | (TEAD-1) (Transcription factor 13) (TCF-13) | TEF1 |
| 1391 | CTBP1_HUMAN | C-terminal-binding protein 1 (CtBP1) (EC 1.1.1.—) | CTBP1 |
| | | | CTBP |
| 1392 | TEAD2_HUMAN | Transcriptional enhancer factor TEF-4 (TEA | TEAD2 |
| | | domain family member 2) (TEAD-2) | TEF4 |
| 1393 | BCL3_HUMAN | B-cell lymphoma 3 protein (BCL-3) (Proto- | BCL3 |
| | | oncogene BCL3) | BCL4 |
| | | | D19S37 |
| 1394 | SMAD1_HUMAN | Mothers against decapentaplegic homolog 1 (MAD | SMAD1 |
| | | homolog 1) (Mothers against DPP homolog 1) | BSP1 |
| | | (JV4-1) (Mad-related protein 1) (SMAD family | MADH1 |
| | | member 1) (SMAD 1) (Smad1) (hSMAD1) | MADR1 |
| | | (Transforming growth factor-beta-signaling protein | |
| | | 1) (BSP-1) | |
| 1395 | SMAD2_HUMAN | Mothers against decapentaplegic homolog 2 (MAD | SMAD2 |
| | | homolog 2) (Mothers against DPP homolog 2) | MADH2 |
| | | (JV18-1) (Mad-related protein 2) (hMAD-2) | MADR2 |
| | | (SMAD family member 2) (SMAD 2) (Smad2) | |
| | | (hSMAD2) | |
| 1396 | GAS7_HUMAN | Growth arrest-specific protein 7 (GAS-7) | GAS7 |
| | | | KIAA0394 |
| 1397 | SUFU_HUMAN | Suppressor of fused homolog (SUFUH) | SUFU |
| | | | UNQ650/ |
| | | | PRO1280 |
| 1398 | RCOR1_HUMAN | REST corepressor 1 (Protein CoREST) | RCOR1 |
| | | | KIAA0071 |
| | | | RCOR |
| 1399 | SMAD4_HUMAN | Mothers against decapentaplegic homolog 4 (MAD | SMAD4 |
| | | homolog 4) (Mothers against DPP homolog 4) | DPC4 |
| | | (Deletion target in pancreatic carcinoma 4) (SMAD | MADH4 |
| | | family member 4) (SMAD 4) (Smad4) (hSMAD4) | |
| 1400 | GMEB1_HUMAN | Glucocorticoid modulatory element-binding protein | GMEB1 |
| | | 1 (GMEB-1) (DNA-binding protein p96PIF) | |
| | | (Parvovirus initiation factor p96) (PIF p96) | |
| 1401 | HIF3A_HUMAN | Hypoxia-inducible factor 3-alpha (HIF-3-alpha) | HIF3A |
| | | (HIF3-alpha) (Basic-helix-loop-helix-PAS protein | BHLHE17 |
| | | MOP7) (Class E basic helix-loop-helix protein 17) | MOP7 |
| | | (bHLHe17) (HIF3 - alpha-1) (Inhibitory PAS | PASD7 |
| | | domain protein) (IPAS) (Member of PAS protein | |
| | | 7) (PAS domain-containing protein 7) | |
| 1402 | SKIL_HUMAN | Ski-like protein (Ski-related oncogene) (Ski-related | SKIL |
| | | protein) | SNO |
| 1403 | BCL6_HUMAN | B-cell lymphoma 6 protein (BCL-6) (B-cell | BCL6 |
| | | lymphoma 5 protein) (BCL-5) (Protein LAZ-3) | BCL5 |
| | | (Zinc finger and BTB domain-containing protein | LAZ3 |
| | | 27) (Zinc finger protein 51) | ZBTB27 |
| | | | ZNF51 |
| 1404 | GAS6_HUMAN | Growth arrest-specific protein 6 (GAS-6) (AXL | GAS6 |
| | | receptor tyrosine kinase ligand) | AXLLG |
| 1405 | PLAK_HUMAN | Junction plakoglobin (Catenin gamma) | JUP |
| | | (Desmoplakin III) (Desmoplakin-3) | CTNNG |
| | | | DP3 |
| 1406 | TIF1B_HUMAN | Transcription intermediary factor 1-beta (TIF1- | TRIM28 |
| | | beta) (E3 SUMO-protein ligase TRIM28) (EC | KAP1 |
| | | 2.3.2.27) (KRAB-associated protein 1) (KAP-1) | RNF96 |
| | | (KRAB-interacting protein 1) (KRIP-1) (Nuclear | TIF1B |
| | | corepressor KAP-1) (RING finger protein 96) | |
| | | (RING-type E3 ubiquitin transferase TIF 1-beta) | |
| | | (Tripartite motif-containing protein 28) | |
| 1407 | VAV_HUMAN | Proto-oncogene vav | VAV1 |
| | | | VAV |
| 1408 | RB_HUMAN | Retinoblastoma-associated protein (p105-Rb) | RB1 |
| | | (pRb) (Rb) (pp110) | |
| 1409 | HIRA_HUMAN | Protein HIRA (TUP1-like enhancer of split protein 1) | HIRA |
| | | | DGCR1 |
| | | | HIR |
| | | | TUPLE1 |
| 1410 | TIF1A_HUMAN | Transcription intermediary factor 1-alpha (TIF1- | TRIM24 |
| | | alpha) (EC 2.3.2.27) (E3 ubiquitin-protein ligase | RNF82 |
| | | TRIM24) (RING finger protein 82) (RING-type E3 | TIF1 |
| | | ubiquitin transferase TIF1-alpha) (Tripartite motif- | TIF1A |
| | | containing protein 24) | |
| 1411 | UBP7_HUMAN | Ubiquitin carboxyl-terminal hydrolase 7 (EC | USP7 |
| | | 3.4.19.12) (Deubiquitinating enzyme 7) | HAUSP |
| | | (Herpesvirus-associated ubiquitin-specific | |
| | | protease) (Ubiquitin thioesterase 7) (Ubiquitin- | |
| | | specific-processing protease 7) | |
| 1412 | SIN3A_HUMAN | Paired amphipathic helix protein Sin3a (Histone | SIN3A |
| | | deacetylase complex subunit Sin3a) | |
| | | (Transcriptional corepressor Sin3a) | |
| 1413 | RERE_HUMAN | Arginine-glutamic acid dipeptide repeats protein | RERE |
| | | (Atrophin-1-like protein) (Atrophin-1-related | ARG |
| | | protein) | ARP |
| | | | ATN1L |
| | | | KIAA0458 |
| 1414 | SMCA4_HUMAN | Transcription activator BRG1 (EC 3.6.4.—) (ATP- | SMARCA4 |
| | | dependent helicase SMARCA4) (BRG1-associated | BAF190A |
| | | factor 190A) (BAF190A) (Mitotic growth and | BRG1 |
| | | transcription activator) (Protein BRG-1) (Protein | SNF2B |
| | | brahma homolog 1) (SNF2-beta) (SWI/SNF-related | SNF2L4 |
| | | matrix-associated actin-dependent regulator of | |
| | | chromatin subfamily A member 4) | |
| 1415 | BCOR_HUMAN | BCL-6 corepressor (BCoR) | BCOR |
| | | | KIAA1575 |
| 1416 | T2AG_HUMAN | Transcription initiation factor IIA subunit 2 | GTF2A2 |
| | | (General transcription factor IIA subunit 2) (TFIIA | TF2A2 |
| | | p12 subunit) (TFIIA-12) (TFIIAS) (Transcription | |
| | | initiation factor IIA gamma chain) (TFIIA-gamma) | |
| 1417 | TAF13_HUMAN | Transcription initiation factor TFIID subunit 13 | TAF13 |
| | | (Transcription initiation factor TFIID 18 kDa | TAF2K |
| | | subunit) (TAF(II)18) (TAFII-18) (TAFII18) | TAFII18 |
| 1418 | TAF12_HUMAN | Transcription initiation factor TFIID subunit 12 | TAF12 |
| | | (Transcription initiation factor TFIID 20/15 kDa | TAF15 |
| | | subunits) (TAFII-20/TAFII-15) | TAF2J |
| | | (TAFII20/TAFII15) | TAFII20 |
| 1419 | TAF5_HUMAN | Transcription initiation factor TFIID subunit 5 | TAF5 |
| | | (Transcription initiation factor TFIID 100 kDa | TAF2D |
| | | subunit) (TAF(II)100) (TAFII-100) (TAFII100) | |
| 1420 | TAF4_HUMAN | Transcription initiation factor TFIID subunit 4 | TAF4 |
| | | (RNA polymerase II TBP-associated factor subunit | TAF2C |
| | | C) (TBP-associated factor 4) (Transcription | TAF2C1 |
| | | initiation factor TFIID 130 kDa subunit) | TAF4A |
| | | (TAF(II)130) (TAFII-130) (TAFII130) | TAFII130 |
| | | (Transcription initiation factor TFIID 135 kDa | TAFII135 |
| | | subunit) (TAF(II)135) (TAFII-135) (TAFII135) | |
| 1421 | TAF1L_HUMAN | Transcription initiation factor TFIID subunit 1-like | TAF1F |
| | | (TAF(II)210) (TBP-associated factor 1-like) (TBP- | |
| | | associated factor 210 kDa) (Transcription initiation | |
| | | factor TFIID 210 kDa subunit) | |
| 1422 | TAF1_HUMAN | Transcription initiation factor TFIID subunit 1 (EC | TAF1 |
| | | 2.3.1.48) (EC 2.7.11.1) (Cell cycle gene 1 protein) | BA2R |
| | | (TBP-associated factor 250 kDa) (p250) | CCG1 |
| | | (Transcription initiation factor TFIID 250 kDa | CCGS |
| | | subunit) (TAF(II)250) (TAFII-250) (TAFII250) | TAF2A |

### Non-Genomic Nucleic Acid Components

In some embodiments, the present disclosure provides technologies for destabilizing or inhibiting genomic complexes (e.g., decreasing incidence of one or more particular genomic complexes) by targeting a non-genomic nucleic acid component of the complex, e.g., using a disrupting agent. In some embodiments, a non-genomic nucleic acid suitable for targeting as described herein is an RNA.

For example, those skilled in the art will be aware that certain genomic complexes (e.g., Type 1, EP subtype loops) may include one or more non-coding RNAs (ncRNAs) such as one or more enhancer RNAs (eRNAs). Those skilled in the art will be aware that eRNAs are typically transcribed from enhancers, and may participate in regulating expression of one or more genes regulated by the enhancer (i.e., target genes of the enhancer). In some embodiments, eRNAs are involved in genomic complexes (e.g., comprising anchor sequence-mediated conjunctions, and particularly Type 1, subtype EP (loops) that include (e.g., co-localize) a given enhancer and a given target gene promoter, for example via interactions with one or more anchor sequence nucleating polypeptides such as CTCF and YY1, general transcription machinery components, Mediator, and/or one or more sequence-specific transcriptional regulatory agents such as p53 or Oct4. In some embodiments, changes in level of one or more eRNAs may result in changes of levels of a given target gene. In some embodiments, disrupting agents may comprise certain components that target one or more eRNAs. In some embodiments, for example, knockdown of an eRNA may cause knockdown of a target gene. As a non-limiting example, targeting of certain eRNAs may result in knockdown of certain target genes. By way of non-limiting example, knockdown of eRNAs listed in Table 3 (below) result in knockdown of particular target genes.

TABLE 3
eRNAs and Known Targets

| eRNA | Target |
|---|---|
| ncRNA-a1 | ECM1 |
| ncRNA-a2 | KLHL12 |
| ncRNA-a3 | TAL1 |
| ncRNA-a4 | CMPK1 |
| ncRNA-a5 | ROCK2 |
| ncRNA-a6 | Snai1 |
| ncRNA-a7 | Snai2 |
| TFF1-eRNA | TFF1 |
| FOXC1-eRNA | FOXCl |
| CA12-eRNA | CA12 |
| PGR-eRNA | PGR |
| SIAH2-eRNA | SIAH2 |
| KCNK5-eRNA | KCNK5 |
| P2RY2-eRNA | P2RY2 |
| SMAD7-eRNA | SMAD7 |
| GREB1-eRNA | GREB1 |
| NRIP1-eRNA | NRIP1 |
| p53BER2 | PAPPA |
| p53BER4 | IER5 |

### Genomic Complex Detection Assays

In some embodiments, certain assays or tests may be conducted to determine presence or extent of one or more genomic complexes (e.g. presence or absence of one or more loops in a given genomic location). In some embodiments, assays are conducted to determine if disruption of a genomic complex has been successful. In some embodiments, localization of genomic complexes may be precisely performed via one or more assays. In some embodiments, assays are structural readouts. In some embodiments, assays are functional readouts. One of skill in the art, reading the present application, will have an understanding as to which assays and visualization techniques would be most appropriate to determine structure and/or function and/or activity (e.g. presence or absence) of genomic complexes.

In some embodiments, assays (e.g., chromatin immunoprecipitation assays) may quantify amount of a particular genomic complex. In some embodiments, assays (e.g., immunostaining assays) may visualize presence of a particular disrupting agent and/or genomic complex. In some embodiments, assays (e.g. fluorescent in situ hybridization assays (FISH) assays) may both visualize and localize presence of a particular disrupting agent and/or genomic complex.

In some embodiments, a disrupting agent will cause a detectable effect on function (e.g. functional assays in which an expected component of a genomic complex is changed in presence of a modulating agent (e.g., disrupting agent), relative to absence of a modulating agent).

In some embodiments, an assay comprises a step of immunoprecipitation, e.g., chromatin immunoprecipitation.

In some embodiments, an assay comprises performing one or more serial chromatin immunoprecipitations, e.g., at least a first chromatin immunoprecipitation using an antibody against a first component of a targeted genomic complex, a second chromatin immunoprecipitation using an antibody against a second component of a targeted genomic complex, and optionally a step to determine presence and/or level of a genomic sequence that is in proximity to the genomic complex (e.g., a PCR assay).

In some embodiments, an assay is a chromosome conformation capture assay. In some embodiments, a chromosome capture assay detects presence and/or level of interactions between a single pair of genomic loci (e.g., a “one vs. one” assay, e.g., a 3C assay). In some embodiments, a chromosome capture assay detects presence and/or level of interactions between one genomic locus and multiple and/or all other genomic loci (e.g., a “one vs. many or all” assay, e.g., a 4C assay). In some embodiments, a chromosome capture assay detects presence and/or level of interactions between multiple and/or many genomic loci within a given region (e.g., a “many vs. many” assay, e.g., a 5C assay). In some embodiments, a chromosome capture assay detects presence and/or level of interactions between all or nearly all genomic loci (e.g., an “all vs. all” assay, e.g., a Hi-C assay).

In some embodiments, an assay comprises a step of cross-linking cell genomes (e.g., using formaldehyde). In some embodiments, an assay comprises a capture step (e.g., using an oligonucleotide) to enrich for specific loci or for a specific locus of interest. In some embodiments, an assay is a single-cell assay.

In some embodiments, an assay detects interactions between genomic loci at a genome-wide level, e.g., a Chromatin Interaction Analysis by Paired-End Tag Sequencing (ChiA-PET) assay.

### Site-Specific Disrupting Agents

As described herein, the present disclosure provides technologies for destabilization and/or inhibiting formation of particular genomic complexes as described herein by contacting a system in which such complexes are to be inhibited or destabilized with a disrupting agent as described herein. As a result of provided technologies, incidence of complex formation and/or stabilization (e.g., number of complexes in a system at a given moment in time, or over a period of time) is decreased by such contacting as compared with extent observed absent such contacting.

In some embodiments, binding to a genomic complex (e.g., a genomic complex component) or genomic site by a disrupting agent as described herein achieves destabilization and/or inhibiting formation of one or more genomic complexes. In some embodiments, destabilization and/or inhibiting formation of a genomic complex comprises destabilization and/or inhibiting formation of a topological structure of the genomic complex. In some embodiments, destabilization and/or inhibiting formation of a topological structure of a genomic complex results in modulated expression of a given target gene. In some embodiments, no detectable destabilization or inhibition of formation of a topological structure is observed, but modulated expression of a given target gene is nonetheless observed.

Those skilled in the art are aware that, in nature, expression of certain genes can be impacted by the presence of an associated genomic complex, and are familiar with the polypeptide and/or nucleic acid components that typically make up such complexes. The present disclosure provides technologies for destabilizing and/or inhibiting formation of such complexes. In some embodiments, provided technologies decrease the incidence of an endogenous genomic complex (i.e., of a complex that naturally forms, to some degree, at a relevant genomic location). Alternatively or additionally, in some embodiments, provided technologies may destabilize and/or inhibit formation of a genomic complex at a location and/or including one or more components, that are not naturally found in a complex at the relevant genomic location, e.g., are not found in a complex at the relevant genomic location in wild-type cells, e.g., are only found in cells comprising or having undergone a gross chromosomal rearrangement or disease cells, e.g., cancer cells.

In some embodiments, provided technologies inhibit recruitment of one or more components of a genomic complex so that complex formation at a particular genomic location or site is inhibited or destabilized. In general, provided technologies achieve decreased incidence of genomic complexes at particular genomic locations.

In some embodiments, a genomic site at which incidence of a genomic complex is decreased in accordance with the present disclosure is or comprises a genomic sequence element such as, for example, an anchor sequence (e.g., that is or comprises a CTCF or YY1 binding site).

In some embodiments, a genomic complex whose incidence is decreased in accordance with the present disclosure comprises or consists of components selected from the group consisting of a genomic sequence element (e.g., a CTCF binding motif, a YY1 binding motif, etc.) recognized by a nucleating component, a plurality of polypeptide components (e.g., CTCF, YY1, cohesion, one or more transcriptional machinery proteins, one or more transcriptional regulatory proteins), and one or more non-genomic nucleic acid components (e.g., non-coding RNA and/or an mRNA, for example, transcribed from a gene associated with the genomic complex). In accordance with the present disclosure, site-specific disrupting agents provided herein include, bind to, and/or otherwise inhibit (e.g., inhibit recruitment of) one or more such components, so that incidence of a genomic complex containing them is decreased at a particular genomic location (e.g., at the genomic sequence element(s), e.g., associated with the target gene). In some particular embodiments, a provided site-specific disrupting agent inhibits (e.g., interacts with, for example binds directly to) a polypeptide that binds to a nucleic acid (e.g., a genomic sequence element such as an anchor sequence element, a non-coding RNA, and/or an mRNA transcribed from an associated gene) at or near the genomic location, and furthermore inhibits (e.g., interacts with, for example binds directly to) one or more other genomic complex components (e.g., one or more polypeptide components of the genomic complex)

In some embodiments, a targeting moiety binds specifically to a genomic site in one or more genomic complexes (e.g., within a cell) and not to non-targeted genomic sites (e.g., within the same cell). In some embodiments, a disrupting agent specifically inhibits formation of and/or destabilizes a genomic complex that is present in only certain cell types and/or only at certain developmental stages or times.

A disrupting agent may bind its target genomic site and destabilize or inhibit formation of a genomic complex (e.g., by altering affinity of the targeted component to one or more other complex components, e.g., by at least 10%, 15%, 20%, 25%, 30%, 35%, 40%, 45%, 50%, 55%, 60%, 65%, 70%, 75%, 80%, 85%, 90%, 95%, or more). Alternatively or additionally, in some embodiments, binding by a disrupting agent alters topology of genomic DNA impacted by a genomic complex, e.g., by at least 10%, 15%, 20%, 25%, 30%, 35%, 40%, 45%, 50%, 55%, 60%, 65%, 70%, 75%, 80%, 85%, 90%, 95%, or more). In some embodiments, a disrupting agent as described herein alters expression of a particular gene associated with a assembled genomic complex, e.g., a target gene, by at least 10%, 15%, 20%, 25%, 30%, 35%, 40%, 45%, 50%, 55%, 60%, 65%, 70%, 75%, 80%, 85%, 90%, 95%, or more.

Embodiments provided herein provide a site-specific disrupting agent that comprises a targeting moiety (e.g., that localizes the disrupting agent to a genomic location or site at which incidence of a genomic complex is decreased in accordance with the present disclosure). In some embodiments, the targeting moiety is also an effector moiety, e.g., disrupting moiety, (e.g., in that it inhibits formation of and/or decreases the presence of the relevant genomic complex); in some embodiments, a site-specific disrupting agent comprises distinct targeting and effector moieties.

Thus, in some embodiments, a provided site-specific disrupting agent is or comprises a targeting moiety and one or more effector moieties. In some embodiments, an effector moiety may be or comprise a disrupting moiety. In some embodiments, an effector moiety may be or comprise a modifying moiety. Alternatively or additionally, in some embodiments, an effector moiety may be or comprises one or more of a tagging moiety, a cleavable moiety, a membrane translocation moiety, a pharmacoagent moiety, etc.

Targeting Moieties

In some embodiments, a disrupting agent is or comprises a targeting moiety. A targeting moiety as described herein targets either (i) a genomic site (e.g., a genomic sequence element) that is or is in the vicinity of the relevant genomic complex being inhibited and/or destabilized; and/or (ii) one or more other genomic complex components that may, for example, represent a partial genomic complex that is destabilized, dissociated, and/or inhibited according to the present disclosure. In some embodiments, a targeting moiety targets DNA and is a DNA-binding moiety. In some embodiments, a targeting moiety targets RNA and is an RNA-binding moiety.

In some embodiments, a targeting moiety targets a genomic site that is or comprises an anchor sequence. In some embodiments, a targeting moiety targets a genomic site that is or comprises a target gene proximal anchor sequence, e.g., a cancer associated anchor sequence. In some embodiments, a targeting moiety targets a genomic site that is not an anchor sequence. In some embodiments, a targeting moiety targets a genomic site that is or comprises a promoter or a transcriptional regulatory sequence. In some embodiments, a targeting moiety targets a genomic site that is or comprises a breakpoint. In some embodiments, a targeting moiety targets a genomic site that has undergone a gross chromosomal rearrangement. In some embodiments, a targeting moiety targets a genomic site comprising a fusion gene, e.g., a fusion oncogene. In some embodiments, a targeting moiety targets a genomic site that is, comprises, or is proximal to a target gene proximal anchor sequence (e.g., a cancer associated anchor sequence).

In some embodiments, a targeting moiety targets a complex component other than a genomic site. For example, in some embodiments, a targeting moiety targets a polypeptide complex component (e.g., a nucleating polypeptide, a transcription machinery polypeptide, a transcription regulator polypeptide, or a combination (e.g., subcomplex) thereof). In some embodiments, a targeting moiety targets a nucleic acid complex component (e.g., other than a genomic sequence element, e.g., a non-genomic nucleic acid component) such as an ncRNA (e.g., an eRNA).

In some embodiments, a targeting moiety targets a genomic site (e.g., a genomic site as described herein) and a complex component other than a genomic site (e.g., as described herein).

In some embodiments, a targeting moiety targets a site listed in Table 9. In some embodiments, a targeting moiety binds to a genomic sequence element proximal to a fusion gene (e.g., fusion oncogene). In some embodiments, a targeting moiety binds to a coding or non-coding sequence of a fusion gene (e.g., fusion oncogene). In some embodiments, a targeting moiety binds to a genomic sequence element situated upstream of a fusion gene (e.g., fusion oncogene). In some embodiments, a targeting moiety binds to an enhancer (e.g., super enhancer) proximal to a fusion gene (e.g., fusion oncogene). In some embodiments, a targeting moiety binds to an enhancer (e.g., super enhancer) situated upstream of a fusion gene (e.g., fusion oncogene). In some embodiments, a targeting moiety binds to a genomic complex (e.g., ASMC), or an anchor sequence associated therewith, comprising the fusion gene (e.g., fusion oncogene). In some embodiments, the fusion gene is a fusion oncogene comprising some or all of CCND1, and the targeting moiety binds to a coding or non-coding sequence of CCND1. In some embodiments, the fusion gene is a fusion oncogene comprising some or all of MYC, and the targeting moiety binds to a coding or non-coding sequence of MYC.

In some embodiments, interaction between a targeting moiety and its targeted component interferes with one or more other interactions that the targeted component would otherwise make. In some embodiments, binding of a targeting moiety to a targeted component prevents the targeted component from interacting with another transcription factor, genomic complex component, or genomic sequence element. In some embodiments, binding of a targeting moiety to a targeted component decreases binding affinity of the targeted component for another transcription factor, genomic complex component, or genomic sequence element. In some embodiments, K_{D} of a targeted component for another transcription factor, genomic complex component, or genomic sequence element increases by at least 1.05× (i.e., 1.05 times), 1.1×, 1.2×, 1.3×, 1.4×, 1.5×, 1.6×, 1.7×, 1.8×, 1.9×, 2×, 3×, 4×, 5×, 6×, 7×, 8×, 9×, 10×, 20×, 50×, or 100× (and optionally no more than 20×, 10×, 9×, 8×, 7×, 6×, 5×, 4×, 3×, 2×, 1.9×, 1.8×, 1.7×, 1.6×, 1.5×, 1.4×, 1.3×, 1.2×, or 1.1×) in presence of a site-specific disrupting agent comprising the targeting moiety than in the absence of the site-specific disrupting agent, comprising the targeting moiety. Changes in K_{D} of a targeted component for another transcription factor, genomic complex component, or genomic sequence element may be evaluated, for example, using ChIP-Seq or ChIP-qPCR.

In some embodiments, binding of a targeting moiety to a targeted component alters, e.g., decreases, the level of a genomic complex (e.g., ASMC) comprising the targeted component. In some embodiments, the level of a genomic complex (e.g., ASMC) comprising the targeted component decreases by at least 10, 20, 30, 40, 50, 60, 70, 80, 90, or 100% (and optionally, up to 100, 90, 80, 70, 60, 50, 40, 30, or 20%) in the presence of a site-specific disrupting agent comprising the targeting moiety relative to the absence of said site-specific disrupting agent. In some embodiments, binding of a targeting moiety to a targeted component alters, e.g., decreases, occupancy of the genomic complex (e.g., ASMC) at a genomic sequence element (e.g., a target gene, or a transcriptional control sequence operably linked thereto). In some embodiments, occupancy decreases by at least 10, 20, 30, 40, 50, 60, 70, 80, 90, or 100% (and optionally, up to 100, 90, 80, 70, 60, 50, 40, 30, or 20%) in the presence of a site-specific disrupting agent comprising the targeting moiety relative to the absence of said site-specific disrupting agent. Changes in genomic complex level and/or occupancy may be evaluated, for example, using HiChIP, ChIAPET, 4C, or 3C, e.g., HiChIP.

In some embodiments, binding of a targeting moiety to a targeted component alters, e.g., decreases, the occupancy of the genomic complex (e.g., ASMC) at a genomic sequence element (e.g., a gene, promoter, or enhancer, e.g., associated with the genomic or transcription complex). In some embodiments, binding of a targeting moiety to a targeted component decreases occupancy of the genomic complex (e.g., ASMC) at a genomic sequence element by at least 10, 20, 30, 40, 50, 60, 70, 80, 90, or 100% (and optionally, up to 100, 90, 80, 70, 60, 50, 40, 30, or 20%) in the presence of a site-specific disrupting agent comprising the targeting moiety relative to the absence of said site-specific disrupting agent. In some embodiments, occupancy refers to the frequency with which an element can be found associated with another element, e.g., as determined by HiC, ChIP, immunoprecipitation, or other association measuring assays known in the art.

In some embodiments, binding of a targeting moiety to a targeted component alters, e.g., decreases the occupancy of the targeted component in/at the genomic complex (e.g., ASMC). In some embodiments, binding of a targeting moiety to a targeted component decreases occupancy of the targeted component in/at the genomic complex (e.g., ASMC) by at least 10, 20, 30, 40, 50, 60, 70, 80, 90, or 100% (and optionally, up to 100, 90, 80, 70, 60, 50, 40, 30, or 20%) in the presence of a site-specific disrupting agent comprising the targeting moiety relative to the absence of said site-specific disrupting agent.

In some embodiments, binding of a targeting moiety to a targeted component alters, e.g., decreases, the expression of a target gene associated with the genomic complex (e.g., ASMC) comprising the targeted component. In some embodiments, the expression of the target gene decreases by at least 10, 20, 30, 40, 50, 60, 70, 80, 90, or 100% (and optionally, up to 100, 90, 80, 70, 60, 50, 40, 30, or 20%) in the presence of a site-specific disrupting agent comprising the targeting moiety relative to the absence of said site-specific disrupting agent.

In some embodiments, a targeting moiety may be or comprise a CRISPR/Cas molecule, a TAL effector molecule, a Zn finger molecule, or a nucleic acid molecule.

In some embodiments, a targeting moiety may also be an effector moiety. For example, a targeting moiety comprising a CRISPR/Cas molecule may specifically bind a target nucleic acid sequence and also act as an effector moiety, e.g., a genetic modifying moiety, with enzymatic activity that acts on a target component (e.g., by cleaving target DNA).

In some embodiments, a targeting moiety is or comprises a nucleic acid (e.g., an oligonucleotide (e.g. a gRNA, etc.) which, in some embodiments, may contain one or more modified residues, linkages, or other features), a polypeptide (e.g., a protein, a protein fragment, an antibody, an antibody fragment [e.g., an antigen-binding fragment], a fusion molecule, etc., any of which, in some embodiments, may include one or more modified residues, linkages, or other features), peptide nucleic acid, small molecule, etc.

As described in greater detail herein, in some embodiments, a targeting moiety as described herein can be or comprise a polymer or polymeric moiety, e.g., a polymer of nucleotides (such as an oligonucleotide), a peptide nucleic acid, a peptide-nucleic acid mixmer, a peptide or polypeptide, a polyamide, a carbohydrate, etc.

In some embodiments, a targeting moiety is or comprises one or more of a nucleic acid, a polypeptide, or a small molecule. In some embodiments, a targeting moiety is or comprises a nucleic acid, e.g., DNA or RNA. In some embodiments, a targeting moiety is or comprises a synthetic nucleic acid. In some embodiments, a targeting moiety is or comprises a gRNA. In some embodiments, a targeting moiety is or comprises a CRISPR/Cas protein. In some embodiments, a Cas protein is or comprises Cas9. In some embodiments, a Cas9 protein is enzymatically inactive. In some embodiments a Cas9 protein is or comprises a variant protein whose amino acid sequence includes substitutions D10A and/or H840A. In some embodiments, a targeting moiety is or comprises dCas9. In some embodiments, a targeting moiety is or comprises a fusion molecule. In some embodiments, a fusion molecule is or comprises two moieties that are not naturally associated with one another but are linked by the hand of man (e.g. fusion proteins, polypeptide-drug conjugates, etc.). In some embodiments, a fusion molecule is or comprises a Cas protein fused to gRNA. In some embodiments, a targeting moiety is or comprises dCas9 fused to a gRNA.

In some embodiments, a targeting moiety is or comprises a peptide nucleic acid (PNA). In some embodiments, a targeting moiety is or comprises a bridged nucleic acid (BNA). In some embodiments, a targeting moiety is or comprises a non-coding RNA (ncRNA). In some embodiments, a targeting moiety is or comprises a ribonucleic acid and targets a nucleic acid, e.g., ribonucleic acid, e.g., functional or noncoding RNA component of a genomic complex.

In some embodiments, a targeting moiety is or comprises an antibody or antigen binding fragment thereof, e.g., specific for a genetic complex component. In some embodiments, a disrupting agent comprising a targeting moiety that is or comprises an antibody or antigen binding fragment thereof (e.g., specific for a genetic complex component), is associated with (e.g., conjugated or operably linked in a fusion protein) an effector moiety (e.g., disrupting moiety) comprising a nucleic acid, e.g., ribonucleic acid. In the same embodiments, the nucleic acid, e.g., ribonucleic acid, may be complementary to a genomic sequence element or to a non-genomic nucleic acid component of a genomic complex.

In some embodiments, a targeting moiety is or comprises a TAL effector molecule. A TAL effector molecule, e.g., a TAL effector molecule that specifically binds a DNA sequence, comprises a plurality of TAL effector domains or fragments thereof, and optionally one or more additional portions of naturally occurring TAL effectors (e.g., N- and/or C-terminal of the plurality of TAL effector domains).

TALEs are natural effector proteins secreted by numerous species of bacterial pathogens including the plant pathogen Xanthomonas which modulates gene expression in host plants and facilitates bacterial colonization and survival. The specific binding of TAL effectors is based on a central repeat domain of tandemly arranged nearly identical repeats of typically 33 or 34 amino acids (the repeat-variable di-residues, RVD domain).

Members of the TAL effectors family differ mainly in the number and order of their repeats. The number of repeats ranges from 1.5 to 33.5 repeats and the C-terminal repeat is usually shorter in length (e.g., about 20 amino acids) and is generally referred to as a “half-repeat”. Each repeat of the TAL effector feature a one-repeat-to-one-base-pair correlation with different repeat types exhibiting different base-pair specificity (one repeat recognizes one base-pair on the target gene sequence). Generally, the smaller the number of repeats, the weaker the protein-DNA interactions. A number of 6.5 repeats has been shown to be sufficient to activate transcription of a reporter gene (Scholze et al., 2010).

Repeat to repeat variations occur predominantly at amino acid positions 12 and 13, which have therefore been termed “hypervariable” and which are responsible for the specificity of the interaction with the target DNA promoter sequence, as shown in Table 4 listing exemplary repeat variable diresidues (RVD) and their correspondence to nucleic acid base targets.

TABLE 4
RVDs and Nucleic Acid Base Specificity

| Target | Possible RVD Amino Acid Combinations | | | | | | | | | | | | |
|---|---|---|---|---|---|---|---|---|---|---|---|---|---|
| A | NI | NN | CI | HI | KI | | | | | | | | |
| G | NN | GN | SN | VN | LN | DN | QN | EN | HN | RH | NK | AN | FN |
| C | HD | RD | KD | ND | AD | | | | | | | | |
| T | NG | HG | VG | IG | EG | MG | YG | AA | EP | VA | QG | KG | RG |

Accordingly, it is possible to modify the repeats of a TAL effector to target specific DNA sequences. Further studies have shown that the RVD NK can target G. Target sites of TAL effectors also tend to include a T flanking the 5′ base targeted by the first repeat, but the exact mechanism of this recognition is not known. More than 113 TAL effector sequences are known to date. Non-limiting examples of TAL effectors from Xanthomonas include, Hax2, Hax3, Hax4, AvrXa7, AvrXa10 and AvrBs3.

Accordingly, the TAL effector domain of the TAL effector molecule of the present invention may be derived from a TAL effector from any bacterial species (e.g., Xanthomonas species such as the African strain of Xanthomonas oryzae pv. Oryzae (Yu et al. 2011), Xanthomonas campestris pv. raphani strain 756C and Xanthomonas oryzae pv. oryzicolastrain BLS256 (Bogdanove et al. 2011). As used herein, the TAL effector domain in accordance with the present invention comprises an RVD domain as well as flanking sequence(s) (sequences on the N-terminal and/or C-terminal side of the RVD domain) also from the naturally occurring TAL effector. It may comprise more or fewer repeats than the RVD of the naturally occurring TAL effector. The TAL effector molecule of the present invention is designed to target a given DNA sequence based on the above code. The number of TAL effector domains (e.g., repeats (monomers or modules)) and their specific sequence are selected based on the desired DNA target sequence. For example, TAL effector domains, e.g., repeats, may be removed or added in order to suit a specific target sequence. In an embodiment, the TAL effector molecule of the present invention comprises between 6.5 and 33.5 TAL effector domains, e.g., repeats. In an embodiment, TAL effector molecule of the present invention comprises between 8 and 33.5 TAL effector domains, e.g., repeats, e.g., between 10 and 25 TAL effector domains, e.g., repeats, e.g., between 10 and 14 TAL effector domains, e.g., repeats.

In some embodiments, the TAL effector molecule comprises TAL effector domains that correspond to a perfect match to the DNA target sequence. In some embodiments, a mismatch between a repeat and a target base-pair on the DNA target sequence is permitted as along as it allows for the function of the expression repression system, e.g., the expression repressor comprising the TAL effector molecule. In general, TALE binding is inversely correlated with the number of mismatches. In some embodiments, the TAL effector molecule of a expression repressor of the present invention comprises no more than 7 mismatches, 6 mismatches, 5 mismatches, 4 mismatches, 3 mismatches, 2 mismatches, or 1 mismatch, and optionally no mismatch, with the target DNA sequence. Without wishing to be bound by theory, in general the smaller the number of TAL effector domains in the TAL effector molecule, the smaller the number of mismatches will be tolerated and still allow for the function of the expression repression system, e.g., the expression repressor comprising the TAL effector molecule. The binding affinity is thought to depend on the sum of matching repeat-DNA combinations. For example, TAL effector molecules having 25 TAL effector domains or more may be able to tolerate up to 7 mismatches.

In addition to the TAL effector domains, the TAL effector molecule of the present invention may comprise additional sequences derived from a naturally occurring TAL effector. The length of the C-terminal and/or N-terminal sequence(s) included on each side of the TAL effector domain portion of the TAL effector molecule can vary and be selected by one skilled in the art, for example based on the studies of Zhang et al. (2011). Zhang et al., have characterized a number of C-terminal and N-terminal truncation mutants in Hax3 derived TAL-effector based proteins and have identified key elements, which contribute to optimal binding to the target sequence and thus activation of transcription. Generally, it was found that transcriptional activity is inversely correlated with the length of N-terminus. Regarding the C-terminus, an important element for DNA binding residues within the first 68 amino acids of the Hax 3 sequence was identified. Accordingly, in some embodiments, the first 68 amino acids on the C-terminal side of the TAL effector domains of the naturally occurring TAL effector is included in the TAL effector molecule of an expression repressor of the present invention. Accordingly, in an embodiment, a TAL effector molecule of the present invention comprises 1) one or more TAL effector domains derived from a naturally occurring TAL effector; 2) at least 70, 80, 90, 100, 110, 120, 130, 140, 150, 170, 180, 190, 200, 220, 230, 240, 250, 260, 270, 280 or more amino acids from the naturally occurring TAL effector on the N-terminal side of the TAL effector domains; and/or 3) at least 68, 80, 90, 100, 110, 120, 130, 140, 150, 170, 180, 190, 200, 220, 230, 240, 250, 260 or more amino acids from the naturally occurring TAL effector on the C-terminal side of the TAL effector domains.

In some embodiments, a targeting moiety is or comprises a Zn finger molecule. A Zn finger molecule comprises a Zn finger protein, e.g., a naturally occurring Zn finger protein or engineered Zn finger protein, or fragment thereof.

In some embodiments, a Zn finger molecule comprises a non-naturally occurring Zn finger protein that is engineered to bind to a target DNA sequence of choice. See, for example, Beerli, et al. (2002) Nature Biotechnol. 20:135-141; Pabo, et al. (2001) Ann. Rev. Biochem. 70:313-340; Isalan, et al. (2001) Nature Biotechnol. 19:656-660; Segal, et al. (2001) Curr. Opin. Biotechnol. 12:632-637; Choo, et al. (2000) Curr. Opin. Struct. Biol. 10:411-416; U.S. Pat. Nos. 6,453,242; 6,534,261; 6,599,692; 6,503,717; 6,689,558; 7,030,215; 6,794,136; 7,067,317; 7,262,054; 7,070,934; 7,361,635; 7,253,273; and U.S. Patent Publication Nos. 2005/0064474; 2007/0218528; 2005/0267061, all incorporated herein by reference in their entireties.

An engineered Zn finger protein may have a novel binding specificity, compared to a naturally-occurring Zn finger protein. Engineering methods include, but are not limited to, rational design and various types of selection. Rational design includes, for example, using databases comprising triplet (or quadruplet) nucleotide sequences and individual Zn finger amino acid sequences, in which each triplet or quadruplet nucleotide sequence is associated with one or more amino acid sequences of zinc fingers which bind the particular triplet or quadruplet sequence. See, for example, U.S. Pat. Nos. 6,453,242 and 6,534,261, incorporated by reference herein in their entireties.

Exemplary selection methods, including phage display and two-hybrid systems, are disclosed in U.S. Pat. Nos. 5,789,538; 5,925,523; 6,007,988; 6,013,453; 6,410,248; 6,140,466; 6,200,759; and 6,242,568; as well as International Patent Publication Nos. WO 98/37186; WO 98/53057; WO 00/27878; and WO 01/88197 and GB 2,338,237. In addition, enhancement of binding specificity for zinc finger proteins has been described, for example, in International Patent Publication No. WO 02/077227.

In addition, as disclosed in these and other references, zinc finger domains and/or multi-fingered zinc finger proteins may be linked together using any suitable linker sequences, including for example, linkers of 5 or more amino acids in length. See, also, U.S. Pat. Nos. 6,479,626; 6,903,185; and 7,153,949 for exemplary linker sequences 6 or more amino acids in length. The proteins described herein may include any combination of suitable linkers between the individual zinc fingers of the protein. In addition, enhancement of binding specificity for zinc finger binding domains has been described, for example, in co-owned International Patent Publication No. WO 02/077227.

Zn finger proteins and methods for design and construction of fusion proteins (and polynucleotides encoding same) are known to those of skill in the art and described in detail in U.S. Pat. Nos. 6,140,0815; 789,538; 6,453,242; 6,534,261; 5,925,523; 6,007,988; 6,013,453; and 6,200,759; International Patent Publication Nos. WO 95/19431; WO 96/06166; WO 98/53057; WO 98/54311; WO 00/27878; WO 01/60970; WO 01/88197; WO 02/099084; WO 98/53058; WO 98/53059; WO 98/53060; WO 02/016536; and WO 03/016496.

In addition, as disclosed in these and other references, Zn finger proteins and/or multi-fingered Zn finger proteins may be linked together, e.g., as a fusion protein, using any suitable linker sequences, including for example, linkers of 5 or more amino acids in length. See, also, U.S. Pat. Nos. 6,479,626; 6,903,185; and 7,153,949 for exemplary linker sequences 6 or more amino acids in length. The Zn finger molecules described herein may include any combination of suitable linkers between the individual zinc finger proteins and/or multi-fingered Zn finger proteins of the Zn finger molecule.

In certain embodiments, the DNA-targeting moiety comprises a Zn finger molecule comprising an engineered zinc finger protein that binds (in a sequence-specific manner) to a target DNA sequence. In some embodiments, the Zn finger molecule comprises one Zn finger protein or fragment thereof. In other embodiments, the Zn finger molecule comprises a plurality of Zn finger proteins (or fragments thereof), e.g., 2, 3, 4, 5, 6 or more Zn finger proteins (and optionally no more than 12, 11, 10, 9, 8, 7, 6, 5, 4, 3, or 2 Zn finger proteins). In some embodiments, the Zn finger molecule comprises at least three Zn finger proteins. In some embodiments, the Zn finger molecule comprises four, five or six fingers. In some embodiments, the Zn finger molecule comprises 8, 9, 10, 11 or 12 fingers. In some embodiments, a Zn finger molecule comprising three Zn finger proteins recognizes a target DNA sequence comprising 9 or 10 nucleotides. In some embodiments, a Zn finger molecule comprising four Zn finger proteins recognizes a target DNA sequence comprising 12 to 14 nucleotides. In some embodiments, a Zn finger molecule comprising six Zn finger proteins recognizes a target DNA sequence comprising 18 to 21 nucleotides.

In some embodiments, a Zn finger molecule comprises a two-handed Zn finger protein. Two handed zinc finger proteins are those proteins in which two clusters of zinc finger proteins are separated by intervening amino acids so that the two zinc finger domains bind to two discontinuous target DNA sequences. An example of a two handed type of zinc finger binding protein is SIP1, where a cluster of four zinc finger proteins is located at the amino terminus of the protein and a cluster of three Zn finger proteins is located at the carboxyl terminus (see Remade, et al. (1999) EMBO Journal 18(18):5073-5084). Each cluster of zinc fingers in these proteins is able to bind to a unique target sequence and the spacing between the two target sequences can comprise many nucleotides.

In some embodiments, a targeting moiety is or comprises a DNA-binding domain from a nuclease. For example, the recognition sequences of homing endonucleases and meganucleases such as I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII are known. See also U.S. Pat. Nos. 5,420,032; 6,833,252; Belfort, et al. (1997) Nucleic Acids Res. 25:3379-3388; Dujon, et al. (1989) Gene 82:115-118; Perler, et al. (1994) Nucleic Acids Res. 22:1125-1127; Jasin (1996) Trends Genet. 12:224-228; Gimble, et al. (1996)J. Mol. Biol. 263:163-180; Argast, et al. (1998)J. Mol. Biol. 280:345-353 and the New England Biolabs catalogue. In addition, the DNA-binding specificity of homing endonucleases and meganucleases can be engineered to bind non-natural target sites. See, for example, Chevalier, et al. (2002) Molec. Cell 10:895-905; Epinat, et al. (2003) Nucleic Acids Res. 31:2952-2962; Ashworth, et al. (2006) Nature 441:656-659; Paques, et al. (2007) Current Gene Therapy 7:49-66; U.S. Patent Publication No. 2007/0117128.

In some embodiments, a targeting moiety may be or comprise anything that is capable of binding to a target.

In some embodiments, a targeting moiety as described herein is designed and/or administered so that it specifically inhibits, inhibits formation of, and/or destabilizes (e.g., inhibits, dissociates, degrades (e.g., a component of), and/or modifies (e.g., a component of)) a particular genomic complex relative to other genomic complexes that may be present in the same system (e.g., cell, tissue, etc.). In some embodiments, a targeting moiety that specifically inhibits, inhibits formation of, and/or destabilizes (e.g., inhibits, dissociates, degrades (e.g., a component of), and/or modifies (e.g., a component of)) a particular genomic complex relative to other genomic complexes that may be present in the same system (e.g., cell, tissue, etc.) sterically inhibits (e.g., by blocking a component binding site) the particular genomic complex. For example, a targeting moiety that binds a genomic sequence element of a genomic complex (e.g., a targeting moiety comprising a nucleic acid, e.g., anti-sense nucleic acid) can prevent or inhibit binding of nucleating polypeptides, thereby inhibiting/inhibiting formation of the genomic complex.

Those skilled in the art will appreciate that, in many embodiments, a targeting moiety that targets a polypeptide component of a genomic complex as described herein may be or comprise a polypeptide agent (e.g., an antibody or antigen binding fragment thereof) that specifically binds with the target polypeptide component. Of course, those skilled in the art will appreciate that, in some embodiments, a targeting moiety that targets a polypeptide component is not necessarily a polypeptide agent, and certainly is not necessarily an antibody or antigen binding fragment thereof. For example, in some embodiments, such a targeting moiety may be or comprise a small molecule or a nucleic acid (e.g., an oligonucleotide) that specifically binds with the targeted component. Alternatively or additionally, in some embodiments, such a targeting moiety may be or comprise a non-antibody polypeptide, such as another protein (e.g., another complex component, or a variant thereof) that interacts with the targeted complex component.

In general, those skilled in the art will appreciate that any entity or agent capable of specific interaction with a target site or target complex component(s) under conditions of their mutual exposure, as described herein, can be utilized as a targeting moiety in certain embodiments of the present disclosure.

Effector Moieties

In some embodiments, an effector moiety comprises a disrupting moiety, a modifying moiety, a tagging/monitoring moiety, a cleavable moiety, a membrane translocating moiety, or a pharmacoagent moiety. In some embodiments, an effector moiety may alter a biological activity, for example increasing or decreasing enzymatic activity, gene expression, cell signaling, and cellular or organ function. Alternatively or additionally, in some embodiments effector activities may also include binding regulatory proteins to alter activity of the regulator, such as transcription or translation. Still further alternatively or additionally, in some embodiments, effector activities also may include activator or inhibitor functions as described herein. In some embodiments, a targeting moiety may inhibit substrate binding to a receptor and inhibit its activation, e.g., naltrexone and naloxone bind opioid receptors without activating them and block receptors' ability to bind opioids. Effector activities may also include altering protein stability/degradation and/or transcript stability/degradation.

Embodiments provided herein provide a site-specific disrupting agent that comprises a targeting moiety (e.g., that localizes the disrupting agent to a genomic location or site at which incidence of a genomic complex is decreased in accordance with the present disclosure). In some embodiments, a targeting moiety is also a disrupting moiety (e.g., in that it inhibits, inhibits formation of, and/or destabilizes the relevant genomic complex); in some embodiments, a site-specific disrupting agent comprises distinct targeting and effector moieties (e.g., disrupting, modifying or other effector moieties).

Thus, in some embodiments, a provided site-specific disrupting agent is or comprises a targeting moiety and one or more effector moieties. In some embodiments, an effector moiety may be or comprise a disrupting moiety. Alternatively or additionally, in some embodiments, an effector moiety may be or comprise one or more of a tagging moiety, a cleavable moiety, a membrane translocation moiety, a pharmacoagent moiety, etc.

In some embodiments, an effector moiety is a chemical, e.g., a chemical that alters a cytosine (C) or an adenine (A) (e.g., Na bisulfite, ammonium bisulfite). In some embodiments, an effector moiety has enzymatic activity (methyl transferase, demethylase, nuclease (e.g., Cas9), a deaminase). In some embodiments, an effector moiety sterically inhibits formation of an anchor sequence-mediated conjunction [e.g., membrane translocating polypeptide+nanoparticle (e.g., having an average diameter of about 1-100 nm)].

An effector moiety with effector activity may be at least one of small molecules, peptides, nucleic acids, nanoparticles, aptamers, and pharmacoagents with poor PK/PD described herein.

Disrupting Moieties

In some embodiments, a disrupting agent comprises a disrupting moiety. In some embodiments, a disrupting moiety inhibits or destabilizes one or more components of a genomic complex. In some embodiments, a disrupting moiety interacts with one or more genomic complex components that is not a disrupting moiety. In some embodiments, a disrupting moiety is or comprises a genomic complex component, e.g., a genomic complex component that has been altered to inhibit or prevent formation of the genomic complex.

In some embodiments, a disrupting moiety sterically inhibits (e.g., by blocking a binding site) association or binding of one or more particular components of the genomic complex so that incidence of the complete complex is less when the disrupting moiety is present than when it is absent. In some embodiments, a disrupting moiety that sterically inhibits a genomic complex binds to a component of the relevant genomic complex, as described herein. In some embodiments, a disrupting moiety that sterically inhibits a genomic complex binds directly to a genomic complex component. In some embodiments, a disrupting moiety that sterically inhibits a genomic complex is a competitive inhibitor of binding, e.g., of one or more components of the genomic complex. In some embodiments, a disrupting moiety that sterically inhibits a genomic complex may comprise any agent of suitable shape and size to sterically inhibit binding of one or more components of the genomic complex. In some embodiments, a disrupting moiety binds indirectly to a genomic complex component (e.g. via direct binding to another agent or entity that then interacts directly or indirectly, with the component).

Modifying Moieties

In some embodiments, an effector moiety is or comprises a modifying moiety. In some embodiments, a modifying moiety is or comprises a genetic modifying moiety. In some embodiments, a modifying moiety modifies a genomic site that is or becomes a genomic sequence element (e.g. a CTCF binding motif, a promoter and/or an enhancer).

In some embodiments, a modifying moiety is or comprises an epigenetic modifying moiety. In some embodiments, the modifying moiety modifies a genomic site in the vicinity of a genomic complex component (e.g., a genomic sequence element).

In some embodiments, a modifying moiety is or comprises a polypeptide modifying moiety. In some embodiments, a modifying moiety modifies a ligand that is or will become a genomic complex component.

Genetic Modifying Moieties

In some embodiments, a disrupting agent (e.g., comprising a site-specific targeting moiety) comprises one or more genetic modifying moieties (e.g. components of a gene editing system). As can be appreciated by those skilled in the art reading the present specification, and as explained further herein, genetic modifying moieties may be used in a variety of contexts including but not limited to gene editing. For example, such moieties may be used to make changes to the sequence of a target site (e.g., mutations, e.g., substitutions, deletions, insertions, etc.).

In some embodiments, a genetic modifying moiety targets one or more nucleotides of an anchor sequence-mediated conjunction such as through a gene editing system (e.g. nucleic acid editing moiety), of a sequence within or related to any component of a genomic complex, e.g., an anchor sequence, e.g., a common nucleotide sequence within an anchor sequence, within an anchor sequence-mediated conjunction for substitution, addition or deletion, within an anchor sequence-mediated conjunction by substitution, addition, or deletion; a nucleotide within an ncRNA/eRNA, a sequence encoding a component (e.g. transcription factor) or a genomic complex, etc. In some embodiments, a targeting moiety binds an anchor sequence-mediated conjunction, e.g., an anchor sequence in an anchor sequence-mediated conjunction, and alters a topology of an anchor sequence-mediated conjunction.

In some embodiments, a genetic modifying moiety may target one or more nucleotides, such as through a gene editing system, of a sequence, e.g., an ncRNA or eRNA. In some embodiments, a nucleic acid editing moiety binds an ncRNA or eRNA and alters a genomic complex, e.g. alters topology of an anchor sequence-mediated conjunction.

In some embodiments, a genetic modifying moiety targets one or more nucleotides, e.g., such as through CRISPR, TALEN, dCas9, oligonucleotide pairing, recombination, transposon, etc., within or as a component of a genomic complex (e.g. within an anchor sequence-mediated conjunction) for substitution, addition or deletion. In some embodiments, a nucleic acid editing moiety targets one or more DNA methylation sites within an anchor sequence-mediated conjunction.

In some embodiments, a genetic modifying moiety introduces a targeted alteration into an anchor sequence-mediated conjunction to modulate transcription, in a human cell, of a gene in an anchor sequence-mediated conjunction. In some embodiments, a genetic modifying moiety introduces a targeted alteration into a ncRNA or eRNA that is part of a genomic complex, wherein the alteration modulates transcription of a gene in an anchor sequence-mediated conjunction. A targeted alteration may include a substitution, addition or deletion of one or more nucleotides, e.g., of an anchor sequence within an anchor sequence-mediated conjunction. A genetic modifying moiety may bind an anchor sequence of an anchor sequence-mediated conjunction and a targeting moiety introduces a targeted alteration into an anchor sequence to modulate transcription (e.g., decrease transcription), in a human cell, of a gene in an anchor sequence-mediated conjunction (e.g., an associated gene, e.g., a fusion gene, e.g., a fusion oncogene). In some embodiments, a targeted alteration alters at least one of a binding site for a nucleating polypeptide, e.g. altering binding affinity for an anchor sequence within an anchor sequence-mediated conjunction, an alternative splicing site, and a binding site for a nontranslated RNA. In some embodiments, a targeted alteration decreases the affinity of a genomic complex component (e.g., nucleating polypeptide) for another genomic complex component (e.g., genomic sequence element, e.g., anchor sequence). In some embodiments, a targeted alteration decreases the affinity of a transcriptional regulatory sequence for one or more transcription factors.

In some embodiments, a genetic modifying moiety edits a component of a genomic complex (e.g. a sequence in an anchor sequence-mediated conjunction) via at least one of the following: providing at least one exogenous anchor sequence; an alteration in at least one nucleating polypeptide binding motif, such as by altering (e.g., decreasing) binding affinity for a nucleating polypeptide; a change in an orientation of at least one common nucleotide sequence, such as a CTCF binding motif; a deletion, substitution, or insertion that disrupts a genome sequence element (e.g., a genome sequence element in the particular targeted genomic complex), e.g., a substitution, addition or deletion in or of at least one anchor sequence, such as a CTCF binding motif.

Exemplary gene editing systems include clustered regulatory interspaced short palindromic repeat (CRISPR) system, zinc finger nucleases (ZFNs), and Transcription Activator-Like Effector-based Nucleases (TALEN). ZFNs, TALENs, and CRISPR-based methods are described, e.g., in Gaj et al. Trends Biotechnol. 31.7(2013):397-405; CRISPR methods of gene editing are described, e.g., in Guan et al., Application of CRISPR-Cas system in gene therapy: Pre-clinical progress in animal model. DNA Repair 2016 Jul. 30 [Epub ahead of print]; Zheng et al., Precise gene deletion and replacement using the CRISPR/Cas9 system in human cells. BioTechniques, Vol. 57, No. 3, September 2014, pp. 115-124.

For example, in some embodiments a genetic modifying moiety is or comprises a CRISPR/Cas molecule. A CRISPR/Cas molecule comprises a protein involved in the clustered regulatory interspaced short palindromic repeat (CRISPR) system, e.g., a Cas protein (e.g., nuclease), and optionally a guide RNA, e.g., single guide RNA (sgRNA).

In some embodiments, a Cas nuclease is enzymatically inactive, e.g., a dCas9, as described further herein. In some embodiments, a targeting moiety comprises a CRISPR/Cas molecule, e.g., an enzymatically inactive (e.g., dCas9) CRISPR/Cas molecule.

In some embodiments, methods and compositions as provided herein can be used with a CRISPR-based gene editing, whereby guide RNA (gRNA) are used in a clustered regulatory interspaced short palindromic repeat (CRISPR) system for gene editing.

CRISPR systems are adaptive defense systems originally discovered in bacteria and archaea. CRISPR systems use RNA-guided nucleases termed CRISPR-associated or “Cas” endonucleases (e. g., Cas9 or Cpf1) to cleave foreign DNA. For example, in a typical CRISPR/Cas system, an endonuclease is directed to a target nucleotide sequence (e. g., a site in the genome that is to be sequence-edited) by sequence-specific, non-coding “guide RNAs” that target single- or double-stranded DNA sequences. Three classes (I-III) of CRISPR systems have been identified. The class II CRISPR systems use a single Cas endonuclease (rather than multiple Cas proteins). One class II CRISPR system includes a type II Cas endonuclease such as Cas9, a CRISPR RNA (“crRNA”), and a trans-activating crRNA (“tracrRNA”). The crRNA contains a “guide RNA”, typically about 20-nucleotide RNA sequence that corresponds to a target DNA sequence. crRNA also contains a region that binds to the tracrRNA to form a partially double-stranded structure which is cleaved by RNase III, resulting in a crRNA/tracrRNA hybrid. A crRNA/tracrRNA hybrid then directs Cas9 endonuclease to recognize and cleave a target DNA sequence. A target DNA sequence must generally be adjacent to a “protospacer adjacent motif” (“PAM”) that is specific for a given Cas endonuclease; however, PAM sequences appear throughout a given genome. CRISPR endonucleases identified from various prokaryotic species have unique PAM sequence requirements; examples of PAM sequences include 5′-NGG (Streptococcus pyogenes), 5′-NNAGAA (Streptococcus thermophilus CRISPR1), 5′-NGGNG (Streptococcus thermophilus CRISPR3), and 5′-NNNGATT (Neisseria meningiditis). Some endonucleases, e. g., Cas9 endonucleases, are associated with G-rich PAM sites, e. g., 5′-NGG, and perform blunt-end cleaving of the target DNA at a location 3 nucleotides upstream from (5′ from) the PAM site. Another class II CRISPR system includes the type V endonuclease Cpf1, which is smaller than Cas9; examples include AsCpf1 (from Acidaminococcus sp.) and LbCpf1 (from Lachnospiraceae sp.). Cpf1-associated CRISPR arrays are processed into mature crRNAs without the requirement of a tracrRNA; in other words a Cpf1 system requires only Cpf1 nuclease and a crRNA to cleave a target DNA sequence. Cpf1 endonucleases, are associated with T-rich PAM sites, e. g., 5′-TTN. Cpf1 can also recognize a 5′-CTA PAM motif. Cpf1 cleaves a target DNA by introducing an offset or staggered double-strand break with a 4- or 5-nucleotide 5′ overhang, for example, cleaving a target DNA with a 5-nucleotide offset or staggered cut located 18 nucleotides downstream from (3′ from) from a PAM site on the coding strand and 23 nucleotides downstream from the PAM site on the complimentary strand; the 5-nucleotide overhang that results from such offset cleavage allows more precise genome editing by DNA insertion by homologous recombination than by insertion at blunt-end cleaved DNA. See, e. g., Zetsche et al. (2015) Cell, 163:759-771.

A variety of CRISPR associated (Cas) genes or proteins can be used in the technologies provided by the present disclosure and the choice of Cas protein will depend upon the particular conditions of the method. Specific examples of Cas proteins include class II systems including Cas1, Cas2, Cas3, Cas4, Cas5, Cas6, Cas7, Cas8, Cas9, Cas10, Cpf1, C2C1, or C2C3. In some embodiments, a Cas protein, e.g., a Cas9 protein, may be from any of a variety of prokaryotic species. In some embodiments a particular Cas protein, e.g., a particular Cas9 protein, is selected to recognize a particular protospacer-adjacent motif (PAM) sequence. In some embodiments, a modulating agent (e.g., site-specific disrupting agent) includes a sequence targeting polypeptide, such as an enzyme, e.g., Cas9. In certain embodiments a Cas protein, e.g., a Cas9 protein, may be obtained from a bacteria or archaea or synthesized using known methods. In certain embodiments, a Cas protein may be from a gram positive bacteria or a gram negative bacteria. In certain embodiments, a Cas protein may be from a Streptococcus, (e.g., a S. pyogenes, a S. thermophilus) a Cryptococcus, a Corynebacterium, a Haemophilus, a Eubacterium, a Pasteurella, a Prevotella, a Veillonella, or a Marinobacter. In some embodiments nucleic acids encoding two or more different Cas proteins, or two or more Cas proteins, may be introduced into a cell, zygote, embryo, or animal, e.g., to allow for recognition and modification of sites comprising the same, similar or different PAM motifs. In some embodiments, the Cas protein is modified to deactivate the nuclease, e.g., nuclease-deficient Cas9, and to recruit transcription activators or repressors, e.g., the w-subunit of the E. coli Pol, VP64, the activation domain of p65, KRAB, or SID4X, to induce epigenetic modifications, e.g., histone acetyltransferase, histone methyltransferase and demethylase, DNA methyltransferase and enzyme with a role in DNA demethylation (e.g., the TET family enzymes catalyze oxidation of 5-methylcytosine to 5-hydroxymethylcytosine and higher oxidative derivatives).

For the purposes of gene editing, CRISPR arrays can be designed to contain one or multiple guide RNA sequences corresponding to a desired target DNA sequence; see, for example, Cong et al. (2013) Science, 339:819-823; Ran et al. (2013) Nature Protocols, 8:2281-2308. At least about 16 or 17 nucleotides of gRNA sequence are required by Cas9 for DNA cleavage to occur; for Cpf1 at least about 16 nucleotides of gRNA sequence is needed to achieve detectable DNA cleavage.

Whereas wild-type Cas9 generates double-strand breaks (DSBs) at specific DNA sequences targeted by a gRNA, a number of CRISPR endonucleases having modified functionalities are available, for example: a “nickase” version of Cas9 generates only a single-strand break; a catalytically inactive Cas9 (“dCas9”) does not cut target DNA but interferes with transcription by steric hindrance. dCas9 can further be fused with a heterologous effector to repress (CRISPRi) or activate (CRISPRa) expression of a target gene. For example, Cas9 can be fused to a transcriptional silencer (e.g., a KRAB domain) or a transcriptional activator (e.g., a dCas9-VP64 fusion). A catalytically inactive Cas9 (dCas9) fused to FokI nuclease (“dCas9-FokI”) can be used to generate DSBs at target sequences homologous to two gRNAs. See, e. g., the numerous CRISPR/Cas9 plasmids disclosed in and publicly available from the Addgene repository (Addgene, 75 Sidney St., Suite 550A, Cambridge, Mass. 02139; addgene.org/crispr/). A “double nickase” Cas9 that introduces two separate double-strand breaks, each directed by a separate guide RNA, is described as achieving more accurate genome editing by Ran et al. (2013) Cell, 154:1380-1389.

CRISPR technology for editing the genes of eukaryotes is disclosed in US Patent Application Publications 2016/0138008A1 and US2015/0344912A1, and in U.S. Pat. Nos. 8,697,359, 8,771,945, 8,945,839, 8,999,641, 8,993,233, 8,895,308, 8,865,406, 8,889,418, 8,871,445, 8,889,356, 8,932,814, 8,795,965, and 8,906,616. Cpf1 endonuclease and corresponding guide RNAs and PAM sites are disclosed in US Patent Application Publication 2016/0208243 A1.

In some embodiments, a desired genome modification involves homologous recombination, wherein one or more double-stranded DNA breaks in a target nucleotide sequence is generated by an RNA-guided nuclease and guide RNA(s), followed by repair of a break(s) using a homologous recombination mechanism (“homology-directed repair”). In such embodiments, a donor template that encodes a desired nucleotide sequence to be inserted or knocked-in at a double-stranded break is provided to a cell or subject; examples of suitable templates include single-stranded DNA templates and double-stranded DNA templates (e. g., linked to the polypeptide described herein). In general, a donor template encoding a nucleotide change over a region of less than about 50 nucleotides is provided in as single-stranded DNA; larger donor templates (e. g., more than 100 nucleotides) are often provided as double-stranded DNA plasmids. In some embodiments, a donor template is provided to a cell or subject in a quantity that is sufficient to achieve desired homology-directed repair but that does not persist in the cell or subject after a given period of time (e. g., after one or more cell division cycles). In some embodiments, a donor template has a core nucleotide sequence that differs from a target nucleotide sequence (e. g., a homologous endogenous genomic region) by at least 1, at least 5, at least 10, at least 20, at least 30, at least 40, at least 50, or more nucleotides. This core sequence is flanked by “homology arms” or regions of high sequence identity with the targeted nucleotide sequence; in embodiments, regions of high identity include at least 10, at least 50, at least 100, at least 150, at least 200, at least 300, at least 400, at least 500, at least 600, at least 750, or at least 1000 nucleotides on each side of a core sequence. In some embodiments where a donor template is single-stranded DNA, a core sequence is flanked by homology arms including at least 10, at least 20, at least 30, at least 40, at least 50, at least 60, at least 70, at least 80, or at least 100 nucleotides on each side of a core sequence. In embodiments where a donor template is double-stranded DNA, a core sequence is flanked by homology arms including at least 500, at least 600, at least 700, at least 800, at least 900, or at least 1000 nucleotides on each side of the core sequence. In some embodiments, two separate double-strand breaks are introduced into a cell or subject's target nucleotide sequence with a “double nickase” Cas9 (see Ran et al. (2013) Cell, 154:1380-1389), followed by delivery of a donor template.

In some embodiments, disrupting agents of the present disclosure may comprise a polypeptide (e.g. peptide or protein moiety) as described herein, linked to a gRNA and a targeted nuclease, e.g., a Cas9, e.g., a wild type Cas9, a nickase Cas9 (e.g., Cas9 D10A), a dead Cas9 (dCas9), eSpCas9, Cpf1, C2C1, or C2C3, or a nucleic acid encoding such a nuclease. Choice of nuclease and gRNA(s) is determined by whether a targeted mutation is a deletion, substitution, or addition of nucleotides, e.g., a deletion, substitution, or addition of nucleotides to a targeted sequence. Fusions of a catalytically inactive endonuclease e.g., a dead Cas9 (dCas9, e.g., D10A; H840A) tethered with all or a portion of (e.g., biologically active portion of) an (one or more) effector domain (e.g., epigenome editors including but not restricted to: DNMT3a, DNMT3L, DNMT3b, KRAB domain, Tet1, p300, VP64 and fusions of the aforementioned) create chimeric proteins that can be linked to a polypeptide to guide a provided disrupting agent to specific DNA sites by one or more RNA sequences (e.g., DNA recognition elements including, but not restricted to zinc finger arrays, sgRNA, TAL arrays, peptide nucleic acids described herein) to modulate activity and/or expression of one or more target nucleic acids sequences (e.g., to methylate or demethylate a DNA sequence).

As used herein, a “biologically active portion of an effector domain” is a portion that maintains function (e.g. completely, partially, minimally) of an effector domain (e.g., a “minimal” or “core” domain). In some embodiments, fusion of a dCas9 with all or a portion of one or more effector domains of an epigenetic modifying moiety (such as a DNA methylase or enzyme with a role in DNA demethylation, e.g., DNMT3a, DNMT3b, DNMT3L, a DNMT inhibitor, combinations thereof, TET family enzymes, protein acetyl transferase or deacetylase, dCas9-DNMT3a/3L, dCas9-DNMT3a/3L/KRAB, dCas9/VP64) creates a chimeric protein that is linked to the polypeptide and useful in the methods described herein.

In some embodiments, a nucleic acid encoding a fusion polypeptide comprising dCas9-methylase is administered to a subject in need thereof in combination with a site-specific gRNA or antisense DNA oligonucleotide that targets a fusion to an anchor sequence (such as a CTCF binding motif), thereby decreasing affinity or ability of an anchor sequence to bind a nucleating polypeptide. In some embodiments, all or a portion of one or more methyltransferase, or enzyme associated with demethylation, effector domains are fused with an inactive nuclease, e.g., dCas9, and linked to a polypeptide. Exemplary dCas9 fusion methods and compositions that are adaptable to methods and compositions as provided herein are known and are described, e.g., in Kearns et al., Functional annotation of native enhancers with a Cas9-histone demethylase fusion. Nature Methods 12, 401-403 (2015); and McDonald et al., Reprogrammable CRISPR/Cas9-based system for inducing site-specific DNA methylation. Biology Open 2016: doi: 10.1242/bio.019067.

In some aspects, 1, 2, 3, 4, 5, 6, 7, 8, 9, 10, 11, 12, 13, 14, 15, 16, 17, 18, 19, 20, or more methyltransferase, or enzyme with a role in DNA demethylation, effector domains (all or a biologically active portion) are fused with dCas9 and linked to a polypeptide. Chimeric proteins described herein may also comprise a linker as described herein, e.g., an amino acid linker. In some aspects, a linker comprises 2 or more amino acids, e.g., one or more GS sequences. In some aspects, fusion of Cas9 (e.g., dCas9) with two or more effector domains (e.g., of a DNA methylase or enzyme with a role in DNA demethylation) comprises one or more interspersed linkers (e.g., GS linkers) between domains and is linked to a polypeptide. In some aspects, dCas9 is fused with a plurality (e.g., 2-5, e.g., 2, 3, 4, 5) of effector domains with interspersed linkers and is linked to a polypeptide.

In some embodiments, a genetic modifying moiety comprises one or more components of a CRISPR system described hereinabove.

For example, in some embodiments, a genetic modifying moiety comprises a gRNA that comprises a targeting domain that hybridizes to a nucleic acid comprising a target anchor sequence and/or has a sequence that is at least 80%, at least 85%, at least 90%, at least 95%, at least 97%, at least 98%, at least 99% identical to the complement of a nucleic acid comprising a target anchor sequence. In some embodiments, a gRNA is a site-specific gRNA in that its targeting domain does not hybridize to at least one nucleic acid comprising a non-target anchor sequence.

In some embodiments, the site-specific gRNA comprises a sequence of structure I:

X—Y—Z, (I)
where X and Z are 5′ and 3′ site-specific targeting sequences for a target CTCF binding motif, respectively, and Y is selected from:
(a) an RNA sequence complementary to a target sequence of interest (e.g. target sequence that is part of or participates in a target genomic complex);
(b) an RNA sequence at least 75%, 80%, 85%, 90%, 95% identical to an RNA sequence complementary to the target sequence of interest;
(c) an RNA sequence complementary to the target sequence of interest having at least 1, 2, 3, 4, 5, but less than 15, 12 or 10 nucleotide additions, substitutions or deletions.

In some embodiments, X and Z are each between 2-50 nucleotides in length, e.g., between 2-20, between 2-10, between 2-5 nucleotides in length.

In some embodiments, provided technologies are described as comprising a gRNA that specifically targets a target gene. In some embodiments, a target gene comprises an oncogene, a tumor suppressor gene, or a gene associated with a disease associated with a nucleotide repeat.

In some embodiments, technologies provided herein include methods of delivering one or more genetic modifying moieties (e.g. CRISPR system components) described herein to a subject, e.g., to a nucleus of a cell or tissue of a subject, by linking such a moiety to a disrupting agent described herein.

Epigenetic Modifying Moieties

In some embodiments, a disrupting agent comprises an epigenetic modifying moiety, e.g., a moiety that modulates two-dimensional structure of chromatin (i.e., that modulate structure of chromatin in a way that would alter its two-dimensional representation).

In some embodiments, an epigenetic modifying moiety comprises a histone modifying functionality, e.g., a histone methyltransferase, histone demethylase, or histone deacetylase activity. In some embodiments, a histone methyltransferase functionality comprises H3K9 targeting methyltransferase activity. In some embodiments, a histone methyltransferase functionality comprises H3K56 targeting methyltransferase activity. In some embodiments, a histone methyltransferase functionality comprises H3K27 targeting methyltransferase activity. In some embodiments, a histone methyltransferase or demethylase functionality transfers one, two, or three methyl groups. In some embodiments, a histone demethylase functionality comprises H3K4 targeting demethylase activity. In some embodiments, an epigenetic modifying moiety is or comprises a protein chosen from SETDB1, SETDB2, EHMT2 (i.e., G9A), EHMT1 (i.e., GLP), SUV39H1, EZH2, EZH1, SUV39H2, SETD8, SUV420H1, SUV420H2, or a functional variant or fragment of any thereof, e.g., a SET domain of any thereof. In some embodiments, an epigenetic modifying moiety is or comprises a protein chosen from KDM1A (i.e., LSD1), KDM1B (i.e., LSD2), KDM2A, KDM2B, KDM5A, KDM5B, KDM5C, KDM5D, KDM4B, NO66, or a functional variant or fragment of any thereof. In some embodiments, an epigenetic modifying moiety is or comprises a protein chosen from HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, or a functional variant or fragment of any thereof.

In some embodiments, an epigenetic modifying moiety comprises a DNA modifying functionality, e.g., a DNA methyltransferase. In some embodiments, an epigenetic modifying moiety is or comprises a protein chosen from MQ1, DNMT1, DNMT3A1, DNMT3A2, DNMT3B1, DNMT3B2, DNMT3B3, DNMT3B4, DNMT3B5, DNMT3B6, DNMT3L, or a functional variant or fragment of any thereof.

In some embodiments, an epigenetic modifying moiety comprises a transcription repressor. In some embodiments the transcription repressor blocks recruitment of a factor that stimulates or promotes transcription, e.g., of the target gene. In some embodiments, the transcription repressor recruits a factor that inhibits transcription, e.g., of the target gene. In some embodiments, an epigenetic modifying moiety, e.g., transcription repressor, is or comprises a protein chosen from KRAB, MeCP2, HP1, RBBP4, REST, FOG1, SUZ12, or a functional variant or fragment of any thereof.

In some embodiments, an epigenetic modifying moiety comprises a protein having a functionality described herein. In some embodiments, an epigenetic modifying moiety is or comprises a protein selected from:
KRAB (e.g., as according to NP_056209.2 or the protein encoded by NM_015394.5);
a SET domain (e.g., the SET domain of:
   SETDB1 (e.g., as according to NP_001353347.1 or the protein encoded by NM_001366418.1);
   EZH2 (e.g., as according to NP-004447.2 or the protein encoded by NM_004456.5);
   G9A (e.g., as according to NP_001350618.1 or the protein encoded by NM_001363689.1); or
   SUV39H1 (e.g., as according to NP_003164.1 or the protein encoded by NM_003173.4));
histone demethylase LSD1 (e.g., as according to NP_055828.2 or the protein encoded by NM_015013.4);
FOG1 (e.g., the N-terminal residues of FOG1) (e.g., as according to NP_722520.2 or the protein encoded by NM_153813.3); or
KAP1 (e.g., as according to NP_005753.1 or the protein encoded by NM_005762.3);
a functional fragment or variant of any thereof, or
   a polypeptide with a sequence that has at least 80, 85, 90, 91, 92, 93, 94, 95, 96, 97, 98, or 99% identity to any of the above-referenced sequences. In some embodiments, an epigenetic modifying moiety is or comprises a protein selected from:
DNMT3A (e.g., human DNMT3A) (e.g., as according to NP_072046.2
or the protein encoded by NM_022552.4);
DNMT3B (e.g., as according to NP_008823.1
or the protein encoded by NM_006892.4);
DNMT3L (e.g., as according to NP_787063.1
or the protein encoded by NM_175867.3);
DNMT3A/3L complex,
bacterial MQ1 (e.g., as according to CAA35058.1 or P15840.3);
a functional fragment of any thereof, or
   a polypeptide with a sequence that has at least 80, 85, 90, 91, 92, 93, 94, 95, 96, 97, 98, or 99% identity to any of the above-referenced sequences.

An exemplary an epigenetic modifying moiety may include, but is not limited to: ubiquitin, bicyclic peptides as ubiquitin ligase inhibitors, transcription factors, DNA and protein modification enzymes such as topoisomerases, topoisomerase inhibitors such as topotecan, DNA methyltransferases such as the DNMT family (e.g., DNMT3A, DNMT3B, DNMT3L), protein methyltransferases (e.g., viral lysine methyltransferase (vSET), protein-lysine N-methyltransferase (SMYD2), deaminases (e.g., APOBEC, UG1), histone methyltransferases such as enhancer of zeste homolog 2 (EZH2), PRMT1, histone-lysine-N-methyltransferase (Setdb1), histone methyltransferase (SET2), euchromatic histone-lysine N-methyltransferase 2 (G9a), histone-lysine N-methyltransferase (SUV39H1), and G9a), histone deacetylase (e.g., HDAC1, HDAC2, HDAC3), enzymes with a role in DNA demethylation (e.g., the TET family enzymes catalyze oxidation of 5-methylcytosine to 5-hydroxymethylcytosine and higher oxidative derivatives), protein demethylases such as KDM1A and lysine-specific histone demethylase 1 (LSD1), helicases such as DHX9, deacetylases (e.g., sirtuin 1, 2, 3, 4, 5, 6, or 7), kinases, phosphatases, DNA-intercalating agents such as ethidium bromide, SYBR green, and proflavine, efflux pump inhibitors such as peptidomimetics like phenylalanine arginyl β-naphthylamide or quinoline derivatives, nuclear receptor activators and inhibitors, proteasome inhibitors, competitive inhibitors for enzymes such as those involved in lysosomal storage diseases, protein synthesis inhibitors, nucleases (e.g., Cpf1, Cas9, zinc finger nuclease), fusions of one or more thereof (e.g., dCas9-DNMT, dCas9-APOBEC, dCas9-UG1), and specific domains from proteins, such as a KRAB domain

In some embodiments, the epigenetic modifying moiety is or comprises MQ1, e.g., bacterial MQ1, or a functional variant or fragment thereof. In some embodiments, MQ1 is Spiroplasma monobiae MQ1, e.g., MQ1 from strain ATCC 33825 and/or corresponding to Uniprot ID P15840. In some embodiments, an MQ1 variant comprises one or more amino acid substitutions, deletions, or insertions relative to wildtype MQ1. In some embodiments, an MQ1 variant comprises a K297P substitution. In some embodiments, an MQ1 variant comprises a N299C substitution. In some embodiments, an MQ1 variant comprises a E301Y substitution. In some embodiments, an MQ1 variant comprises a Q147L substitution (e.g., and has reduced DNA methyltransferase activity relative to wildtype MQ1). In some embodiments, an MQ1 variant comprises K297P, N299C, and E301Y substitutions (e.g., and has reduced DNA binding affinity relative to wildtype MQ1). In some embodiments, an MQ1 variant comprises Q147L, K297P, N299C, and E301Y substitutions (e.g., and has reduced DNA methyltransferase activity and DNA binding affinity relative to wildtype MQ1). In some embodiments, a disrupting agent comprises one or more linkers described herein, e.g., connecting a moiety/domain to another moiety/domain In some embodiments, a disrupting agent comprises a DNA-targeting moiety that is or comprises a CRISPR/Cas molecule, e.g., comprising a CRISPR/Cas protein, e.g., a dCas9 protein. In some embodiments, a disrupting agent is a fusion protein comprising an epigenetic modifying moiety that is or comprises MQ1 and a DNA-targeting moiety that is or comprises a CRISPR/Cas molecule, e.g., comprising a CRISPR/Cas protein, e.g., a dCas9 protein. In some embodiments, the disrupting agent comprises an additional moiety described herein. In some embodiments, the disrupting agent decreases expression of a target gene (e.g., a target gene described herein). In some embodiments, the disrupting agent may be used in methods of modulating, e.g., decreasing, gene expression, methods of treating a condition, or methods of epigenetically modifying a target gene or transcription control element described herein.

In some embodiments, a candidate domain may be determined to be suitable for use as an epigenetic modifying moiety by methods known to those of skill in the art. For example, a candidate epigenetic modifying moiety may be tested by assaying whether, when the candidate epigenetic modifying moiety is present in the nucleus of a cell and appropriately localized (e.g., to a target gene or transcription control element operably linked to said target gene, e.g., via a DNA-targeting moiety), the candidate epigenetic modifying moiety decreases expression of the target gene in the cell, e.g., decreases the level of RNA transcript encoded by the target gene (e.g., as measured by RNASeq or Northern blot) or decreases the level of protein encoded by the target gene (e.g., as measured by ELISA).

Epigenetic modifying moieties useful in methods and compositions of the present disclosure include agents that affect epigenetic markers, e.g., DNA methylation, histone methylation, histone acetylation, histone sumoylation, histone phosphorylation, and RNA-associated silencing. Exemplary epigenetic enzymes that can be targeted to a genomic sequence element as described herein include DNA methylases (e.g., DNMT3a, DNMT3b, DNMTL), DNA demethylation (e.g., the TET family), histone methyltransferases, histone deacetylase (e.g., HDAC1, HDAC2, HDAC3), sirtuin 1, 2, 3, 4, 5, 6, or 7, lysine-specific histone demethylase 1 (LSD1), histone-lysine-N-methyltransferase (Setdb1), euchromatic histone-lysine N-methyltransferase 2 (G9a), histone-lysine N-methyltransferase (SUV39H1), enhancer of zeste homolog 2 (EZH2), viral lysine methyltransferase (vSET), histone methyltransferase (SET2), and protein-lysine N-methyltransferase (SMYD2). Examples of such epigenetic modifying agents are described, e.g., in de Groote et al. Nuc. Acids Res. (2012):1-18.

In some embodiments, a disrupting agent, e.g., comprising an epigenetic modifying moiety, useful herein comprises or is a construct described in Koferle et al. Genome Medicine 7.59 (2015):1-3incorporated herein by reference. For example, in some embodiments, a disrupting agent comprises or is a construct found in Table 1 of Koferle et al., e.g., histone deacetylase, histone methyltransferase, DNA demethylation, or H3K4 and/or H3K9 histone demethylase described in Table 1 (e.g., dCas9-p300, TALE-TET1, ZF-DNMT3A, or TALE-LSD1).

Polypeptide Modifying Moieties

In some embodiments, a disrupting agent may comprise a polypeptide modifying moiety. In some embodiments, a polypeptide modifying moiety is or comprises an enzyme. In some embodiments, an enzyme participates in a polypeptide post-translational modification reaction (e.g. polypeptide phosphorylation, glycosylation). In some embodiments, modification of a polypeptide by a polypeptide modifying moiety impacts polypeptide inclusion in a genomic complex.

In some embodiments, a polypeptide modifying moiety is or comprises a kinase. In some embodiments, a kinase catalyzes the transfer of phosphate groups to a ligand (e.g. phosphorylation of a ligand). In some embodiments, a polypeptide modifying moiety is or comprises a phosphorylase. In some embodiments, a phosphorylase catalyzes addition of inorganic phosphate to a ligand.

In some embodiments, a polypeptide modifying moiety is or comprises a phosphatase. In some embodiments, a phosphatase catalyzes the removal of a phosphate group from a ligand.

Other Effector Moieties

Tagging or Monitoring Moieties

A site-specific disrupting agent may comprise a tag to label or monitor a polypeptide described herein or another moiety linked to a polypeptide. A tagging or monitoring moiety may be removable by chemical agents or enzymatic cleavage, such as proteolysis or intein splicing. An affinity tag may be useful to purify a tagged polypeptide using an affinity technique. Some examples include, chitin binding protein (CBP), maltose binding protein (MBP), glutathione-S-transferase (GST), and poly(His) tag. A solubilization tag may be useful to aid recombinant proteins expressed in chaperone-deficient species such as E. coli to assist in the proper folding in proteins and keep them from precipitating. Some examples include thioredoxin (TRX) and poly(NANP). A tagging or monitoring moiety may include a light sensitive tag, e.g., fluorescence. Fluorescent tags are useful for visualization. GFP and its variants are some examples commonly used as fluorescent tags. Protein tags may allow specific enzymatic modifications (such as biotinylation by biotin ligase) or chemical modifications (such as reaction with FlAsH-EDT2 for fluorescence imaging) to occur. Often tagging or monitoring moieties are combined, in order to connect proteins to multiple other components. A tagging or monitoring moiety may also be removed by specific proteolysis or enzymatic cleavage (e.g. by TEV protease, Thrombin, Factor Xa or Enteropeptidase).

In some embodiments, a tagging or monitoring moiety may be a small molecule, peptide, protein (including, e.g. protein fragment, antibody, antibody fragment, etc.), nucleic acid, nanoparticle, aptamer, or other agent or portion thereof.

Cleavable Moieties

In some embodiments, a site-specific disrupting agent comprises a moiety that may be cleaved from a polypeptide (e.g., after administration) by specific proteolysis or enzymatic cleavage (e.g. by TEV protease, Thrombin, Factor Xa or Enteropeptidase).

Membrane Translocating Moieties

Site-specific disrupting agents of the present disclosure may be or comprise a moiety linked to a membrane translocating polypeptide of the targeting moiety, such as through covalent bonds or non-covalent bonds or a linker as described herein. In some embodiments, a composition comprises a moiety linked to a membrane translocating moiety through a peptide bond. For example, in some embodiments, an amino terminal of a polypeptide is linked to membrane translocating moiety, such as through a peptide bond with an optional linker. In some embodiments, a carboxyl terminal of a polypeptide is linked to a membrane translocating moiety as described herein.

In some embodiments, a disrupting agent may comprise a membrane translocating polypeptide linked to two or more other (optional) moieties. For example, in some embodiments, an amino terminal and carboxyl terminal of a polypeptide are linked to other (optional) moieties, which may be the same or different from one another.

In some embodiments, one or more amino acids of a membrane translocating polypeptide are linked with another moiety, such as through disulfide bonds between cysteine side chains, hydrogen bonding, or any other another moiety may be a ligand or antibody to target a composition to a specific cell expressing a particular receptor. For example, in some embodiments, a chemotherapeutic agent, such as topotecan (a topoisomerase inhibitor), is linked to one end of a polypeptide, and a ligand or antibody is linked to another end of a polypeptide to target a composition to a specific cell or tissue. In some embodiments, other moieties are both effectors with biological activity.

In some embodiments, a plurality of membrane translocating polypeptides, either the same or different membrane translocating polypeptides, are comprised within, e.g., linked to, a single disrupting agent. Polypeptides may act as a coating that surrounds a disrupting agent and aids in its membrane penetration. Membrane translocating polypeptides may have a molecular weight greater than about 500 grams per mole or daltons, e.g., comprises organic or inorganic compounds that have a molecular weight greater than about 1,000, 2,000, 3,000, 4,000, or 5,000 grams per mole, e.g., with salts, esters, and other pharmaceutically acceptable forms of such compounds included.

In some embodiments, agents of the present disclosure may comprise a membrane translocating polypeptide comprised by, e.g., linked to, a disrupting agent on one or both ends and another separate moiety may be linked to another site on a polypeptide. One or both of amino terminal and carboxyl terminal of a polypeptide may be linked to a disrupting agent and one or more amino acid units in a moiety separate from a disrupting agent, either amino acids or nucleic acids, is linked to one or more additional moieties, such as through disulfide bonds or hydrogen bonding. In some embodiments, for example, a DNA modification enzyme is linked to a polypeptide, and a nucleic acid having an unmethylated CTCF binding motif that is complementary to a target methylated gene is hybridized to a nucleic acid side chain of the polypeptide. In some embodiments, upon administration, a composition may targets a CTCF genomic binding motif to modulate transcription of a gene. In some embodiments, a double stranded nucleic acid having an unmethylated CTCF binding motif with gene specific flanking sequences is linked to a polypeptide. In some embodiments, upon administration, an unmethylated CTCF binding motif serves as an alternate anchor sequence for CTCF protein to bind. In some embodiments, ubiquitin and another moiety, such as an effector, are linked to a disrupting agent. In some embodiments, upon administration, a disrupting agent penetrates a cell membrane and performs a function, e.g., the targeting and/or effector domain(s) perform a function. In some embodiments, after an performing a function, the disrupting agent is targeted by ubiquitin for degradation. In some embodiments, upon administration, a disrupting agent may target a non-CTCF genomic sequence (e.g. ncRNA, eRNA) to modulate transcription of a gene. In some embodiments, a disrupting agent may target a non-CTCF component of a genomic complex (e.g. transcription factor, transcription regulator, etc.) to modulate transcription of a gene.

In some embodiments, agents provided by the present disclosure may comprise a membrane translocating polypeptide comprised by or linked to a disrupting agent through covalent bonds and another optional moiety linked to nucleic acids in a polypeptide. In some embodiments, for example, a protein synthesis inhibitor is covalently linked to a polypeptide, and an siRNA or other target specific nucleic acid is hybridized to nucleic acids in a polypeptide. Upon administration, an siRNA targets a disrupting agent to an mRNA transcript and a protein synthesis inhibitor and siRNA act to inhibit expression of an mRNA.

Membrane translocating polypeptides as described herein can be linked to a disrupting agent by employing standard ligation techniques, such as those described herein to link polypeptides.

Pharmacoagent Moieties

In some embodiments, a disrupting agent may be or comprise a pharmacoagent moiety. In some embodiments, such a moiety may have an undesirable pharmacokinetic or pharmacodynamics (PK/PD) parameter. Linking such a pharmacoagent to a disrupting agent may improve at least one PK/PD parameter, such as targeting, absorption, and transport of the pharmacoagent, or reduce at least one undesirable PK/PD parameter, such as diffusion to off-target sites, and toxic metabolism. For example, linking a pharmacoagent to a disrupting agent as described herein to an agent with poor targeting/transport, e.g., doxorubicin, beta-lactams such as penicillin, improves its specificity. In some embodiments, linking a pharmacoagent to a disrupting agent as described herein to an agent with poor absorption properties, e.g., insulin, human growth hormone, improves its minimum dosage. In some embodiments, linking a pharmacoagent to a disrupting agent as described herein to an agent that has toxic metabolic properties, e.g., acetaminophen at higher doses, improves its maximum dosage.

Localization of Disrupting Agents

In some embodiments, agents of the present disclosure may comprise one or more targeting moieties that is or comprises a particular nucleic acid molecule (e.g. gRNA, PNA, BNA, etc.). In some embodiments, nucleic acid molecule comprises a sequence of structure I:

X—Y—Z, (II)
where X and Z are 5′ and 3′ site-specific targeting sequences, respectively, and Y is selected from:
(a) an RNA sequence complementary to a target sequence of interest (e.g. target sequence that is part of or participates in a target genomic complex)a target sequence of interest
(b) an RNA sequence at least 75%, 80%, 85%, 90%, 95% identical to an RNA sequence complementary to the target sequence of interest;
(c) an RNA sequence complementary to the target sequence of interest having at least 1, 2, 3, 4, 5, but less than 15, 12 or 10 nucleotide additions, substitutions or deletions.

In some embodiments, X and Z are each between 2-50 nucleotides in length, e.g., between 2-20, between 2-10, between 2-5 nucleotides in length.

In some embodiments, a nucleic acid molecule comprises a specific targeting sequence for at least one component of a genomic complex associated with a target gene. In some embodiments, a target gene comprises an oncogene, a tumor suppressor, or a disease associated with a nucleotide repeat.

For introducing small mutations or a single-point mutation, a homologous recombination (HR) template can be linked to a disrupting agent. In some embodiments, an HR template is a single stranded DNA (ssDNA) oligo or a plasmid. For ssDNA oligo design, one may use around 100-150 bp total homology with a mutation introduced roughly in the middle, giving 50-75 bp homology arms.

In some embodiments, a gRNA or antisense DNA oligonucleotide for targeting a target component of the genomic complex (e.g. a sequence that is part of a particular genomic complex), is linked to a targeting moiety in combination with an HR template selected from:
(a) a nucleotide sequence comprising a target sequence of interest (e.g. target sequence that is part of or participates in a target genomic complex);
(b) a nucleotide sequence at least 75%, 80%, 85%, 90%, 95% identical to a target sequence of interest
(c) a nucleotide sequence comprising a target sequence of interest having at least 1, 2, 3, 4, 5, but less than 15, 12 or 10 nucleotide additions, substitutions or deletions.

### Structure

As described herein, a disrupting agent and/or any moiety(ies) that comprise it, may have any appropriate chemical structure (e.g., may be comprised of, for example, one or more polypeptide, nucleic acid, small molecule, carbohydrate, lipid, and/or metal moiety(ies) or entity(ies) as well as, optionally, one or more linkers).

Polypeptides

Peptide or Protein Disrupting Agents

In some embodiments, a site-specific disrupting agent is or comprises a peptide or protein moiety. In some embodiments, a peptide or protein moiety is a targeting moiety. In some embodiments a protein moiety comprises an entire protein. In some embodiments, a protein moiety comprises a protein fragment. In some embodiments, a protein moiety comprises an antibody. In some embodiments, a protein moiety comprises an antibody fragment. As used herein, a protein moiety may comprise an entire protein or a portion or fragment of a protein. For example, in some embodiments, a targeting moiety comprises a DNA-binding protein, a CRISPR component protein, nucleating polypeptide, a dominant negative nucleating polypeptide, an epigenetic modifying moiety, or any combination thereof.

In some embodiments, a peptide or protein moiety may include, but is not limited to, a peptide ligand, a full-length protein, a protein fragment, an antibody, an antibody fragment, and/or a targeting aptamer. In some embodiments, a protein moiety may bind a receptor such as an extracellular receptor, neuropeptide, hormone peptide, peptide drug, toxic peptide, viral or microbial peptide, synthetic peptide, and agonist or antagonist peptide.

In some embodiments, a peptide or protein moiety may be linear or branched. A peptide or protein moiety may have a length from about 5 to about 200 amino acids, about 15 to about 150 amino acids, about 20 to about 125 amino acids, about 25 to about 100 amino acids, or any range therebetween.

In some embodiments, an exemplary peptide or protein moiety of methods and compositions as provided herein may include, but not be limited to, ubiquitin, bicyclic peptides as ubiquitin ligase inhibitors, transcription factors, DNA and protein modification enzymes such as topoisomerases, topoisomerase inhibitors such as topotecan, DNA methyltransferases such as the DNMT family (e.g., DNMT3a, DNMT3b, DNMTL), protein methyltransferases (e.g., viral lysine methyltransferase (vSET), protein-lysine N-methyltransferase (SMYD2), deaminases (e.g., APOBEC, UG1), histone methyltransferases such as enhancer of zeste homolog 2 (EZH2), PRMT1, histone-lysine-N-methyltransferase (Setdb1), histone methyltransferase (SET2), euchromatic histone-lysine N-methyltransferase 2 (G9a), histone-lysine N-methyltransferase (SUV39H1), and G9a), histone deacetylase (e.g., HDAC1, HDAC2, HDAC3), enzymes with a role in DNA demethylation (e.g., the TET family enzymes catalyze oxidation of 5-methylcytosine to 5-hydroxymethylcytosine and higher oxidative derivatives), protein demethylases such as KDM1A and lysine-specific histone demethylase 1 (LSD1), helicases such as DHX9, acetyltransferases, deacetylases (e.g., sirtuin 1, 2, 3, 4, 5, 6, or 7), kinases, phosphatases, DNA-intercalating agents such as ethidium bromide, SYBR green, and proflavine, efflux pump inhibitors such as peptidomimetics like phenylalanine arginyl β-naphthylamide or quinoline derivatives, nuclear receptor activators and inhibitors, proteasome inhibitors, competitive inhibitors for enzymes such as those involved in lysosomal storage diseases, protein synthesis inhibitors, nucleases (e.g., Cpf1, Cas9, zinc finger nuclease), fusions of one or more thereof (e.g., dCas9-DNMT, dCas9-APOBEC, dCas9-UG1), and specific domains from proteins, such as KRAB domain.

In some embodiments, peptide or protein moieties may include, but are not limited to, fluorescent tags or markers, antigens, antibodies, antibody fragments such as, e.g. single domain antibodies, ligands, and receptors such as, e.g., glucagon-like peptide-1 (GLP-1), GLP-2 receptor 2, cholecystokinin B (CCKB), and somatostatin receptor, peptide therapeutics such as, e.g., those that bind to specific cell surface receptors such as G protein-coupled receptors (GPCRs) or ion channels, synthetic or analog peptides from naturally-bioactive peptides, anti-microbial peptides, pore-forming peptides, tumor targeting or cytotoxic peptides, and degradation or self-destruction peptides such as an apoptosis-inducing peptide signal or photosensitizer peptide.

Peptide or protein moieties as described herein may also include small antigen-binding peptides, e.g., antigen binding antibody or antibody-like fragments, such as, e.g., single chain antibodies, nanobodies (see, e.g., Steeland et al. 2016. Nanobodies as therapeutics: big opportunities for small antibodies. Drug Discov Today: 21(7):1076-113). Such small antigen binding peptides may bind, e.g. a cytosolic antigen, a nuclear antigen, an intra-organellar antigen.

In some aspects, the present disclosure provides cells or tissues comprising any one of the peptides or protein moieties described herein.

In some aspects, the present disclosure provides methods of altering expression of a gene by administering a disrupting agent comprising a peptide or protein moiety described herein.

In some embodiments, a disrupting agent is or comprises a membrane translocating polypeptide as described herein.

Exemplary Polypeptide Disrupting Agents

(i) Protein Disrupting Agents

In some aspects, a disrupting agent is or comprises a protein. In some embodiments, gene expression is decreased via use of disrupting agents that are or comprise one or more proteins and dCas9. In some embodiments, one or more proteins is/are targeted to particular genomic complexes via dCas9 and target-specific guide RNA. As will be understood by one of skill in the art, proteins used for targeting may be the same or different depending on a given target. In some embodiments, gene expression is decreased in genomic complexes that comprise type 1, EP subtype complexes.

In some embodiments, gene expression is decreased in genomic complexes that are or comprise type 4 genomic complexes (e.g. ER, METTL3).

In some embodiments, gene expression is decreased via use of disrupting agents that are or comprise one or more proteins and dCas9, e.g., a fusion protein comprising dCas9 and a KRAB domain. In some embodiments, proteins is/are targeted to a particular genomic complex via dCas9 and target-specific guide RNA. In some embodiments, gene expression is decreased in genomic complexes that are or comprise type 1 (e.g. type 1, subtype 1) genomic complexes. In some embodiments, gene expression is decreased in genomic complexes that are or comprise type 3 genomic complexes.

(ii) Protein Fragment Disrupting Agents

In some aspects, a disrupting agent is or comprises a protein fragment. In some embodiments, gene expression is decreased via use of disrupting agents that are or comprise one or more protein fragments. In some embodiments, a protein fragment is targeted to assist in forming and/or stabilizing a particular genomic complex. In some embodiments, more than one protein fragment (e.g. more than one of identical protein fragments or one or more distinct protein fragments (e.g. at least two protein fragments, where each fragment is a different protein or different portions of a protein)) is targeted to a particular genomic complex. In some embodiments, gene expression is decreased via use of disrupting agents that are or comprise one or more protein fragments and dCas9. In some embodiments, protein is targeted to particular genomic complexes via dCas9 and target-specific guide RNA. As will be understood by one of skill in the art, protein fragments used for targeting may be the same or different depending on a given target.

In some embodiments, gene expression is increased in genomic complexes that are or comprise type 4 genomic complexes.

In some embodiments, gene expression is decreased via use of disrupting agents that are or comprise one or more protein fragments and dCas9. In some embodiments, one or more protein fragments is/are targeted to a particular genomic complex via dCas9 and target-specific guide RNA. In some embodiments, gene expression is decreased in genomic complexes that are or comprise type 1 genomic complexes. In some embodiments, gene expression is decreased in genomic complexes that are or comprise type 3 genomic complexes.

(iii)Antibody Disrupting Agents

In some aspects, a disrupting agent is or comprises an antibody. In some embodiments, gene expression is decreased via use of disrupting agents that are or comprise one or more antibodies. In some embodiments, gene expression is decreased via use of disrupting agents that are or comprise one or more antibodies and dCas9. In some embodiments, an antibody is targeted to particular genomic complex. In some embodiments, more than one antibody (e.g. more than one of identical antibodies or one or more distinct antibodies (e.g. at least two antibodies, where each antibody is a different antibody)) is targeted to a particular genomic complex. As will be understood by one of skill in the art, antibodies used for targeting may be the same or different depending on a given target. In some embodiments, one or more antibodies is/are targeted to particular genomic complexes via dCas9 and target-specific guide RNA. As will be understood by one of skill in the art, antibodies used for targeting may be the same or different depending on a given target. In some embodiments, gene expression is decreased in genomic complexes that comprise type 1, EP subtype complexes.

In some embodiments, gene expression is decreased in genomic complexes that are or comprise type 4 genomic complexes.

In some embodiments, gene expression is decreased via use of disrupting agents that are or comprise one or more antibodies and dCas9. In some embodiments, one or more antibodies is/are targeted to a particular genomic complex via dCas9 and target-specific guide RNA. In some embodiments, gene expression is decreased in genomic complexes that are or comprise type 1 genomic complexes. In some embodiments, gene expression is decreased in genomic complexes that are or comprise type 3 genomic complexes.

(iv)Antibody Fragment Disrupting Agents

In some aspects, a disrupting agent is or comprises an antibody fragment. In some embodiments, gene expression is decreased via use of disrupting agents that are or comprise one or more antibody fragments. In some embodiments, an antibody fragment is targeted to particular genomic complex. In some embodiments, more than one antibody fragment (e.g. more than one of identical antibody fragments or one or more distinct antibody fragments (e.g. at least two antibody fragments, where each antibody fragment is a different antibody fragment)) is targeted to a particular genomic complex. As will be understood by one of skill in the art, antibody fragments used for targeting may be the same or different depending on a given target. In some embodiments, gene expression is decreased via use of disrupting agents that are or comprise one or more antibody fragments and dCas9. In some embodiments, one or more antibody fragments is/are targeted to particular genomic complexes via dCas9 and target-specific guide RNA. In some embodiments, gene expression is decreased in genomic complexes that comprise type 1, EP subtype complexes.

In some embodiments, gene expression is decreased in genomic complexes that are or comprise type 4 genomic complexes.

In some embodiments, gene expression is decreased via use of disrupting agents that are or comprise one or more antibody fragments and dCas9. In some embodiments, one or more antibody fragments is/are targeted to a particular genomic complex via dCas9 and target-specific guide RNA. In some embodiments, gene expression is decreased in genomic complexes that are or comprise type 1 genomic complexes. In some embodiments, gene expression is decreased in genomic complexes that are or comprise type 3 genomic complexes.

(v) Antigen-Binding Fragment Disrupting Agents

In some aspects, a disrupting agent is or comprises an antigen-binding fragment. In some embodiments, gene expression is decreased via use of disrupting agents that are or comprise one or more antigen-binding fragments. In some embodiments, an antigen-binding fragment is targeted to particular genomic complex. In some embodiments, more than one antigen-binding fragment (e.g. more than one of identical antigen-binding fragments or one or more distinct antigen-binding fragments (e.g. at least two antigen-binding fragments, where each antigen-binding fragment is a different antigen-binding fragment)) is targeted to a particular genomic complex. As will be understood by one of skill in the art, antigen-binding fragments used for targeting may be the same or different depending on a given target.

(vi)Antibody Formats

In some aspects, a disrupting agent is or comprises an antibody that may be in one or more formats. In some embodiments, an antibody may be monoclonal or polyclonal. An antibody may be a fusion, a chimeric antibody, a non-humanized antibody, a partially or fully humanized antibody, etc. As will be understood by one of skill in the art, format of antibody(ies) used for targeting may be the same or different depending on a given target.

(vii) Nucleating Polypeptides

In some embodiments, a disrupting agent comprises a nucleating polypeptide or a portion thereof. In some embodiments, an anchor sequence-mediated conjunction is mediated by a first nucleating polypeptide bound to a first anchor sequence, a second nucleating polypeptide bound to a non-contiguous second anchor sequence, and an association between first and second nucleating polypeptides. In some embodiments, the disrupting agent may alter a genomic complex by destabilizing or inhibiting formation of the genomic complex.

(viii) DNA-Binding Domains

In some embodiments, a disrupting agent is or comprises a DNA-binding domain of a protein. In some such embodiments, the targeting moiety of the disrupting agent may be or comprise the DNA-binding domain. Alternatively or additionally, in some embodiments, one or more of a targeting moiety, and/or an effector moiety is or comprises a DNA-binding domain.

In some embodiments, DNA binding domains enhance or alter the effect of targeting by a disrupting agent, but do not alone achieve complete targeting by a disrupting agent. In some embodiments, DNA binding domains enhance targeting of a disrupting agent. In some embodiments, DNA binding domains enhance efficacy of a disrupting agent. DNA-binding proteins have distinct structural motifs that play a key role in binding DNA. A helix-turn-helix (HTH) motif is a common DNA recognition motif in repressor proteins. Such a motif comprises two helices, one of which recognizes DNA (aka recognition helix) with side chains providing binding specificity. Such motifs are commonly used to regulate proteins that are involved in developmental processes. Sometimes more than one protein competes for the same sequence or recognizes the same DNA fragment. Different proteins may differ in their affinity for the same sequence, or DNA conformation, respectively through H-bonds, salt bridges and Van der Waals interactions.

DNA-binding proteins with a helix-hairpin-helix HhH structural motif may be involved in non-sequence-specific DNA binding that occurs via the formation of hydrogen bonds between protein backbone nitrogens and DNA phosphate groups.

DNA-binding proteins with an HLH structural motif are transcriptional regulatory proteins and are principally related to a wide array of developmental processes. An HLH structural motif is longer, in terms of residues, than HTH or HhH motifs. Many of these proteins interact to form homo- and hetero-dimers. A structural motif is composed of two long helix regions, with an N-terminal helix binding to DNA, while a loop region allows the protein to dimerize.

In some transcription factors, a dimer binding site with DNA forms a leucine zipper. This motif includes two amphipathic helices, one from each subunit, interacting with each other resulting in a left handed coiled-coil super secondary structure. A leucine zipper is an interdigitation of regularly spaced leucine residues in one helix with leucines from an adjacent helix. Mostly, helices involved in leucine zippers exhibit a heptad sequence (abcdefg) with residues a and d being hydrophobic and other residues being hydrophilic. Leucine zipper motifs can mediate either homo- or heterodimer formation.

Some eukaryotic transcription factors show a unique motif called a Zn-finger, where a Zn⁺⁺ ion is coordinated by 2 Cys and 2 His residues. Such a transcription factor includes a trimer with the stoichiometry ββ′α. An apparent effect of Zn⁺⁺ coordination is stabilization of a small loop structure instead of hydrophobic core residues. Each Zn-finger interacts in a conformationally identical manner with successive triple base pair segments in the major groove of the double helix. Protein-DNA interaction is determined by two factors: (i) H-bonding interaction between α-helix and DNA segment, mostly between Arg residues and Guanine bases. (ii) H-bonding interaction with DNA phosphate backbone, mostly with Arg and His. An alternative Zn-finger motif chelates Zn⁺⁺ with 6 Cys.

DNA-binding proteins also include TATA box binding proteins (TBP), first identified as a component of the class II initiation factor TFIID. These binding proteins participate in transcription by all three nuclear RNA polymerases acting as subunit in each of them. Structure of TBP shows two α/β structural domains of 89-90 amino acids. The C-terminal or core region of TBP binds with high affinity to a TATA consensus sequence (TATAa/tAa/t, SEQ ID NO: 3) recognizing minor groove determinants and promoting DNA bending. TBP resemble a molecular saddle. The binding side is lined with central 8 strands of a 10-stranded anti-parallel β-sheet. The upper surface contains four α-helices and binds to various components of transcription machinery.

DNA provides base specificity via nitrogen bases. R-groups of amino acids, with basic residues such as Lysine, Arginine, Histidine, Asparagine and Glutamine can easily interact with adenine of an A: T base pair, and guanine of a G: C base pair, where NH2 and X═O groups of base pairs can preferably form hydrogen bonds with amino acid residues of Glutamine, Aspargine, Arginine and Lysine.

In some embodiments, a DNA-binding protein is a transcription factor. Transcription factors (TFs) may be modular proteins containing a DNA-binding domain that is responsible for specific recognition of base sequences and one or more effector domains that can activate or repress transcription. TFs interact with chromatin and recruit protein complexes that serve as coactivators or corepressors.

Production of Proteins or Polypeptides

As will be appreciated by one of skill, methods of making proteins or polypeptides (which may be included in disrupting agents as described herein) are routine in the art. See, in general, Smales & James (Eds.), Therapeutic Proteins: Methods and Protocols (Methods in Molecular Biology), Humana Press (2005); and Crommelin, Sindelar & Meibohm (Eds.), Pharmaceutical Biotechnology: Fundamentals and Applications, Springer (2013).

A protein or polypeptide of compositions of the present disclosure can be biochemically synthesized, e.g., by employing standard solid phase techniques. Such methods include exclusive solid phase synthesis, partial solid phase synthesis methods, fragment condensation, classical solution synthesis. These methods can be used when a peptide is relatively short (i.e., 10 kDa) and/or when it cannot be produced by recombinant techniques (e.g., not encoded by a nucleic acid sequence) and therefore involves different chemistry.

Solid phase synthesis procedures are well known in the art and further described by John Morrow Stewart and Janis Dillaha Young, Solid Phase Peptide Syntheses, 2nd Ed., Pierce Chemical Company, 1984; and Coin, I., et al., Nature Protocols, 2:3247-3256, 2007.

For longer peptides, recombinant methods may be used. Methods of making a recombinant therapeutic polypeptide are routine in the art. See, in general, Smales & James (Eds.), Therapeutic Proteins: Methods and Protocols (Methods in Molecular Biology), Humana Press (2005); and Crommelin, Sindelar & Meibohm (Eds.), Pharmaceutical Biotechnology: Fundamentals and Applications, Springer (2013).

Exemplary methods for producing a therapeutic pharmaceutical protein or polypeptide involve expression in mammalian cells, although recombinant proteins can also be produced using insect cells, yeast, bacteria, or other cells under control of appropriate promoters. Mammalian expression vectors may comprise nontranscribed elements such as an origin of replication, a suitable promoter, and other 5′ or 3′ flanking nontranscribed sequences, and 5′ or 3′ nontranslated sequences such as necessary ribosome binding sites, a polyadenylation site, splice donor and acceptor sites, and termination sequences. DNA sequences derived from the SV40 viral genome, for example, SV40 origin, early promoter, splice, and polyadenylation sites may be used to provide other genetic elements required for expression of a heterologous DNA sequence. Appropriate cloning and expression vectors for use with bacterial, fungal, yeast, and mammalian cellular hosts are described in Green & Sambrook, Molecular Cloning: A Laboratory Manual (Fourth Edition), Cold Spring Harbor Laboratory Press (2012).

In cases where large amounts of the protein or polypeptide are desired, it can be generated using techniques such as described by Brian Bray, Nature Reviews Drug Discovery, 2:587-593, 2003; and Weissbach & Weissbach, 1988, Methods for Plant Molecular Biology, Academic Press, NY, Section VIII, pp 421-463.

Various mammalian cell culture systems can be employed to express and manufacture recombinant protein. Examples of mammalian expression systems include CHO cells, COS cells, HeLA and BHK cell lines. Processes of host cell culture for production of protein therapeutics are described in Zhou and Kantardjieff (Eds.), Mammalian Cell Cultures for Biologics Manufacturing (Advances in Biochemical Engineering/Biotechnology), Springer (2014). Compositions described herein may include a vector, such as a viral vector, e.g., a lentiviral vector, encoding a recombinant protein. In some embodiments, a vector, e.g., a viral vector, may comprise a nucleic acid encoding a recombinant protein.

Purification of protein therapeutics is described in Franks, Protein Biotechnology: Isolation, Characterization, and Stabilization, Humana Press (2013); and in Cutler, Protein Purification Protocols (Methods in Molecular Biology), Humana Press (2010).

Formulation of protein therapeutics is described in Meyer (Ed.), Therapeutic Protein Drug Products: Practical Approaches to formulation in the Laboratory, Manufacturing, and the Clinic, Woodhead Publishing Series (2012).

Protein Encoding Nucleic Acids

In some embodiments, a disrupting agent is or comprises a vector, e.g., a viral vector comprising one or more nucleic acids encoding one or more components of a modulating agent (e.g., disrupting agent) as described herein.

Nucleic acids as described herein or nucleic acids encoding a protein described herein, may be incorporated into a vector. Vectors, including those derived from retroviruses such as lentivirus, are suitable tools to achieve long-term gene transfer since they allow long-term, stable integration of a transgene and its propagation in daughter cells. Examples of vectors include expression vectors, replication vectors, probe generation vectors, and sequencing vectors. An expression vector may be provided to a cell in the form of a viral vector. Viral vector technology is well known in the art, and described in a variety of virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers.

Expression of natural or synthetic nucleic acids is typically achieved by operably linking a nucleic acid encoding the gene of interest to a promoter, and incorporating the construct into an expression vector. Vectors can be suitable for replication and integration in eukaryotes. Typical cloning vectors contain transcription and translation terminators, initiation sequences, and promoters useful for expression of the desired nucleic acid sequence.

Additional promoter elements, e.g., enhancing sequences, may regulate frequency of transcriptional initiation. Typically, these sequences are located in a region 30-110 bp upstream of a transcription start site, although a number of promoters have recently been shown to contain functional elements downstream of transcription start sites as well. Spacing between promoter elements frequently is flexible, so that promoter function is preserved when elements are inverted or moved relative to one another. In a thymidine kinase (tk) promoter, spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. In some embodiments of a suitable promoter is Elongation Growth Factor-1a (EF-1a). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, an actin promoter, a myosin promoter, a hemoglobin promoter, and a creatine kinase promoter.

The present disclosure should not interpreted to be limited to use of any particular promoter or category of promoters (e.g. constitutive promoters). For example, in some embodiments, inducible promoters are contemplated as part of the present disclosure. In some embodiments, use of an inducible promoter provides a molecular switch capable of turning on expression of a polynucleotide sequence to which it is operatively linked, when such expression is desired. In some embodiments, use of an inducible promoter provides a molecular switch capable of turning off expression when expression is not desired. Examples of inducible promoters include, but are not limited to a metallothionine promoter, a glucocorticoid promoter, a progesterone promoter, and a tetracycline promoter.

In some embodiments, an expression vector to be introduced can also contain either a selectable marker gene or a reporter gene or both to facilitate identification and selection of expressing cells from the population of cells sought to be transfected or infected through viral vectors. In some aspects, a selectable marker may be carried on a separate piece of DNA and used in a co-transfection procedure. Both selectable markers and reporter genes may be flanked with appropriate transcriptional control sequences to enable expression in the host cells. Useful selectable markers may include, for example, antibiotic-resistance genes, such as neo, etc. In some embodiments, reporter genes may be used for identifying potentially transfected cells and/or for evaluating the functionality of transcriptional control sequences. In general, a reporter gene is a gene that is not present in or expressed by a recipient source (of a reporter gene) and that encodes a polypeptide whose expression is manifested by some easily detectable property, e.g., enzymatic activity or visualizable fluorescence. Expression of a reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, a construct with a minimal 5′ flanking region that shows highest level of expression of reporter gene is identified as a promoter. Such promoter regions may be linked to a reporter gene and used to evaluate agents for ability to alter promoter-driven transcription.

Nucleic Acids

A disrupting agent may be or comprise a moiety (e.g., a moiety described herein) comprising one or more nucleic acids, e.g., a nucleic acid moiety, or entity. In some embodiments, a nucleic acid that may be included in a nucleic acid moiety or entity as described herein, may be or comprise DNA, RNA, and/or an artificial or synthetic nucleic acid or nucleic acid analog or mimic. For example, in some embodiments, a nucleic acid included in a nucleic acid moiety as described herein may be or include one or more of genomic DNA (gDNA), complementary DNA (cDNA), a peptide nucleic acid (PNA), a peptide-oligonucleotide conjugate, a locked nucleic acid (LNA), a bridged nucleic acid (BNA), a polyamide, a triplex-forming oligonucleotide, an antisense oligonucleotide, tRNA, mRNA, rRNA, miRNA, gRNA, siRNA or other RNAi molecule (e.g., that targets a non-coding RNA as described herein and/or that targets an expression product of a particular gene associated with genomic complex as described herein), etc. In some embodiments, a nucleic acid included in a nucleic acid moiety or entity as described herein may include one or more residues that is not a naturally-occurring DNA or RNA residue, may include one or more linkages that is/are not phosphodiester bonds (e.g., that may be, for example, phosphorothioate bonds, etc.), and/or may include one or more modifications such as, for example, a 2′O modification such as 2′-OMeP. A variety of nucleic acid structures useful in preparing synthetic nucleic acids is known in the art (see, for example, WO2017/0628621 and WO2014/012081) those skilled in the art will appreciate that these may be utilized in accordance with the present disclosure.

In some embodiments, nucleic acids included in a nucleic acid moiety or entity as described herein may have a length from about 2 to about 5000 nts, about 10 to about 100 nts, about 50 to about 150 nts, about 100 to about 200 nts, about 150 to about 250 nts, about 200 to about 300 nts, about 250 to about 350 nts, about 300 to about 500 nts, about 10 to about 1000 nts, about 50 to about 1000 nts, about 100 to about 1000 nts, about 1000 to about 2000 nts, about 2000 to about 3000 nts, about 3000 to about 4000 nts, about 4000 to about 5000 nts, or any range therebetween.

Some examples of nucleic acids that may be utilized in a nucleic acid moiety or entity as described herein include, but are not limited to, a nucleic acid that hybridizes to an endogenous gene (e.g., gRNA or antisense ssDNA as described herein elsewhere), a nucleic acid that hybridizes to an exogenous nucleic acid such as a viral DNA or RNA, nucleic acid that hybridizes to an RNA, a nucleic acid that interferes with gene transcription, a nucleic acid that interferes with RNA translation, a nucleic acid that stabilizes RNA or destabilizes RNA such as through targeting for degradation, a nucleic acid that interferes with a DNA or RNA binding factor through interference of its expression or its function, a nucleic acid that is linked to a intracellular protein or protein complex and modulates its function, etc.

The present disclosure contemplates disrupting agents comprising RNA therapeutics (e.g., modified RNAs) as useful components of provided compositions as described herein. For example, in some embodiments, a modified mRNA encoding a protein of interest may be linked to a polypeptide described herein and expressed in vivo in a subject.

Nucleic Acid Analogs

In some aspects, a disrupting agent may be or comprise one or more nucleoside analogs. In some embodiments, a nucleic acid sequence may include in addition or as an alternative to one or more natural nucleosides nucleosides, e.g., purines or pyrimidines, e.g., adenine, cytosine, guanine, thymine and uracil. In some embodiments, a nucleic acid sequence includes one or more nucleoside analogs. A nucleoside analog may include, but is not limited to, a nucleoside analog, such as 5-fluorouracil; 5-bromouracil, 5-chlorouracil, 5-iodouracil, hypoxanthine, xanthine, 4-acetylcytosine, 4-methylbenzimidazole, 5-(carboxyhydroxylmethyl) uracil, 5-carboxymethylaminomethyl-2-thiouridine, 5-carboxymethylaminomethyluracil, dihydrouracil, dihydrouridine, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, 2,6-diaminopurine, 3-nitropyrrole, inosine, thiouridine, queuosine, wyosine, diaminopurine, isoguanine, isocytosine, diaminopyrimidine, 2,4-difluorotoluene, isoquinoline, pyrrolo[2,3-β]pyridine, and any others that can base pair with a purine or a pyrimidine side chain.

Peptide Oligonucleotide Conjugates

In some embodiments, a disrupting agent may be or comprise a peptide oligonucleotide conjugate moiety or entity. Peptide oligonucleotide conjugates include chimeric molecules comprising a nucleic acid moiety linked to a peptide moiety (such as a peptide/nucleic acid mixmer). In some embodiments, a peptide moiety may include any peptide or protein moiety described herein. In some embodiments, a nucleic acid moiety may include any nucleic acid or oligonucleotide, e.g., DNA or RNA or modified DNA or RNA, described herein.

In some embodiments, a peptide oligonucleotide conjugate comprises a peptide antisense oligonucleotide conjugate. In some embodiments, a peptide oligonucleotide conjugate is a synthetic oligonucleotide with a chemically modified backbone. A peptide oligonucleotide conjugate can bind to both DNA and RNA targets in a sequence-specific manner to form a duplex structure. When bound to double-stranded DNA (dsDNA) target, a peptide oligonucleotide conjugate replaces one DNA strand in a duplex by strand invasion to form a triplex structure and a displaced DNA strand may exist as a single-stranded D-loop.

In some embodiments, a peptide oligonucleotide conjugate may be cell- and/or tissue-specific. In some embodiments, such a conjugate may be conjugated directly to, e.g. oligos, peptides, and/or proteins, etc.

In some embodiments, a peptide oligonucleotide conjugate comprises a membrane translocating polypeptide, for example, a membrane translocating polypeptides as described elsewhere herein.

Solid-phase synthesis of several peptide-oligonucleotide conjugates has been described in, for example, Williams, et al., 2010, Curr. Protoc. Nucleic Acid Chem., Chapter Unit 4.41, doi: 10.1002/0471142700.nc0441s42. Synthesis and characterization of very short peptide-oligonucleotide conjugates and stepwise solid-phase synthesis of peptide-oligonucleotide conjugates on new solid supports have been described in, for example, Bongardt, et al., Innovation Perspect. Solid Phase Synth. Comb. Libr., Collect. Pap., Int. Symp., 5th, 1999, 267-270; Antopolsky, et al., Helv. Chim. Acta, 1999, 82, 2130-2140.

Aptamers

A disrupting agent may be or comprise an aptamer, such as an oligonucleotide aptamer or a peptide aptamer. Aptamer moieties are oligonucleotide or peptide aptamers.

A disrupting agent may be or comprise an oligonucleotide aptamer. Oligonucleotide aptamers are single-stranded DNA or RNA (ssDNA or ssRNA) molecules that can bind to pre-selected targets including proteins and peptides with high affinity and specificity.

Oligonucleotide aptamers are nucleic acid species that may be engineered through repeated rounds of in vitro selection or equivalently, SELEX (systematic evolution of ligands by exponential enrichment) to bind to various molecular targets such as small molecules, proteins, nucleic acids, and even cells, tissues and organisms. Aptamers provide discriminate molecular recognition, and can be produced by chemical synthesis. In addition, aptamers possess desirable storage properties, and elicit little or no immunogenicity in therapeutic applications.

Both DNA and RNA aptamers show robust binding affinities for various targets. For example, DNA and RNA aptamers have been selected for t lysozyme, thrombin, human immunodeficiency virus trans-acting responsive element (HIV TAR), available on the world wide web at en.wikipedia.org/wiki/Aptamer—cite_note-10 hemin, interferon γ, vascular endothelial growth factor (VEGF), prostate specific antigen (PSA), dopamine, and the non-classical oncogene, heat shock factor 1 (HSF1).

Diagnostic techniques for aptamer based plasma protein profiling includes aptamer plasma proteomics. This technology will enable future multi-biomarker protein measurements that can aid diagnostic distinction of disease versus healthy states.

A disrupting agent may be or comprise a peptide aptamer moiety. Peptide aptamers have one (or more) short variable peptide domains, including peptides having low molecular weight, 12-14 kDa. Peptide aptamers may be designed to specifically bind to and interfere with protein-protein interactions inside cells.

Peptide aptamers are artificial proteins selected or engineered to bind specific target molecules. These proteins include of one or more peptide loops of variable sequence. They are typically isolated from combinatorial libraries and often subsequently improved by directed mutation or rounds of variable region mutagenesis and selection. In vivo, peptide aptamers can bind cellular protein targets and exert biological effects, including interference with the normal protein interactions of their targeted molecules with other proteins. In particular, a variable peptide aptamer loop attached to a transcription factor binding domain is screened against a target protein attached to a transcription factor activating domain. In vivo binding of a peptide aptamer to its target via this selection strategy is detected as expression of a downstream yeast marker gene. Such experiments identify particular proteins bound by aptamers, and protein interactions that aptamers modulate, to cause a given phenotype. In addition, peptide aptamers derivatized with appropriate functional moieties can cause specific post-translational modification of their target proteins, or change subcellular localization of the targets.

Peptide aptamers can also recognize targets in vitro. They have found use in lieu of antibodies in biosensors and used to detect active isoforms of proteins from populations containing both inactive and active protein forms. Derivatives known as tadpoles, in which peptide aptamer “heads” are covalently linked to unique sequence double-stranded DNA “tails”, allow quantification of scarce target molecules in mixtures by PCR (using, for example, the quantitative real-time polymerase chain reaction) of their DNA tails.

Peptide aptamer selection can be made using different systems, but the most used is currently a yeast two-hybrid system. Peptide aptamers can also be selected from combinatorial peptide libraries constructed by phage display and other surface display technologies such as mRNA display, ribosome display, bacterial display and yeast display. These experimental procedures are also known as biopannings. Among peptides obtained from biopannings, mimotopes can be considered as a kind of peptide aptamers. Peptides panned from combinatorial peptide libraries have been stored in a special database with named MimoDB.

In some embodiments, a disrupting agent is or comprises a nucleic acid sequence. In some embodiments, a nucleic acid encodes a gene expression product.

As will be readily understood by those skilled in the art reading the present disclosure, a targeting moiety can comprise a nucleic acid that does not encode a gene expression product. For example, in some embodiments, a targeting moiety may comprise an oligonucleotide that hybridizes to a target anchor sequence. For example, in some embodiments, a sequence of an oligonucleotide comprises a complement of a target anchor sequence, or has a sequence that is at least 80%, at least 85%, at least 90%, at least 95%, at least 97%, at least 98%, at least 99% identical to the complement of a target anchor sequence.

A nucleic acid sequence may include, but is not limited to, DNA, RNA, modified oligonucleotides (e.g., chemical modifications, such as modifications that alter backbone linkages, sugar molecules, and/or nucleic acid bases), and artificial nucleic acids. In some embodiments, a nucleic acid sequence includes, but is not limited to, genomic DNA, cDNA, peptide nucleic acids (PNA) or peptide oligonucleotide conjugates, locked nucleic acids (LNA), bridged nucleic acids (BNA), polyamides, triplex forming oligonucleotides, modified DNA, antisense DNA oligonucleotides, tRNA, mRNA, rRNA, modified RNA, miRNA, gRNA, and siRNA or other RNA or DNA molecules.

In some embodiments, a nucleic acid sequence has a length from about 2 to about 5000 nts, about 10 to about 100 nts, about 50 to about 150 nts, about 100 to about 200 nts, about 150 to about 250 nts, about 200 to about 300 nts, about 250 to about 350 nts, about 300 to about 500 nts, about 10 to about 1000 nts, about 50 to about 1000 nts, about 100 to about 1000 nts, about 1000 to about 2000 nts, about 2000 to about 3000 nts, about 3000 to about 4000 nts, about 4000 to about 5000 nts, or any range therebetween.

In some aspects, the present disclosure provides a synthetic nucleic acid comprising a plurality of anchor sequences, a gene sequence, and/or a transcriptional control sequence. In some embodiments, a synthetic nucleic acid comprises a plurality of anchor sequence, a gene sequence, and a transcriptional control sequence; in some such embodiments, a gene sequence and a transcriptional control sequence are between anchor sequences in the plurality of anchor sequences. In some embodiments, a synthetic nucleic acid comprises, in order, (a) an anchor sequence, a gene sequence, a transcriptional control sequence, and an anchor sequence or (b) an anchor sequence, a transcriptional control sequence, a gene sequence, and an anchor sequence. In some embodiments, sequences are separated by linker sequences. In some embodiments, anchor sequences are between 7-100 nts, 10-100 nts, 10-80 nts, 10-70 nts, 10-60 nts, 10-50 nts, 20-80 nts, or any range therebetween. In some embodiments, a nucleic acid is between 3,000-50,000 bp, 3,000-40,000 bp, 3,000-30,000 bp, 3,000-20,000 bp, 3,000-15,000 bp, 3,000-12,000 bp, 3,000-10,000 bp, 3,000-8,000 bp, 5,000-30,000 bp, 5,000-20,000 bp, 5,000-15,000 bp, 5,000-12,000 bp, 5,000-10,000 bp or any range therebetween.

In some embodiments, a genomic complex may be or comprise one or more synthetic nucleic acids (e.g., one or more components of a genomic complex may be or comprise a synthetic nucleic acid). In some embodiments, all nucleic acid components of a genomic complex are synthetic nucleic acids. In some embodiments, all non-genomic nucleic acid components of a genomic complex are synthetic nucleic acids.

In some embodiments, a genomic complex component that is or is comprised of synthetic nucleic acids may be exogenously provided [e.g. to a subject, a cell, etc. (e.g. in vitro, ex vivo, in vivo)] such that the provided component may bind to/complex with one or more endogenous genomic complex components.

In some embodiments, an exogenously added component (including, for example, an exogenously-added synthetic nucleic acid) may have a modified structure as compared with an endogenous genomic complex component (e.g., may be an analog or structural variant of a corresponding endogenous genomic complex component), which modified structure alters an interaction that the modified, exogenously-added component has with one or more other complex components relative to that interaction had by the corresponding endogenous component.

In some embodiments, a genomic complex component comprised of synthetic nucleic acids may be exogenously provided [e.g. to a subject, a cell, etc. (e.g. in vitro, ex vivo, in vivo)] such that the provided component may bind to/complex with one or more endogenous genomic complex components. In some embodiments, a genomic complex component comprised of synthetic nucleic acids may be altered, e.g., in its activity or binding affinity/preference, such that when it is exogenously provided [e.g. to a subject, a cell, etc. (e.g. in vitro, ex vivo, in vivo)] the provided component destabilizes or inhibits formation of a target genomic complex.

Exemplary Nucleic Acid Disrupting Agents

In some embodiments, gene expression is increased via use of disrupting agents that are or comprise one or more nucleic acid moieties. In some embodiments, a disrupting agent is or comprises one or more RNAs (e.g. gRNA) and dCas9. In some embodiments, one or more RNAs is/are targeted to particular genomic complexes via dCas9 and target-specific guide RNA. As will be understood by one of skill in the art, RNAs used for targeting may be the same or different depending on a given target. In some embodiments, gene expression is decreased in genomic complexes that comprise type 1, EP subtype complexes.

In some embodiments, gene expression is decreased in genomic complexes that are or comprise type 4 genomic complexes (e.g. ER sequence, CTCF sequence, YY1 sequence).

In some embodiments, gene expression is decreased via use of disrupting agents that are or comprise one or more antibody fragments and dCas9. In some embodiments, one or more RNAs is/are targeted to a particular genomic complex via dCas9 and target-specific guide RNA. In some embodiments, gene expression is decreased in genomic complexes that are or comprise type 1 genomic complexes. In some embodiments, gene expression is decreased in genomic complexes that are or comprise type 3 genomic complexes.

(ix) gRNA

In some embodiments, a disrupting agent comprises a nucleic acid sequence, e.g., a guide RNA (gRNA). In some embodiments, a disrupting agent comprises a guide RNA or nucleic acid encoding the guide RNA. A gRNA short synthetic RNA composed of a “scaffold” sequence necessary for Cas9-binding and a user-defined ˜20 nucleotide targeting sequence for a genomic target. In practice, guide RNA sequences are generally designed to have a length of between 17-24 nucleotides (e.g., 19, 20, or 21 nucleotides) and complementary to the targeted nucleic acid sequence. Custom gRNA generators and algorithms are available commercially for use in the design of effective guide RNAs. Gene editing has also been achieved using a chimeric “single guide RNA” (“sgRNA”), an engineered (synthetic) single RNA molecule that mimics a naturally occurring crRNA-tracrRNA complex and contains both a tracrRNA (for binding the nuclease) and at least one crRNA (to guide the nuclease to the sequence targeted for editing). Chemically modified sgRNAs have also been demonstrated to be effective in genome editing; see, for example, Hendel et al. (2015) Nature Biotechnol., 985-991.

In some embodiments, a gRNA is complementary to a region on a particular anchor sequence-mediated conjunction (e.g. genomic loop). In some embodiments, a gRNA is complementary to a region on a particular anchor sequence-mediated conjunction (e.g. genomic loop) that is not a nucleating polypeptide binding motif (e.g. CTCF binding motif).

In some embodiments, a gRNA is complementary to part of a genomic complex. In some embodiments, a gRNA is complementary to a genomic sequence element. In some embodiments, a gRNA is complementary to genomic sequence that is not itself part of an anchor sequence-mediated conjunction and/or genomic complex. For example, in some such embodiments, a gRNA may be complementary to genomic sequence encoding a transcription factor, wherein the transcription factor is part of a genomic complex, but the genomic sequence encoding the transcription factor is, e.g. on a different chromosome.

In some embodiments, a nucleic acid sequence comprises a sequence complementary to an anchor sequence. In some embodiments, an anchor sequence comprises a CTCF-binding motif or consensus sequence: N(T/C/G)N(G/A/T)CC(A/T/G)(C/G)(C/T/A)AG(G/A)(G/T)GG(C/A/T)(G/A)(C/G)(C/T/A)(G/A/C) (SEQ ID NO:1), where N is any nucleotide. A CTCF-binding motif or consensus sequence may also be in the opposite orientation, e.g., (G/A/C)(C/T/A)(C/G)(G/A)(C/A/T)GG(G/T)(G/A)GA(C/T/A)(C/G)(A/T/G)CC(G/A/T)N(T/C/G)N (SEQ ID NO:2). In some embodiments, a nucleic acid sequence comprises a sequence complementary to a CTCF-binding motif or consensus sequence.

In some embodiments, a nucleic acid sequence comprises a sequence complementary to a sequence within a particular anchor sequence-mediated conjunction (e.g. genomic loop). In some embodiments, a nucleic acid sequence comprises a sequence complementary to a sequence within a particular anchor sequence-mediated conjunction (e.g. genomic loop) that is not an anchor sequence or a nucleating polypeptide binding motif. In some embodiments, a nucleic acid sequence comprises a sequence complementary to a sequence produced by a gross chromosomal rearrangement, e.g., that is specific to cells comprising or having undergone a gross chromosomal rearrangement, e.g., that is not normally present in wildtype cells. In some embodiments, a nucleic acid sequence comprises a sequence complementary to a breakpoint, a fusion gene (e.g., fusion oncogene), or both. In some embodiments, a nucleic acid sequence comprises a sequence complementary to a cancer-specific anchor sequence.

In some embodiments, a nucleic acid sequence comprises a sequence at least 80%, at least 90%, at least 95%, at least 96%, at least 97%, at least 98%, at least 99% complementary to an anchor sequence or sequence within an anchor sequence-mediated conjunction. In some embodiments, a nucleic acid sequence comprises a sequence at least 80%, at least 90%, at least 95%, at least 96%, at least 97%, at least 98%, at least 99% complementary to a CTCF-binding motif, consensus sequence, or sequence within an anchor sequence-mediated conjunction. In some embodiments, a nucleic acid sequence is selected from the group consisting of a gRNA, and a sequence complementary or a sequence comprising at least 80%, at least 90%, at least 95%, at least 96%, at least 97%, at least 98%, at least 99% complementary sequence to an anchor sequence or sequence within an anchor sequence-mediated conjunction. In some embodiments, a nucleic acid sequence comprises a sequence at least 80%, at least 90%, at least 95%, at least 96%, at least 97%, at least 98%, at least 99% complementary to a sequence produced by a gross chromosomal rearrangement, e.g., that is specific to cells comprising or having undergone a gross chromosomal rearrangement, e.g., that is not normally present in wild-type cells. In some embodiments, a nucleic acid sequence comprises a sequence at least 80%, at least 90%, at least 95%, at least 96%, at least 97%, at least 98%, at least 99% complementary to a breakpoint, a fusion gene (e.g., fusion oncogene), or both. In some embodiments, a nucleic acid sequence comprises a sequence at least 80%, at least 90%, at least 95%, at least 96%, at least 97%, at least 98%, at least 99% complementary to a cancer-specific anchor sequence.

In some embodiments, an epigenetic modifying moiety is a gRNA, antisense DNA, or triplex forming oligonucleotide used as a DNA target and steric presence in the vicinity of the anchoring sequence. A gRNA recognizes specific DNA sequences (e.g., an anchor sequence, a CTCF anchor sequence, flanked by sequences that confer sequence specificity). A gRNA may include additional sequences that interfere with nucleating polypeptide binding motif to act as a steric blocker. In some embodiments, a gRNA is combined with one or more peptides, e.g., S-adenosyl methionine (SAM), that acts as a steric presence to interfere with a nucleating polypeptide.

(x) RNAi

In some embodiments, a disrupting agent comprises an RNAi molecule. Certain RNA agents can inhibit gene expression through a biological process using RNA interference (RNAi). RNAi molecules comprise RNA or RNA-like structures typically containing 15-50 base pairs (such as about 18-25 base pairs) and having a nucleobase sequence identical (complementary) or nearly identical (substantially complementary) to a coding sequence in an expressed target gene within the cell. RNAi molecules include, but are not limited to: short interfering RNAs (siRNAs), double-strand RNAs (dsRNA), micro RNAs (miRNAs), short hairpin RNAs (shRNA), meroduplexes, and dicer substrates (U.S. Pat. Nos. 8,084,599 8,349,809 and 8,513,207).

In some embodiments, the RNAi molecule binds to an eRNA, e.g., to decrease its activity or levels. In some embodiments, binding of the RNAi molecule to the eRNA disrupts the genomic complex.

RNAi molecules comprise a sequence substantially complementary, or fully complementary, to all or a fragment of a target gene. RNAi molecules may complement sequences at a boundary between introns and exons to prevent maturation of newly-generated nuclear RNA transcripts of specific genes into mRNA for transcription. RNAi molecules complementary to specific genes can hybridize with an mRNA for that gene and prevent its translation. An antisense molecule can be, for example, DNA, RNA, or a derivative or hybrid thereof. Examples of such derivative molecules include, but are not limited to, peptide nucleic acid (PNA) and phosphorothioate-based molecules such as deoxyribonucleic guanidine (DNG) or ribonucleic guanidine (RNG). An antisense molecule may be comprised of synthetic nucleotides.

RNAi molecules can be provided to the cell as “ready-to-use” RNA synthesized in vitro or as an antisense gene transfected into cells which will yield RNAi molecules upon transcription. Hybridization with mRNA results in degradation of a hybridized molecule by RNAse H and/or inhibition of formation of translation complexes. Both result in a failure to produce a product of an original gene.

Length of an RNAi molecule that hybridizes to a transcript of interest should be around 10 nucleotides, between about 15 or 30 nucleotides, or about 15, 16, 17, 18, 19, 20, 21, 22, 23, 24, 25, 26, 27, 28, 29, 30 or more nucleotides. Degree of identity of an antisense sequence to a targeted transcript should be at least 75%, at least 80%, at least 85%, at least 90%, or at least 95%.

RNAi molecules may also comprise overhangs, typically unpaired, overhanging nucleotides which are not directly involved in a double helical structure normally formed by a core sequences of herein defined pair of sense strand and antisense strand. RNAi molecules may contain 3′ and/or 5′ overhangs of about 1-5 bases independently on each of a sense and antisense strand. In some embodiments, both sense and antisense strands contain 3′ and 5′ overhangs. In some embodiments, one or more 3′ overhang nucleotides of one strand base (e.g. sense) pairs with one or more 5′ overhang nucleotides of the other strand (e.g. antisense). In some embodiments, one or more 3′ overhang nucleotides of one strand base (e.g. sense) do not pair with the one or more 5′ overhang nucleotides of the other strand (e.g. antisense). Sense and antisense strands of an RNAi molecule may or may not contain the same number of nucleotide bases. Antisense and sense strands may form a duplex wherein a 5′ end only has a blunt end, a 3′ end only has a blunt end, both a 5′ and 3′ ends are blunt ended, or neither a 5′ end nor the 3′ end are blunt ended. In some embodiments, one or more nucleotides in an overhang contains a thiophosphate, phosphorothioate, deoxynucleotide inverted (3′ to 3′ linked) nucleotide or is a modified ribonucleotide or deoxynucleotide.

Small interfering RNA (siRNA) molecules comprise a nucleotide sequence that is identical to about 15 to about 25 contiguous nucleotides of a target mRNA. In some embodiments, an siRNA sequence commences with a dinucleotide AA, comprises a GC-content of about 30-70% (about 30-60%, about 40-60%, or about 45%-55%), and does not have a high percentage identity to any nucleotide sequence other than a target in a genome of a mammal in which it is to be introduced, for example as determined by standard BLAST search.

siRNAs and shRNAs resemble intermediates in processing pathway(s) of endogenous microRNA (miRNA) genes (Bartel, Cell 116:281-297, 2004). In some embodiments, siRNAs can function as miRNAs and vice versa (Zeng et al., Mol Cell 9:1327-1333, 2002; Doench et al., Genes Dev 17:438-442, 2003). MicroRNAs, like siRNAs, use RISC to downregulate target genes, but unlike siRNAs, most animal miRNAs do not cleave an mRNA. Instead, miRNAs reduce protein output through translational suppression or polyA removal and mRNA degradation (Wu et al., Proc Natl Acad Sci USA 103:4034-4039, 2006). Known miRNA binding sites are within mRNA 3′ UTRs; miRNAs seem to target sites with near-perfect complementarity to nucleotides 2-8 from an miRNA's 5′ end (Rajewsky, Nat Genet 38 Suppl:S8-13, 2006; Lim et al., Nature 433:769-773, 2005). This region is known as a seed region. Because siRNAs and miRNAs are interchangeable, exogenous siRNAs downregulate mRNAs with seed complementarity to an siRNA (Birmingham et al., Nat Methods 3:199-204, 2006. Multiple target sites within a 3′ UTR give stronger downregulation (Doench et al., Genes Dev 17:438-442, 2003).

Lists of known miRNA sequences can be found in databases maintained by research organizations, such as Wellcome Trust Sanger Institute, Penn Center for Bioinformatics, Memorial Sloan Kettering Cancer Center, and European Molecule Biology Laboratory, among others. Known effective siRNA sequences and cognate binding sites are also well represented in relevant literature. RNAi molecules are readily designed and produced by technologies known in the art. In addition, there are computational tools that increase chances of finding effective and specific sequence motifs (Pei et al. 2006, Reynolds et al. 2004, Khvorova et al. 2003, Schwarz et al. 2003, Ui-Tei et al. 2004, Heale et al. 2005, Chalk et al. 2004, Amarzguioui et al. 2004).

The RNAi molecule modulates expression of RNA encoded by a gene. Because multiple genes can share some degree of sequence homology with each other, in some embodiments, the RNAi molecule can be designed to target a class of genes with sufficient sequence homology. In some embodiments, an RNAi molecule can contain a sequence that has complementarity to sequences that are shared amongst different gene targets or are unique for a specific gene target. In some embodiments, an RNAi molecule can be designed to target conserved regions of an RNA sequence having homology between several genes thereby targeting several genes in a gene family (e.g., different gene isoforms, splice variants, mutant genes, etc.). In some embodiments, an RNAi molecule can be designed to target a sequence that is unique to a specific RNA sequence of a single gene, e.g., a fusion gene (e.g., a breakpoint within or proximal to a fusion gene), e.g., a fusion oncogene.

In some embodiments, an RNAi molecule targets a sequence in a nucleating polypeptide, e.g., CTCF, cohesin, USF1, YY1, TATA-box binding protein associated factor 3 (TAF3), ZNF143, or another polypeptide that promotes the formation of an anchor sequence-mediated conjunction, or an epigenetic modifying moiety, e.g., an enzyme involved in post-translational modifications including, but are not limited to, DNA methylases (e.g., DNMT3a, DNMT3b, DNMTL), DNA demethylation (e.g., the TET family enzymes catalyze oxidation of 5-methylcytosine to 5-hydroxymethylcytosine and higher oxidative derivatives), histone methyltransferases, histone deacetylase (e.g., HDAC1, HDAC2, HDAC3), sirtuin 1, 2, 3, 4, 5, 6, or 7, lysine-specific histone demethylase 1 (LSD1), histone-lysine-N-methyltransferase (Setdb1), euchromatic histone-lysine N-methyltransferase 2 (G9a), histone-lysine N-methyltransferase (SUV39H1), enhancer of zeste homolog 2 (EZH2), viral lysine methyltransferase (vSET), histone methyltransferase (SET2), protein-lysine N-methyltransferase (SMYD2), and others. In some embodiments, the RNAi molecule targets a protein deacetylase, e.g., sirtuin 1, 2, 3, 4, 5, 6, or 7. In some embodiments, the present disclosure provides a composition comprising an RNAi that targets a nucleating polypeptide, e.g., CTCF.

In some embodiments, an RNAi molecule targets a sequence that is part of a genomic complex (e.g. transcription factor or subunit/portion thereof, transcription machinery or subunit/portion thereof, ncRNA/eRNA, etc.). In some embodiments, an RNAi molecule targets a sequence produced by a gross chromosomal rearrangement, e.g., that is specific to cells comprising or having undergone a gross chromosomal rearrangement, e.g., that is not normally present in wildtype cells. In some embodiments, an RNAi molecule targets a sequence comprising a breakpoint, a fusion gene (e.g., fusion oncogene), or both. In some embodiments, an RNAi molecule targets a sequence comprising a cancer-specific anchor sequence.

In some embodiments, a target is present on a non-genomic entity of interest. For example, in some embodiments, a target may be or comprise a portion of a complex (e.g. a partial complex, wherein a complex has at least two components and wherein a partial complex is or comprises at least one component of a complex). In some embodiments, a complex may be related to cellular activities and/or machinery (e.g. transcription). In some embodiments, a complex may participate in or increase expression of a given gene. In some embodiments, a complex may be or participate in repression of a given gene. In some embodiments, a complex may be related to methylation. In some embodiments, a complex may increase methylation in areas surrounding a given gene. In some embodiments, a complex may decrease methylation in areas surrounding a given gene.

In some aspects, the present disclosure provides compositions, e.g., disrupting agents, that alter structure of (e.g. inhibit formation of or destabilize) one or more genomic complexes. For example, in some embodiments, when a cell is contacted with a composition of the present disclosure, one or more genomic complexes are inhibited (e.g., formation of the complex is inhibited) and/or destabilized. In some embodiments, when a cell is contacted with a composition of the present disclosure, function of one or more genomic complexes is inhibited or decreased. In some embodiments, inhibition of formation and/or destabilization of structure and function occur together. In some embodiments, inhibition of formation and/or destabilization of structure and function are independent of one another.

By way of non-limiting example, in some embodiments, compositions, e.g., disrupting agents, provided in the present disclosure may include, e.g. certain proteins and/or nucleic acids, which target certain sequences.

In some embodiments, compositions, e.g., disrupting agents, may be or comprise Cas9. In some embodiments, compositions comprising Cas9 may target binding sites by way of guide RNA molecules (gRNAs). As will be appreciated by one of skill in the art, gRNAs may be designed to particularly target certain regions of a given genome. In some embodiments, compositions comprising Cas9 may target CTCF binding motifs. In some embodiments, such CTCF binding motifs will be specific for a given genomic complex.

In some embodiments, compositions e.g., disrupting agents, of the present disclosure may be or comprise synthetic nucleic acids.

In some embodiments, compositions e.g., disrupting agents, of the present disclosure may be or comprise dCas9. As will be appreciated by one of skill in the art, gRNAs may be designed to particularly target certain regions of a given genome. In some embodiments, compositions comprising dCas9 may target CTCF binding motif methylation and/or chromatin structure. In some embodiments, such CTCF binding motifs will be specific for a given genomic complex.

In some embodiments, provided compositions, e.g., disrupting agents, may be or comprise nucleic acid based moieties.

In some embodiments, provided nucleic acid based moieties may induce degradation of resident non-coding RNAs. In some embodiments, degradation of resident non-coding RNAs causes genomic complex destabilization and or inhibits formation of genomic complex.

In some embodiments, nucleic acid based moieties may interfere with activity of resident non-coding RNAs. In some embodiments, presence of nucleic acid moieties interferes with activity of resident non-coding RNAs and results in destabilization and/or inhibition of formation of genomic complexes.

Fusion Molecules

In some embodiments, site-specific disrupting agents of the present disclosure may be or comprise a fusion molecule, such as a fusion molecule that comprises a peptide or polypeptide. In some embodiments, a protein fusion comprises one or more moieties described herein, e.g., a targeting moiety and/or effector moiety (e.g. a nucleic acid moiety, a peptide or protein moiety, a membrane translocating polypeptide, or other moiety described herein).

For example, in some embodiments, provided compositions, e.g., disrupting agents, are fusion molecules comprising a site-specific targeting moiety (such as any one of the targeting moieties as described herein) and a deaminating agent, wherein a site-specific targeting moiety targets a fusion molecule to a target anchor sequence but not to at least one non-target anchor sequence. A variety of deaminating agents can be used, such as deaminating agents that do not have enzymatic activity (e.g., chemical agents such as sodium bisulfite), and/or deaminating agents that have enzymatic activity (e.g., a deaminase or functional portion thereof).

In some embodiments, provided compositions, e.g., disrupting agents, are pharmaceutical compositions comprising fusion molecules as described herein.

In some aspects, the present disclosure provides cells or tissues comprising protein fusions as described herein.

In some aspects, the present disclosure provides pharmaceutical compositions comprising protein fusions as described herein.

In some aspects, the present disclosure provides methods of modulating expression of a gene by administering a composition, e.g., disrupting agents, comprising a protein fusion described herein. In some embodiments, for example, a protein fusion may be dCas9-DNMT, dCas9-DNMT-3a-3L, dCas9-DNMT-3a-3a, dCas9-DNMT-3a-3L-3a, dCas9-DNMT-3a-3L-KRAB, dCas9-KRAB, dCas9-APOBEC, APOBEC-dCas9, dCas9-APOBEC-UGI, dCas9-UGI, UGI-dCas9-APOBEC, UGI-APOBEC-dCas9, any variation of protein fusions as described herein, or other fusions of proteins or protein domains described herein.

Exemplary dCas9 fusion methods and compositions that are adaptable to methods and compositions, e.g., disrupting agents, provided by the present disclosure are known and are described, e.g., in Kearns et al., Functional annotation of native enhancers with a Cas9-histone demethylase fusion. Nature Methods 12, 401-403 (2015); and McDonald et al., Reprogrammable CRISPR/Cas9-based system for inducing site-specific DNA methylation. Biology Open 2016: doi: 10.1242/bio.019067. Using methods known in the art, dCas9 can be fused to any of a variety of agents and/or molecules as described herein; such resulting fusion molecules can be useful in various disclosed methods.

In some aspects, the present disclosure provides compositions, e.g., disrupting agents, comprising a fusion protein comprising a domain, e.g., an enzyme domain, that acts on DNA (e.g., a nuclease domain, e.g., a Cas9 domain, e.g., a dCas9 domain; a DNA methyltransferase, a demethylase, a deaminase), in combination with at least one guide RNA (gRNA) or antisense DNA oligonucleotide that targets a protein to an anchor sequence of a target anchor sequence-mediated conjunction, wherein a composition is effective to inhibit or destabilize, in a human cell, a target anchor sequence-mediated conjunction. In some embodiments, an enzyme domain is a Cas9 or a dCas9. In some embodiments, a protein comprises two enzyme domains, e.g., a dCas9 and a methylase or demethylase domain.

In some aspects, the present disclosure provides compositions, e.g., disrupting agents, comprising a fusion protein comprising a domain, e.g., an enzyme domain, that acts on DNA (e.g., a nuclease domain, e.g., a Cas9 domain, e.g., a dCas9 domain; a DNA methyltransferase, a demethylase, a deaminase), in combination with at least one guide RNA (gRNA) or antisense DNA oligonucleotide that targets a protein to sequence within a genomic complex that is not an anchor sequence. In some embodiments, targeting by the composition, e.g., disrupting agent, is effective to inhibit (e.g., formation of) or destabilize, in a human cell, a target anchor sequence-mediated conjunction. In some embodiments, a sequence is targeted to a component of a genomic complex that is, e.g. a transcription factor, transcription regulation, ncRNA, eRNA, etc. In some embodiments, an enzyme domain is a Cas9 or a dCas9. In some embodiments, a protein comprises two enzyme domains, e.g., a dCas9 and a methylase or demethylase domain.

In some embodiments, for example, a disrupting agent may comprise a fusion of a sequence targeting polypeptide and another molecule, e.g. a targeting polypeptide (e.g. dCas9) and a genomic complex component (e.g. transcription factor), e.g. a targeting polypeptide and an effector polypeptide, e.g. a fusion of dCas9 and a nucleating polypeptide, e.g., one gRNA or antisense DNA oligonucleotides fused with a nuclease, or a nucleic acid encoding the fusion, etc. Fusions of a catalytically inactive endonuclease e.g., a dead Cas9 (dCas9, e.g., D10A; H840A) tethered with all or a portion of (e.g., biologically active portion of) an (one or more) effector domain and/or other agent create chimeric proteins or fusion molecules that can be guided to specific DNA sites by one or more RNA sequences (sgRNA) or antisense DNA oligonucleotides to modulate activity and/or expression of one or more target nucleic acids sequences (e.g., to methylate or demethylate a DNA sequence).

As used herein, a “biologically active portion of an effector domain” is a portion that maintains function (e.g. completely, partially, minimally) of an effector domain (e.g., a “minimal” or “core” domain). In some embodiments, fusion of a dCas9 with all or a portion of one or more effector domains of an epigenetic modifying moiety (such as a DNA methylase or enzyme with a role in DNA demethylation, e.g., DNMT3a, DNMT3b, DNMT3L, a DNMT inhibitor, TET family enzymes, and combinations thereof, or protein acetyl transferase or deacetylase) creates a chimeric protein that is useful in methods provided herein. Accordingly, in some embodiments, a targeting moiety includes a dCas9-methylase fusion in combination with a site-specific gRNA or antisense DNA oligonucleotide that targets a fusion to a conjunction anchor sequence (such as a CTCF binding motif), thereby decreasing affinity or ability of an anchor sequence to bind a conjunction nucleating polypeptide. In some embodiments, all or a portion of one or more epigenetic modifying moiety effector domains (e.g., DNA methylase or enzyme with a role in DNA demethylation, or protein acetyl transferase or deacetylase, or deaminase) are fused with an inactive nuclease, e.g., dCas9. In some aspects, 1, 2, 3, 4, 5, 6, 7, 8, 9, 10, 11, 12, 13, 14, 15, 16, 17, 18, 19, 20, or more effector domains (all or a biologically active portion) are fused with dCas9.

Chimeric proteins described herein may also comprise a linker as described herein, e.g., an amino acid linker. In some aspects, a linker comprises 2 or more amino acids, e.g., one or more GS sequences. In some aspects, fusion of Cas9 (e.g., dCas9) with two or more effector domains (e.g., of a DNA methylase or enzyme with a role in DNA demethylation or protein acetyl transferase or deacetylase) comprises one or more interspersed linkers (e.g., GS linkers) between the domains. In some aspects, dCas9 is fused with 2-5 effector domains with interspersed linkers.

Small Molecules

In some embodiments, a disrupting agent as described herein is or comprises one or more small molecules.

In some embodiments, a disrupting agent (i.e., a targeting, effector, and/or other moiety thereof) comprises a small molecule that intercalates into a nucleic acid structure, e.g., at a specific site.

In some embodiments, a disrupting agent comprises a small molecule pharmacoagent.

In some embodiments, a disrupting agent may be or comprise a small molecule that alters one or more DNA methylation sites, e.g., mutates methylated cysteine to thymine, within an anchor sequence-mediated conjunction. For example, bisulfite compounds, e.g., sodium bisulfite, ammonium bisulfite, or other bisulfite salts, may be used to alter one or more DNA methylation sites, e.g., altering a nucleotide sequence from a cysteine to a thymine.

In some embodiments, a small molecule may include, but not be limited to, small peptides, peptidomimetics (e.g., peptoids), amino acids, amino acid analogs, synthetic polynucleotides, polynucleotide analogs, nucleotides, nucleotide analogs, organic and inorganic compounds (including heterorganic and organometallic compounds) generally having a molecular weight less than about 5,000 grams per mole, e.g., organic or inorganic compounds having a molecular weight less than about 2,000 grams per mole, e.g., organic or inorganic compounds having a molecular weight less than about 1,000 grams per mole, e.g., organic or inorganic compounds having a molecular weight less than about 500 grams per mole, and salts, esters, and other pharmaceutically acceptable forms of such compounds. Small molecules may include, but are not limited to, a neurotransmitter, a hormone, a drug, a toxin, a viral or microbial particle, a synthetic molecule, and agonists or antagonists.

Examples of suitable small molecules include those described in, “The Pharmacological Basis of Therapeutics,” Goodman and Gilman, McGraw-Hill, New York, N.Y., (1996), Ninth edition, under the sections: Drugs Acting at Synaptic and Neuroeffector Junctional Sites; Drugs Acting on the Central Nervous System; Autacoids: Drug Therapy of Inflammation; Water, Salts and Ions; Drugs Affecting Renal Function and Electrolyte Metabolism; Cardiovascular Drugs; Drugs Affecting Gastrointestinal Function; Drugs Affecting Uterine Motility; Chemotherapy of Parasitic Infections; Chemotherapy of Microbial Diseases; Chemotherapy of Neoplastic Diseases; Drugs Used for Immunosuppression; Drugs Acting on Blood-Forming organs; Hormones and Hormone Antagonists; Vitamins, Dermatology; and Toxicology, all incorporated herein by reference. Some examples of small molecules may include, but are not limited to, prion drugs such as tacrolimus, ubiquitin ligase or HECT ligase inhibitors such as heclin, histone modifying drugs such as sodium butyrate, enzymatic inhibitors such as 5-aza-cytidine, anthracyclines such as doxorubicin, beta-lactams such as penicillin, anti-bacterials, chemotherapy agents, anti-virals, modulators from other organisms such as VP64, and drugs with insufficient bioavailability such as chemotherapeutics with deficient pharmacokinetics.

In some embodiments, a small molecule is an epigenetic modifying moiety, for example such as those described in de Groote et al. Nuc. Acids Res. (2012):1-18. Exemplary small molecule epigenetic modifying moieties are described, e.g., in Lu et al. J. Biomolecular Screening 17.5(2012):555-71, e.g., at Table 1 or 2, incorporated herein by reference. In some embodiments, an epigenetic modifying moiety comprises vorinostat, romidepsin. In some embodiments, an epigenetic modifying moiety comprises an inhibitor of class I, II, III, and/or IV histone deacetylase (HDAC). In some embodiments, an epigenetic modifying moiety comprises an activator of SirTI. In some embodiments, an epigenetic modifying moiety comprises Garcinol, Lys-CoA, C646, (+)-JQI, I-BET, BICI, MS120, DZNep, UNC0321, EPZ004777, AZ505, AMI-I, pyrazole amide 7b, benzo[d]imidazole 17b, acylated dapsone derivative (e.g., PRMTI), methylstat, 4,4′-dicarboxy-2,2′-bipyridine, SID 85736331, hydroxamate analog 8, tanylcypromie, bisguanidine and biguanide polyamine analogs, UNC669, Vidaza, decitabine, sodium phenyl butyrate (SDB), lipoic acid (LA), quercetin, valproic acid, hydralazine, bactrim, green tea extract (e.g., epigallocatechin gallate (EGCG)), curcumin, sulforphane and/or allicin/diallyl disulfide. In some embodiments, an epigenetic modifying moiety inhibits DNA methylation, e.g., is an inhibitor of DNA methyltransferase (e.g., is 5-azacitidine and/or decitabine). In some embodiments, an epigenetic modifying moiety modifies histone modification, e.g., histone acetylation, histone methylation, histone sumoylation, and/or histone phosphorylation. In some embodiments, an epigenetic modifying moiety is an inhibitor of a histone deacetylase (e.g., is vorinostat and/or trichostatin A).

In some embodiments, a small molecule is a pharmaceutically active agent. In some embodiments, a small molecule is an inhibitor of a metabolic activity or component. Useful classes of pharmaceutically active agents include, but are not limited to, antibiotics, anti-inflammatory drugs, angiogenic or vasoactive agents, growth factors and/or chemotherapeutic agents. One or a combination of molecules from categories and examples as described herein or from (Orme-Johnson 2007, Methods Cell Biol. 2007; 80:813-26) can be used. In some embodiments, the present disclosure provides compositions comprising one or more antibiotics, anti-inflammatory drugs, angiogenic or vasoactive agents, growth factors and/or chemotherapeutic agents.

In some embodiments, a disrupting agent comprises a small molecule moiety (e.g., a peptidomimetic or a small organic molecule with a molecular weight of less than 2000 daltons), a peptide or polypeptide (e.g., a non ABXⁿC polypeptide, e.g., an antibody or antigen-binding fragment thereof), a nucleic acid (e.g., siRNA, mRNA, RNA, DNA, modified DNA or RNA, antisense DNA oligonucleotides, an antisense RNA, a ribozyme, a therapeutic mRNA encoding a protein), a nanoparticle, an aptamer, or pharmacoagent with poor PK/PD.

Intercalators

In some embodiments, a disrupting agent comprises one or more intercalating agents. In some embodiments, an intercalating agent inserts between bases of genomic material (e.g. DNA). In some embodiments, intercalation causes inhibition of formation and/or destabilization in a particular anchor-mediated sequence conjunction and, accordingly, modulation of gene expression. Intercalating agents may comprise, but not be limited to berberine, ethidium bromide, proflavine, daunomycin, doxorubicin, and/or thalidomide. In some embodiments, intercalating agents may result in cell death (e.g. intercalation into a particular cell may ultimately result in cell death of that cell by disrupting DNA synthesis and cellular replication).

Exemplary Small Molecule Disrupting Agents

In some embodiments, a disrupting agent is or comprises a small molecule. In some embodiments, gene expression is decreased via use of disrupting agents that are or comprise one or more small molecules and dCas9. In some embodiments, one or more small molecules is/are targeted to particular genomic complexes via dCas9 and target-specific guide RNA. As will be understood by one of skill in the art, small molecules used for targeting may be the same or different depending on a given target. In some embodiments, gene expression is decreased in genomic complexes that comprise type 1, EP subtype complexes.

In some embodiments, gene expression is decreased in genomic complexes that are or comprise type 4 genomic complexes (e.g., ER sequence, CTCF sequence, YY1 sequence).

In some embodiments, gene expression is decreased via use of site-specific disrupting agents that are or comprise one or more antibody fragments and dCas9. In some embodiments, one or more small molecules is/are targeted to a particular genomic complex via dCas9 and target-specific guide RNA. In some embodiments, gene expression is decreased in genomic complexes that are or comprise type 1 genomic complexes.

Nanoparticles

A disrupting agent may be or comprise a nanoparticle. Nanoparticles include inorganic materials with a size between about 1 and about 1000 nanometers, between about 1 and about 500 nanometers in size, between about 1 and about 100 nm, between about 30 nm and about 200 nm, between about 50 nm and about 300 nm, between about 75 nm and about 200 nm, between about 100 nm and about 200 nm, and any range therebetween. In some embodiments, a nanoparticle has a composite structure of nanoscale dimensions. In some embodiments, nanoparticles are typically spherical although different morphologies are possible depending on the nanoparticle composition. A portion of a nanoparticle contacting an environment external to a nanoparticle is generally identified as the surface of the nanoparticle. In nanoparticles described herein, a size limitation can be restricted to two dimensions and so that nanoparticles include composite structure having a diameter from about 1 to about 1000 nm, where a specific diameter depends on a nanoparticle composition and on intended use of a nanoparticle according to the experimental design. For example, nanoparticles used in therapeutic applications typically have a size of about 200 nm or below.

Additional desirable properties of a nanoparticle, such as surface charges and steric stabilization, can also vary in view of the specific application of interest. Certain useful properties are identifiable by a skilled person upon reading of the present disclosure. Nanoparticle dimensions and properties can be detected by techniques known in the art. Exemplary techniques to detect particles dimensions include but are not limited to dynamic light scattering (DLS) and a variety of microscopies such at transmission electron microscopy (TEM) and atomic force microscopy (AFM). Exemplary techniques to detect particle morphology include but are not limited to TEM and AFM. Exemplary techniques to detect surface charges of the nanoparticle include but are not limited to zeta potential method. Additional techniques suitable to detect other chemical properties comprise by ¹H, ¹¹B, and ¹³C and ¹⁹F NMR, UV/Vis and infrared/Raman spectroscopies and fluorescence spectroscopy (when nanoparticle is used in combination with fluorescent labels) and additional techniques identifiable by a skilled person.

Linkers

In some embodiments, disrupting agents may include one or more linkers. In some embodiments, a disrupting agent as described herein, e.g., comprising a first polypeptide domain that comprises a Cas or modified Cas protein and a second polypeptide domain that comprises a polypeptide having DNA methyltransferase activity [or associated with demethylation or deaminase activity], has a linker between the first and second polypeptide. A linker may be a chemical bond, e.g., one or more covalent bonds or non-covalent bonds. In some embodiments links are covalent. In some embodiments, links are non-covalent. In some embodiments, a linker is a peptide linker (e.g., a non ABXⁿC peptide). Such a linker may be between 2-30 amino acids, or longer. In some embodiments, a linker can be used, e.g., to space a targeting moiety from an effector moiety of a disrupting agent. In some embodiments, for example, a linker can be positioned between a targeting moiety and an effector moiety of a disrupting agent, e.g., to provide molecular flexibility of secondary and tertiary structures. A linker may comprise flexible, rigid, and/or cleavable linkers described herein. In some embodiments, a linker includes at least one glycine, alanine, and serine amino acids to provide for flexibility. In some embodiments, a linker is a hydrophobic linker, such as including a negatively charged sulfonate group, polyethylene glycol (PEG) group, or pyrophosphate diester group. In some embodiments, a linker is cleavable to selectively release a moiety (e.g. polypeptide) from a disrupting agent, but sufficiently stable to prevent premature cleavage.

In some embodiments, one or more components of a disrupting agent described herein are linked with a linker.

As will be known by one of skill in the art, commonly used flexible linkers have sequences consisting primarily of stretches of Gly and Ser residues (“GS” linker). Flexible linkers may be useful for joining domains that require a certain degree of movement or interaction and may include small, non-polar (e.g. Gly) or polar (e.g. Ser or Thr) amino acids. Incorporation of Ser or Thr can also maintain the stability of a linker in aqueous solutions by forming hydrogen bonds with water molecules, and therefore reduce unfavorable interactions between a linker and protein moieties.

Rigid linkers are useful to keep a fixed distance between domains and to maintain their independent functions. Rigid linkers may also be useful when a spatial separation of domains is critical to preserve the stability or bioactivity of one or more components in the fusion. Rigid linkers may have an alpha helix-structure or Pro-rich sequence, (XP)ₙ, with X designating any amino acid, preferably Ala, Lys, or Glu.

Cleavable linkers may release free functional domains in vivo. In some embodiments, linkers may be cleaved under specific conditions, such as presence of reducing reagents or proteases. In vivo cleavable linkers may utilize reversible nature of a disulfide bond. One example includes a thrombin-sensitive sequence (e.g., PRS) between the two Cys residues. In vitro thrombin treatment of CPRSC results in the cleavage of a thrombin-sensitive sequence, while a reversible disulfide linkage remains intact. Such linkers are known and described, e.g., in Chen et al. 2013. Fusion Protein Linkers: Property, Design and Functionality. Adv Drug Deliv Rev. 65(10): 1357-1369. In vivo cleavage of linkers in fusions may also be carried out by proteases that are expressed in vivo under certain conditions, in specific cells or tissues, or constrained within certain cellular compartments. Specificity of many proteases offers slower cleavage of the linker in constrained compartments.

Examples of linking molecules include a hydrophobic linker, such as a negatively charged sulfonate group; lipids, such as a poly (—CH2—) hydrocarbon chains, such as polyethylene glycol (PEG) group, unsaturated variants thereof, hydroxylated variants thereof, amidated or otherwise N-containing variants thereof, noncarbon linkers; carbohydrate linkers; phosphodiester linkers, or other molecule capable of covalently linking two or more components of a disrupting agent (e.g. two polypeptides). Non-covalent linkers are also included, such as hydrophobic lipid globules to which the polypeptide is linked, for example through a hydrophobic region of a polypeptide or a hydrophobic extension of a polypeptide, such as a series of residues rich in leucine, isoleucine, valine, or perhaps also alanine, phenylalanine, or even tyrosine, methionine, glycine or other hydrophobic residue. Components of a disrupting agent may be linked using charge-based chemistry, such that a positively charged component of a disrupting agent is linked to a negative charge of another component or nucleic acid.

### Certain Exemplary Site-Specific Disrupting Agents

In some embodiments, a disrupting agent comprises a nucleic acid targeting moiety (e.g., a gRNA) that targets a particular genomic sequence, and is associated with a polypeptide disrupting moiety that directly binds to, competes for, and/or blocks other complex components, e.g., thereby inhibiting formation of or destabilizing a genomic complex.

In some embodiments, a disrupting agent comprises a nucleic acid targeting moiety (e.g., a gRNA) that targets a particular genomic sequence and is associated with an oligonucleotide disrupting moiety that directly binds to, competes for, and/or blocks other complex components, e.g., thereby inhibiting formation of or destabilizing a genomic complex.

In some embodiments, a disrupting agent comprises a nucleic acid targeting moiety (e.g. a gRNA) that targets a particular genomic sequence and is associated with an oligonucleotide disrupting moiety that directly binds to, competes for, and/or blocks other components. In this example, a complex component bound to the oligonucleotide disrupting moiety has decreased binding to other genomic complex components, e.g., its binding is inhibited, e.g., prevented.

In some embodiments, a disrupting agent comprises a nucleic acid targeting moiety (e.g. gRNA) that targets a particular genomic sequence and is associated with an antibody, antibody fragment, or antibody mimetic disrupting moiety that directly binds to, competes for, and/or blocks other complex components. Alternatively or additionally, in some embodiments, the disrupting moiety may be covalently linked with another oligonucleotide agent (e.g. DNA, RNA, gRNA, PNA, etc.).

In some embodiments, a disrupting agent comprises a nucleic acid targeting moiety (e.g. gRNA) that targets a particular genomic sequence and is associated with a disrupting moiety comprising a single stranded ribonucleic acid comprising a sequence identical to at least a of a portion of a particular non-coding RNA (ncRNA, e.g. siRNA, eRNA, etc.) that is normally a component of the genomic complex, which single stranded ribonucleic acid is covalently attached to the 3′ end of a tracr RNA (e.g., from a CRISPR gene editing system).

In some embodiments, a disrupting agent inhibits formation of and/or destabilizes a genomic complex (e.g. an anchor sequence-mediated conjunction within a genomic complex).

### Formulation, Delivery, and Administration

The present disclosure, among other things, provides compositions that comprise or deliver a disrupting agent. For example, in some embodiments, a disrupting agent that is or comprises a polypeptide moiety or entity may be provided via a composition that includes the polypeptide moiety or entity, or alternatively via a composition that includes a nucleic acid encoding the polypeptide moiety or entity, and associated with sufficient other sequences to achieve expression of the polypeptide moiety or entity in a system of interest (e.g., in a particular cell, tissue, organism, etc).

Thus, in some embodiments, the present disclosure provides compositions comprising a disrupting agent, or a production intermediate thereof. In some particular embodiments, the present disclosure provides compositions of nucleic acids that encode a disrupting agent or polypeptide portion thereof. In some such embodiments, provided nucleic acids may be or include DNA, RNA, or any other nucleic acid moiety or entity as described herein, and may be prepared by any technology described herein or otherwise available in the art (e.g., synthesis, cloning, amplification, in vitro or in vivo transcription, etc). In some embodiments, provided nucleic acids that encode a disrupting agent or polypeptide portion thereof may be operationally associated with one or more replication, integration, and/or expression signals appropriate and/or sufficient to achieve integration, replication, and/or expression of the provided nucleic acid in a system of interest (e.g., in a particular cell, tissue, organism, etc).

In some embodiments, a provided composition may be a pharmaceutical composition whose active ingredient comprises or delivers a disrupting agent as described herein and is provided in combination with one or more pharmaceutically acceptable excipients, optionally formulated for administration to a subject (e.g., to a cell, tissue, or other site thereof).

Pharmaceutical compositions described herein may be formulated for example including a carrier, such as a pharmaceutical carrier and/or a polymeric carrier, e.g., a liposome, and delivered by known methods to a subject in need thereof (e.g., a human or non-human agricultural or domestic animal, e.g., cattle, dog, cat, horse, poultry). Such methods include transfection (e.g., lipid-mediated, cationic polymers, calcium phosphate); electroporation or other methods of membrane disruption (e.g., nucleofection) and viral delivery (e.g., lentivirus, retrovirus, adenovirus, AAV). Methods of delivery are also described, e.g., in Gori et al., Delivery and Specificity of CRISPR/Cas9 Genome Editing Technologies for Human Gene Therapy. Human Gene Therapy. July 2015, 26(7): 443-451. doi:10.1089/hum.2015.074; and Zuris et al. Cationic lipid-mediated delivery of proteins enables efficient protein-based genome editing in vitro and in vivo. Nat Biotechnol. 2014 Oct 30;33(1):73-80.

In various embodiments, the present disclosure provides pharmaceutical compositions described herein with a pharmaceutically acceptable excipient. Pharmaceutically acceptable excipient includes an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous.

Pharmaceutical compositions described herein can also be tableted or prepared in an emulsion or syrup for oral administration. Pharmaceutically acceptable solid or liquid carriers may be added to enhance or stabilize the composition, or to facilitate preparation of the composition.

Pharmaceutical compositions according to the present disclosure may be delivered in a therapeutically effective amount. A precise therapeutically effective amount is an amount of a composition, e.g., disrupting agent, that will yield the most effective results in terms of efficacy of treatment in a given subject. This amount will vary depending upon a variety of factors, including but not limited to characteristics of a therapeutic compound (including activity, pharmacokinetics, pharmacodynamics, and bioavailability), physiological condition of a subject (including age, sex, disease type and stage, general physical condition, responsiveness to a given dosage, and type of medication), nature of a pharmaceutically acceptable carrier or carriers in a formulation, and/or route of administration.

In various embodiments compositions described herein are pharmaceutical compositions. In some embodiments, compositions (e.g. pharmaceutical compositions) described herein may be formulated for delivery to a cell and/or to a subject via any route of administration. Modes of administration to a subject may include injection, infusion, inhalation, intranasal, intraocular, topical delivery, intercannular delivery, or ingestion. Injection includes, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebrospinal, and intrasternal injection and infusion. In some embodiments, administration includes aerosol inhalation, e.g., with nebulization. In some embodiments, administration is systemic (e.g., oral, rectal, nasal, sublingual, buccal, or parenteral), enteral (e.g., system-wide effect, but delivered through the gastrointestinal tract), or local (e.g., local application on the skin, intravitreal injection). In some embodiments, one or more compositions is administered systemically. In some embodiments, administration is non-parenteral and a therapeutic is a parenteral therapeutic.

In some embodiments, a composition as provided herein is administered systemically.

In some embodiments, administration is non-parenteral and a therapeutic is a parenteral therapeutic.

Administration of a composition may be, e.g., to a subject (e.g., a human subject) or system. For example, in some embodiments, administration may be ocular, oral, parenteral, topical, etc. In some particular embodiments, administration may be bronchial (e.g., by bronchial instillation), buccal, dermal (which may be or comprise, for example, one or more of topical administration to the dermis, intradermal, intradermal, transdermal, etc.), enteral, intra-arterial, intradermal, intragastric, intramedullary, intramuscular, intranasal, intraperitoneal, intrathecal, intravenous, intraventricular, within a specific organ (e. g. intrahepatic), mucosal, nasal, oral, rectal, subcutaneous, sublingual, topical, tracheal (e.g., by intratracheal instillation), vaginal, vitreal, etc. In some embodiments, administration may be a single dose. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

Methods as provided in various embodiments herein may be utilized in any some aspects delineated herein. In some embodiments, one or more compositions is/are targeted to specific cells, or one or more specific tissues.

For example, in some embodiments one or more compositions is/are targeted to epithelial, connective, muscular, and/or nervous tissue or cells. In some embodiments a composition is targeted to a cell or tissue of a particular organ system, e.g., cardiovascular system (heart, vasculature); digestive system (esophagus, stomach, liver, gallbladder, pancreas, intestines, colon, rectum and anus); endocrine system (hypothalamus, pituitary gland, pineal body or pineal gland, thyroid, parathyroids, adrenal glands); excretory system (kidneys, ureters, bladder); lymphatic system (lymph, lymph nodes, lymph vessels, tonsils, adenoids, thymus, spleen); integumentary system (skin, hair, nails); muscular system (e.g., skeletal muscle); nervous system (brain, spinal cord, nerves); reproductive system (ovaries, uterus, mammary glands, testes, vas deferens, seminal vesicles, prostate); respiratory system (pharynx, larynx, trachea, bronchi, lungs, diaphragm); skeletal system (bone, cartilage); and/or combinations thereof.

In some embodiments, a composition of the present disclosure crosses a blood-brain-barrier, a placental membrane, or a blood-testis barrier.

Methods and compositions provided herein may comprise a pharmaceutical composition administered by a regimen sufficient to alleviate a symptom of a disease, disorder, and/or condition. In some aspects, the present disclosure provides methods of delivering a therapeutic by administering compositions as described herein.

In some aspects, a system for pharmaceutical use comprises a composition that disrupts a genomic complex by binding an anchor sequence of an anchor sequence-mediated conjunction and disrupts the anchor sequence-mediated conjunction, wherein such a composition modulates transcription, in a human cell, of a target gene associated with the anchor sequence-mediated conjunction.

In some aspects, a system for pharmaceutical use comprises a composition that disrupts a genomic complex by binding a sequence within an anchor sequence-mediated conjunction that is not an anchor sequence, for example, an ncRNA, and disrupts an anchor sequence-mediated conjunction, wherein such a composition modulates transcription, in a human cell, of a target gene associated with the anchor sequence-mediated conjunction.

In some aspects, a system for altering, e.g., inhibiting, in a human cell, expression of a target gene by disrupting a genomic complex comprises a targeting moiety (e.g., a gRNA, a membrane translocating polypeptide) that associates with an anchor sequence associated with a target gene, and an effector moiety, e.g., disrupting moiety. Optionally, another moiety (e.g., an effector moiety which may be, e.g. an enzyme, e.g., a nuclease or deactivated nuclease (e.g., a Cas9, dCas9), a methylase, a de-methylase, a deaminase) operably linked to a targeting moiety may be included, wherein a system is effective to inhibit and/or destabilize a conjunction mediated by an anchor sequence and alter expression of a target gene. A targeting moiety and an effector moiety, e.g., disrupting moiety, may be different and/or separate moieties. A targeting moiety and a disrupting moiety may be identical moieties, but not one and the same (e.g. if a targeting moiety and a disrupting moiety are both present and the same, there will be at least two moieties present). A targeting moiety and an effector moiety, e.g., disrupting moiety, may be linked. In some embodiments, a system comprises a synthetic polypeptide comprising a targeting moiety and an effector moiety, e.g., disrupting moiety. In some embodiments, a system comprises a nucleic acid vector or vectors encoding at least one of a targeting moiety and an effector moiety, e.g., disrupting moiety.

In some aspects, pharmaceutical compositions may comprise a composition, e.g., comprising a disrupting agent, that disrupts a genomic complex by binding an anchor sequence of an anchor sequence-mediated conjunction and disrupting an anchor sequence-mediated conjunction, wherein the composition decreases transcription, in a human cell, of a target gene associated with an anchor sequence-mediated conjunction. In some embodiments, compositions of the present disclosure may disrupt an anchor sequence-mediated conjunction (e.g., decreases affinity of an anchor sequence to a nucleating polypeptide, e.g., at least 10%, 15%, 20%, 25%, 30%, 35%, 40%, 45%, 50%, 55%, 60%, 65%, 70%, 75%, 80%, 85%, 90%, 95%, or more). Disrupting a genomic complex may comprise reducing the affinity of an anchor sequence to a nucleating polypeptide, e.g., at least 10%, 15%, 20%, 25%, 30%, 35%, 40%, 45%, 50%, 55%, 60%, 65%, 70%, 75%, 80%, 85%, 90%, 95%, or more.

In some aspects, the present disclosure provides a pharmaceutical composition comprising (a) a targeting moiety and (b) a DNA sequence comprising an anchor sequence.

In some aspects, the present disclosure provides a composition, e.g., comprising a disrupting agent, comprising a targeting moiety that binds an anchor sequence within a genomic complex and disrupts an anchor sequence-mediated conjunction (e.g., decreases affinity of the anchor sequence to a nucleating polypeptide, e.g., at least 10%, 15%, 20%, 25%, 30%, 35%, 40%, 45%, 50%, 55%, 60%, 65%, 70%, 75%, 80%, 85%, 90%, 95%, or more).

In some aspects, a pharmaceutical composition includes a Cas protein and at least one guide RNA (gRNA) that targets a Cas protein to an anchor sequence of a target anchor sequence-mediated conjunction. The Cas protein should be effective to cause a mutation of the target anchor sequence that decreases formation of an anchor sequence-mediated conjunction associated with a target anchor sequence.

In some embodiments, a gRNA is administered in combination with a targeted nuclease, e.g., a Cas9, e.g., a wild type Cas9, a nickase Cas9 (e.g., Cas9 D10A), a dead Cas9 (dCas9), eSpCas9, Cpf1, C2C1, or C2C3, or a nucleic acid encoding such a nuclease. Choice of nuclease and gRNA(s) is determined by whether a targeted mutation is a deletion, substitution, or addition of nucleotides, e.g., a deletion, substitution, or addition of nucleotides to a targeted anchor sequence, e.g., a CTCF binding motif. For example, in some embodiments, one gRNA is administered, e.g., to produce an inactivating indel mutation in an anchor sequence, e.g., a CTCF motif, e.g., one gRNA is administered in combination with a nuclease, e.g., wtCas9. In some embodiments, two gRNAs are administered, e.g., in combination with an insertion cassette and a nucleic acid encoding a nuclease to produce a replacement sequence at a targeted anchor sequence. A replacement sequence may have weaker affinity to a target, e.g., a replacement sequence may have less identity to a provided gRNA than a target sequence, e.g., to produce a destabilized loop. In some embodiments, a replacement sequence has less than 10%, 15%, 20%, 25%, 30%, 35%, 40%, 45%, 50%, 55%, 60%, 65%, 70%, 75%, 80%, 85%, 90%, or 95% identity to a provided gRNA. For example, in some embodiments, a replacement sequence may have a weaker affinity to a nucleating polypeptide, e.g., a replacement sequence may have less identity to SEQ ID NO:1 or SEQ ID NO: 2 than a target sequence, e.g., to produce a destabilized loop. In other embodiments, a replacement sequence has less than 10%, 15%, 20%, 25%, 30%, 35%, 40%, 45%, 50%, 55%, 60%, 65%, 70%, 75%, 80%, 85%, 90%, or 95% identity to SEQ ID NO:1 or SEQ ID NO: 2. In some embodiments, a nucleating polypeptide may be, e.g., CTCF, cohesin, USF1, YY1, TAF3, ZNF143 binding motif, or another polypeptide that promotes formation of an anchor sequence-mediated conjunction.

In some embodiments, nucleic acids comprising: a gRNA, a nucleic acid sequence encoding a nuclease, and an insertion cassette are administered to change the orientation of a target sequence (e.g. in a target genomic complex), e.g., from being in tandem with a partner sequence to being convergent with a partner sequence, e.g., to create a destabilized loop, e.g., a gRNA, a nuclease and an insertion cassette are administered to replace an anchor sequence having a particular consensus sequence.

In some aspects, the present disclosure provides a composition, e.g., disrupting agent, comprising a nucleic acid or combination of nucleic acids that when administered to a subject in need thereof introduce a site specific alteration (e.g., insertion, deletion (e.g., knockout), translocation, inversion, single point mutation) in a target sequence of a target genomic complex or of a component of a target genomic complex, e.g., an ncRNA, eRNA, a CTCF-binding motif, genomic sequence of a transcription factor that itself is part of a target genomic complex, etc., thereby altering gene expression in a subject.

In some aspects, the present disclosure provides a pharmaceutical composition comprising a guide RNA (gRNA) for use in a clustered regulatory interspaced short palindromic repeat (CRISPR) system for gene editing. For example, a gRNA can be administered in combination with a nuclease (e.g., Cpf1 or Cas9) or a nucleic acid encoding the nuclease, to specifically cleave double-stranded DNA. Alternatively, precise mutations and knock-ins to a target CTCF binding motif can be made by providing a homologous repair template and exploiting homology directed repair pathway. Alternatively, double nicking with paired Cas9 nickases can be used to introduce a staggered double-stranded break which can then undergo homology directed repair to introduce one more nucleotides into a target sequence in a site specific manner. Custom gRNA generators and algorithms are available commercially for use in developing methods and compositions provided herein.

In some embodiments, pharmaceutical compositions of the present disclosure comprise a zinc finger nuclease (ZFN), or a mRNA encoding a ZFN, that targets (e.g., cleaves) a CTCF-binding motif or a sequence within or outside of a sequence

### Uses

Compositions and methods described herein can be used to treat various cancers. In some embodiments, the cancer cell comprises a breakpoint, e.g., leading to formation of a fusion oncogene. In some embodiments, the fusion oncogene comprises CCDCl₆-RET and the cancer comprises a thyroid cancer or a lung cancer. In some embodiments, the fusion oncogene comprises PAX3-FOXO and the cancer comprises a rhabdomyosarcoma, e.g., an alveolar rhabdomyosarcoma and/or a pediatric rhabdomyosarcoma. In some embodiments, the fusion oncogene comprises BRC-ABL1 and the cancer comprises a leukemia, e.g., a CML. In some embodiments, the fusion oncogene comprises EML4-ALK and the cancer comprises a lung cancer. In some embodiments, the fusion oncogene comprises ETV6-RUNX1 and the cancer comprises a leukemia, e.g., an ALL, e.g., a pediatric ALL. In some embodiments, the fusion oncogene comprises TMPRSS2-ERG and the cancer comprises prostate cancer. In some embodiments, the fusion oncogene comprises TCF3-PBX1 and the cancer comprises a lung cancer or a leukemia, e.g., ALL (e.g., pediatric ALL). In some embodiments, the fusion oncogene comprises KMT2A-AFF1 and the cancer comprises a leukemia, e.g., ALL, e.g., pediatric ALL. In some embodiments, the fusion oncogene comprises EWSR1-FLI1 and the cancer comprises a sarcoma, e.g., Ewing sarcoma.

In some embodiments, the fusion oncogene is an IGH fusion oncogene wherein an IGH fusion oncogene comprises an IGH encoding sequence and/or a genomic sequence element (e.g., promoter, enhancer, and/or super enhancer) proximal to an IGH encoding sequence or portion of either thereof. In some embodiments, the fusion oncogene (e.g., IGH fusion oncogene) comprises the coding sequence of the IGH gene or a portion thereof. In some embodiments, the fusion oncogene (e.g., IGH fusion oncogene) comprises a non-coding sequence of the IGH gene or a portion thereof. In some embodiments, the fusion oncogene (e.g., IGH fusion oncogene) comprises a regulatory element (e.g., an enhancer (e.g., super enhancer) and/or a promoter) of the IGH gene or a portion thereof.

In some embodiments the fusion oncogene is a fusion between a first fusion partner gene and a second fusion partner gene. In some embodiments, the first fusion partner gene is IGH. In some embodiments, the fusion oncogene (e.g., IGH fusion oncogene) comprises a portion of a coding, non-coding, and/or regulatory element (e.g., an enhancer and/or promoter) of the IGH gene sufficient for the fusion oncogene to be transcribed at a higher level (e.g., 10, 20, 30, 40, 50, 60, 70, 80, 90, 100, 125, 150, 175, or 200% higher) than the second fusion partner gene is normally (e.g., in a wildtype and/or non-disease cell) expressed, e.g., when not subjected to the gross chromosomal rearrangement that formed the fusion oncogene.

In some embodiments, an IGH fusion oncogene comprises the BCL2 gene or a functional variant or fragment thereof (an IGH-BCL2 fusion oncogene). In some embodiments, an IGH fusion oncogene comprises the CCND1 gene or a functional variant or fragment thereof (an IGH-CCND1 fusion oncogene). In some embodiments, an IGH fusion oncogene comprises the BCL6 gene or a functional variant or fragment thereof (an IGH-BCL6 fusion oncogene).

In some embodiments, an IGH fusion oncogene comprises a MYC gene (e.g., c-MYC, 1-MYC, or n-MYC, e.g., c-MYC) or a functional variant or fragment thereof (an IGH-MYC fusion oncogene). Without wishing to be bound by theory, c-MYC is thought to contain three exons: a first non-coding exon, and second and third coding exons. Translational initiation is thought to begin in exon 2. Exon 1 is thought to contain a first and a second promoter (wherein the first promoter is upstream of the second promoter), wherein transcription is initiated primarily from the second promoter in wildtype cells. In some embodiments, the IGH-MYC fusion oncogene comprises all or a portion of exon 2. In some embodiments, the IGH-MYC fusion oncogene comprises all or a portion of exon 3. In some embodiments, the IGH-MYC fusion oncogene comprises all or a portion of exons 2 and 3. In some embodiments the IGH-MYC fusion oncogene is produced by a gross chromosomal rearrangement, e.g., wherein the breakpoint is situated in exon 1 of c-MYC. In some embodiments, the IGH-MYC fusion oncogene comprises a portion of exon 1, e.g., a portion comprising the first and/or second promoter. In some embodiments, transcription of the IGH-MYC fusion oncogene is initiated primarily from the first promoter of Exon 1 (e.g., in the absence of a disrupting agent described herein).

In some embodiments, the cancer is a hematologic cancer. In some embodiments, the cancer comprises a solid tumor. In some embodiments, the cancer is a lymphoma. In some embodiments, the cancer is diffuse large B cell lymphoma (DLBCL). In some embodiments, the cancer is Burkitt's lymphoma. In some embodiments, the cancer is Non-Hodgkin's Lymphoma (NHL). In some embodiments, the cancer is mantle cell lymphoma (MCL). In some embodiments, the cancer is a lymphoma that cannot be classified or is indeterminate (e.g., the cancer is classified as either DLBCL or Burkitt's lymphoma). The compositions and methods described herein may be used to treat cancer. The methods described herein may also improve existing cancer therapeutics to increase bioavailability and/or reduce toxicokinetics. Cancer or neoplasm includes solid or liquid cancer and includes benign or malignant tumors, and hyperplasias, including gastrointestinal cancer (such as non-metastatic or metastatic colorectal cancer, pancreatic cancer, gastric cancer, esophageal cancer, hepatocellular cancer, cholangiocellular cancer, oral cancer, lip cancer); urogenital cancer (such as hormone sensitive or hormone refractory prostate cancer, renal cell cancer, bladder cancer, penile cancer); gynecological cancer (such as ovarian cancer, cervical cancer, endometrial cancer); lung cancer (such as small-cell lung cancer and non-small-cell lung cancer); head and neck cancer (e.g. head and neck squamous cell cancer); CNS cancer including malignant glioma, astrocytomas, retinoblastomas and brain metastases; malignant mesothelioma; non-metastatic or metastatic breast cancer (e.g. hormone refractory metastatic breast cancer); skin cancer (such as malignant melanoma, basal and squamous cell skin cancers, Merkel Cell Carcinoma, lymphoma of the skin, Kaposi Sarcoma); thyroid cancer; bone and soft tissue sarcoma; and hematologic neoplasias (such as multiple myeloma, acute myelogenous leukemia, chronic myelogenous leukemia, myelodysplastic syndrome, acute lymphoblastic leukemia, Hodgkin's lymphoma).

In some embodiments, a site-specific disrupting agent described herein is administered in combination with one or more additional cancer therapies, such as chemotherapy, radiation, or an antibody molecule. In some embodiments, the additional cancer therapy comprises an RNAi molecule, e.g., one that reduces expression of an oncogene, e.g., fusion oncogene. In some embodiments, the oncogene targeted by the RNAi molecule is the oncogene in the anchor sequence mediated conjunction formed by the first anchor sequence and the second anchor sequence.

Technologies provided herein achieve destabilization and/or inhibition of formation of structure and/or function of genomic complexes. Among other things, in some embodiments such provided technologies achieve modulation of gene expression and, for example, enable breadth over controlling gene activity, delivery, and penetrance, e.g., in a cell. In some embodiments, a cell is a mammalian cell. In some embodiments, a cell is a somatic cell. In some embodiments, a cell is a primary cell.

For example, in some embodiments, a cell is a mammalian somatic cell. In some embodiments, a mammalian somatic cell is a primary cell. In some embodiments, a mammalian somatic cell is a non-embryonic cell.

In some embodiments, provided methods comprise a step of: delivering a site-specific disrupting agent to a cell. In some embodiments, a step of delivering is performed ex vivo. In some embodiments, methods further comprise, prior to the step of delivering, a step of removing a cell (e.g., a mammalian cell) from a subject. In some embodiments, methods further comprise, after the step of delivering, a step of (b) administering cells (e.g., mammalian cells) to a subject. In some embodiments, the step of delivering comprises administering a composition comprising a site-specific disrupting agent to a subject. In some embodiments, a subject has a disease or condition.

In some embodiments, the step of delivering comprises delivery across a cell membrane.

In some embodiments, provided methods comprise a step of (a) substituting, adding, or deleting one or more nucleotides of an anchor sequence within a cell, e.g., a mammalian somatic cell.

In some embodiments, the step of substituting, adding, or deleting is performed in vivo. In some embodiments, the step of substituting, adding, or deleting is performed ex vivo.

In some embodiments, an anchor sequence is a genomic anchor sequence in that an anchor sequence is located in a genome of a cell.

Compositions and methods provided herein can be used to treat a disease or disorder in human and non-human animals. In some aspects, the present disclosure provides methods of altering expression of a target gene in a genome, comprising: administering to a human or non-human animal a pharmaceutical composition comprising (a) a site-specific disrupting agent, wherein the disrupting agent inhibits formation of a conjunction that brings a gene expression factor (e.g., an enhancing sequence) out of operable linkage with a target gene, or a gene expression factor (e.g., a silencing/repressor sequence) into operable linkage with a target gene.

In some embodiments, compositions and methods provided herein can be used to treat a lymphoma (e.g., NHL, MCL, DLBCL, or Burkitt's) associated with an IGH fusion oncogene (e.g., IGH-BCL2, IGH-MYC, or IGH-CCND1). In one aspect, the disclosure is directed, in part, to a method of decreasing expression of the IGH fusion oncogene and/or treating the cancer by disrupting an anchor site, e.g., CTCF binding motif, proximal to the IGH fusion oncogene, e.g., by introducing a mutation (e.g., a substitution, insertion, or deletion) into the anchor site. In some embodiments, said methods utilizes a site-specific disrupting agent comprising a targeting moiety that binds to the anchor site, e.g., CTCF binding motif.

In one aspect, the disclosure is directed, in part, to a method of decreasing expression of the IGH fusion oncogene and/or treating the cancer by excising an anchor site, e.g., CTCF binding motif, proximal to the IGH fusion oncogene, e.g., by introducing a deletion that removes the anchor site. In some embodiments, said methods utilizes a site-specific disrupting agent comprising a targeting moiety that binds to the nucleic acid sequence(s) adjacent to (e.g., surrounding) the anchor site, e.g., CTCF binding motif.

In one aspect, the disclosure is directed, in part, to a method of decreasing expression of the IGH fusion oncogene and/or treating the cancer by epigenetically modifying (e.g., methylating the DNA and/or histones associated with) a regulatory element (e.g., an enhancer (e.g., super enhancer) or promoter) proximal to the IGH fusion oncogene. In some embodiments, said methods utilizes a site-specific disrupting agent comprising a targeting moiety that binds to the regulatory element, e.g., upstream of the IGH gene, e.g., a promoter operably linked to the IGH gene.

In one aspect, the disclosure is directed, in part, to a method of decreasing expression of the IGH fusion oncogene and/or treating the cancer by epigenetically modifying (e.g., compacting the chromatin comprising) a regulatory element (e.g., an enhancer (e.g., super enhancer)) proximal to the IGH fusion oncogene. In some embodiments, said methods utilizes a site-specific disrupting agent comprising a targeting moiety that binds to the enhancer, e.g., duplicated enhancers in the 3′Ca, operably linked to the IGH gene.

In some embodiments, the site specific disrupting agent is effective at decreasing expression of the IGH fusion oncogene and/or inhibiting growth/proliferation of SU-DHL-6, U-2946, or GRANTA519 cells. Compositions and methods provided herein can be used to treat disease in human and non-human animals. In some aspects, methods of treating a disease or condition comprises administering one or more compositions as described herein to a subject in need thereof.

In some embodiments, provided methods comprise a step of delivering a mammalian somatic cell to a subject having a disease or condition, wherein the anchor sequence within a mammalian somatic cell is targeted by a disrupting agent. In some embodiments, a subject is a mammal, e.g., a human. In some embodiments, a subject has a disease or condition.

In some embodiments, provided methods comprise a step of: (a) administering somatic mammalian cells to a subject, wherein somatic mammalian cells were obtained from a subject, and a site-specific disrupting agent as described herein had been delivered ex vivo to somatic mammalian cells. In some embodiments, the ex vivo treatment is performed in combination with a CART therapy. In some embodiments, the ex vivo treatment is performed in combination with a bone marrow transplant, e.g., for a subject having a leukemia, e.g., AML. For instance, in some embodiments, the method comprises one or more of, e.g., all of: (i) obtaining a sample of bone marrow cells (e.g., by removing the bone marrow cells from the subject), (ii) treating the bone marrow cells ex vivo with the site-specific disrupting agent, (iii) ablating bone marrow cells in the subject, e.g., by chemotherapy, and (iv) administering the treated bone marrow cells to the subject.

In some aspects, provided methods comprise altering gene expression or destabilizing and/or inhibiting formation of an anchor sequence-mediated conjunction in a mammalian subject. Methods may include administering to a subject (separately or in a single pharmaceutical composition): a protein comprising a first polypeptide domain that comprises a Cas or modified Cas protein and a second polypeptide domain that comprises a polypeptide having DNA methyltransferase activity [or associated with demethylation or deaminase activity], or a nucleic acid encoding a protein comprising a first polypeptide domain that comprises a Cas or modified Cas protein and a second polypeptide domain that comprises a polypeptide having DNA methyltransferase activity [or associated with demethylation or deaminase activity], and at least one guide RNA (gRNA) that targets an anchor sequence of an anchor sequence-mediated conjunction. In some embodiments, a gRNA targets a sequence that is not an anchor sequence. In some embodiments, a gRNA targets a component of a genomic complex, such as an ncRNA or eRNA. In some embodiments, a gRNA targets a sequence within an anchor sequence-mediated conjunction comprising a gene to be modulated. In some embodiments, a gRNA targets a transcription factor or regulator or portion thereof, wherein targeting occurs by targeting a sequence encoding a transcription factor, regulator or portion thereof.

Methods and compositions as provided herein may treat disease by inhibiting formation of and/or destabilizing an anchor sequence-mediated conjunction or modulating (e.g., reducing) transcription of a nucleic acid sequence. In some embodiments, chromatin structure or topology of an anchor sequence-mediated conjunction is altered to result in a stable modulation (e.g., decrease) of transcription, such as a modulation that persists for at least about 1 hr to about 30 days, or at least about 2 hrs, 6 hrs, 12 hrs, 18 hrs, 24 hrs, 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or longer or any time therebetween. In some other embodiments, chromatin structure or topology of an anchor sequence-mediated conjunction is altered to result in a transient modulation (e.g., decrease) of transcription, such as a modulation that persists for no more than about 30 mins to about 7 days, or no more than about 1 hr, 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 24 hrs, 36 hrs, 48 hrs, 60 hrs, 72 hrs, 4 days, 5 days, 6 days, 7 days, or any time therebetween.

In some aspects, methods provided by the present disclosure may comprise modifying expression of a target gene, comprising administering to a cell, tissue or subject a genomic complex modulating agent (e.g., disrupting agent) as described herein.

In some aspects, the present disclosure provides methods of modifying expression of a target gene, comprising inhibiting formation of and/or stabilization destabilizing of an anchor sequence-mediated conjunction associated with a target gene, wherein an alteration modulates (e.g., decreases) transcription of a target gene.

In some embodiments, provided technologies may comprise inducibly altering an anchor sequence-mediated conjunction or other portion of a genomic complex (e.g. ncRNA, eRNA, transcription factor, transcription regulator, etc.) with a disrupting agent. Use of an inducible alteration to an anchor sequence-mediated conjunction or other component of a genomic complex (e.g. ncRNA, transcription factor, etc.) provides a molecular switch. In some embodiments, a molecular switch is capable of turning on an alteration when desired. In some embodiments, a molecular switch is capable of turning off an alteration when it is not desired. In some embodiments, a molecular switch is capable of both turning on and turning off an alteration, as desired. Examples of systems used for inducing alterations include, but are not limited to an inducible targeting moiety based on a prokaryotic operon, e.g., the lac operon, transposon Tn10, tetracycline operon, and the like, and an inducible targeting moiety based on a eukaryotic signaling pathway, e.g. steroid receptor-based expression systems, e.g. the estrogen receptor or progesterone-based expression system, the metallothionein-based expression system, the ecdysone-based expression system. In some embodiments, provided methods and compositions may include an inducible nucleating polypeptide or other protein that interacts with an anchor sequence-mediated conjunction.

In some embodiments, cells or tissue may be excised from a subject and gene expression, e.g., endogenous or exogenous gene expression, may be altered ex vivo prior to transplantation of cells or tissues back into a subject. Any cell or tissue may be excised and used for re-transplantation. Some examples of cells and tissues include, but are not limited to, stem cells, adipocytes, immune cells, myocytes, bone marrow derived cells, cells from the kidney capsule, fibroblasts, endothelial cells, and hepatocytes.

Current delivery technologies may also have inadvertent effects, e.g., genome wide removal of transcription factors from DNA. In some embodiments, methods provided herein modulate transcription of a gene by delivering a composition, e.g., disrupting agent, as provided herein across a membrane without off-target, e.g., widespread or genome-wide, effects, e.g., removal of transcription factors. In some embodiments, delivering a composition, e.g., disrupting agent, provided herein at doses sufficient to increase penetration of a disrupting agent across a membrane does not significantly alter off-target transcriptional activity, e.g., an increase of less than 50%,40%, 20%, 15%, 10%, 5%, 4%, 3%, 2%, 1%, or any percentage therebetween of transcriptional activity of one or more off-targets as compared to activity after delivery of a disrupting agent alone.

In some aspects, the present disclosure provides technologies for delivering a composition, e.g., disrupting agent, as provided herein to a target tissue or cell, where a composition, e.g., disrupting agent, includes a targeting moiety, e.g., a receptor ligand, that targets a specific tissue or cell and a therapeutic moiety. Upon administration, a composition increases targeted delivery of a therapeutic moiety as compared to a therapeutic moiety alone. When a composition of the present disclosure is used in combination with an existing therapeutic that suffers from diffusion or off-target effects, specificity of the therapeutic is increased. For example, a composition described herein includes a disrupting agent comprising (e.g. linked to) a particular agent and a ligand that specifically binds a receptor on a particular target cell type. Administration of such a composition increases specificity of the agent to the target cells through a ligand-receptor interaction.

In some aspects, the present disclosure provides technologies for intracellular delivery of a therapeutic comprising contacting a cell or tissue with compositions described herein. In some embodiments, a therapeutic is a disrupting agent or moiety thereof as described herein, and a composition increases intracellular delivery of a therapeutic as compared to a therapeutic alone.

In some aspects, a kit is described that includes a disrupting agent comprising: (a) a nucleic acid encoding a protein comprising a first polypeptide domain that comprises a Cas or modified Cas protein and a second polypeptide domain, e.g., a polypeptide having DNA methyltransferase activity or associated with demethylation or deaminase activity, and (b) at least one guide RNA (gRNA) for targeting a protein to a target genomic sequence element, e.g., an anchor sequence of a target anchor sequence-mediated conjunction in a target cell. In some embodiments, a nucleic acid encoding a protein and a gRNA are in the same vector, e.g., a plasmid, an AAV vector, an AAV9 vector. In some embodiments, a nucleic acid encoding a protein and a gRNA are in separate vectors.

### Modulating Gene Expression

In some embodiments, particular genes are associated with complexes and in many cases affect gene expression in a given genomic complex. Thus, in some embodiments, as described herein, complex inhibition inhibits expression of an associated gene. In some embodiments, as described herein, complex inhibition promotes expression of an associated gene.

In some embodiments, transcription of a nucleic acid sequence is modulated, e.g., transcription of a target nucleic acid sequence, as compared with a reference value, e.g., transcription of a target sequence in absence of an altered anchor sequence-mediated conjunction.

In some embodiments, provided are technologies for inhibiting formation of or destabilizing a genomic complex which modulates expression of a gene associated with the genomic complex, which comprises a first anchor sequence and a second anchor sequence. A gene that is associated with the genomic complex may be associated with an anchor sequence-mediated conjunction at least partially within the conjunction (that is, situated sequence-wise between first and second anchor sequences), or it may be external to the conjunction in that it is not situated sequence-wise between a first and second anchor sequences, but is located on the same chromosome and in sufficient proximity to at least a first or a second anchor sequence such that its expression can be modulated by inhibiting the formation of or destabilizing the genomic complex. Those of ordinary skill in the art will understand that distance in three-dimensional space between two elements (e.g., between the gene and the anchor sequence-mediated conjunction) may, in some embodiments, be more relevant than distance in terms of basepairs.

In some embodiments, inhibition of formation of or destabilization of a genomic complex modulates expression of a gene comprising altering accessibility of a transcriptional control sequence to a gene. A transcriptional control sequence, whether internal or external to an anchor sequence-mediated conjunction, can be an enhancing sequence or a silencing (or repressor) sequence.

For example, in some embodiments, methods are provided for destabilizing and/or inhibiting forming or a genomic complex to modulate expression of a gene within an anchor sequence-mediated conjunction comprising a step of: contacting the first and/or second anchor sequence with a genomic complex modulating agent (e.g., disrupting agent) as described herein. In some embodiments, an anchor sequence-mediated conjunction comprises at least one transcriptional control sequence that is “internal” to a conjunction in that it is at least partially located sequence-wise between first and second anchor sequences. Thus, in some embodiments, both a gene whose expression is to be modulated (the “target gene”) and a transcriptional control sequence are within an anchor sequence-mediated conjunction. See, e.g., a Type 1 anchor sequence-mediated conjunction as depicted in FIG. 6.

In some embodiments, a gene is separated from an internal transcriptional control sequence by at least 300, at least 400, at least 500, at least 600, at least 700, at least 800, or at least 900 base pairs. In some embodiments, a gene is separated from an internal transcriptional control sequence by at least 1.0, at least 1.2, at least 1.4, at least 1.6, or at least 1.8 kb. In some embodiments, a gene is separated from an internal transcriptional control sequence by at least 2 kb, at least 3 kb, at least 4 kb, at least 5 kb, at least 6 kb, at least 7 kb, at least 8 kb, at least 9 kb, or at least 10 kb. In some embodiments, a gene is separated from an internal transcriptional control sequence by at least 20 kb, at least 30 kb, at least 40 kb, at least 50 kb, at least 60 kb, at least 70 kb, at least 80 kb, at least 90 kb, or at least 100 kb. In some embodiments, a gene is separated from an internal transcriptional control sequence by at least 150 kb, at least 200 kb, at least 250 kb, at least 300 kb, at least 350 kb, at least 400 kb, at least 450 kb, or at least 500 kb. In some embodiments, the gene is separated from an internal transcriptional control sequence by at least 600 kb, at least 700 kb, at least 800 kb, at least 900 kb, or at least 1 Mb.

In some embodiments, an anchor sequence-mediated conjunction comprises at least one transcriptional control sequence that is “external” to the conjunction in that it is not located sequence-wise between first and second anchor sequences. (See, e.g., Types 2, 3, and 4 anchor sequence-mediated conjunctions depicted in FIG. 6.) In some embodiments, a first and/or a second anchor sequence is located within 1 Mb, within 900 kb, within 800 kb, within 700 kb, within 600 kb, within 500 kb, within 450 kb, within 400 kb, within 350 kb, within 300 kb, within 250 kb, within 200 kb, within 180 kb, within 160 kb, within 140 kb, within 120 kb, within 100 kb, within 90 kb, within 80 kb, within 70 kb, within 60 kb, within 50 kb, within 40 kb, within 30 kb, within 20 kb, or within 10 kb of an external transcriptional control sequence. In some embodiments, the first and/or the second anchor sequence is located within 9 kb, within 8 kb, within 7 kb, within 6 kb, within 5 kb, within 4 kb, within 3 kb, within 2 kb, or within 1 kb of an external transcriptional control sequence.

For example, in some embodiments, methods are provided for modulating expression of a gene external to an anchor sequence-mediated conjunction comprising a step of: contacting a first and/or second anchor sequence with a genomic complex modulating agent (e.g., disrupting agent) as described herein. In some embodiments, an anchor sequence-mediated conjunction comprises at least one internal transcriptional control sequence.

In some embodiments, an anchor sequence-mediated conjunction comprises at least one external transcriptional control sequence.

The compositions and methods described herein may be used to inhibit genomic complex formation or decrease stability to modulate (e.g., decrease) expression of a gene, for example at least one of CCDCl₆-RET or PAX3-FOXO1 gene.

Thus, among other things, the present application provides technologies for modulating gene expression by destabilizing and/or inhibiting formation of genomic complexes as described herein.

In some embodiments, modulation may include inhibiting formation of and/or destabilizing insulated neighborhoods. In some embodiments, modulating insulated neighborhoods affects transcription by interfering with formation/reducing frequency of assembly/inducing dissociation of a genomic complex.

In some aspects, the present disclosure provides methods that destabilize and/or inhibit formation of one or more genomic complexes. By way of non-limiting example, in some embodiments destabilization and/or formation inhibition may refer to changes in structural topology of one or more genomic complexes. In some embodiments, destabilization and/or formation inhibition, as used herein, may refer to changes in function of one or more genomic complexes without requiring impact or change to structural topology. For example, in some embodiments, methods may include destabilization and/or formation inhibition of structural topology of one or more genomic complexes. Without wishing to be bound by any theory, in some embodiments, destabilization and/or formation inhibition of genomic complexes may alter gene expression. Gene expression alteration may be or comprise downregulation of one or more genes relative to expression levels in absence of genomic complex destabilization and/or formation inhibition.

In some embodiments, destabilization and/or formation inhibition may comprise deleting one or more CTCF binding motifs.

In some embodiments, destabilization and/or formation inhibition may comprise methylating one or more CTCF binding motifs.

In some embodiments, destabilization and/or formation inhibition may comprise inducing degradation of non-coding RNA that is part of a genomic complex (e.g. between two CTCF binding motifs/anchor sites).

In some embodiments, destabilization and/or formation inhibition may comprise interfering with assembly of one or more genomic complexes (e.g. a genomic complex that would otherwise form in absence of exogenous interference) by blocking resident non-coding RNA.

### Genetic Modification

In some embodiments, technologies (e.g. methods and/or compositions) provided by the present disclosure for altering a target gene may include site specific editing or mutating of a genomic sequence element (e.g., that participates in the genomic complex and/or is part of an gene associated therewith). For example, in some embodiments, an endogenous or naturally occurring anchor sequence may be altered to inhibit targeting to an anchor sequence (e.g., thereby destabilizing and/or inhibiting formation of an anchor sequence-mediated conjunction), or may be altered to mutate or replace an anchor sequence (e.g., to mutate or replace an anchor sequence with an altered anchor sequence that has an altered affinity, e.g., decreased affinity, to a nucleating polypeptide) to modulate (e.g., decrease) strength of a targeted conjunction. A nucleating polypeptide may be, e.g., CTCF, cohesin, USF1, YY1, TAF3, ZNF143 binding motif, or another polypeptide that promotes formation of an anchor sequence-mediated conjunction.

In some embodiments, technologies as provided herein may include those that alter a target sequence (e.g. a sequence that is part of or participates in a targeted genomic complex).

An alteration can be introduced in a gene of a cell, e.g., in vitro, ex vivo, or in vivo.

In some cases, compositions, e.g., disrupting agents, and/or methods of the present disclosure are for altering chromatin structure, e.g., such that a two-dimensional representation of chromatin structure may change from that of a loop to a non-loop (or favor a non-loop over a loop) or vice versa, to alter a component of a genomic complex (e.g. a transcription factor and, e.g. its interaction with a genomic sequence), to inactivate a targeted CTCF-binding motif, e.g., an alteration inhibits CTCF binding thereby inhibiting formation of a targeted conjunction, etc. In other examples, an alteration inhibits (e.g., increases the level of) activity of a particular genomic complex component thereby decreasing or inhibiting formation of a genomic complex (e.g., by altering a CTCF sequence to bind with lower affinity to a nucleating polypeptide). In some embodiments, a targeted alteration decreases activity of a particular genomic complex component thereby destabilizing or inhibiting formation of a genomic complex (e.g., by altering the CTCF sequence to bind with less affinity to a nucleating polypeptide), thereby inhibiting formation of a targeted conjunction.

In some embodiments, provided disrupting agents may comprise (i) a fusion molecule comprising an enzymatically inactive Cas polypeptide and a deaminating agent, or a nucleic acid encoding the fusion molecule; and (ii) a nucleic acid molecule (e.g. gRNA, PNA, BNA, etc), wherein the nucleic acid molecule targets a fusion molecule to a target sequence (e.g. in a genomic complex, e.g. in an anchor sequence-mediated conjunction within a genomic complex) but not to at least one non-target anchor sequence (a “site-specific nucleic acid molecule”, such as described further herein).

In some embodiments, in order to introduce small mutations or a single-point mutation, a homologous recombination (HR) template can also be used. In some embodiments, an HR template is a single stranded DNA (ssDNA) oligo or a plasmid. In some embodiments, for example, for ssDNA oligo design, one may use around 100-150 bp total homology with a mutation introduced roughly in the middle, giving 50-75 bp homology arms.

In some embodiments, a nucleic acid molecule for targeting a target anchor sequence, e.g., a target sequence, is administered in combination with an HR template selected from:
(a) a nucleotide sequence comprising a target sequence of interest (e.g. target sequence that is part of or participates in a target genomic complex);
(b) a nucleotide sequence at least 75%, 80%, 85%, 90%, 95% identical to a target sequence of interest;
(c) a nucleotide sequence comprising a target sequence of interest having at least 1, 2, 3, 4, 5, but less than 15, 12 or 10 nucleotide additions, substitutions or deletions.

### Modifying Chromatin Structure

In some embodiments, methods provided herein modulate chromatin structure (e.g., anchor sequence-mediated conjunctions) in order to modulate (e.g., decrease) gene expression in a subject, e.g., by modifying anchor sequence-mediated conjunctions in DNA. Those skilled in the art reading the present specification will appreciate that modulations described herein may modulate chromatin structure in a way that would alter its two-dimensional representation (e.g., would add, alter, or delete a loop or other anchor sequence-mediated conjunction); such modulations are referred to herein, in accordance with common parlance, as modulations or modification of a two-dimensional structure.

In some aspects, methods provided herein may comprise modifying a two-dimensional structure by altering a topology of an anchor sequence-mediated conjunction, e.g., a loop, to modulate transcription of a nucleic acid sequence, wherein altered topology of an anchor sequence-mediated conjunction modulates transcription of a nucleic acid sequence.

In some aspects, methods provided herein may comprise modifying a two-dimensional structure chromatin structure by altering a topology of a plurality of anchor sequence-mediated conjunctions, e.g., multiple loops, to modulate transcription of a nucleic acid sequence, wherein altered topology modulates transcription of a nucleic acid sequence.

In some aspects, methods provided herein may comprise modulating transcription of a nucleic acid sequence by altering an anchor sequence-mediated conjunction, e.g., a loop, that influences transcription of a nucleic acid sequence, wherein altering an anchor sequence-mediated conjunction modulates transcription of a nucleic acid sequence.

In some embodiments, altering an anchor sequence-mediated conjunction comprises modifying a chromatin structure, e.g., inhibiting forming of or destabilizing [e.g., reversible or irreversible] a topology of a genomic complex, e.g., an anchor sequence-mediated conjunction, by altering one or more nucleotides in an anchor sequence-mediated conjunction [e.g., genetically modifying the sequence] or epigenetically modifying [e.g., modulating DNA methylation at one or more sites] an anchor sequence-mediated conjunction. In some embodiments, altering an anchor sequence-mediated conjunction comprises modifying a chromatin structure.

### Modifying Chromatin Structure

In some embodiments, provided compositions and/or methods are described herein for altering a genomic complex by site specific epigenetic modification (e.g., methylation or demethylation).

In some embodiments, a disrupting agent may cause epigenetic modification. For example, an endogenous or naturally occurring target sequence (e.g. a sequence within a target genomic complex) may be altered to increase its methylation (e.g., interaction of a component of a genomic complex (e.g. a transcription factor) with a portion of a genomic sequence, decreasing binding of a nucleating polypeptide to an anchor sequence and inhibiting or decreasing strength of an anchor sequence-mediated conjunction, etc.).

In some particular embodiments, a disrupting agent may be or comprise a fusion molecule, for example comprising a site-specific targeting moiety (such as any one of targeting moieties as described herein) and an epigenetic modifying moiety, wherein a site-specific targeting moiety targets a fusion molecule to a target anchor sequence but not to at least one non-target anchor sequence. An epigenetic modifying moiety can be any one of or any combination of epigenetic modifying moieties as disclosed herein.

In some embodiments, for example, fusions of a catalytically inactive endonuclease e.g., a dead Cas9 (dCas9, e.g., D10A; H840A) tethered with all or a portion of (e.g., biologically active portion of) an (one or more) effector domain create chimeric proteins that can be guided to specific DNA sites by one or more RNA sequences (sgRNA) to modulate activity and/or expression of one or more target nucleic acids sequences (e.g., to methylate or demethylate a DNA sequence).

In some embodiments, fusion of a dCas9 with all or a portion of one or more effector domains of an epigenetic modifying moiety (such as a DNA methylase or enzyme with a role in DNA demethylation) creates a chimeric protein that is useful in methods provided by the present disclosure. Accordingly, for example, in some embodiments, a nucleic acid encoding a dCas9-methylase fusion in combination with a site-specific gRNA or antisense DNA oligonucleotide that targets a fusion to a genomic complex component (such as a transcription factor, ncRNA, CTCF binding motif, etc.), may together decrease affinity or ability of a component of a genomic complex to interact with a particular genomic sequence.

In some embodiments, all or a portion of one or more methylase, or enzyme with a role in DNA demethylation, effector domains are fused with an inactive nuclease, e.g., dCas9. In some embodiments, 1, 2, 3, 4, 5, 6, 7, 8, 9, 10, 11, 12, 13, 14, 15, 16, 17, 18, 19, 20, or more methylase, or enzyme with a role in DNA demethylation, effector domains (all or a biologically active portion) are fused with dCas9. Chimeric proteins as described herein may also comprise a linker, e.g., an amino acid linker. In some embodiments, a linker comprises 2 or more amino acids, e.g., one or more GS sequences. In some embodiment, fusion of Cas9 (e.g., dCas9) with two or more effector domains (e.g., of a DNA methylase or enzyme with a role in DNA demethylation) comprises one or more interspersed linkers (e.g., GS linkers) between domains. In some aspects, dCas9 is fused with 2-5 effector domains with interspersed linkers.

In embodiments, compositions and/or methods of the present disclosure may comprise a gRNA that specifically targets a sequence or component of a genomic complex (e.g. CTCF binding motif, ncRNA/eRNA, transcription factor, transcription regulator, etc.). In some embodiments, the sequence or component is associated with a particular type of gene or sequence, which may be associated with one or more diseases, disorders and/or conditions.

Epigenetic modifying moieties useful in provided methods and/or compositions include agents that affect, e.g., DNA methylation, histone acetylation, and RNA-associated silencing. In some embodiments, methods provided herein may involve sequence-specific targeting of an epigenetic enzyme (e.g., an enzyme that generates or removes epigenetic marks, e.g., acetylation and/or methylation). In some embodiments, exemplary epigenetic enzymes that can be targeted to an anchor sequence using the CRISPR methods described herein include DNA methylases (e.g., DNMT3a, DNMT3b, DNMTL), enzymes with a role in DNA demethylation (e.g., the TET family enzymes catalyze oxidation of 5-methylcytosine to 5-hydroxymethylcytosine and higher oxidative derivatives), histone methyltransferases, histone deacetylase (e.g., HDAC1, HDAC2, HDAC3), sirtuin 1, 2, 3, 4, 5, 6, or 7, lysine-specific histone demethylase 1 (LSD1), histone-lysine-N-methyltransferase (Setdb1), euchromatic histone-lysine N-methyltransferase 2 (G9a), histone-lysine N-methyltransferase (SUV39H1), enhancer of zeste homolog 2 (EZH2), viral lysine methyltransferase (vSET), histone methyltransferase (SET2), and protein-lysine N-methyltransferase (SMYD2). Examples of such epigenetic modifying moieties are described, e.g., in de Groote et al. Nuc. Acids Res. (2012):1-18.

In some embodiments, an epigenetic modifying moiety useful herein comprises a construct described in Koferle et al. Genome Medicine 7.59 (2015):1-3 (e.g., at Table 1), incorporated herein by reference.

Exemplary dCas9 fusion methods and compositions that are adaptable to methods and/or compositions of the present disclosure are known and are described, e.g., in Kearns et al., Functional annotation of native enhancers with a Cas9-histone demethylase fusion. Nature Methods 12, 401-403 (2015); and McDonald et al., Reprogrammable CRISPR/Cas9-based system for inducing site-specific DNA methylation. Biology Open 2016: doi: 10.1242/bio.019067.

All references and publications cited herein are hereby incorporated by reference.

### EXAMPLES

The following examples are provided to further illustrate some embodiments of the present disclosure, but are not intended to limit the scope of the disclosure; it will be understood by their exemplary nature that other procedures, methodologies, or techniques known to those skilled in the art may alternatively be used.

### Example 1

This example demonstrates the downregulation of the fusion oncogene CCDC6-RET by using CRISPR/Cas9 to genetically modify the CTCF anchor sequence-mediated conjunctions involved in the formation of CFLs.

CCDC6-RET is a fusion oncogene caused by a translocation that is recurrently found in thyroid and lung cancers. The 5′ partner of this fusion, CCDC6, is a gene encoding a coiled-coil domain-containing protein that may function as a tumor suppressor. The 3′ partner of this fusion, RET, is a proto-oncogene that encodes a transmembrane receptor and member of the tyrosine kinase family of proteins. RET plays a role in cellular differentiation, proliferation, migration and survival. The chromosomal translocation resulting in CCDC6-RET causes the production of a fusion oncoprotein that juxtaposes the amino-terminal portion of CCDC6 protein with the intracellular kinase-encoding domain of RET, causing oncogenic activation. RET inhibition has been explored as a cancer therapeutic and demonstrated some tumor regression. However, no RET-specific inhibitors are currently clinically available, though several promiscuous kinase inhibitors target RET and other kinases.

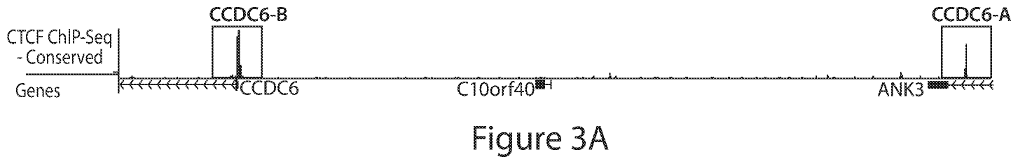

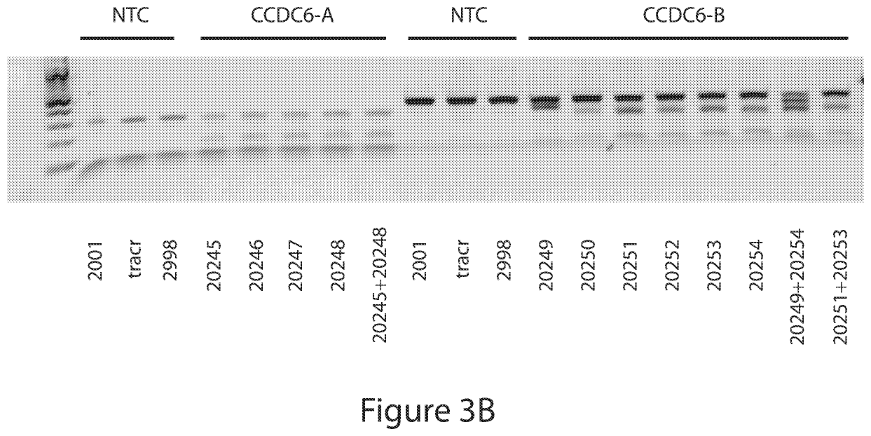

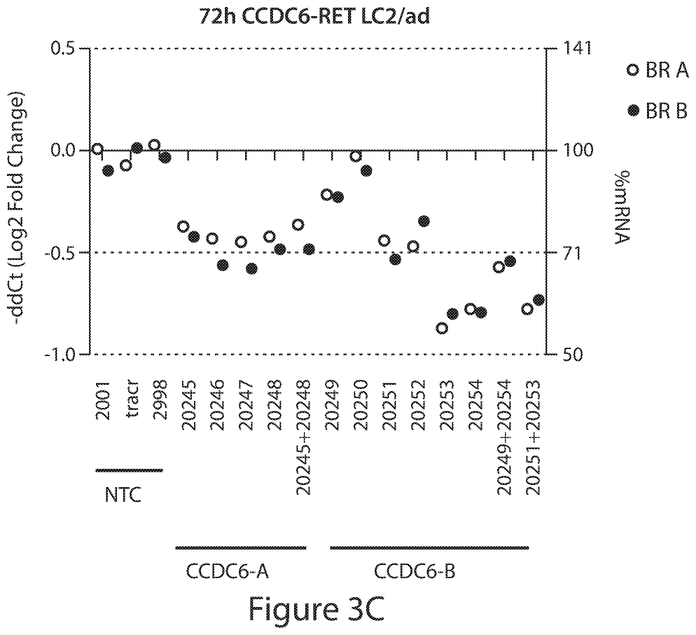

First, anchor sequences were identified. CTCF-ChIP-SEQ data sets were analyzed to identify CTCF binding sites proximal to CCDC6. CTCF occupies two anchor sequences, CCDC6-A and CCDC6-B, located upstream of the CCDC6 gene in a highly conserved manner across multiple cell types (FIG. 3A). According to the non-limiting theory herein, these CTCF proteins act as anchors for novel CFLs containing the CCDC6-RET fusion oncogene, ensuring the high expression of CCDC6-RET in cancer cells.

Next, gRNA constructs were designed to target these anchor sequences (Table 5).

TABLE 5

| Sequences of guide RNAs (gRNAs) targeting | |
|---|---|
| CTCF anchor sequences associated | |
| with the CFL containing CCDC6-RET. | |
| Name | gRNA sequence (5′-3′) |
| 2001 | ATGATCTCTGCTGCCAGTAG |
| 2998 | GTATTACTGATATTGGTGGG |
| GD-20245 | GTGATGACAGCGCCATCTGA |
| GD-20246 | TGATGACAGCGCCATCTGAT |
| GD-20247 | CCTCACACCTTCCCATCAGA |
| GD-20248 | GACAGCGCCATCTGATGGGA |
| GD-20249 | TTTCAGCCAGCTTTGCTGGG |
| GD-20250 | CGTGGTCACCAGACGGCGGC |
| GD-20251 | GGGACCCGCCCGCCGCCGTC |
| GD-20252 | CGCCCGTGGTCACCAGACGG |
| GD-20253 | GCCCGCCCGTGGTCACCAGA |
| GD-20254 | CGCCGCCGTCTGGTGACCAC |

Cas9 and gRNAs were then introduced into LC2/ad cells, which contain the CCDC6-RET fusion oncogene in a novel CFL. Specifically, LC2/ad cells were transduced overnight with lentivirus encoding Cas9 and a puromycin resistance gene cassette. The following day, the transduced cells were passaged into puromycin-containing culture medium (RPMI 1640:Ham's F-12 1:1 mixture, supplemented with 10% fetal bovine serum, 1% penicillin/streptomycin and 2 μg/ml puromycin). Puromycin-resistant LC2/ad cells were maintained under selection for 3 days to establish a population of cells stably expressing Cas9.

LC2/ad-Cas9 cells were transfected with a gRNA or combination of chemically synthesized gRNAs (Table 5) targeted to the CTCF anchor sequence using commercially available transfection reagents (Thermo Fisher Scientific). At 72-hr post-transfection, cells were harvested for genomic DNA and RNA extraction using commercially available reagents and protocols (Lucigen; Thermo Fisher Scientific).

The cells were then assayed to determine whether Cas9-mediated editing had been successful. Targeted genomic regions were PCR-amplified using specific primers (Table 6) and commercially available polymerase mixes (Takara Bio), heteroduplexed (denaturing and reannealing the PCR product) and subsequently analyzed by T7E1 endonuclease assay (Integrated DNA Technologies). T7E1 preferentially cleaves DNA duplexes having mismatch regions (e.g., a duplex between a wild-type oligonucleotide and an oligonucleotide with a deletion) compared to perfectly complementary duplexes. T7E1 products were separated by agarose gel electrophoresis, and DNA bands were visualized by ethidium bromide staining. gRNAs targeting the CTCF anchor sequences showed Cas9-mediated editing as shown by the presence of high-mobility T7E1 cleavage products, whereas non-targeting control (NTC) gRNAs showed only the lower-mobility, unedited product (FIG. 3B). The data indicate that all target specific gRNAs tested were sufficient to direct some level of Cas9-mediated cleavage of the target sequences.

TABLE 6
Sequences of primers used to amplify targeted
genomic regions corresponding to the
CTCF anchor sequences associated with the
CFL containing CCDC6-RET.

| Name | gRNA sequence (5′-3′) |
|---|---|
| CCDC6-A-F | CCACACTGGGTACAGGAAGG |
| CCDC6-A-R | CCCAAAGCAAGACAGATTCC |
| CCDC6-B-F | TTGGGCAGTATTGCACTGG |
| CCDC6-B-R | GCCACAACACGGTAGAGGAT |

Finally, expression of CCDC6-RET was quantified. cDNA synthesis was performed on total RNA extracted from the edited cells and control cells, and subsequently used for quantitative real-time PCR (Thermo Fisher Scientific). Taqman probes/primers specific for CCDC6-RET (Assay ID Hs04396844_ft, Thermo Fisher Scientific) were multiplexed with internal control probes/primers for PPIB (Assay ID Hs00168719_m1, Thermo Fisher Scientific) using the FAM-MGB and VIC-MGB dyes, respectively, and gene expression was analyzed by a real-time Taqman PCR kit (Thermo Fisher Scientific). gRNAs targeting the CTCF anchor sequences showed reduction in CCDC6-RET expression at 72 hr compared to NTC gRNAs (FIG. 3C). Each biological replicate (BR) is shown as a gray (A) or black (B) data point. This result indicates that modifying conserved CTCF anchor sequences in a cancer associated fusion gene can lower expression of the cancer associated fusion gene and may be useful in treating the associated cancer in patients. Without wishing to be bound by theory, the reduction in expression may be due to disruption of the CFL.

### Example 2

This example demonstrates the downregulation of the fusion oncogene PAX3-FOXO1 by using CRISPR/Cas9 to genetically modify the CTCF anchor sequence-mediated conjunctions involved in the formation of CFLs. This example also demonstrates that the level of PAX3-FOXO1 downregulation in the rhabdomyosarcoma cell line, RH30, leads to an impairment in the rate of cell proliferation in vitro.

PAX3-FOXO1 is a fusion oncogene caused by a translocation that is recurrently found in alveolar rhabdomyosarcomas. The 5′ partner of this fusion, PAX3, is a gene encoding a paired box domain-containing transcription factor that plays critical roles in muscle development as well as the development of other tissues and cell types. The 3′ partner of this fusion, FOXO1, is a forkhead transcription factor that may play a role in myogenic growth and differentiation. The chromosomal translocation resulting in PAX3-FOXO1 causes the production of a fusion oncoprotein that fuses the DNA-binding domain of the PAX3 transcription factor with the transactivating domain of the FOXO1 transcription factor, creating a novel and highly potent transcription factor that can establish an oncogenic program through activation of its target genes. PAX3-FOXO1 is believed to be the single genetic alteration capable of driving the pathogenesis of alveolar rhabdomyosarcoma. However, it has not been extensively explored as a therapeutic target given the challenge of pharmacologically targeting transcription factors.

First, anchor sequences were identified. CTCF-ChIP-SEQ data sets were analyzed to identify CTCF binding sites proximal and internal to PAX3. In addition, rhabdomyosarcoma-specific CTCF binding at anchor sequences was determined by using CTCF-ChIP-Seq on RH30 cells. RH30 cells were fixed with 1% formaldehyde in 99% of growth medium (DMEM supplemented with 10% fetal bovine serum and 1% penicillin/streptomycin). Following the addition of glycine to quench the fixation, cells were pelleted by centrifugation, washed with phosphate-buffered saline (PBS) and sonicated using a E220 evolution instrument (Covaris) to shear the chromatin. Following centrifugation, the sheared chromatin supernatant was collected and added to Protein G magnetic beads (Thermo Fisher Scientific) complexed with a CTCF-specific antibody (Cell Signaling Technology). Following overnight incubation at 4 degrees Celsius, the CTCF-chromatin complexes bound to beads were washed in high and low salt buffers and subsequently resuspended in the elution buffer. CTCF-chromatin complexes were eluted from beads at 65 degrees Celsius for 15 min. The crosslinks were then reversed by incubating overnight at 65 degrees Celsius, and DNA was purified and concentrated using clean-and-concentrate columns (Zymo Research). The resulting DNA was quantified by Qubit (Thermo Scientific) and analyzed by using a fragment analyzer (Agilent) prior to library preparation and next-generation sequencing (Illumina). Sequencing reads were computationally processed and mapped to the human genome (hg19) to identify CTCF peaks.

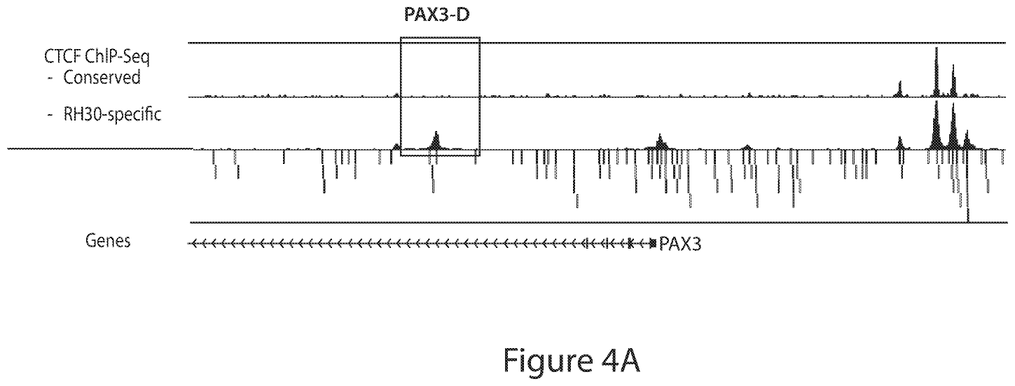

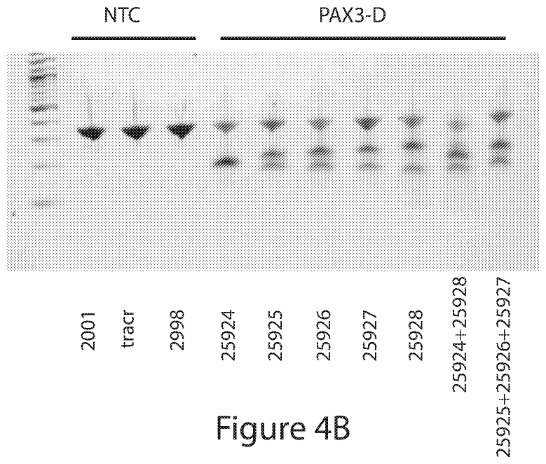

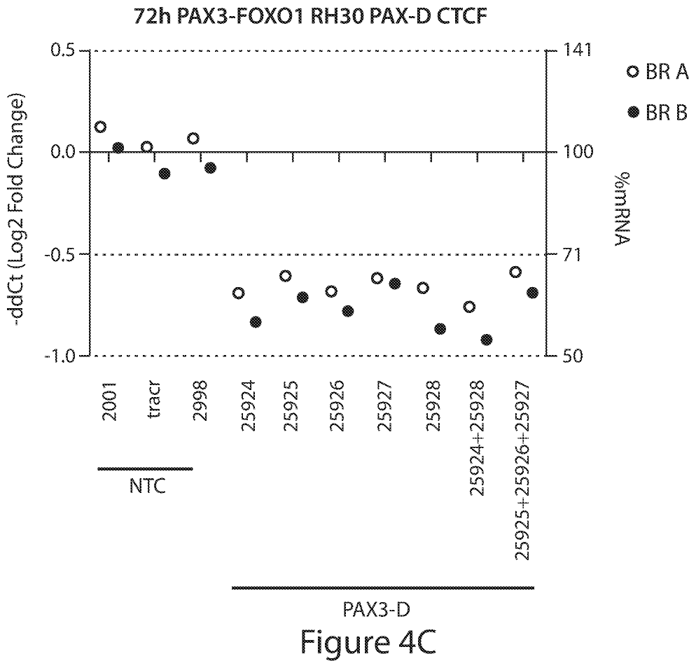

This analysis indicates that CTCF occupies an anchor sequence located intronically within the PAX3 gene (FIG. 4A, PAX3-D). CTCF occupancy at this site is specific to rhabdomyosarcoma cells and is not highly conserved across other cell types (FIG. 4A). CTCF occupancy specific to this site as observed in rhabdomyosaroma may act as an anchor for novel CFLs containing the PAX3-FOXO1 fusion oncogene, ensuring the high expression of PAX3-FOXO1 in cancer cells.

Next, gRNA constructs were designed to target this anchor sequence (Table 7).

TABLE 7
Sequences of guide RNAs (gRNAs) targeting
CTCF anchor sequences associated with the
CFL containing PAX3-FOX01.

| Name | gRNA sequence (5′-3′) |
|---|---|
| 2001 | ATGATCTCTGCTGCCAGTAG |
| 2998 | GTATTACTGATATTGGTGGG |
| GD-25924 | ACAACCTTCCTTGCAGCCAG |
| GD-25925 | TTTCTCCCTCTGGCGCAGCT |
| GD-25926 | CACTGCCAAGCTGCGCCAGA |
| GD-25927 | TGCCCCCATGTTTCTCCCTC |
| GD-25928 | CGCCAGAGGGAGAAACATGG |

Cas9 and gRNAs were then introduced into RH30 cells, which contain the PAX3-FOXO1 fusion oncogene in a novel CFL. Specifically, RH30 cells were transduced overnight with lentivirus encoding Cas9 and a puromycin resistance gene cassette. The following day, the transduced cells were passaged into puromycin-containing culture medium (DMEM supplemented with 10% fetal bovine serum, 1% penicillin/streptomycin and 2 μg/ml puromycin). Puromycin-resistant RH30 cells were maintained under selection for 3 days to establish a population of cells stably expressing Cas9.

RH30-Cas9 cells were transfected with a gRNA or combination of chemically synthesized gRNAs (Table 7) targeted to the CTCF anchor sequence using commercially available transfection reagents (Thermo Fisher Scientific). At 72-hr post-transfection, cells were harvested for genomic DNA and RNA extraction using commercially available reagents and protocols (Lucigen; Thermo Fisher Scientific).

The cells were then assayed to determine whether Cas9-mediated editing had been successful. Targeted genomic regions were PCR-amplified using specific primers (Table 8) and commercially available polymerase mixes (Takara Bio), heteroduplexed and subsequently analyzed by T7E1 endonuclease assay (Integrated DNA Technologies). T7E1 products were separated by agarose gel electrophoresis, and DNA bands were visualized by ethidium bromide staining. gRNAs targeting the CTCF anchor sequences showed Cas9-mediated editing as shown by the presence of high-mobility T7E1 cleavage products, whereas non-targeting control (NTC) gRNAs showed only the lower-mobility, unedited product (FIG. 4B). The data indicate that all target specific gRNAs tested were sufficient to direct some level of Cas9-mediated cleavage of the target sequences.

TABLE 8
Sequences of primers used to amplify
targeted genomic regions corresponding
to the CTCF anchor sequences associated
with the CFL containing PAX3-FOX01.

| Name | gRNA sequence (5′-3′) |
|---|---|
| PAX3-D-F | GCTCACCAGCGAATTTTTATCA |
| PAX3-D-R | ACCGTTCTGTTCCATTTGCC |

Finally, expression of PAX3-FOXO1 was quantified. cDNA synthesis was performed on total RNA extracted from the edited cells and control cells, and subsequently used for quantitative real-time PCR (Thermo Fisher Scientific). Taqman probes/primers specific for PAX3-FOXO1 (Assay ID Hs03024825_ft, Thermo Fisher Scientific) were multiplexed with internal control probes/primers for PPIB (Assay ID Hs00168719_m1, Thermo Fisher Scientific) using the FAM-MGB and VIC-MGB dyes, respectively, and gene expression was analyzed by a real-time Taqman PCR kit (Thermo Fisher Scientific). gRNAs targeting the CTCF anchor sequence showed reduction in PAX3-FOXO1 expression at 72 hr compared to NTC gRNAs (FIG. 4C). Each biological replicate is shown as a gray or black data point. This result indicates that modifying CTCF anchor sequences unique to a cancer associated fusion gene can lower expression of the cancer associated fusion gene and may be useful in treating the associated cancer in patients. Without wishing to be bound by theory, the reduction in expression may be due to disruption of the CFL.

To evaluate the effect of targeting the PAX3-FOXO1 associated CTCF anchor sequence on rhabdomyosarcoma cell viability and proliferation, RH30-Cas9 cells were transfected with a gRNA or combination of chemically synthesized gRNAs (Table 7) targeted to the CTCF anchor sequence using commercially available transfection reagents (Thermo Fisher Scientific). At 96-hr post-transfection, the cells were trypsinized and split into two fractions. One fraction of cells was processed for RNA extraction using commercially available reagents and protocols (Qiagen) to evaluate PAX3-FOXO1 expression at 96-hr post-transfection. The other fraction of cells was plated, incubated, and evaluated for viability and proliferation (Promega).

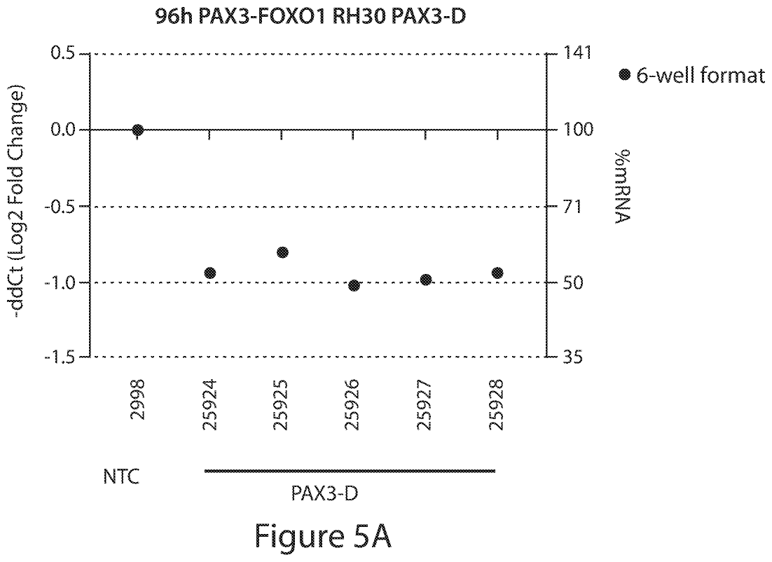

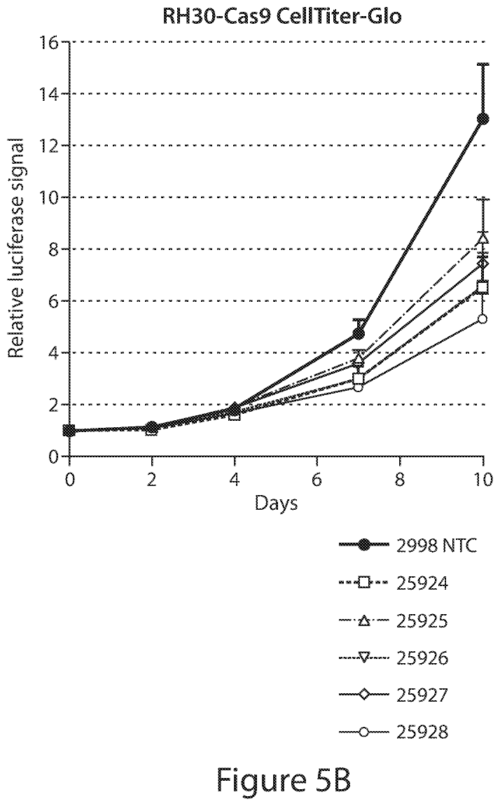

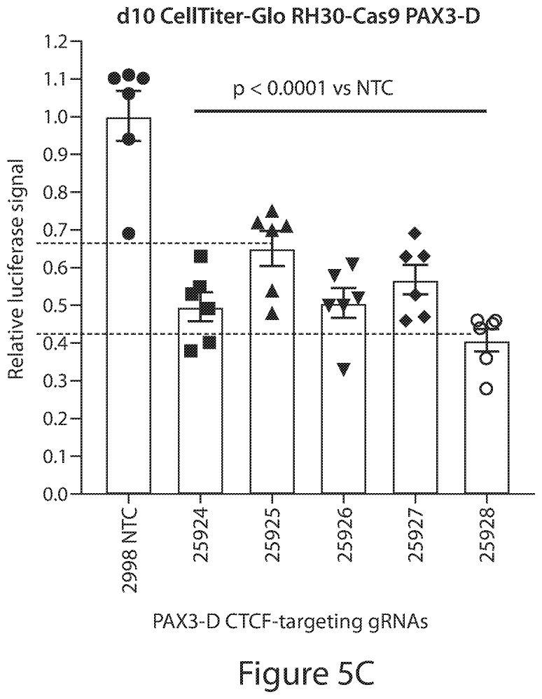

Using the extracted total RNA from the first fraction of cells, cDNA synthesis was performed and the cDNA subsequently used for quantitative real-time PCR (Thermo Fisher Scientific). Taqman probes/primers specific for PAX3-FOXO1 (Assay ID Hs03024825_ft, Thermo Fisher Scientific) were multiplexed with internal control probes/primers for PPIB (Assay ID Hs00168719_m1, Thermo Fisher Scientific) using the FAM-MGB and VIC-MGB dyes, respectively, and gene expression was analyzed by a real-time Taqman PCR kit (Thermo Fisher Scientific). gRNAs targeting the CTCF anchor sequence showed reduction in PAX3-FOXO1 expression at 96 hr compared to NTC gRNA, 2998 (FIG. 5A). This shows that at 96-hr post-transfection, targeting the PAX3-FOXO1 associated CTCF anchor sequence decreases expression of PAX3-FOXO1.

The other fraction of cells was subsequently plated evenly into 96-well, white-walled, clear-bottom plates at a concentration of 1.0×10⁴ cells per well. These cells were allowed to seed and grow in low serum media (DMEM+0.1% FBS). Without wishing to be bound by theory, it is thought that low serum media mimics growth factor-independent conditions and thus increases cellular dependency on the expression level of the PAX3-FOXO1 oncogene for proliferation. At various time points (FIG. 5B), plates were processed to examine cell viability using the commercially available CellTiter-Glo assay (Promega) and a GloMax luminescence plate reader (Promega). An impairment of cell proliferation over time was observed for all gRNAs targeting the PAX3-FOXO1 associated CTCF anchor sequence relative to the non-targeting gRNA, 2998 (FIG. 5B). By ten days, cell numbers were 35-60% reduced in CTCF-targeted cells compared to the control cells (FIG. 5C). Statistical significance was determined by one-way ANOVA and was corrected for multiple comparisons. This shows that targeting the PAX3-FOXO1 associated CTCF anchor sequence impairs rhabdomyosarcoma cell proliferation and viability.

### Example 3

This example describes experiments to demonstrate the downregulation of fusion oncogenes such as IGH-CCND1, IGH-MYC or IGH-BCL2 by genetically modifying the CTCF anchor sequence-mediated conjunctions involved in the formation of CFLs. The example further describes a protocol to demonstrate the downregulation of fusion oncogenes such as IGH-CCND1, IGH-MYC or IGH-BCL2 by epigenetic effectors targeting IGH regulatory sites.

An IGH fusion oncogene can be caused by a translocation of the IGH locus with one of several oncogenes (e.g., CCND1, MYC, or BCL2). The 5′ end of the translocation may comprise a region of chromosome 14 that codes for one or more of the heavy chains of human antibodies and also contains one or more super enhancers. The 3′ partner of an IGH fusion oncogene contains one of many different oncogenes that, when partnered with the IGH locus via translocation, become constitutively and/or highly overexpressed, leading to a leukemic phenotype. It is thought that this newly created insulated genomic domain (IGD) containing an active super enhancer element and the IGH fusion oncogene could be manipulated via perturbation of CTCF binding at the anchor sites surrounding the translocation.

Utilizing CTCF binding data from two IGH fusion cancer cell lines (Granta-519, an IGH-CCND1 fusion and U2646, an IGH-MYC fusion) regions likely to influence the oncogenic fusion were identified (Table 9).

TABLE 9
Exemplary IGH Fusion Oncogene Target Sites

| Description | Peaks | Coordinates |
|---|---|---|
| Upstream Boundary 1 | Multiple | chr14:106002300- |
| | Peaks | 106028000 |
| Upstream Boundary 2 | Multiple | chr14:106143500- |
| | Peaks | 106148500 |
| IGHJ Loop upstream | Single Peak | chr14:106296400- |
| | | 106297200 |
| IGHJ Loop Downstream | Two Peaks | chr14:106410700- |
| | | 106412500 |
| Upstream SE1 | Multiple | chr14:106465000- |
| | Peaks | 106468000 |
| Upstream SE1 | Single Peak | chr14:106501000- |
| | | 106502000 |
| Upstream SE1 | Single Peak | chr14:106517500- |
| | | 106519000 |
| Upstream SE2 | Single Peak | chr14:106723500- |
| | | 106726000 |
| Upstream SE2 | Single Peak | chr14:106733000- |
| | | 106735000 |
| Upstream SE2 | Single Peak | chr14:106764000- |
| | | 106766000 |
| Upstream SE3 | Single Peak | chr14:106933000- |
| | | 106935000 |
| Upstream SE3 | Single Peak | chr14:106985000- |
| | | 106988000 |
| CCND1 site 1 | Two Peaks | chr11:69457500-69460800 |
| CCND1 site 2 | Single Peak | chr11:69484500-69485500 |
| CCND1 site 3 | Two Peaks | chr11:69498000-69501000 |
| CCND1 site 4 | Two Peaks | chr11:69532000-69537000 |
| MYC site 1 | Single Peak | chr8:128902000-128903000 |
| MYC Site 2 | Single Peak | chr8:128906500-128907500 |

Three different types of regions were identified. (1) First, several CTCF binding sites on the 5′ side of the translocation were identified as potential target sites. Loss of CTCF binding at these anchor sites would disrupt the IGD upstream of the super enhancer influencing transcriptional activity of the oncogene at the opposite side of the translocation. (2) Possible anchor sites on the 3′ side of the translocation downstream from two oncogenes known to be fusion partners with the IGH locus were also identified. CCND1 and MYC were noted here as they were the fusion oncogenes contained in the cell lines on which genomic data were collected. These sites potentially represent the 3′ side of the IGD causing the increased transcriptional activity of these oncogenes. (3) Disruption of the super enhancer element may also be a method of down-regulating oncogene overexpression.

Experiments will utilize site-specific disrupting agents comprising, e.g., a genetic modifying moiety, to individually disrupt these sites and/or disrupt combinations of sites to direct precise excision of sequence(s) relevant to disease-associated dysregulation. Site-specific disrupting agents comprising, e.g., an epigenetic modifying moiety, may also be targeted to the one or more sites in order to, e.g., methylate and/or silence regulatory regions. Techniques such as HiC, 4C, CTCF ChIP-Seq, and RNA-Seq may be used to determine the effect the site-specific disrupting agent(s) have on DNA topology (e.g., CFL disruption), sequence/presence of an anchor sequence, CTCF binding, and/or fusion oncogene expression. In some embodiments, a site-specific disrupting agent will decrease expression of the fusion oncogene, disrupts (e.g., mutates) an anchor sequence, decreases CTCF binding, and/or disrupts CFL formation or maintenance. In some embodiments, methylation of upstream CpG residues of IGH will decrease expression of IGH fusion oncogenes. In some embodiments, chromatin compaction, e.g., by a site-specific disrupting agent comprising KRAB or a functional fragment or variant thereof, of one or more enhancers operably linked to the IGH fusion oncogene, will decrease expression of the IGH fusion oncogene.

An exemplary experiment establishes methods, e.g., 4C or HiC, to evaluate anchor site CTCF interactions to determine looping patterns for various IGH fusion oncogenes.

An exemplary experiment examines the effects of a site-specific disrupting agent comprising a genetic modifying moiety that mediates disruption/excision of an IGH proximal anchor sequence, e.g., CTCF binding site, on the down-regulation of expression of IGH fusion oncogenes. Disruption/excision of an IGH proximal anchor sequence, e.g., CTCF binding site, may decrease IGH fusion oncogene expression. Disruption/excision may be implemented using a site-specific disrupting agent comprising a CRISPR/Cas9 molecule (e.g., a genetic modifying moiety).

An exemplary experiment examines the effects of a site-specific disrupting agent comprising an epigenetic modifying moiety targeted to specific IGH proximal regulatory elements on down-regulation of IGH fusion oncogenes. The effects of methylation of two CpG residues upstream of IGH are evaluated; in some embodiments, methylation decreases IGH fusion oncogene expression. In some embodiments, methylation is implemented using a site-specific disrupting agent comprising an epigenetic modifying moiety (e.g., MQ1 or a functional variant or fragment thereof) and optionally a targeting moiety comprising a CRISPR/Cas9 molecule (e.g., a dCas9). The effects of chromatin compaction of the region containing duplicated enhancers-3′C a of IGH are evaluated; in some embodiments, compaction decreases IGH fusion oncogene expression. In some embodiments, chromatin compaction is implemented using a site-specific disrupting agent comprising an epigenetic modifying moiety (e.g., KRAB or a functional variant or fragment thereof) and optionally a targeting moiety comprising a CRISPR/Cas9 molecule (e.g., a dCas9).

An exemplary experiment evaluates the effects of excising the IGH fusion oncogene using two guides targeted to flanking loop anchor regions (e.g., an IGH proximal anchor sequence (e.g., CTCF site) and a downstream oncogene (e.g., MYC, CCND1, or BCL2) proximal anchor sequence (e.g., CTCF site)). In some embodiments, excision of the IGH fusion oncogene decreases IGH fusion oncogene expression.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

1. A method of decreasing expression, (e.g., transcription) of a gene (e.g., an oncogene, e.g., a fusion oncogene) in a cell (e.g., a cancer cell), comprising:
contacting the cell with a site-specific disrupting agent that binds to a first and/or second anchor sequence, or a component of a genomic complex associated with the first and/or second anchor sequence, in the cell, in an amount sufficient to decrease expression of the gene,
wherein the cell comprises a nucleic acid, said nucleic acid comprising:
i) the gene;
ii) a breakpoint (e.g., a breakpoint resulting from a gross chromosomal rearrangement), located proximal to the gene;
iii) the first anchor sequence, which is located proximal to the breakpoint and/or the gene, and
iv) the second anchor sequence, which is located proximal to the breakpoint and/or the gene,
thereby decreasing expression of the gene.

2. A method of decreasing expression (e.g., transcription) of a fusion oncogene in a cell (e.g., a cancer cell), comprising:
contacting the cell with a site-specific disrupting agent comprising a targeting moiety that binds, e.g., binds specifically, to a genomic sequence element (e.g., anchor sequence, enhancer, or promoter) proximal to the fusion oncogene,
wherein the fusion oncogene is an IGH fusion oncogene (e.g., formed by a gross chromosomal rearrangement and/or proximal or comprising a breakpoint),
thereby decreasing expression of the fusion oncogene in the cell.

3. A cell made or modified by the method of either of claim 1 or 2.

4. A cell comprising a nucleic acid, said nucleic acid comprising:
i) a gene;
ii) a breakpoint (e.g., a breakpoint resulting from a gross chromosomal rearrangement), located proximal to the gene;
iii) a first anchor sequence, which is located proximal to the breakpoint and/or the gene; and
iv) a second anchor sequence, which is located proximal to the breakpoint and/or the gene;
wherein the cell comprises a non-naturally occurring, site-specific modification to the first and/or second anchor sequence, or to a component of a genomic complex associated with the first and/or second anchor sequence (e.g., compared to the cell prior to the modification), wherein the site-specific modification occurs preferentially at the first and/or second anchor sequence or the component of the genomic complex,
wherein the site-specific modification leads to downregulation of the gene.

5. A method of treating a cancer in a subject, comprising:
administering to the subject a site-specific disrupting agent that binds to a first anchor sequence, or a component of a genomic complex associated with the first anchor sequence, in a cell, in an amount sufficient to treat the cancer,
wherein the cell comprises a nucleic acid, said nucleic acid comprising:
i) an oncogene (e.g., a fusion oncogene);
ii) a breakpoint (e.g., a breakpoint resulting from a gross chromosomal rearrangement), located proximal to the oncogene;
v) a first anchor sequence, which is located proximal to the breakpoint and/or the oncogene; and
vi) a second anchor sequence, which is located proximal to the breakpoint and/or the oncogene;
wherein the site-specific disrupting agent is administered in an amount sufficient to decrease expression of the oncogene,
thereby treating the cancer.

6. A site-specific disrupting agent, comprising:
a DNA- or RNA-binding moiety that binds to a target anchor sequence or to a component of a genomic complex associated with the target anchor sequence, wherein the target anchor sequence is proximal to a breakpoint (e.g., a breakpoint resulting from a gross chromosomal rearrangement), e.g., with sufficient affinity that it competes with binding of an endogenous nucleating polypeptide to the target anchor sequence.

7. A site-specific disrupting agent, comprising:
a targeting moiety that binds, e.g., binds specifically, to a genomic sequence element (e.g., an anchor sequence, enhancer, or promoter) proximal to an IGH fusion oncogene (e.g., formed by a gross chromosomal rearrangement and/or proximal to or comprising a breakpoint),
wherein binding of the site-specific disrupting agent decreases expression of the IGH fusion oncogene.

8. The site-specific disrupting agent or method of either of claim 2, 3, or 6, wherein the genomic sequence element is upstream from the IGH fusion oncogene.

9. The site-specific disrupting agent or method of any of claim 2, 3, 6, or 8, wherein the genomic sequence element is an enhancer, e.g., that is or is part of a super enhancer.

10. The site-specific disrupting agent or method of any of claim 2, 3, 6, 8, or 9, wherein the targeting moiety is or comprises a CRISPR/Cas molecule, a TAL effector molecule, or a Zn finger molecule.

11. The site-specific disrupting agent or method of any of claim 2, 3, 8, or 10, wherein the genomic sequence element is an anchor sequence.

12. A reaction mixture comprising:
a) a nucleic acid comprising:
i) a gene (e.g., an oncogene, e.g., a fusion oncogene);
ii) a breakpoint (e.g., a breakpoint resulting from a gross chromosomal rearrangement), located proximal to the gene; and
iii) a target anchor sequence (e.g., target cancer-specific anchor sequence), which is located proximal to the breakpoint and/or the gene, and
b) a first agent (e.g., a probe or a site-specific disrupting agent) that binds to the target anchor sequence or to a component of a genomic complex associated with the anchor sequence.

13. A method of decreasing expression (e.g., transcription) of a gene (e.g., an oncogene, e.g., a fusion oncogene) in a cell (e.g., a cancer cell), comprising:
contacting the cell with a site-specific disrupting agent that binds to a cancer-specific anchor sequence or a component of a genomic complex associated with the cancer-specific anchor sequence, in the cell, in an amount sufficient to decrease expression of the gene,
wherein the cell comprises a nucleic acid, said nucleic acid comprising:
i) the gene;
ii) the cancer-specific anchor sequence, which is located proximal to the gene; and
iii) a second anchor sequence, which is located proximal to the gene;
thereby decreasing expression of the gene.

14. A cell made or modified by the method of claim 13.

15. The method, cell, site-specific disrupting agent, reaction mixture, or composition of any of the preceding claims, wherein the anchor sequence (e.g., the first and/or second anchor sequence) is a cancer-specific anchor sequence.

16. The method, cell, site-specific disrupting agent, reaction mixture, or composition of any of claims 1-15, wherein the gross chromosomal rearrangement comprises a translocation, deletion (e.g., interstitial deletion or terminal deletion), inversion, insertion, amplification (e.g., duplication), e.g., a tandem amplification or tandem duplication, chromosome end-to-end fusion, chromothripsis, or any combination thereof.

17. The method, cell, site-specific disrupting agent, reaction mixture, or composition of any of claims 1-16, wherein the breakpoint is located in a transcribed region (e.g., in an intron, an exon, a 5′ UTR, or a 3′ UTR) or in a non-transcribed region.

18. The method, cell, site-specific disrupting agent, reaction mixture, or composition of any of claims 1-17 wherein the gross chromosomal rearrangement results in formation of a fusion oncogene.

19. The method or cell of any of claims 1-18 wherein the nucleic acid further comprises an internal enhancing sequence which is located at least partially between the first anchor sequence (e.g., the cancer-specific anchor sequence) and the second anchor sequence.

20. The method or cell of any of claims 1-19, wherein the nucleic acid further comprises one or more repressor signals, e.g., one or more silencing sequences, wherein the one or more repressor signals are located outside an anchor-sequence mediated conjunction formed by the first anchor sequence (e.g., the cancer-specific anchor sequence) and the second anchor sequence.

21. The method, cell, or reaction mixture, of any of claims 1-20, wherein the nucleic acid comprises an anchor sequence mediated conjunction, e.g., a loop.

22. The method, cell, site-specific disrupting agent, reaction mixture, or composition of any of the preceding claims, wherein the anchor sequence (e.g., first and/or second anchor sequence, e.g., cancer-specific anchor sequence) is at least 3, 4, 5, 6, 7, 8, 9, or 10 kb away from a transcriptional start site.

23. The method, cell, site-specific disrupting agent, reaction mixture, or composition of any of the preceding claims, wherein the anchor sequence (e.g., the first anchor sequence and/or cancer-specific anchor sequence) comprises a CTCF binding motif, BORIS binding motif, cohesin binding motif, USF 1 binding motif, YY1 binding motif, TATA-box, or ZNF143 binding motif.

24. The method, cell, or reaction mixture of any of claims 1-23, wherein the second anchor sequence comprises a CTCF binding motif, BORIS binding motif, cohesin binding motif, USF 1 binding motif, YY1 binding motif, TATA-box, or ZNF143 binding motif.

25. The method, cell, site-specific disrupting agent, reaction mixture, or composition of any of the preceding claims, wherein the anchor sequence (e.g., the first anchor sequence and/or cancer-specific anchor sequence) is adjacent to a CTCF binding motif, BORIS binding motif, cohesin binding motif, USF 1 binding motif, YY1 binding motif, TATA-box, or ZNF143 binding motif.

26. The method, cell, or reaction mixture of any of claims 1-25, wherein the second anchor sequence is adjacent to a CTCF binding motif, BORIS binding motif, cohesin binding motif, USF 1 binding motif, YY1 binding motif, TATA-box, or ZNF143 binding motif.

27. The method, cell, or reaction mixture of any of claims 1-26, wherein the gene comprises a transcription factor, e.g., a full length transcription factor or a transcriptionally active fragment thereof.

28. The method, cell, or reaction mixture of any of claims 1-27, wherein the gene comprises a kinase, e.g., a full length kinase or a fragment thereof having kinase activity.

29. The method, cell, or reaction mixture of any of claims 1-28, wherein expression of the gene in the cell, e.g., cancer cell, is reduced to less than 80%, 70%, 60%, 50%, 40%, 30%, or 20% of a reference level, e.g., wherein the reference is expression level of the same gene in an otherwise similar, untreated cell (e.g., untreated cancer cell).

30. The method, cell, or reaction mixture of any of claims 1-29, wherein expression of the gene in a non-cancer cell contacted with the site-specific binding agent changes (e.g., increases or decreases) less than 10%, 20%, or 30% relative to a reference level, e.g., wherein the reference is expression level of the same gene in an otherwise similar, untreated non-cancer cell.

31. The method, cell, or reaction mixture of any of claim 30, wherein the gene is a fusion oncogene, and wherein the non-cancer cell comprises first and second endogenous genes corresponding to the fusion oncogene, and wherein expression of the first and/or second endogenous genes in the non-cancer cell changes (e.g., increases or decreases) less than 10%, 20%, or 30% relative to a reference level, e.g., wherein the reference is expression level of the endogenous gene an otherwise similar, untreated non-cancer cell.

32. The method, site specific disrupting agent, or cell of any of claim 1-11 or 13-31, wherein the site-specific disrupting agent binds specifically to a first anchor sequence, e.g., a target cancer-specific anchor sequence, or a component of a genomic complex associated with the first anchor sequence, e.g., target cancer-specific anchor sequence, and wherein the site-specific disrupting agent alters (e.g., decreases) expression of the gene in a cancer cell more than the site-specific disrupting agent alters (e.g., decreases) expression of the gene (or one or two endogenous genes corresponding to the gene, e.g., fusion oncogene) in a non-cancer cell.

33. The method, site specific disrupting agent, or cell of claim 32, wherein the percentage decrease in the cancer cell is at least 10%, 20%, 30%, 40%, 50%, 60%, 70%, 80%, 90%, 2-fold, 3-fold, 4-fold, 5-fold, or 10-fold larger than the percentage decrease in the non-cancer cell.

34. The method, site specific disrupting agent, or cell of either of claim 32 or 33, wherein the site-specific disrupting agent does not alter (e.g., does not decrease) the expression of a gene (e.g., proto-oncogene and/or an endogenous gene corresponding to the fusion oncogene) in a non-cancerous cell.

35. The method, cell, site-specific disrupting agent, reaction mixture, or composition of any of the preceding claims, wherein the DNA sequence of the first and/or second anchor sequence, e.g., target anchor sequence, is altered.

36. The method, cell, site-specific disrupting agent, reaction mixture, or composition of any of the preceding claims, wherein the chromatin structure of the first and/or second anchor sequence, e.g., target anchor sequence, is altered.

37. The method, cell, site-specific disrupting agent, reaction mixture, or composition of any of the preceding claims, wherein DNA methylation of the first and/or second anchor sequence (e.g., target anchor sequence) is altered (e.g., increased or decreased).

38. The method, cell, reaction mixture, or site-specific disrupting agent of any of claim 1-3, or 5-37 wherein the site-specific disrupting agent comprises a DNA-binding moiety that binds the anchor sequence.

39. The method, cell, reaction mixture, or site-specific disrupting agent of any of claim 1-3, or 5-38 wherein the site-specific disrupting agent comprises an RNA-binding moiety that binds a non-coding RNA comprised by the genomic complex.

40. The method, cell, reaction mixture, or site-specific disrupting agent of any of claim 1-3 or 5-39, wherein the site-specific disrupting agent comprises a protein-binding moiety that binds a nucleating protein comprised by the genomic complex, wherein optionally the site specific disrupting agent also binds DNA of the genomic complex.

41. The method of any of claim 1, 2, 5, 13, or 15-40, which further comprises contacting the cell or nucleic acid with a second site-specific disrupting agent.

42. The method, cell, site-specific disrupting agent, reaction mixture, or composition of any of claims 1-41, wherein the gene or oncogene is a fusion gene or fusion oncogene and comprises a fusion between a first fusion partner gene and a second fusion partner gene, e.g., wherein the fusion gene or fusion oncogene comprises one or more exons from the first fusion partner gene and one or more exons from the second fusion partner gene.

43. The method, cell, site-specific disrupting agent, reaction mixture, or composition of claim 42, wherein the first or second fusion partner gene comprises IGH or a functional fragment or variant thereof.

44. The method, cell, site-specific disrupting agent, reaction mixture, or composition of either of claim 42 or 43, wherein the first or second fusion partner gene comprises MYC, BCL2, CCND1, or BCL6, or a functional fragment or variant of any thereof.


SEQUENCE LISTING (30 sequences)

SEQ ID NO: 1 - DNA, 20 bp, Artificial Sequence
  Description of Artificial Sequence Synthetic oligonucleotide
  modified_base (1)..(1): a, c, t, g, unknown or other
  modified_base (3)..(3): a, c, t, g, unknown or other
nbndccdshagrkgghrshv

SEQ ID NO: 2 - DNA, 20 bp, Artificial Sequence
  Description of Artificial Sequence Synthetic oligonucleotide
  modified_base (1)..(1): a, c, t, g, unknown or other
  modified_base (3)..(3): a, c, t, g, unknown or other
nbndccdshagrkgghrshv

SEQ ID NO: 3 - DNA, 7 bp, Unknown
  Description of Unknown TATA consensus sequence
tatawaw

SEQ ID NO: 4 - DNA, 20 bp, Artificial Sequence
  Description of Artificial Sequence Synthetic oligonucleotide
atgatctctgctgccagtag

SEQ ID NO: 5 - DNA, 20 bp, Artificial Sequence
  Description of Artificial Sequence Synthetic oligonucleotide
gtattactgatattggtggg

SEQ ID NO: 6 - DNA, 20 bp, Artificial Sequence
  Description of Artificial Sequence Synthetic oligonucleotide
gtgatgacagcgccatctga

SEQ ID NO: 7 - DNA, 20 bp, Artificial Sequence
  Description of Artificial Sequence Synthetic oligonucleotide
tgatgacagcgccatctgat

SEQ ID NO: 8 - DNA, 20 bp, Artificial Sequence
  Description of Artificial Sequence Synthetic oligonucleotide
cctcacaccttcccatcaga

SEQ ID NO: 9 - DNA, 20 bp, Artificial Sequence
  Description of Artificial Sequence Synthetic oligonucleotide
gacagcgccatctgatggga

SEQ ID NO: 10 - DNA, 20 bp, Artificial Sequence
  Description of Artificial Sequence Synthetic oligonucleotide
tttcagccagctttgctggg

SEQ ID NO: 11 - DNA, 20 bp, Artificial Sequence
  Description of Artificial Sequence Synthetic oligonucleotide
cgtggtcaccagacggcggc

SEQ ID NO: 12 - DNA, 20 bp, Artificial Sequence
  Description of Artificial Sequence Synthetic oligonucleotide
gggacccgcccgccgccgtc

SEQ ID NO: 13 - DNA, 20 bp, Artificial Sequence
  Description of Artificial Sequence Synthetic oligonucleotide
cgcccgtggtcaccagacgg

SEQ ID NO: 14 - DNA, 20 bp, Artificial Sequence
  Description of Artificial Sequence Synthetic oligonucleotide
gcccgcccgtggtcaccaga

SEQ ID NO: 15 - DNA, 20 bp, Artificial Sequence
  Description of Artificial Sequence Synthetic oligonucleotide
cgccgccgtctggtgaccac

SEQ ID NO: 16 - DNA, 20 bp, Artificial Sequence
  Description of Artificial Sequence Synthetic primer
ccacactgggtacaggaagg

SEQ ID NO: 17 - DNA, 20 bp, Artificial Sequence
  Description of Artificial Sequence Synthetic primer
cccaaagcaagacagattcc

SEQ ID NO: 18 - DNA, 19 bp, Artificial Sequence
  Description of Artificial Sequence Synthetic primer
ttgggcagtattgcactgg

SEQ ID NO: 19 - DNA, 20 bp, Artificial Sequence
  Description of Artificial Sequence Synthetic primer
gccacaacacggtagaggat

SEQ ID NO: 20 - DNA, 20 bp, Artificial Sequence
  Description of Artificial Sequence Synthetic oligonucleotide
atgatctctgctgccagtag

SEQ ID NO: 21 - DNA, 20 bp, Artificial Sequence
  Description of Artificial Sequence Synthetic oligonucleotide
gtattactgatattggtggg

SEQ ID NO: 22 - DNA, 20 bp, Artificial Sequence
  Description of Artificial Sequence Synthetic oligonucleotide
acaaccttccttgcagccag

SEQ ID NO: 23 - DNA, 20 bp, Artificial Sequence
  Description of Artificial Sequence Synthetic oligonucleotide
tttctccctctggcgcagct

SEQ ID NO: 24 - DNA, 20 bp, Artificial Sequence
  Description of Artificial Sequence Synthetic oligonucleotide
cactgccaagctgcgccaga

SEQ ID NO: 25 - DNA, 20 bp, Artificial Sequence
  Description of Artificial Sequence Synthetic oligonucleotide
tgcccccatgtttctccctc

SEQ ID NO: 26 - DNA, 20 bp, Artificial Sequence
  Description of Artificial Sequence Synthetic oligonucleotide
cgccagagggagaaacatgg

SEQ ID NO: 27 - DNA, 22 bp, Artificial Sequence
  Description of Artificial Sequence Synthetic primer
gctcaccagcgaatttttatca

SEQ ID NO: 28 - DNA, 20 bp, Artificial Sequence
  Description of Artificial Sequence Synthetic primer
accgttctgttccatttgcc

SEQ ID NO: 29 - protein, 4 aa, Homo sapiens
DEAD

SEQ ID NO: 30 - protein, 4 aa, Homo sapiens
DEAH